(19) United States
(12) Patent Application Publication
(10) Pub. No.: US 2025/0249056 A1
(43) Pub. Date: Aug. 7, 2025

(54) CANCER THERAPIES COMPRISING A NUCLEAR EXPORT INHIBITOR AND AN ONCOLYTIC VIRUS

(71) Applicant: Arizona Board of Regents on behalf of Arizona State University, Scottsdale, AZ (US)

(72) Inventors: Douglas McFadden, Tempe, AZ (US); Mohammed Masmudur Rahman, Chandler, AZ (US)

(21) Appl. No.: 18/852,540

(22) PCT Filed: Mar. 30, 2023
(86) PCT No.: PCT/US2023/065165

§ 371 Date: Sep. 30, 2024

Related U.S. Application Data
(60) Provisional application No. 63/325,309, filed on Mar. 30, 2022.

(51) Int. Cl. A61K 35/768; A61K 9/00; A61K 31/497; A61P 35/00; C12N 7/00

(52) U.S. Cl. CPC A61K 35/768; A61K 9/0019; A61K 9/0053; A61K 31/497; A61P 35/00; C12N 7/00; C12N 2710/24022; C12N 2710/24032

(57) ABSTRACT

Disclosed herein are compositions and methods for treating cancer. The methods can comprise administrating to a subject with cancer a therapeutically effective amount of an oncolytic virus and a therapeutically-effective amount of a nuclear export inhibitor. Methods disclosed herein can convert nonpermissive or semi-permissive cancers to permissive cancers that are susceptible to infection and killing by oncolytic viruses.

### CROSS-REFERENCE TO RELATED APPLICATIONS

This application claims to and the benefit of the U.S. Provisional Application No. 63/325,309, filed on Mar. 30, 2022, the disclosure of which is incorporated herein by reference in its entirety.

### STATEMENT AS TO FEDERALLY SPONSORED RESEARCH

This invention was made with government support under R01 AI080607 awarded by the National Institutes of Health. The government has certain rights in the invention.

### BACKGROUND

Oncolytic viruses, such as from the Poxviridae family of viruses, can be mammalian viruses that are designed and/or selected for their ability to selectively infect and kill transformed cancer cells, and by their ability to activate host's immune system against the virus and also tumor antigens. However, the application of oncolytic viruses can be limited in certain tumor cells, for example nonpermissive tumor cells. Therefore, there remains a need to improve therapies based on oncolytic viruses.

### SUMMARY

Disclosed herein, in some aspects, is a method of treating cancer in a subject in need thereof, the method comprising administering to the subject an effective amount of a myxoma virus (MYXV) and an effective amount of a nuclear export inhibitor, wherein the nuclear export inhibitor is administered orally.

Disclosed herein, in some aspects, is a method of treating cancer in a subject in need thereof, the method comprising administering to the subject an effective amount of a myxoma virus (MYXV) and an effective amount of a nuclear export inhibitor, wherein the MYXV is genetically modified to express a heterologous transgene.

Disclosed herein, in some aspects, is a method of treating cancer in a subject in need thereof, the method comprising administering to the subject an effective amount of a myxoma virus (MYXV) and an effective amount of a nuclear export inhibitor, wherein the nuclear export inhibitor is selinexor and is administered at a dose per kilogram of subject body weight of between about 0.001 mg/kg and about 1000 mg/kg.

In some embodiments, the nuclear export inhibitor: is a selective inhibitor of nuclear export (SINE), binds to and/or inhibits exportin 1 (XPO1/CRM1), binds to and/or inhibits a factor that binds to a nuclear export signal, binds to and/or inhibits a factor that binds to RAN, RAN-GTP, and/or RAN-GDP, binds to and/or inhibits a factor that docks to the nuclear pore complex, and/or binds to and/or inhibits a factor that mediates leucine-rich nuclear export signal (NES)-dependent protein transport.

In some embodiments, the nuclear export inhibitor is not rapamycin or a structural analog thereof. In some embodiments, the nuclear export inhibitor is one or more selected from the group consisting of selinexor, leptomycin A, leptomycin B, ratjadone A, ratjadone B, ratjadone C, ratjadone D, anguinomycin A, goniothalamin, piperlongumine, plumbagin, curcumin, valtrate, acetoxychavicol acetate, prenylcoumarin osthol, KOS 2464, PKF050-638, or CBS9106. In some embodiments, the nuclear export inhibitor is not rapamycin or a structural analog thereof.

In some embodiments, the nuclear export inhibitor is selinexor and is administered at a dose per kilogram of subject body weight of between about 0.001 mg/kg and about 1000 mg/kg. In some embodiments, the selinexor is administered in a tablet or a capsule. In some embodiments, at least two doses of selinexor are administered.

In some embodiments, the MYXV is administered locally, systemically, intratumorally, intravenously, via injection, or via infusion. In some embodiments, the MYXV is administered at a dose of from about 1×10³ focus-forming units (FFU) to about 1×10¹⁴ FFU. In some embodiments, at least two doses of the MYXV are administered. In some embodiments, the MYXV and the selinexor are administered simultaneously or sequentially.

In some embodiments, the method increases replication of the MYXV in cancer cells of the subject by at least 10%. In some embodiments, the method is effective to reduce average cancer load by at least 10% and/or prolong survival by at least 5% relative to an otherwise comparable treatment regimen that lacks either the MYXV or the selinexor as determined by a cohort study. In some embodiments, the cancer load comprises a tumor volume or circulating hematological cancer cells. In some embodiments, upon local administration of the MYXV, the MYXV reduces incidence of metastasis at a site distal from the site of administration at least 10% more than in a corresponding method that lacks either the MYXV or the selinexor as determined by a cohort study.

In some embodiments, the heterologous transgene encodes a cytokine, interleukin, cell matrix protein, antibody, checkpoint inhibitor, multi-specific immune cell engager, or a functional fragment thereof. In some embodiments, the heterologous transgene encodes an anti-PD-L1 antibody, decorin, IL-12, LIGHT, p14 FAST, TNF-α, a functional fragment thereof, or a combination thereof. In some embodiments, the multi-specific immune cell engager is a bispecific killer cell engager (BiKE) or a bispecific T cell engager (BiTE).

In some embodiments, the cancer is selected from the group consisting of a solid tumor, hematological tumor, sarcoma or a carcinoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, retinoblastoma, adenocarcinoma, pancreatic cancer, and melanoma.

In some embodiments, the subject is immunocompetent, immunocompromised, or immunodeficient.

In some embodiments, the subject is a mammal. In some embodiments, the subject is a human. In some embodiments, the method further comprises adsorbing the MYXV to a leukocyte ex vivo and administering the leukocyte to the subject.

Disclosed herein, in some aspects, is a therapeutic regimen comprising administering a myxoma virus (MYXV) and a nuclear export inhibitor to a subject with cancer, wherein the therapeutic regimen is effective to reduce average cancer load by at least 5% and/or prolong average survival by at least 5% relative to an otherwise comparable treatment regimen that lacks either the MYXV or the nuclear export inhibitor as determined by a cohort study. In some embodiments, the nuclear export inhibitor is administered orally. In some embodiments, the MYXV is genetically modified. In some embodiments, the MYXV is genetically modified to express a heterologous transgene. In some embodiments, the nuclear export inhibitor is a selective inhibitor of nuclear export (SINE), binds to and/or inhibits exportin 1 (XPO1/CRM1), binds to and/or inhibits a factor that binds to a nuclear export signal, binds to and/or inhibits a factor that binds to RAN, RAN-GTP, and/or RAN-GDP, binds to and/or inhibits a factor that docks to the nuclear pore complex, and/or binds to and/or inhibits a factor that mediates leucine-rich nuclear export signal (NES)-dependent protein transport. In some embodiments, the nuclear export inhibitor is not rapamycin or a structural analog thereof. In some embodiments, the nuclear export inhibitor is one or more selected from the group consisting of selinexor, leptomycin A, leptomycin B, ratjadone A, ratjadone B, ratjadone C, ratjadone D, anguinomycin A, goniothalamin, piperlongumine, plumbagin, curcumin, valtrate, acetoxychavicol acetate, prenylcoumarin osthol, KOS 2464, PKF050-638, or CBS9106.

In some embodiments, the nuclear export inhibitor is selinexor and is administered at a dose per kilogram of subject body weight of between about 0.0001 mg/kg and about 1000 mg/kg. In some embodiments, the selinexor is administered at a dose of between about 0.01 mg/kg and about 100 mg/kg.

In some embodiments, the MYXV is administered locally, systemically, intratumorally, intravenously, via injection, or via infusion. In some embodiments, the MYXV is administered at a dose of from about 1×10³ focus-forming units (FFU) to about 1×10¹⁴ FFU. In some embodiments, the MYXV and selinexor are administered simultaneously or sequentially. In some embodiments, the cancer load comprises a tumor volume or concentration of circulating hematological cancer cells. In some embodiments, the therapeutic regimen is effective to reduce the average cancer load by at least about 20% and/or prolong average survival by at least about 20% relative to an otherwise comparable treatment regimen. In some embodiments, the MYXV is administered locally and the therapeutic regimen reduces incidence of metastasis at least 10% more than in a corresponding treatment regimen that lacks selinexor as determined by a cohort study and/or reduces cancer growth at a site distal from the site of administration at least 10% more than in a corresponding treatment regimen that lacks selinexor as determined by a cohort study.

In some embodiments, the cancer is selected from the group consisting of a solid tumor, hematological tumor, sarcoma, carcinoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, retinoblastoma, colorectal adenocarcinoma, pancreatic cancer, and melanoma.

In some embodiments, the subject is immunocompetent, immunocompromised, or immunodeficient. In some embodiments, the subject is a mammal. In some embodiments, the subject is a human. In some embodiments, the therapeutic regimen further comprises adsorbing the MYXV to a leukocyte ex vivo and administering the leukocyte to the subject.

### BRIEF DESCRIPTION OF THE DRAWINGS

The patent application contains at least one drawing executed in color. Copies of this patent or patent application with color drawings will be provided by the Office upon request and payment of the necessary fee.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1, comprising FIG. 1A through FIG. 1F, depicts representative experimental results demonstrating that leptomycin B treatment significantly enhances MYXV replication in human cancer cells by reducing DHX9 antiviral granules. FIG. 1A depicts representative images of PANC-1 cells treated with different concentrations of leptomycin B for 1 hour, infected with vMyx-GFP-TdTomato (MOI=5; scale bar=25 μm) for 1 hour, and replaced with fresh media containing the same doses of leptomycin B for 48 hours prior to imaging. FIG. 1B depicts representative images of HT29 cells treated as the PANC-1 cells of FIG. 1A. FIG. 1C depicts representative quantification of virus production in PANC-1 by titration assays in permissive RK13 cells 48- or 72-hours post-infection. FIG. 1D depicts representative quantification of virus production in HT29 by titration assays in permissive RK13 cells infected with an MOI of 0.05 or 0.5. FIG. 1E depicts representative images of A549 cells seeded on glass bottom 35 mm petri dishes, incubated overnight, treated with leptomycin B for one hour, infected with vMyx-GFP (MOI=1), incubated for 24 hours, fixed, and stained with anti-DHX9 antibodies and DAPI. FIG. 1F depicts representative quantification of inhibition of DHX9 nuclear localization by leptomycin B as imaged in FIG. 1E for at least 100 cells. For FIG. 1C and FIG. 1D, n=3; ****, p<0.0001; ***, p<0.001; **, p<0.01; *, p<0.05.

FIG. 2, comprising FIG. 2A through FIG. 2G, depicts representative experimental results demonstrating that selinexor treatment significantly enhances MYXV gene expression and replication in multiple human cancer cell lines. FIG. 2A depicts representative images of PANC-1 cells that had been treated with different concentrations of selinexor for 1 hour, infected with vMyx-GFP-TdTomato (MOI=5) for one hour, had the media replaced with fresh media with the same doses of selinexor, and incubated for 48 hours. FIG. 2B depicts representative images of MDA-MB435 cells treated as the PANC-1 cells were in FIG. 2A with a viral MOI of 5. FIG. 2C depicts representative images of Colo205 cells treated as the PANC-1 cells were in FIG. 2A with a viral MOI of 1. FIG. 2D depicts representative images of HCT116 cells treated as the PANC-1 cells were in FIG. 2A with a viral MOI of 5. FIG. 2E depicts representative quantification of viral production in PANC-1 cells after 24, 48, or 72 hours of treatment as in FIG. 2A determined by titration assays in permissive RK13 cells. FIG. 2F depicts representative quantification of viral production in Colo205 cells after 24, 48, or 72 hours of treatment as in FIG. 2B. FIG. 2G depicts representative quantification of viral production in MDA-MB435 cells after 24, 48, or 72 hours of treatment as in FIG. 2C.

FIG. 3, comprising FIG. 3A through FIG. 3E, depicts representative results demonstrating that CRM1 knockdown significantly enhances MYXV replication in human cancer cells. FIG. 3A depicts representative imaging of PANC-1 cells mock treated or transfected with non-targeting (NT siRNA) or CRM1 siRNA and incubated for 48 hours, infected with vMyx-GFP at an MOI of 0.5 or 5 for 1 hour, and incubated in fresh media for 48 or 72 hours. FIG. 3B depicts representative quantification of viral production in cells treated as in FIG. 3A with an MOI of 0.5 by virus titration assays in permissive RK13 cells. FIG. 3C depicts representative quantification of viral production in cells treated as in FIG. 3A with an MOI of 5 by virus titration assays in permissive RK13 cells. FIG. 3D depicts representative images of A549 cells seeded on glass bottom 35 mm petri dishes and incubated overnight, transfected with CRM1 siRNA and infected with vMyx-GFP at an MOI of 0.1 or 3, incubated for 24 hours, and stained with anti-DHX9 antibodies and DAPI. FIG. 3E depicts representative imaging of Western blot analysis of CRM1 protein levels in A549 cells after 48 hours of transfection with siRNAs. For FIG. 3A and FIG. 3D, scale bar=25 μm. For FIG. 3B and FIG. 3C, n=3; ****, p<0.0001; ***, p<0.001; **, p<0.01; *, p<0.05.

FIG. 4, comprising FIG. 4A through FIG. 4G, depicts representative results demonstrating that the combination of selinexor and oncolytic MYXV significantly reduces human cancer cell proliferation. FIG. 4A depicts a schematic representation of a cell proliferation assay protocol using a Click-iT™ EdU kit. FIG. 4B depicts representative images of PANC-1 cells that were treated with nothing (mock), selinexor, vMyx-GFP, or selinexor+vMyx-GFP, and subjected to Click-iT™ EdU kit cell proliferation assays. FIG. 4C depicts representative quantification cells labeled with EdU 594 as depicted in FIG. 4B for at least 100 cells. FIG. 4D depicts representative quantification of PANC-1 cell proliferation with 1 μM selinexor as determined by using CyQUANT NF (No Freeze) cell proliferation assay kit. FIG. 4E depicts representative quantification of PANC-1 cell proliferation with 0.1 μM selinexor as determined by using CyQUANT NF (No Freeze) cell proliferation assay kit. FIG. 4F depicts representative quantification of Colo205 cell proliferation with 1 μM selinexor as determined by using CyQUANT NF (No Freeze) cell proliferation assay kit. FIG. 4G depicts representative quantification of Colo205 cell proliferation with 0.1 μM selinexor as determined by using CyQUANT NF (No Freeze) cell proliferation assay kit. For FIG. 4D-FIG. 4G, n=4. For FIG. 4C-FIG. 4G, ***, p<0.001; **, p<0.01; *, p<0.05; ns, not significant.

FIG. 5, comprising FIG. 5A through FIG. 5H, depicts representative experimental results demonstrating that the combination of selinexor and oncolytic MYXV significantly reduce the viability of human cancer cells. FIG. 5A depicts representative results of PANC-1 cells treated with nothing (mock), MYXV at various MOIs, 1 μM selinexor, and combinations of 1 μM selinexor and various MOIs of MYXV. FIG. 5B depicts representative results of PANC-1 cells treated with nothing (mock), MYXV at various MOIs, 0.5 μM selinexor, and combinations of 0.5 μM selinexor and various MOIs of MYXV. FIG. 5C depicts representative results of PANC-1 cells treated with nothing (mock), MYXV at various MOIs, 0.1 μM selinexor, and combinations of 0.1 μM selinexor and various MOIs of MYXV. FIG. 5D depicts representative results of PANC-1 cells treated with nothing (mock), MYXV at various MOIs, 0.05 μM selinexor, and combinations of 0.05 μM selinexor and various MOIs of MYXV. FIG. 5E depicts representative results of Colo205 cells treated with nothing (mock), MYXV at various MOIs, 1 μM selinexor, and combinations of 1 μM selinexor and various MOIs of MYXV. FIG. 5F depicts representative results of Colo205 cells treated with nothing (mock), MYXV at various MOIs, 0.5 M selinexor, and combinations of 0.5 μM selinexor and various MOIs of MYXV. FIG. 5G depicts representative results of Colo205 cells treated with nothing (mock), MYXV at various MOIs, 0.1 μM selinexor, and combinations of 0.1 μM selinexor and various MOIs of MYXV. FIG. 5H depicts representative results of Colo205 cells treated with nothing (mock), MYXV at various MOIs, 0.05 μM selinexor, and combinations of 0.05 μM selinexor and various MOIs of MYXV. For FIG. 5A-FIG. 5H, n=4.

FIG. 6, comprising FIG. 6A through FIG. 6H, depicts representative results demonstrating that selinexor enhances MYXV infection and replication in 3D cultures of human cancer cells. FIG. 6A depicts representative images of 3D Colo205 cell culture treated with 1 μM selinexor for 1 hour, infected with vMyx-GFP-TdTomato in the presence of selinexor, and incubated in fresh media with 1 μM selinexor for 96 h. FIG. 6B depicts representative quantification of the level of GFP fluorescence in 3D Colo205 cells 96 h post-infection. FIG. 6C depicts representative images of 3D PANC-1 cell culture treated with different concentrations of selinexor for 1 hour, infected with vMyx-GFP-TdTomato in the presence of selinexor, and incubated in fresh media with the same concentration of selinexor for 96 h. FIG. 6D depicts representative quantification of the level of GFP fluorescence in 3D PANC-1 cells 96 h post-infection. FIG. 6E depicts representative images of 3D MDA-MB435 cell culture treated with 1 μM selinexor for 1 hour, infected with vMyx-GFP-TdTomato in the presence of selinexor, and incubated in fresh media with 1 μM selinexor for 96 h. FIG. 6F depicts representative quantification of the level of GFP fluorescence in 3D MDA-MB435 cells 72- or 96-hours post-infection. FIG. 6G depicts representative images of 3D HT29 cell culture treated with selinexor for 1 hour, infected with vMyx-GFP-TdTomato in the presence of selinexor, and incubated in fresh media with the same concentration of selinexor for 96 h. FIG. 6H depicts representative quantification of the level of GFP fluorescence in 3D HT29 cells 72- or 96-hours post-infection. For FIG. 6B, FIG. 6D, FIG. 6F, and FIG. 6H, n=3; ***, p<0.001; **, p<0.01; *, p<0.05.

FIG. 7, comprising FIG. 7A through FIG. 7G, depicts representative experimental results demonstrating that selinexor enhances MYXV replication in vivo in xenograft tumors in NSG mice and reduces tumor burden. FIG. 7A depicts a schematic diagram of an experimental setup for examining MYXV replication. Mice were inoculated with Colo205 or HT29 cells at day 0 via SQ injection on both flanks. Mice were treated with three doses of PBS (mock, G1), 15 mg/kg oral selinexor (G2), 1×10⁷ FFU intratumoral (IT) vMyx-FLuc (G3), or a combination of selinexor and vMyx-FLuc and imaged for luciferase 48 hours after the first and second injection of vMyx-FLuc. FIG. 7B depicts representative images of mice with Colo205 tumors taken using IVIS imaging 48 hours after the first injection of vMyx-FLuc. FIG. 7C depicts quantification of the luminescence signals from FIG. 7B. FIG. 7D depicts representative images of mice with Colo205 tumors taken using IVIS imaging 48 hours after the second injection of vMyx-FLuc. FIG. 7E depicts quantification of the luminescence signals from FIG. 7D. FIG. 7F depicts tumor volumes on the left flank of mice after treatment. FIG. 7G depicts tumor volumes on the right flank of mice after treatment. For FIG. 7C and FIG. 7E-FIG. 7G, n=5; ****, p<0.0001; ***, p<0.001; **, p<0.01; *, p<0.05; ns, not significant.

FIG. 8, comprising FIG. 8A through FIG. 8F, depicts representative experimental results demonstrating that selinexor enhances MYXV replication in vivo in HT29 xenograft tumors in NSG mice as outlined in FIG. 7A. FIG. 8A depicts representative images of mice with HT29 tumors taken using IVIS imaging 48 hours after the first injection of vMyx-FLuc. FIG. 8B depicts quantification of the luminescence signals from FIG. 8A. FIG. 8C depicts representative images of mice with HT29 tumors taken using IVIS imaging 48 hours after the second injection of vMyx-FLuc. FIG. 8D depicts quantification of the luminescence signals from FIG. 8C. FIG. 8E depicts tumor volumes on the left flank of mice after treatment. FIG. 8F depicts tumor volumes on the right flank of mice after treatment. For FIG. 8B and FIG. 8D-FIG. 8F, n=5; *, p<0.05; ns, not significant.

FIG. 9, comprising FIG. 9A through FIG. 9H, depicts representative tumor burdens in mice with a Colo205 xenograft. FIG. 9A depicts representative change in tumor volume in the left flank of control mice treated with PBS. FIG. 9B depicts representative change in tumor volume in the left flank of mice treated with oral selinexor. FIG. 9C depicts representative change in tumor volume in the left flank of mice treated with MYXV. FIG. 9D depicts representative change in tumor volume in the left flank of mice treated with MYXV and selinexor. FIG. 9E depicts representative change in tumor volume in the right flank of control mice treated with PBS. FIG. 9F depicts representative change in tumor volume in the right flank of mice treated with oral selinexor. FIG. 9G depicts representative change in tumor volume in the right flank of mice treated with MYXV. FIG. 9H depicts representative change in tumor volume in the right flank of mice treated with MYXV and selinexor.

FIG. 10, comprising FIG. 10A and FIG. 10B, depicts representative experimental results demonstrating that the combination of selinexor and MYXV prolongs survival in a xenograft tumor model in NSG mice. FIG. 10A depicts a representative survival curve for mice with Colo205 xenograft tumors treated with three doses of PBS (control), 15 mg/kg oral selinexor, 1×10⁷ FFU IT vMyx-FLuc, or a combination of selinexor and vMyx-FLuc (Seli+MYXV). FIG. 10B depicts a representative survival curve for mice with HT29 xenograft tumors treated with PBS, selinexor, vMyx-FLuc, or selinexor+vMyx-FLuc. For FIG. 10A and FIG. 10B, n=5; **, p<0.01; *, p<0.05; ns, not significant.

FIG. 11, comprising FIG. 11A through FIG. 11F, depicts representative experimental results demonstrating that selinexor enhances MYXV replication in PANC-1 xenograft tumors in NSG mice, reduces tumor burden, and prolongs survival. FIG. 11A depicts a schematic diagram of an experimental setup for examining MYXV replication. Mice were inoculated with PANC-1 cells at day 0 via SQ injection on both flanks. Mice were treated with three doses of PBS (mock, G1), 15 mg/kg oral selinexor (G2), 1×10⁷ FFU intratumoral (IT) vMyx-FLuc (G3), or a combination of selinexor and vMyx-FLuc and imaged for luciferase 24 or 72 hours after the first and second injection of vMyx-FLuc. FIG. 11B depicts representative images of mice with PANC-1 tumors taken using IVIS imaging 24 or 72 hours after the first injection of vMyx-FLuc. FIG. 11C depicts quantification of the luminescence signals from FIG. 11B. FIG. 11D depicts tumor volumes in the left flank of mice after treatment. FIG. 11E depicts tumor volumes in the right flank of mice after treatment. FIG. 11F depicts a representative survival curve for mice with PANC-1 xenograft tumors after treatment with PBS, selinexor, vMyx-FLuc, or selinexor+vMyx-FLuc. For FIG. 11C-FIG. 11F, n=5; ****, p<0.0001; ***, p<0.001; **, p<0.01; *, p<0.05; ns, not significant.

FIG. 12, comprising FIG. 12A through FIG. 12H, depicts representative tumor burdens in mice with a PANC-1 xenograft. FIG. 12A depicts representative change in tumor volume in the left flank of control mice treated with PBS. FIG. 12B depicts representative change in tumor volume in the left flank of mice treated with oral selinexor. FIG. 12C depicts representative change in tumor volume in the left flank of mice treated with MYXV. FIG. 12D depicts representative change in tumor volume in the left flank of mice treated with MYXV and selinexor. FIG. 12E depicts representative change in tumor volume in the right flank of control mice treated with PBS. FIG. 12F depicts representative change in tumor volume in the right flank of mice treated with oral selinexor. FIG. 12G depicts representative change in tumor volume in the right flank of mice treated with MYXV. FIG. 12H depicts representative change in tumor volume in the right flank of mice treated with MYXV and selinexor.

FIG. 13, comprising FIG. 13A through FIG. 13D, depicts representative experimental results demonstrating prolonged replication of MYXV in the tumor bed of selinexor-treated mice. NSG mice with SQ tumors of PANC-1 cells in both flanks received four doses of oral selinexor (15 mg/kg) and three IT injections of vMyx-FLuc in the right flank and were imaged 10 and 23 days after the last injection of MYXV. FIG. 13A depicts luminescence images taken using IVIS imaging 10 days after the last MYXV injection. FIG. 13B depicts quantification of the luminescence in FIG. 13A, n=6. FIG. 13C depicts representative luminescence images taken using IVIS imaging 23 days after the last MYXV injection. FIG. 13D depicts representative quantification of virus production in the tumors of mice taken at the endpoint of the experiment by titration using permissive RK13 cells, n=3.

FIG. 14, comprising FIG. 14A through FIG. 14C, depicts representative experimental results demonstrating that the combination of selinexor and MYXV reduces tumor burden in immunocompetent mice. FIG. 14A depicts a schematic representation of an experimental setup where C57BL/6 mice were subcutaneously inoculated with Lewis lung carcinoma (LLC1) cells and treated with PBS (control, G1), 15 mg/kg selinexor (oral, G2), 5×10⁷ FFU MYXV expressing IL15/IL15Ra (vMyx-IL15Ra, G3), or selinexor+vMyx-IL15Ra. FIG. 14B depicts representative tumor volumes in the left flank of mice after treatment. FIG. 14C depicts representative tumor volumes in the right flank of mice after treatment. For FIG. 14B and FIG. 14C, n=10; ****, p<0.0001; ***, p<0.001; **, p<0.01; *, p<0.05; ns, not significant.

FIG. 15 depicts representative mass spectrometry quantification of host and viral proteins in the cytoplasm and nucleus of Colo205 cells 48 hours after treatment with selinexor, MYXV, or selinexor+MYXV. Samples were prepared for nuclear and cytosolic fractions and analyzed by LC-MS with a quadrupole mass spectrometer.

### DETAILED DESCRIPTION

The ability of viruses to infect and replicate in host cells can vary based on cell species, cell type, and other cell attributes. Oncolytic viruses selectively or preferentially replicate in cancer cells, however while some cancer cells can be permissive to a given oncolytic virus, others may be only semi-permissive, or non-permissive, reducing the efficacy of the oncolytic virus as a therapeutic. The present disclosure provides compositions and methods for converting non-permissive or semi-permissive cancer cells into permissive cells, promoting replication of the oncolytic virus in the cancer cells and thereby enhancing cancer cell killing and/or anti-cancer immunity.

As demonstrated herein, nuclear export pathways can restrict viral replication, and inhibition of nuclear export pathways can enhance viral replication in such semi-permissive and non-permissive human cancer cells.

### I. NUCLEAR EXPORT INHIBITORS

As demonstrated herein, nuclear export inhibitors can enhance oncolytic virus replication and gene expression in cancer cells that are normally not susceptible, substantially not susceptible, or only exhibit limited susceptibility to infection by an oncolytic virus in the absence of the nuclear export inhibitor. As demonstrated herein, a treatment regimen combining an oncolytic virus with a nuclear export inhibitor can exhibit strikingly superior therapeutic efficacy compared to either agent alone.

A nuclear export inhibitor can be an agent that inhibits transport of molecules through the nuclear export pathway. In certain embodiments, the nuclear export inhibitor is a selective inhibitor. In certain embodiments, the nuclear export inhibitor is non-selective. In some embodiments, a nuclear export inhibitor is an agent that is capable of interfering with nucleocytoplasmic trafficking. In some embodiments, a nuclear export inhibitor alters nuclear export by interfering with protein trafficking.

Nuclear export inhibitors (NEIs) can be classified into four groups as follows: bacterial products, herbal ingredients, fungal or animal NEIs, and synthetic NEIs. Bacterial NEIs include leptomycin A/B, ratjadone A/C and anguinomycin A/B/C/D, which all have a long polyketide chain with a lactone ring. Several plant NEIs were discovered from South/Southeast Asia herbs and food additives, including valtrate, oridonin, acetoxychavicol acetate, curcumin, gonionthalamin, piperlongumine and plumbagin. Wortmannin and cyclopentenone prostaglandin (15d-PGJ2) were known for other functions before they were discovered as CRM1 inhibitors. Synthetic inhibitors include PKF050-638, 5219668, SINEs, compound3/4, CBS9106 and S109.

In some embodiments, the nuclear export inhibitor comprises a selective inhibitor of the nuclear export (SINE). A SINE can be a nuclear export inhibitor that inhibits exportin 1 (XPO1/CRM1). Examples of SINEs include selinexor, KPT-185, KPT-249, KPT-251, KPT-276, KPT-330 and KPT-335.

In some embodiments, the nuclear export inhibitor inhibits exportin 1. XPO1 is a cell-cycle-regulated gene that encodes exportin 1, which mediates leucine-rich nuclear export signal (NES)-dependent protein transport. Exportin 1 mediates the nuclear export of cellular proteins (cargos) bearing a leucine-rich nuclear export signal (NES) and of RNAs. In the nucleus, in association with RANBP3, exportin 1 binds cooperatively to the NES on its target protein and to the GTPase RAN in its active GTP-bound form (Ran-GTP). Docking of this complex to the nuclear pore complex (NPC) is mediated through binding to nucleoporins. Upon transit of a nuclear export complex into the cytoplasm, disassembling of the complex and hydrolysis of Ran-GTP to Ran-GDP (induced by RANBP1 and RANGAP1, respectively) cause release of the cargo from the export receptor. The directionality of nuclear export is thought to be conferred by an asymmetric distribution of the GTP- and GDP-bound forms of Ran between the cytoplasm and nucleus. Exportin 1 is involved in U3 snoRNA transport from Cajal bodies to nucleoli. Exportin 1 binds to late precursor U3 snoRNA bearing a TMG cap. Exportin 1 can specifically inhibit the nuclear export of Rev and U snRNAs. It is involved in the control of several cellular processes by controlling the localization of cyclin B, MPAK, and MAPKAP kinase 2. Exportin 1 also regulates NFAT and AP-1.

In some embodiments, the nuclear export inhibitor binds to and/or inhibits a factor (such as a protein) that binds to a nuclear export signal. In some embodiments, the nuclear export inhibitor binds to and/or inhibits a factor that binds to RAN, RAN-GTP, and/or RAN-GDP. In some embodiments, the nuclear export inhibitor binds to and/or inhibits a factor that docks to the nuclear pore complex. In some embodiments, the nuclear export inhibitor binds to and/or inhibits a factor that mediates leucine-rich nuclear export signal (NES)-dependent protein transport.

In some embodiments, the nuclear export inhibitor comprises a non-covalent nuclear export inhibitor.

In some embodiments, the nuclear export inhibitor inhibits RNA helicase family proteins. In some embodiments, the nuclear export inhibitor inhibits an RNA helicase family protein. In some embodiments, the nuclear export inhibitor inhibits a nuclear protein.

In some embodiments, the nuclear export inhibitor comprises a small molecule compound. In some embodiments, the nuclear export inhibitor comprises a natural compound such as Ratjadone, valtrate and acetoxychavicol acetate. In some embodiments, the nuclear export inhibitor comprises a reversible nuclear export inhibitor such as CBS9106.

In some embodiments, the nuclear export inhibitor is an anti-cancer therapeutic.

Examples of nuclear export inhibitors include but are not limited to Selinexor, Leptomycin A, Leptomycin B, Ratjadone A, Ratjadone B, Ratjadone C, Ratjadone D, Anguinomycin A, Goniothalamin, piperlongumine, plumbagin, curcumin, valtrate, acetoxychavicol acetate, prenylcoumarin osthol, KOS 2464, PKF050-638, and CBS9106. In some embodiments, a nuclear export inhibitor comprises or is Trifuoperazine hydrochloride, W13, ETP-45648, Vinblastine, Akt inhibitor X, INCAs, SMIP001/004, Resveratrol, Elliticine, WGA, cSN50 peptide, bimax1/2 peptide, Leptomycin B, Anguinomycins, Goniothalamin, Ratjadone, Valtrate, Acetoxychavicol acetate, 15d-PGJ2, Peumusolide A, PKF050-638, KOS-2464, CBS9106, or a combination thereof.

In certain embodiments, the nuclear export inhibitor comprises one or more of Leptomycin A, Leptomycin B, Ratjadone A, B, C and D, Anguinomycin A, Goniothalamin, piperlongumine, plumbagin, curcumin, valtrate, acetoxychavicol acetate, prenylcoumarin osthol, or synthetic nuclear export inhibitors such as KOS 2464, PKF050-638 (N-azolylacrylate analog), CBS9106, Selinexor, and those found in Mathew and Ghildyal, CRM1 inhibitors for antiviral therapy, Frontiers in Microbiology 2017, Vol 8, article 1171, which is incorporated herein by reference for such disclosure. In some embodiments, the nuclear export inhibitor comprises Leptomycin A. In some embodiments, the nuclear export inhibitor comprises Leptomycin B. In some embodiments, the nuclear export inhibitor comprises Ratjadone A. In some embodiments, the nuclear export inhibitor comprises Ratjadone B. In some embodiments, the nuclear export inhibitor comprises Ratjadone C. In some embodiments, the nuclear export inhibitor comprises Ratjadone D. In some embodiments, the nuclear export inhibitor comprises Anguinomycin A. In some embodiments, the nuclear export inhibitor comprises Goniothalamin. In some embodiments, the nuclear export inhibitor comprises piperlongumine. In some embodiments, the nuclear export inhibitor comprises plumbagin. In some embodiments, the nuclear export inhibitor comprises curcumin. In some embodiments, the nuclear export inhibitor comprises valtrate. In some embodiments, the nuclear export inhibitor comprises acetoxychavicol acetate. In some embodiments, the nuclear export inhibitor comprises prenylcoumarin osthol. In some embodiments, the nuclear export inhibitor comprises KOS 2464. In some embodiments, the nuclear export inhibitor comprises PKF050-638. In some embodiments, the nuclear export inhibitor comprises CBS9106. In some embodiments, the nuclear export inhibitor comprises or consists of Selinexor. In some embodiments, the nuclear export inhibitor is Leptomycin B.

In some embodiments, the nuclear export inhibitor is not rapamycin or an analog (e.g., structural analog) thereof.

### II. ONCOLYTIC VIRUSES

Compositions and methods of the disclosure utilize oncolytic viruses. In some embodiments, an oncolytic virus is a mammalian virus that is engineered and/or selected for its ability to selectively infect and kill cancer cells, and for an ability to activate the host immune system against the virus and/or tumor antigens.

An oncolytic virus described herein can be a virus capable of selectively or preferentially replicating in cancer cells. An oncolytic virus described herein can be a virus capable of selectively or preferentially replicating in dividing cells (e.g., a proliferative cell such as a cancer cell). Infection of and replication in a cancer cell can slow the growth of the proliferative cell and/or kill the proliferative cell, while showing no, substantially no, or less replication in non-dividing cells. An oncolytic virus can contain a viral genome packaged into a viral particle or virion and can be infectious (e.g., capable of entering into a host cell or subject). An oncolytic virus can be a DNA virus. An oncolytic virus can be an RNA virus.

An oncolytic virus can be a poxvirus from the Poxviridae family. Poxviruses are double-stranded DNA viruses that collectively are capable of infecting both vertebrates and invertebrates. Members of Poxviridae family of viruses are a diverse group of large, complex double-stranded DNA viruses that can replicate in the cytoplasm of infected permissive cells. The genomes of most poxviruses are about 150,000 to 300,000 base pairs in length and encode approximately 150 to 300 proteins. About half of these viral proteins can be highly conserved between different poxvirus members and perform essential functions like cell binding and entry, genome replication, transcription and virion assembly. Other viral proteins can be involved in evading many host defense functions, for example, can be required for the inhibition or manipulation of diverse intracellular anti-viral signaling pathways functioning in the cytoplasm and nucleus. The poxviral genes can be expressed in distinct phases. For example, the early gene products can include proteins that are necessary for viral DNA replication and are expressed before the DNA is replicated. Intermediate/late gene products expressed during or after DNA replication can include the structural proteins required for virion maturation. Some evidence suggests that the steps of this complex viral replication process (starting from un-coating the genome, early gene expression, DNA replication, late gene expression and an even more complex virion maturation processes) can occur exclusively in the cytoplasm of the infected cells. However, there is also evidence that host cell proteins from cytoplasm and nuclear compartments participate in at least some steps of poxvirus replication. Many diverse cellular proteins and signaling pathways have been implicated in defending the cell against the infection and replication of poxviruses.

Poxviruses include, for example, species and genera of viruses that are classified as being a part of the Chordopoxvirinae subfamily such as Orthopoxvirus, Parapoxvirus, Avipoxvirus, Capripoxvirus, Leporipoxvirus, Suipoxvirus, Molluscipoxvirus, and Yatapoxvirus genera, and the Entomopoxvirinae subfamily, including Alphaentomopoxvirus, Betaentomopoxvirus, and Gammaentopoxvirus genera.

In some embodiments, the poxvirus is genetically modified. In some embodiments, the poxvirus is a Leporipoxvirus. In some embodiments, the Leporipoxvirus is a myxoma virus (MYXV). In some embodiments, the poxvirus is an Orthopoxvirus. In some embodiments, the Orthopoxvirus is a vaccinia virus. In some embodiments, the vaccinia virus is a vaccinia virus strain selected from the group consisting of Lister, Wyeth, Western Reserve, Modified Vaccinia virus Ankara, and LC16m series. In some embodiments, the Orthopoxvirus is a Raccoonpox virus. In some embodiments, the poxvirus is a Capripox virus. In some embodiments, the Capripox virus is an Orf virus.

In some embodiments, the oncolytic virus is a myxoma virus (MYXV) or is derived from a MYXV. MYXV is a member of the family poxviridae and genus Leporipoxvirus. In some embodiments, the MYXV is a wild-type strain of MYXV or is derived from a wild-type strain of MYXV. In some embodiments, the MYXV is a genetically modified strain of MYXV or is derived from a genetically modified strain of MYXV. In some instances, the MYXV is Lausanne strain or is derived from Lausanne strain. In some instances, the MYXV is a South American MYXV strain that circulates in Sylvilagus brasiliensis or is derived from a South American MYXV strain that circulates in Sylvilagus brasiliensis. In some instances, the MYXV is a Californian MYXV strain that circulates in Sylvilagus bachmani or is derived from a Californian MYXV strain that circulates in Sylvilagus bachmani. In some instances, the MYXV is 6918, an attenuated Spanish field strain that comprises modifications in genes M009L, M036L, M135R, and M148R (GenBank Accession number EU552530 which is hereby incorporated by reference as provided by GenBank on Jul. 27, 2019) or is derived from 6918. In some instances, the MYXV is 6918VP60-T2 (GenBank Accession Number EU552531 which is hereby incorporated by reference as provided by GenBank on Jul. 27, 2019) or is derived from 6918VP60-T2. In some instances, the MYXV is a strain termed the Standard laboratory Strain (SLS) or is derived from SLS.

In some embodiments, the MYXV is able to preferentially or selectively infect and kill permissive human cancer cells derived from different tissues. In normal primary human cells, the replication of MYXV can be restricted by multiple factors such as, for example, the cellular binding determinants, the intracellular anti-viral signaling pathways, type I IFN signaling pathways, and/other cytokine-mediated cellular anti-viral states. In human cancer cells, these self-defense cell pathways are commonly defective. MYXV replication in some human cancer cells can depend on cellular RNA helicase family proteins. Without wishing to be bound by any particular theory, RNA helicases which shuttle between nuclear and cytoplasmic compartments of cells may influence MYXV replication in virus-infected cells. Beside RNA helicases, other nuclear proteins may contribute to the replication cycle of MYXV and other poxviruses. For example, nuclear proteins might affect the replication efficiency of poxviruses in transformed human host cell lines.

MYXV late gene expression, replication, and progeny virus formation can be limited in certain human cancer cells or cancer cell types. These cancer cells and cancer cell types can be classified as semi-permissive and non-permissive human cancer cells.

In some embodiments, the oncolytic virus is from a virus family consisting of: Poxviridae, Herpesviridae, Reoviridae, Paramyxoviridae, Retroviridae, Adenoviridae, Rhabdoviridae, Picornaviridae, Parvoviridae, and Picornaviridae, or is derived from a virus family consisting of: Poxviridae, Herpesviridae, Reoviridae, Paramyxoviridae, Retroviridae, Adenoviridae, Rhabdoviridae, Picornaviridae, Parvoviridae, and Picornaviridae. In some embodiments, the oncolytic virus is from the Herpesviridae family or is derived from the Herpesviridae family. In some embodiments, the oncolytic virus is from the Reoviridae family or is derived from the Reoviridae family. In some embodiments, the oncolytic virus is from the Paramyxoviridae family or is derived from Paramyxoviridae family. In some embodiments, the oncolytic virus is from the Retroviridae family or is derived from is from the Retroviridae family. In some embodiments, the oncolytic virus is from the Adenoviridae family or is derived from the Adenoviridae family. In some embodiments, the oncolytic virus is from the Rhabdoviridae family or is derived from the Rhabdoviridae family. In some embodiments, the oncolytic virus is from the Picornaviridae family or is derived from the Picornaviridae family. In some embodiments, the oncolytic virus is from the Parvoviridae family or is derived from the Parvoviridae family. In some embodiments, the oncolytic virus is from the Picornaviridae family. In some embodiments, the oncolytic virus is from a genus that is Simplexvirus, Rubulavirus, or Senecavirus or is derived from a genus that is Simplexvirus, Rubulavirus, or Senecavirus. In some embodiments, the oncolytic virus is from genus Simplexvirus or is derived from genus Simplexvirus. In some embodiments, the oncolytic virus is from genus Rubulavirus or is derived from genus Rubulavirus. In some embodiments, the oncolytic virus is from genus Senecavirus or is derived from genus Senecavirus. In some embodiments, the oncolytic virus is from a species of virus that is Measles, Fowlpox, Vesicular Stomatitis Virus, Mumps rubulavirus, Coxsackie Virus, and Vaccinia or is derived from a species of virus that is Measles, Fowlpox, Vesicular Stomatitis Virus, Mumps rubulavirus, Coxsackie Virus, and Vaccinia. In some embodiments, the oncolytic virus is a Measles virus or is derived from a Measles virus. In some embodiments, the oncolytic virus is a Fowlpox virus or is derived from a Fowlpox virus. In some embodiments, the oncolytic virus is a Vesicular Stomatitis Virus or is derived from a Vesicular Stomatitis Virus. In some embodiments, the oncolytic virus is a Mumps rubulavirus or is derived from Mumps rubulavirus. In some embodiments, the oncolytic virus is a Coxsackie Virus or is derived from is a Coxsackie Virus. In some embodiments, the oncolytic virus is a Vaccinia virus or is derived from is a Vaccinia virus.

In some embodiments, the oncolytic virus is a virus from Chordopoxvirinae subfamily or Entomopoxvirinae subfamily or is derived from Chordopoxvirinae subfamily or Entomopoxvirinae subfamily. In some embodiments, the oncolytic virus is from a genus that is Orthopoxvirus, Cervidpoxvirus, Parapoxvirus, Avipoxvirus, Capripoxvirus, Leporipoxvirus, Suipoxvirus, Molluscipoxvirus, Yatapoxvirus, Alphaentomopoxvirus, Betaentomopoxvirus, or Gammaentopoxvirus. In some embodiments, the oncolytic virus is derived from a virus from a genus that is Orthopoxvirus, Cervidpoxvirus, Parapoxvirus, Avipoxvirus, Capripoxvirus, Leporipoxvirus, Suipoxvirus, Molluscipoxvirus, Yatapoxvirus, Alphaentomopoxvirus, Betaentomopoxvirus, or Gammaentopoxvirus. In some embodiments, the oncolytic virus is from genus Orthopoxvirus or is derived from a virus of the genus Orthopoxvirus. In some embodiments, the oncolytic virus is a vaccinia virus or is derived from a vaccinia virus. In some embodiments, the vaccinia virus is a vaccinia virus strain selected from the group consisting of Lister, Wyeth, Western Reserve, Modified Vaccinia virus Ankara, and LC16m series. In some embodiments, the oncolytic virus is a Raccoonpox virus or is derived from a Raccoonpox virus. In some embodiments, the oncolytic virus is from genus Cervidpoxvirus or is derived from a virus of the genus Cervidpoxvirus. In some embodiments, the oncolytic virus is an Orf virus or is derived from an Orf virus. In some embodiments, the oncolytic virus is from genus Parapoxvirus or is derived from a virus of the genus Parapoxvirus. In some embodiments, the oncolytic virus is from genus Avipoxvirus or is derived from a virus of the genus Avipoxvirus. In some embodiments, the oncolytic virus is from genus Capripoxvirus or is derived from a virus of the genus Capripoxvirus. In some embodiments, the oncolytic virus is from genus Suipoxvirus or is derived from a virus of the genus Suipoxvirus. In some embodiments, the oncolytic virus is from genus Molluscipoxvirus or is derived from a virus of the genus Molluscipoxvirus. In some embodiments, the oncolytic virus is from genus Yatapoxvirus or is derived from a virus of the genus Yatapoxvirus. In some embodiments, the oncolytic virus is from genus Alphaentomopoxvirus or is derived from a virus of the genus Alphaentomopoxvirus. In some embodiments, the oncolytic virus is from genus Betaentomopoxvirus or is derived from a virus of the genus Betaentomopoxvirus. In some embodiments, the oncolytic virus is from genus Gammaentopoxvirus or is derived from a virus of the genus Gammaentopoxvirus. In some embodiments, the oncolytic virus is from genus Leporipoxvirus or is derived from a virus of the genus Leporipoxvirus. In some embodiments, the oncolytic virus is replication-competent.

### A. Genetic Modifications

An oncolytic virus disclosed herein can be genetically modified. For example, the virus can be modified to comprise a heterologous transgene, such as a therapeutic or immunomodulatory gene, and/or to delete or disrupt one or more endogenous viral genes.

A heterologous transgene can be selected to enhance the anticancer effect of an oncolytic virus. In some embodiments a heterologous transgene triggers cell death, for example, apoptosis, necrosis, or necroptosis. In some embodiments a heterologous transgene targets the infected cell for immune destruction, such as a gene that repairs a lack of response to interferon, or that results in the expression of a cell surface marker that stimulates an antibody response, such as a bacterial cell surface antigen. In some embodiments a heterologous transgene reduces a cancer cell's proliferation.

In some embodiments, the heterologous transgene encodes a cytokine or a functional fragment thereof, for example, IL-12, IL-15, IL-15Ra, IL15/IL15Ra (a fusion protein of interleukin-15 (IL15) and IL 15 receptor alpha), LIGHT, p14 FAST, TNF-α. In some embodiments, the heterologous transgene encodes an interleukin or a functional fragment thereof, for example, IL-12, IL-15, or IL15/IL15Ra. In some embodiments, the heterologous transgene encodes a cell matrix protein or a functional fragment thereof, for example, decorin. In some embodiments, the heterologous transgene encodes an antibody or a functional fragment thereof, for example, an anti-PD-L1 or anti-PD-1 antibody or antigen-binding fragment thereof, or another immune checkpoint inhibitor. In some embodiments, the heterologous transgene encodes an anti-PD-L1 antibody, decorin, IL-12, LIGHT, p14 FAST, TNF-α, a functional fragment thereof, or a combination thereof. In some embodiments, the heterologous transgene encodes a checkpoint inhibitor or a functional fragment thereof. In some embodiments, the heterologous transgene encodes a multi-specific immune cell engager, for example, a bispecific killer cell engager (BiKE) or a bispecific T cell engager (BiTE).

An endogenous viral gene can be deleted, disrupted, or modified to enhance the anticancer effect of an oncolytic virus.

### III. METHODS AND REGIMENS

As disclosed herein, nuclear export inhibitors can be used to convert cancer cells that are not susceptible to an oncolytic virus (e.g., MYXV) to cancer cells that are relatively more susceptible to infection by the oncolytic virus, e.g., to induce susceptibility of a cancer cell to infection by the oncolytic virus. Addition of the nuclear export inhibitor to a method of treatment or therapeutic regimen can enhance the therapeutic efficacy of the treatment or regimen.

The nuclear export inhibitor can be used to convert a cancer cell that has low susceptibility to infection, replication, and/or killing by oncolytic virus into a cancer cell that has relatively higher susceptibility to infection, replication, and/or killing by oncolytic virus. Therefore, the nuclear export inhibitor can be administered in combination with oncolytic virus to improve the efficacy of the oncolytic virus in treating cancer and killing tumor cells.

Disclosed herein, in some embodiments, are methods for treating cancer by administering to a subject a therapeutically-effective amount of an oncolytic virus (e.g., MYXV) and a therapeutically-effective amount of a nuclear export inhibitor to a subject in need thereof. In some embodiments, the subject is a mammal. In some embodiments, the subject is a primate. In some embodiments, the subject is a human. In some embodiments, the subject is a canine. In some embodiments, the subject is a non-rodent mammal.

In one aspect, disclosed herein are methods of converting a nonpermissive or semi-permissive cell to a permissive cell comprising: contacting a cancer cell that is nonpermissive to an oncolytic virus with the oncolytic virus and a nuclear export inhibitor, thereby converting said cancer cell. In another aspect, disclosed herein are methods of killing a cancer cell comprising: contacting a cancer cell with an oncolytic virus and a nuclear export inhibitor, thereby killing said cancer cell. Accordingly, in some embodiments, this disclosure provides methods of increasing virus replication in a nonpermissive cancer cell by treating the nonpermissive or semi-permissive cell with a nuclear export inhibitor.

In some embodiments, the cancer cell, e.g., the nonpermissive or semi-permissive cancer cell, is an animal cell such as a mammalian cell. In some embodiments, the cancer cell, e.g., the nonpermissive cancer cell, is a human cell. In certain embodiments, the cell is an immortalized human or primate cell. In some embodiments, the cancer cell, e.g., the nonpermissive cancer cell, is a canine cell. In some embodiments, the cancer cell is a cell of a cancer tissue of a subject.

### B. Administration and Dosing

An oncolytic virus disclosed herein, such as a MYXV, can be administered to a subject in a therapeutically-effective amount by various forms and routes including, for example, systemic, oral, topical, parenteral, intravenous injection, intravenous infusion, intratumoral injection, subcutaneous injection, intramuscular injection, intradermal injection, intraperitoneal injection, intracerebral injection, subarachnoid injection, intraspinal injection, intrasternal injection, intraarticular injection, endothelial administration, local administration, intranasal administration, intrapulmonary administration, intraarterial administration, intrathecal administration, inhalation, intralesional administration, intradermal administration, epidural administration, absorption through epithelial or mucocutaneous linings (e.g., oral mucosa, rectal and intestinal mucosa), intracapsular administration, subcapsular administration, intracardiac administration, transtracheal administration, subcuticular administration, subarachnoid administration, subcapsular administration, intraspinal administration, or intrasternal administration.

In some embodiments, the virus is administered systemically. In some embodiments, the virus is administered by injection at a disease site. In some embodiments, the virus is administered orally. In some embodiments, the virus is administered parenterally.

An oncolytic virus disclosed herein, such as a MYXV, can be administered at any interval desired. In some embodiments, the virus can be administered hourly. In some embodiments, the virus is administered about every 1, 2, 3, 4, 5, 6, 7, 8, 9, 10, 11, 12, 13, 14, 15, 16, 18, 20, 22, 24, 26, 28, 30, 32, 36, 40, 44, or 48 hours. In some embodiments, the virus can be administered twice a day, once a day, five times a week, four times a week, three times a week, two times a week, once a week, once every two weeks, once every three weeks, once every four weeks, once a month, once every five weeks, once every six weeks, once every eight weeks, once every two months, once every twelve weeks, once every three months, once every four months, once every six months, once a year, or less frequently.

In some embodiments, the oncolytic virus, the nuclear export inhibitor, or both are administered to the subject from 1 to 4 weeks apart, for examples, about 1 week apart, about 2 weeks apart or about 3 weeks apart. In some embodiments, the oncolytic virus, the nuclear export inhibitor, or both are administered to the subject from 1 to 4 months apart, for examples, about 1 months apart, about 2 months apart or about 3 months apart.

An oncolytic virus disclosed herein, such as a MYXV, can be administered in combination with one or more other therapies. An oncolytic virus (e.g., MYXV) of the disclosure can be administered in combination with a nuclear export inhibitor and in combination with one or more additional other therapies. In some embodiments, an oncolytic virus (e.g., MYXV) of the disclosure is administered in combination with a chemotherapy, an immunotherapy, a cell therapy, a radiation therapy, a stem cell transplant (such as an autologous stem cell transplant), or a combination thereof. For example, the oncolytic virus (e.g., MYXV) can be administered either prior to or following another treatment, such as administration of radiotherapy or conventional chemotherapeutic drugs and/or a stem cell transplant, such as an autologous stem cell transplant or an allogenic stem cell transplant (e.g., a HLA-matched, HLA-mismatched, or haploidentical transplant). In some embodiments, a oncolytic virus (e.g., MYXV) of the disclosure can be in combination with an immune checkpoint modulator.

A nuclear export inhibitor of the disclosure can be administered to a subject in an effective amount. In some embodiments, the nuclear export inhibitor is administered to a subject at a dose of about 20 mg-100 mg, about 20-60 mg, or about 60 mg-100 mg. In some embodiments, the nuclear export inhibitor is administered to a subject at a dose per kilogram of the subject's body weight, for example, at a dose of about 0.001-1000 mg/kg, about 0.01-100 mg/kg, about 5-20 mg/kg or about 0.01-10 mg/kg.

A nuclear export inhibitor of the disclosure can be administered to a subject in a therapeutically-effective amount by various forms and routes including, for example, systemic, oral, topical, parenteral, intravenous injection, intravenous infusion, intratumoral injection, subcutaneous injection, intramuscular injection, intradermal injection, intraperitoneal injection, intracerebral injection, subarachnoid injection, intraspinal injection, intrasternal injection, intraarticular injection, endothelial administration, local administration, intranasal administration, intrapulmonary administration, intraarterial administration, intrathecal administration, inhalation, intralesional administration, intradermal administration, epidural administration, absorption through epithelial or mucocutaneous linings (e.g., oral mucosa, rectal and/or intestinal mucosa), intracapsular administration, subcapsular administration, intracardiac administration, transtracheal administration, subcuticular administration, subarachnoid administration, subcapsular administration, intraspinal administration, or intrasternal administration.

In some embodiments, the nuclear export inhibitor is administered orally. In some embodiments, the nuclear export inhibitor is administered systemically. In some embodiments, the nuclear export inhibitor is administered by injection at a disease site. In some embodiments, the nuclear export inhibitor is administered parenterally.

In some embodiments, the method comprises administering a therapeutically effective amount of a nuclear export inhibitor at or near the cancer tissue. In some embodiments, the method comprises contacting the cancer cell or cancer tissue with a media comprising a therapeutically effective amount of the nuclear export inhibitor. In some embodiments, the method comprises incubating the cancer cell or cancer tissue with a composition comprising a therapeutically effective amount of the nuclear export inhibitor.

A nuclear export inhibitor of the disclosure can be administered at any interval desired. In some embodiments, the nuclear export inhibitor is administered hourly. In some embodiments, the nuclear export inhibitor is administered about every 1, 2, 3, 4, 5, 6, 7, 8, 9, 10, 11, 12, 13, 14, 15, 16, 18, 20, 22, 24, 26, 28, 30, 32, 36, 40, 44, or 48 hours. In some embodiments, the nuclear export inhibitor is administered twice a day, once a day, five times a week, four times a week, three times a week, two times a week, once a week, once every two weeks, once every three weeks, once every four weeks, once a month, once every five weeks, once every six weeks, once every eight weeks, once every two months, once every twelve weeks, once every three months, once every four months, once every six months, once a year, or less frequently.

A nuclear export inhibitor of the disclosure can be administered in combination with one or more other therapies. A nuclear export inhibitor of the disclosure can be administered in combination with an oncolytic virus and in combination with one or more additional other therapies. In some embodiments, a nuclear export inhibitor of the disclosure is administered in combination with a chemotherapy, an immunotherapy, a cell therapy, a radiation therapy, a stem cell transplant (such as an autologous stem cell transplant), or a combination thereof. For example, the nuclear export inhibitor can be administered either prior to or following another treatment, such as administration of radiotherapy or conventional chemotherapeutic drugs and/or a stem cell transplant, such as an autologous stem cell transplant or an allogenic stem cell transplant (e.g., a HLA-matched, HLA-mismatched, or haploidentical transplant). In some embodiments, a nuclear export inhibitor of the disclosure can be in combination with an immune checkpoint modulator.

In some embodiments, the method comprises a systemic administration. For example, in some embodiments, the oncolytic virus, the nuclear export inhibitor, or both are administered systemically.

In some embodiments, the method comprises a local administration to the cancer tissue to be treated. For example, in some embodiments, the oncolytic virus, the nuclear export inhibitor, or both are administered locally to a cancer tissue to be treated. In some embodiments, the oncolytic virus is administered locally and the nuclear export inhibitor is administered systemically. In some embodiments, the oncolytic virus is administered locally and the nuclear export inhibitor is administered orally. In some embodiments, the oncolytic virus, the nuclear export inhibitor, or both are administered parenterally. In some embodiments, the nuclear export inhibitor, or both are administered by injection. In some embodiments, the oncolytic virus, the nuclear export inhibitor, or both are administered by cutaneous injection, subcutaneous injection, or injection to a nodal lesion. In some embodiments, the oncolytic virus, the nuclear export inhibitor, or both are administered by an injection to the cancer tissue or a cancer organ that contains the cancer tissue.

In some embodiments, the oncolytic virus (e.g., MYXV) is administered intratumorally and the nuclear export inhibitor is administered orally. In some embodiments, the oncolytic virus (e.g., MYXV) is administered intravenously and the nuclear export inhibitor is administered orally. In some embodiments, the oncolytic virus (e.g., MYXV) is administered locally and the nuclear export inhibitor is administered orally. In some embodiments, the oncolytic virus (e.g., MYXV) is administered systemically and the nuclear export inhibitor is administered orally.

In some embodiments, the oncolytic virus (e.g., MYXV) is administered intratumorally and the nuclear export inhibitor is administered parenterally. In some embodiments, the oncolytic virus (e.g., MYXV) is administered intravenously and the nuclear export inhibitor is administered parenterally. In some embodiments, the oncolytic virus (e.g., MYXV) is administered locally and the nuclear export inhibitor is administered parenterally. In some embodiments, the oncolytic virus (e.g., MYXV) is administered systemically and the nuclear export inhibitor is administered parenterally.

In some embodiments, the oncolytic virus (e.g., MYXV) is administered intratumorally and the nuclear export inhibitor is administered locally. In some embodiments, the oncolytic virus (e.g., MYXV) is administered intravenously and the nuclear export inhibitor is administered locally. In some embodiments, the oncolytic virus (e.g., MYXV) is administered locally and the nuclear export inhibitor is administered locally. In some embodiments, the oncolytic virus (e.g., MYXV) is administered systemically and the nuclear export inhibitor is administered locally.

In some embodiments, the oncolytic virus (e.g., MYXV) is administered intratumorally and the nuclear export inhibitor is administered systemically. In some embodiments, the oncolytic virus (e.g., MYXV) is administered intravenously and the nuclear export inhibitor is administered systemically. In some embodiments, the oncolytic virus (e.g., MYXV) is administered locally and the nuclear export inhibitor is administered systemically. In some embodiments, the oncolytic virus (e.g., MYXV) is administered systemically and the nuclear export inhibitor is administered systemically.

In some embodiments, the oncolytic virus (e.g., MYXV) is administered intratumorally and the nuclear export inhibitor is administered topically. In some embodiments, the oncolytic virus (e.g., MYXV) is administered intravenously and the nuclear export inhibitor is administered topically. In some embodiments, the oncolytic virus (e.g., MYXV) is administered locally and the nuclear export inhibitor is administered topically. In some embodiments, the oncolytic virus (e.g., MYXV) is administered systemically and the nuclear export inhibitor is administered topically.

In some embodiments, the method comprises administering the oncolytic virus, the nuclear export inhibitor, or both to the subject for a period of time. In some embodiments, the period of time is at least 1 day, at least 2 days, at least 3 days, at least 1 week, at least 2 weeks, at least 3 weeks, at least 4 weeks, at least 1 month, at least 3 months, at least 6 months, or at least 1 year. In some embodiments, the period of time is at most 1 day, at most 2 days, at most 3 days, at most 1 week, at most 2 weeks, at most 3 weeks, at most 4 weeks, at most 1 month, at most 3 months, at most 6 months, at most 1 year, or at most 10 years. In some embodiments, the period of time is from about 1 day to about 1 year, from about 1 month to about 12 months, or from 1 month to about 6 months.

The oncolytic virus and the nuclear export inhibitor can be administered together or separately. In some embodiments, the oncolytic virus and the nuclear export inhibitor are administered together. In some embodiments, the oncolytic virus and the nuclear export inhibitor are administered separately. When the oncolytic virus and the nuclear export inhibitor are administered separately, the oncolytic virus can be administered prior to the nuclear export inhibitor. When the oncolytic virus and the nuclear export inhibitor are administered separately, the oncolytic virus can be administered after the nuclear export inhibitor.

In some embodiments, the method comprises administering the oncolytic virus, the nuclear export inhibitor, or both according to an initial dose schedule and a subsequent dose schedule. In some embodiments, the initial dose schedule comprises a different dosing schedule from the subsequent dose schedule. In some embodiments, the initial dose schedule comprises a less frequent administration than the subsequent dose schedule. In some embodiments, the initial dose schedule comprises 1 to 10 treatments, such as 1 to 4 treatments or 2 to 3 treatments of the oncolytic virus, the nuclear export inhibitor, or both. In some embodiments, each treatment of the oncolytic virus, the nuclear export inhibitor, or both is administered from 1 week to about 6 weeks apart according to the initial dose schedule. In some embodiments, each treatment of the oncolytic virus, the nuclear export inhibitor, or both is administered about 1 week apart, about 2 weeks apart, about 3 weeks apart, or about 4 weeks apart according to the initial dose schedule. In some embodiments, each treatment of the oncolytic virus, the nuclear export inhibitor, or both is administered from 1 week to about 6 weeks apart according to the subsequent dose schedule. In some embodiments, each treatment of the oncolytic virus, the nuclear export inhibitor, or both is administered about 1 week apart, about 2 weeks apart, about 3 weeks apart, or about 4 weeks apart according to the subsequent dose schedule. In some embodiments, the oncolytic virus, the nuclear export inhibitor, or both are administered about 3 weeks apart in the initial dose schedule and about 2 weeks apart in the subsequent dose schedule.

The oncolytic virus and the nuclear export inhibitor can be administered to the subject with cancer simultaneously or sequentially. In some embodiments, the oncolytic virus and the nuclear export inhibitor are administered to the subject simultaneously. In some embodiments, the oncolytic virus and the nuclear export inhibitor are pre-mixed before their administration to the subject. In some embodiments, the oncolytic virus and the nuclear export inhibitor are administered to the subject separately. In some embodiments, the oncolytic virus is administered before the nuclear export inhibitor. In some embodiments, the oncolytic virus is administered after the nuclear export inhibitor. In some embodiments, the method comprises contacting the cancer cell or cancer tissue with the oncolytic virus and the nuclear export inhibitor simultaneously or sequentially. In some embodiments, the cancer cell or cancer tissue is contacted with the oncolytic virus before its contact with the nuclear export inhibitor. In some embodiments, the cancer cell or cancer tissue is contacted with the nuclear export inhibitor before its contact with the oncolytic virus. In some embodiments, the method comprises contacting the cancer cell or cancer tissue with a pre-mix of the oncolytic virus and the nuclear export inhibitor. In some embodiments, the cancer cell or cancer tissue is contacted with the oncolytic virus and the nuclear export inhibitor separately

In some embodiments, the method comprises pre-treating the cancer cell or cancer tissue with the nuclear export inhibitor. In some embodiments, the cancer cell or cancer tissue is pre-treated with the nuclear export inhibitor for at least 1 minute, at least 2 minutes, at least 5 minutes, at least 10 minutes, at least 30 minutes, at least 1 hour, at least 2 hours, at least 6 hours, at least 12 hours, at least 24 hours, or at least 1 week before contacting the cell or tissue with the oncolytic virus. In some embodiments, the cancer cell or cancer tissue is pre-treated with the nuclear export inhibitor for at most 1 minute, at most 2 minutes, at most 5 minutes, at most 10 minutes, at most 30 minutes, at most 1 hour, at most 2 hours, at most 6 hours, at most 12 hours, at most 24 hours, or at most 1 week before contacting the cell or tissue with the oncolytic virus. In some embodiments, the cancer cell or cancer tissue is pre-treated with the nuclear export inhibitor for a period of from about 1 minute to about 1 day, from about 5 minutes to about 12 hours, from about 10 minutes to about 2 hours, or from about 30 minutes to about 90 minutes before contacting the cell or tissue with the oncolytic virus. In some embodiments, the cancer cell or cancer tissue is pre-treated with the nuclear export inhibitor for about 1 hour before contacting the cell or tissue with the oncolytic virus.

The compositions can be administered once daily, twice daily, once every two days, once every three days, once every four days, once every five days, once every six days, once every seven days, once every two weeks, once every three weeks, once every four weeks, once every two months, once every six months, or once per year. The dosing interval can be adjusted according to the needs of individual subject. In certain embodiments, the therapeutic agents of the disclosure are administered for time periods exceeding two weeks, three weeks, one month, two months, three months, four months, five months, six months, one year, two years, three years, four years, or five years, ten years, or fifteen years; or for example, any time period range in days, months or years in which the low end of the range is any time period between 14 days and 15 years and the upper end of the range is between 15 days and 20 years (e.g., 4 weeks and 15 years, 6 months and 20 years). In some cases, it may be advantageous for the therapeutic agents to be administered for the remainder of the patient's life. In some embodiments, the patient is monitored to check the progression of the disease or disorder, and the dose is adjusted accordingly. In some embodiments, treatment according to the invention is effective for at least two weeks, three weeks, one month, two months, three months, four months, five months, six months, one year, two years, three years, four years, or five years, ten years, fifteen years, twenty years, or for the remainder of the subject's life.

Further disclosed is a delivery strategy where the therapeutic oncolytic virus (e.g., MYXV) is first incubated with leukocytes ex vivo from bone marrow and/or peripheral blood mononuclear cells prior to introducing the cells into a subject with cancer. In some embodiments, the leukocytes and the oncolytic virus (e.g., MYXV) are incubated together with a nuclear export inhibitor ex vivo. In this strategy, oncolytic virus (e.g., MYXV) may be delivered to cancer sites via migration of leukocytes pre-infected with virus ex vivo. This systemic delivery method is sometimes called “ex vivo virotherapy”, or EVV (aka EV2), because the virus is first delivered to isolated leukocytes prior to infusion into the patient. In some embodiments, the leukocytes are incubated with oncolytic virus (e.g., for one hour ex vivo), and then the oncolytic virus (e.g., MYXV)-loaded leukocytes are infused back into the recipient. In some embodiments, incubation with the nuclear export inhibitor increases uptake of oncolytic virus (e.g., MYXV) by the leukocytes and/or increases delivery of virus to the tumor sites. In some embodiments, the nuclear export inhibitor is not added to the leukocytes and oncolytic virus (e.g., MYXV) ex vivo. In some embodiments, the nuclear export inhibitor is administered to the subject after administering the leukocytes with adsorbed oncolytic virus. In some embodiments, the nuclear export inhibitor is not administered to the subject after administering the leukocytes with adsorbed oncolytic virus (e.g., MYXV).

In certain embodiments, the mononuclear leukocytes are peripheral blood cells and/or bone marrow cells obtained from the subject, for example as autologous cells. In some embodiments, the leukocytes are mononuclear peripheral blood cells and/or bone marrow cells obtained from one or more allogeneic donors, for example, a donor that is matched to the recipient for at least 1, at least 2, at least 3, at least 4, at least 5, at least 6, at least 7, or at least 8 HLA alleles (such as one or both copies of HLA-A, HLA-B, HLA-A, and/or HLA-DR alleles). HLA alleles can be typed, for example, using DNA-based methods. In some embodiments, the mononuclear peripheral blood cells and/or bone marrow cells are obtained from one or more heterologous donors.

In some embodiments, the cancer cell is allowed to incubate with the oncolytic virus (e.g., MYXV or VACV) for a period of time to allow the virus of interest to adsorb to the surface of the cell, such as about 20 minutes to about 5 hours, for example about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 2 hours, about 2.5 hours, about 3 hours, about 3.5 hours, about 4 hours, about 4.5 hours, about 5 hours, 12 hours, 18 hours, 20 hours, 24 hours, 30 hours, 36 hours, or even longer.

In some embodiments, the method comprises contacting the cancer cell or cancer tissue with the oncolytic virus at an MOI as described herein. The MOI can be determined for a given cell or tissue, for example, by titrating the ratio of virus to the cell or tissue, and quantifying the replication of viral progeny and/or the cell viability as disclosed herein. In some embodiments, an effective MOI can minimize drug-specific cellular toxicity, while enhancing replication of the virus and reducing cancer cell viability. In certain embodiments, the MOI of the oncolytic virus to the cancer cell or cancer tissue is from about 0.01 to about 10, from about 0.05 to about 5, and/or ranges therebetween.

### C. Indications

Compositions and methods disclosed herein can be useful in methods of treating cancer in a subject in need thereof. The cancer can be a solid tumor or a blood tumor. In some embodiments, the cancer is leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease, non-Hodgkin's disease), Waldenstrom's macroglobulinemia, heavy chain disease, or a solid tumor. In some embodiments, the cancer is a solid tumor. In some embodiments, the solid tumor is a sarcoma or a carcinoma. In some embodiments, the solid tumor is fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, or retinoblastoma. In some embodiments, the solid tumor is colorectal adenocarcinoma, pancreatic cancer, or melanoma. In some embodiments, the solid tumor is a bone cancer such as chondrosarcoma, Ewing sarcoma, and osteosarcoma. In some embodiments, the solid tumor is osteosarcoma.

### D. Therapeutic Effects

Methods of treatment and therapeutic regimens disclosed herein that combine a nuclear export inhibitor with an oncolytic virus can exhibit surprising and unexpected therapeutic effects.

In some embodiments, a method of treatment or therapeutic regimen disclosed herein that comprises a nuclear export inhibitor and an oncolytic virus (e.g., MYXV) is effective to reduce cancer load by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 2-fold, at least about 3-fold, at least about 5-fold, at least about 10-fold, at least about 20-fold, at least about 30-fold, or at least about 50-fold, e.g., relative to before the treatment or therapeutic regimen. The reduction in cancer load can be an average reduction in cancer load as determined by a cohort study. The cancer load can comprise or can be a tumor volume, for example, a volume of one tumor or a volume of multiple tumors. The cancer load can comprise or can be a concentration of circulating hematological cancer cells.

In some embodiments, a method of treatment or therapeutic regimen disclosed herein that comprises a nuclear export inhibitor and an oncolytic virus (e.g., MYXV) is effective to reduce cancer growth at a site distal from the site of administration by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 2-fold, at least about 3-fold, at least about 5-fold, at least about 10-fold, at least about 20-fold, at least about 30-fold, or at least about 50-fold, e.g., relative to before the treatment or therapeutic regimen. The reduction in cancer growth at a distal site can be as determined by a cohort study. In some embodiments, the oncolytic virus (e.g., MYXV) is administered locally and nonetheless reduces cancer growth at the distal site.

In some embodiments, a method of treatment or therapeutic regimen disclosed herein that comprises a nuclear export inhibitor and an oncolytic virus (e.g., MYXV) is effective to reduce incidence of metastasis at distal sites from the site of administration by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 2-fold, at least about 3-fold, at least about 5-fold, at least about 10-fold, at least about 20-fold, at least about 30-fold, or at least about 50-fold, e.g., relative to before the treatment or therapeutic regimen. The reduction in metastasis at distal sites can be as determined by a cohort study. In some embodiments, the oncolytic virus (e.g., MYXV) is administered locally and nonetheless reduces metastasis at the distal sites.

In some embodiments, a method of treatment or therapeutic regimen disclosed herein that comprises a nuclear export inhibitor and an oncolytic virus (e.g., MYXV) is effective to increase a rate of survival by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80% relative to without the treatment or therapeutic regimen. The increase in survival rate can be as determined by a cohort study.

In some embodiments, a method of treatment or therapeutic regimen disclosed herein that comprises a nuclear export inhibitor and an oncolytic virus (e.g., MYXV) is effective to reduce cancer load by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 2-fold, at least about 3-fold, at least about 5-fold, at least about 10-fold, at least about 20-fold, at least about 30-fold, or at least about 50-fold, relative to an otherwise comparable treatment regimen that lacks the nuclear export inhibitor. The reduction in cancer load can be an average reduction in cancer load as determined by a cohort study. The cancer load can comprise or can be a tumor volume, for example, a volume of one tumor or a volume of multiple tumors. The cancer load can comprise or can be a concentration of circulating hematological cancer cells.

In some embodiments, a method of treatment or therapeutic regimen disclosed herein that comprises a nuclear export inhibitor and an oncolytic virus (e.g., MYXV) is effective to reduce cancer growth at a site distal from the site of administration by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 2-fold, at least about 3-fold, at least about 5-fold, at least about 10-fold, at least about 20-fold, at least about 30-fold, or at least about 50-fold, relative to an otherwise comparable treatment regimen that lacks the nuclear export inhibitor. The reduction in cancer growth at a distal site can be an average reduction in cancer growth at the distal site as determined by a cohort study. In some embodiments, the oncolytic virus (e.g., MYXV) is administered locally and nonetheless reduces cancer growth at the distal site.

In some embodiments, a method of treatment or therapeutic regimen disclosed herein that comprises a nuclear export inhibitor and an oncolytic virus (e.g., MYXV) is effective to reduce incidence of metastasis at distal sites from the site of administration by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 2-fold, at least about 3-fold, at least about 5-fold, at least about 10-fold, at least about 20-fold, at least about 30-fold, or at least about 50-fold, relative to an otherwise comparable treatment regimen that lacks the nuclear export inhibitor. The reduction in metastasis incidence at distal sites can be as determined by a cohort study. In some embodiments, the oncolytic virus (e.g., MYXV) is administered locally and nonetheless reduces metastasis at the distal sites.

In some embodiments, a method of treatment or therapeutic regimen disclosed herein that comprises a nuclear export inhibitor and an oncolytic virus (e.g., MYXV) is effective to prolong survival by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 2-fold, at least about 3-fold, at least about 5-fold, at least about 10-fold, at least about 20-fold, at least about 30-fold, or at least about 50-fold relative to an otherwise comparable treatment regimen that lacks the nuclear export inhibitor. The prolonged survival can be an increase in average survival as determined by a cohort study.

In some embodiments, a method of treatment or therapeutic regimen disclosed herein that comprises a nuclear export inhibitor and an oncolytic virus (e.g., MYXV) is effective to increase a rate of survival by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80% relative to an otherwise comparable treatment regimen that lacks the nuclear export inhibitor. The increase in survival rate can be as determined by a cohort study.

In some embodiments, a method of treatment or therapeutic regimen disclosed herein that comprises a nuclear export inhibitor and an oncolytic virus (e.g., MYXV) is effective to reduce cancer load by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 2-fold, at least about 3-fold, at least about 5-fold, at least about 10-fold, at least about 20-fold, at least about 30-fold, or at least about 50-fold, relative to an otherwise comparable treatment regimen that lacks the oncolytic virus (e.g., lacks the MYXV). The reduction in cancer load can be an average reduction in cancer load as determined by a cohort study. The cancer load can comprise or can be a tumor volume, for example, a volume of one tumor or a volume of multiple tumors. The cancer load can comprise or can be a concentration of circulating hematological cancer cells.

In some embodiments, a method of treatment or therapeutic regimen disclosed herein that comprises a nuclear export inhibitor and an oncolytic virus (e.g., MYXV) is effective to reduce cancer growth at a site distal from the site of administration by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 2-fold, at least about 3-fold, at least about 5-fold, at least about 10-fold, at least about 20-fold, at least about 30-fold, or at least about 50-fold, relative to an otherwise comparable treatment regimen that lacks the oncolytic virus (e.g., lacks the MYXV). The reduction in cancer growth at a distal site can be an average reduction in cancer growth at the distal site as determined by a cohort study. In some embodiments, the oncolytic virus (e.g., MYXV) is administered locally and nonetheless reduces cancer growth at the distal site.

In some embodiments, a method of treatment or therapeutic regimen disclosed herein that comprises a nuclear export inhibitor and an oncolytic virus (e.g., MYXV) is effective to reduce incidence of metastasis at distal sites from the site of administration by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 2-fold, at least about 3-fold, at least about 5-fold, at least about 10-fold, at least about 20-fold, at least about 30-fold, or at least about 50-fold, relative to an otherwise comparable treatment regimen that lacks the oncolytic virus (e.g., lacks the MYXV). The reduction in metastasis incidence at distal sites can be as determined by a cohort study. In some embodiments, the oncolytic virus (e.g., MYXV) is administered locally and nonetheless reduces metastasis at the distal sites.

In some embodiments, a method of treatment or therapeutic regimen disclosed herein that comprises a nuclear export inhibitor and an oncolytic virus (e.g., MYXV) is effective to prolong survival by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 2-fold, at least about 3-fold, at least about 5-fold, at least about 10-fold, at least about 20-fold, at least about 30-fold, or at least about 50-fold relative to an otherwise comparable treatment regimen that lacks the oncolytic virus (e.g., lacks the MYXV). The prolonged survival can be an increase in average survival as determined by a cohort study.

In some embodiments, a method of treatment or therapeutic regimen disclosed herein that comprises a nuclear export inhibitor and an oncolytic virus (e.g., MYXV) is effective to increase a rate of survival by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80% relative to an otherwise comparable treatment regimen that lacks the oncolytic virus (e.g., lacks the MYXV). The increase in survival rate can be as determined by a cohort study.

In some embodiments, the use of the nuclear export inhibitor increases the replication of the oncolytic virus (e.g., MYXV) in the cancer cell or cancer tissue. In some embodiments, the oncolytic virus is replicated at a rate that is at least 5%, at least 10%, at least 20%, at least 30%, at least 40%, at least 50%, at least 60%, at least 70%, at least 80%, or at least 90% faster than the replication rate in the absence of the nuclear export inhibitor. In some embodiments, the oncolytic virus is replicated at a rate that is at least 1.5 times, 2 times, 2.5 times, 3 times, 3.5 times, 4 times, 5 times, 6 times, 7 times, 8 times, 9 times, or 10 times faster than the replication rate in the absence of the nuclear export inhibitor. In some embodiments, the nuclear export inhibitor increases the replication of the oncolytic virus in the cancer cell or cancer tissue to a viral load that is at least 30%, at least 50%, at least 70%, at least 90%, at least 2 fold, at least 3 fold, at least 5 fold, at least 7 fold, at least 9 fold, at least 10 fold, at least 12 fold, or at least 15 fold higher than in the absence of the nuclear export inhibitor after 24 hours, 48 hours, 72 hours, or 96 hours post infection. In some embodiments, the nuclear export inhibitor increases the replication of the oncolytic virus in the cancer cell or cancer tissue by no more than 5 fold, no more than 10 fold, no more than 20 fold, no more than 50 fold or no more than 100 fold after 24 hours, 48 hours, 72 hours, or 96 hours post infection.

In some embodiments, the oncolytic virus is replicated at a rate that is at least 20%, at least 30%, at least 40%, at least 50%, at least 60%, at least 70%, at least 80%, or at least 90% faster than the replication rate prior to administering the nuclear export inhibitor. In some embodiments, the oncolytic virus is replicated at a rate that is at least 1.5 times, 2 times, 2.5 times, 3 times, 3.5 times, 4 times, 5 times, 6 times, 7 times, 8 times, 9 times, or 10 times faster than the replication rate prior to administering the nuclear export inhibitor.

In some embodiments, the use of the nuclear export inhibitor in combination with an oncolytic virus reduces the viability of the cancer cell or cancer tissue compared to the use of the oncolytic virus in the absence of the nuclear export inhibitor. In some embodiments, the cell viability is reduced by at least about 10%, at least about 20%, at least about 30%, at least about 40%, at least about 50%, at least about 60%, at least about 70%, at least about 80%, or at least about 90%. In some embodiments, the cell viability is reduced by at least about 10%, at least about 20%, at least about 30%, at least about 40%, at least about 50%, at least about 60%, at least about 70%, at least about 80%, at least about 90%, at least 2 about fold, at least about 3 fold, at least about 5 fold, at least about 7 fold, at least about 9 fold, or at least about 10 fold more compared to the cell viability when using the oncolytic virus in the absence of the nuclear export inhibitor.

### E. Cohort Studies

In some embodiments, an effect (e.g., therapeutic effect) disclosed herein can be determined in a cohort study. The cohort study can utilize groups of suitable sizes to determine the effect of a treatment on a therapeutic parameter, for example, cancer load, tumor volume, concentration of circulating hematological cancer cells, cancer growth (e.g., at a distal site from the site of administration), metastasis incidence, duration of survival, rate of survival, and the like). The groups can be matched, e.g., by age, sex, and/or disease staging.

Each cohort or group can comprise a suitable number of subjects for determining the effect of a treatment on the therapeutic parameter, for example, a cohort or group can comprise at least 3, at least 4, at least 5, at least 6, at least 7, at least 8, at least 9, at least 10, at least 15, at least 20, at least 25, or at least 50 subjects.

Data can be collected at a suitable timepoint for evaluation of an effect on the therapeutic parameter at any suitable amount of time, for example, about 1 hour, about 12 hours, about 24 hours, about 48 hours, about 3 days, about 4 days, about 5 days, about 7 days, about 10 days, about 14 days, about three weeks, about four weeks, about five weeks, about six weeks, about eight weeks, about ten weeks, about 12 weeks, about 15 weeks, about 26 weeks, or about 52 weeks after a first dose or last dose of a nuclear export inhibitor or oncolytic virus.

### IV. PHARMACEUTICAL COMPOSITIONS

Disclosed herein are pharmaceutical compositions comprising an oncolytic virus, a nuclear export inhibitor, a pharmaceutically acceptable excipient or carrier, or a combination thereof. When administered as a combination, the therapeutic agents (i.e., the oncolytic virus and the nuclear export inhibitor) can be formulated as separate compositions that are given at the same time or different times, or the therapeutic agents can be formulated as a single composition.

To prepare the pharmaceutical compositions according to the present disclosure, a therapeutically effective amount of one or more of the therapeutic agents can be admixed with a pharmaceutically acceptable carrier according to conventional pharmaceutical compounding techniques to produce a dose. A carrier may take a wide variety of forms depending on the form of preparation desired for administration, e.g., oral, topical ocular, or parenteral, including gels, creams ointments, lotions and time released implantable preparations, among numerous others. In certain embodiments, the pharmaceutically acceptable carrier is an aqueous solvent, i.e., a solvent comprising water, optionally with additional co-solvents. Exemplary pharmaceutically acceptable carriers include water, buffer solutions in water (such as phosphate-buffered saline (PBS)), and sugar alcohols such as sorbitol. Compositions suitable for parenteral administration can be enclosed in ampoules, disposable syringes or multiple dose vials made of glass or plastic. In some embodiments, formulations suitable for parenteral administration comprise aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described.

In some embodiments, the nuclear export inhibitor is administered as a pharmaceutically acceptable salt, complex, or prodrug. Pharmaceutically acceptable salts or complexes can refer to appropriate salts or complexes of the active compounds according to the present disclosure which retain the desired biological activity of the parent compound and exhibit limited toxicological effects to normal cells. Non-limiting examples of such salts are (a) acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, and polyglutamic acid, among others; (b) base addition salts formed with metal cations such as zinc, calcium, sodium, potassium, and the like, among numerous others.

In some embodiments, the pharmaceutical composition is in a unit dosage form. In some embodiments, the unit dosage form is suitable to be administered orally. In some embodiments, the unit dosage form is suitable to be administered topically. In some embodiments, the unit dosage form is suitable to be administered intratumorally or parenterally, e.g., intravenously. Unit dosage formulations can be those containing a dose or unit, e.g., daily dose, two or three times daily dose, daily sub-dose, a weekly dose or unit, or an appropriate fraction thereof, of the administered ingredient. In some embodiments, a unit dose comprises from about 0.1 mL to about 100 mL deliverable volume, and/or ranges therebetween. In some embodiments, a unit dose comprises from about 0.5 mL to about 5 mL, from about 0.75 mL to about 2.5 mL, from about 0.9 mL to about 1.1 mL deliverable volume. In some embodiments, a unit dose comprises about 0.5 mL, about 1 mL, about 1.5 mL, or about 2 mL deliverable volume.

In some embodiments, a unit dose comprises from about 1×10³ plaque-forming units (FFU) to about 1×10¹⁰ FFU of the oncolytic virus per mL, and/or ranges therebetween. In some embodiments, a unit dose comprises from about 1×10⁴ FFU to about 1×10⁹ FFU or from about 1×10⁶ FFU to about 1×10⁸ FFU of the oncolytic virus per mL, and/or ranges therebetween. In some embodiments, a unit dose comprises from about 1×10⁵ FFU to about 1×10¹⁰ FFU, from about 1×10⁶ FFU to about 1×10¹⁰ FFU, from about 1×10⁵ FFU to about 1×10¹¹ FFU, from about 1×10⁵ FFU to about 1×10⁹ FFU, from about 1×10⁶ FFU to about 1×10⁹ FFU, or from about 1×10⁷ FFU to about 1×10⁸ FFU of the oncolytic virus per mL, and/or ranges therebetween.

In some embodiments, a unit dose comprises about 20 mg-100 mg, about 20-60 mg, or about 60 mg-100 mg of a nuclear export inhibitor. In a unit dose is per kilogram of the subject's body weight, for example, about 0.001-1000 mg/kg, about 0.01-100 mg/kg, about 5-20 mg/kg or about 0.01-10 mg/kg of a subject's body weight.

As used herein, the singular forms “a,” “an,” and “the” are intended to include the plural forms as well, unless the context clearly indicates otherwise.

As used herein, the term “and/or” refers to and encompasses any and all possible combinations of one or more of the associated listed items, as well as the lack of combinations when interpreted in the alternative (“or”).

### V. EXAMPLES

The following examples are included for illustrative purposes only and are not intended to limit the scope of the disclosure.

Oncolytic viruses (OVs) have emerged as novel anti-cancer immunotherapies for treating standard therapy-resistant and metastatic cancers (Rahman, M. M., et al., 2021, Cancers (Basel), 13(21):5452; Zhang, S., et al., 2020, Expert Opinion on Drug Discovery, 16(4):391-410; Kaufman, H. L., et al., Nature Reviews Drug Discovery, 14:642-662). An ideal replication-competent OV is expected to selectively infect, replicate, and generate progeny virions in infected cancer cells, which subsequently infect neighboring cancer cells in the tumor bed (Bell, J., et al., 2014, Cell Host Microbe, 15:260-265; Davola, M. E., et al., 2019, Oncoimmunology, 8:e1581528). This successful replication of OVs is thought to mediate antitumoral activity in multiple ways, such as direct killing of infected cancer cells, exposing and presenting novel tumor-specific neoantigens, activation of systemic antitumor and antiviral immunity, and recruitment of activated immune cells to the tumor microenvironment (TME) (Boagni, D. A., et al., 2021, Molecular Therapy-Oncolytics, 22:98-113; Kooti, W., et al., 2021, Frontiers in Oncology, 11:761015). In addition to their own multi-mechanistic antitumor activity, OVs can be combined with most currently approved cancer therapeutics, such as chemotherapies, immune checkpoint inhibitors, and cell therapies, for additional therapeutic benefits (Malfitano, A. M., et al., 2020, Biochemical Pharmacology, 177:113986; Zheng, N., et al., 2022, Cancer Cell, 40:973-985; Zhang, Y., et al., 2021, Cancer Cell International, 13(7):1271).

Myxoma virus (MYXV) has been developed as an OV against diverse malignancies (Rahman, M. M., et al., 2020, Journal of Clinical Medicine, 9(1):171; Chan, W. M., et al., 2013, Vaccine, 31:4252-4258). MYXV is a prototypic member of the Leporipoxvirus genus in the Poxviridae family of viruses. Different isolates of MYXV cause disease only in European rabbits, but are completely non-pathogenic to all other non-leporid species, including mice and humans. However, MYXV can productively infect many (but not all) classes of human cancer cells originating from different tissues, both in vitro and in vivo. This natural and selective tropism of MYXV for cancer cells and tissues allows its exploitation as an oncolytic virotherapeutic in several preclinical cancer models for various cancer types, such as pancreatic cancer, lung cancer, glioblastoma, ovarian cancer, melanoma, and hematological malignancies (Rahman, M. M., et al., 2020, Journal of Clinical Medicine, 9(1):171; Chan, W. M., et al., 2013, Vaccine, 31:4252-4258; Rahman, M. M., et al., 2010, Cytokine Growth Factor Reviews, 21: 169-175).

Similar to other poxviruses, MYXV can promiscuously bind, enter, and initiate infection in a broad diversity of cancerous and non-cancerous cells from most vertebrate species. However, the ability of MYXV to productively replicate and produce progeny in any cell type outside the rabbit largely depends on whether the virus can successfully overcome diverse intrinsic and innate antiviral cellular barriers (McFadden, G., 2005, Nature Reviews Microbiology, 3:201-213). These barriers are sufficiently robust to restrict MYXV replication post-entry in normal primary somatic human or mouse cells, but tend to become compromised when cells are immortalized, transformed, or cancerous. Thus, unlike rabbit cells, where MYXV can counteract every aspect of these cellular barriers, in non-rabbit normal cells and a subset of cancer cells, complete replication of MYXV can be restricted to different levels by multiple factors. In human cancer cells, activation of these intrinsic cellular restriction factors and virus-induced signaling pathways can limit the replication and oncolytic ability of MYXV in specific cancer cell types, referred to as either non-permissive or semi-permissive. Several cellular pathways that are currently known to contribute to MYXV's ability of MYXV to replicate in human cancer cells include i) endogenously activated protein kinase B (PKB)/AKT, ii) antiviral pathway activated by protein kinase R (PKR), iii) status of tumor suppressors such as p53, retinoblastoma (Rb), and ataxia-telangiectasia (ATM), and iv) antiviral states induced by interferons (IFNs) or tumor necrosis factor (TNF) (Wang, G., et al., 2006, Proceedings of the National Academy of Sciences USA, 103:4640-4645; Rahman, M. M., et al., 2013, PLOS Pathology, 9:e1003465; Kim, M., et al., 2010, Oncogene, 29:3990-3996; Bartee, E., et al., 2009, Cytokine, 47:199-205). In addition to these cellular barriers, it has been reported that members of the cellular DEAD-box RNA helicase superfamily have potent antiviral and/or proviral functions that regulate MYXV replication in diverse human cancer cell types (Rahman, M. M., et al., 2017, Scientific Reports, 7:15710). Among these antiviral RNA helicases, it was also reported that RNA helicase A (RHA) or DHX9 exits the nucleus in response to MYXV infection to form unique antiviral granules in the cytoplasm of infected human cancer cells. These antiviral granules are formed during the late replication phase of MYXV, which reduces MYXV late protein synthesis and limits MYXV replication and the generation of progeny virions (Rahman, M. M., et al., 2021, Journal of Virology, 95:e0015121). Furthermore, DHX9 knockdown significantly enhanced MYXV replication in both semi-permissive and non-permissive human cancer cell lines.

Here, it is investigated if inhibition of the nuclear export pathway in diverse human cancer cell types, where MYXV replication is restricted, enhances virus replication and progeny virus formation by reducing the appearance of cytoplasmic antiviral granules. The FDA-approved nuclear export inhibitor selinexor also significantly enhanced MYXV replication in diverse human cancer cells. A combination of selinexor and MYXV treatment significantly reduced cancer cell proliferation and enhanced cell death. Furthermore, using 3D spheroid cultures of human cancer cells, it was showed that selinexor enhanced MYXV replication and penetrative spread in spheroid cultures of cancer cells. Human cancer cell-derived xenograft (CDX) models were tested in NSG mice to determine the in vivo effect of selinexor on oncolytic MYXV replication. Similar to in vitro cultures, selinexor enhanced MYXV gene expression and replication in Colo205 and HT29 cell-derived CDX models in NSG mice. In addition, using PANC-1 cell-derived CDX models, it was shown that selinexor plus MYXV treatment significantly reduced the tumor burden compared to the control or MYXV treatments. Furthermore, selinexor plus MYXV treatment significantly enhanced the survival of the mice. These results suggest that selinexor and the oncolytic MYXV can be developed as a novel combination therapy for cancer.

### VI. EXAMPLE 1: NUCLEAR EXPORT INHIBITOR SELINEXOR ENHANCE MYXV GENE EXPRESSION AND REPLICATION IN HUMAN CANCER CELL LINES

This example demonstrates that the nuclear export pathway can be targeted to enhance MYXV replication in semi-permissive or non-permissive human cancer cells. MXYC infection of human cancer cells results in the formation of cytosolic antiviral granules composed of RNA helicase DHX9 and reduced MYXV replication (Rahman, M. M., et al., 2021, Journal of Virology, 95:e0015121). Knockdown of DHX9 significantly enhances MYXV replication and progeny virus production in cancer cells, where viral replication is restricted. In uninfected cells, DXH9 was mainly localized in the nucleus; however, in MYXV-infected cells, DHX9 was detected in the cytoplasm associated with antiviral granules. Nuclear export and import pathways play major roles in the localization and function of many cellular proteins, such as RNA helicases (Mor, A., et al., 2014, Current Opinion in Cell Biology, 28:28-35; Sloan, K. E., 2016, Journal of Molecular Biology, 428:2050-2059).

Inhibitors that target the CRM1/XPO1 mediated nuclear export pathway were examined for their ability to block the formation of DHX9 antiviral granules in the cytoplasm. Initially, the effect of leptomycin B (LMB) on MYXV replication in human cancer cells, such as PANC-1 (pancreatic cancer cell line) and HT29 (colorectal cancer cell line) was examined.

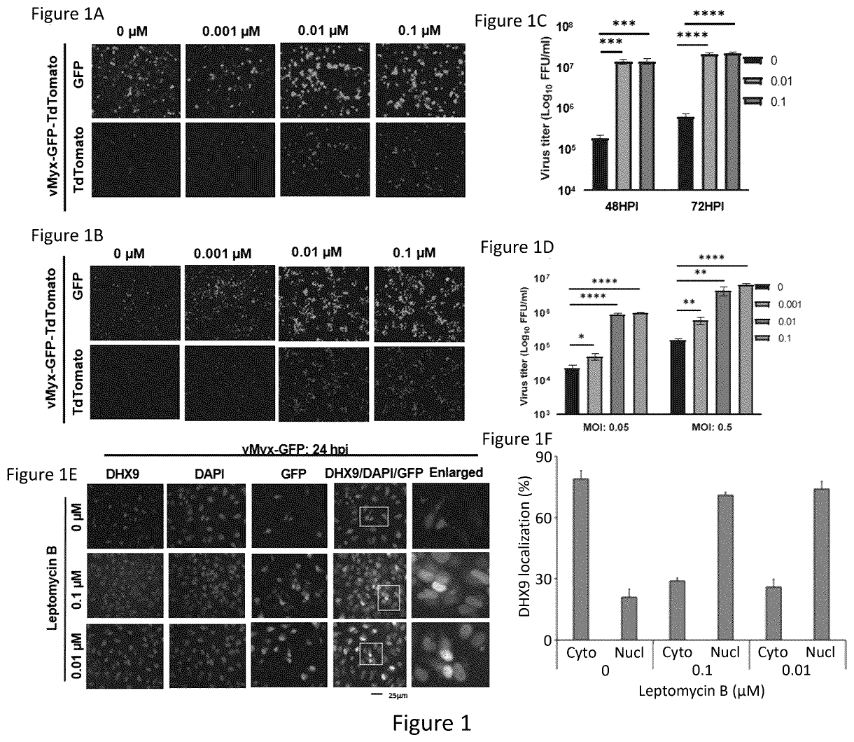

In these cell lines, pretreatment with a lower concentration of LMB (0.001-0.1 μM) enhanced viral gene expression, as observed with increased early/late GFP and late TdTomato reporter protein expression (FIG. 1A and FIG. 1B). This increased viral protein expression and significantly enhanced viral production, as measured by a titration assay (FIG. 1C and FIG. 1D). In addition to LMB, a similar effect on MYXV replication was observed with related SINEs (selected inhibitors of nuclear export), such as leptomycin A, ratjodone A, and anguinomycin A. Immunofluorescence staining of cells with anti-DHX9 antibody revealed that in most LMB plus MYXV-treated cells (>80%), DHX9 remained in the nucleus and blocked the formation of antiviral granules (FIG. 1E and FIG. 1F).

Another nuclear export inhibitor called selinexor was developed as a less toxic SINE for the inhibition of the CRM1/XPO1 mediated nuclear export pathway (Mutka, S. C., et al., 2009, Cancer Research, 69:510-517). As such, Selinexor was selected for future examination, and was tested in multiple human cancer cell lines, including colorectal adenocarcinoma cell line Colo 205, pancreatic cancer cell line PANC-1, human colon cancer cell line HCT116, and melanoma cell line MDA-MB-435, in which MYXV replicates poorly. Different concentrations of selinexor (KPT330) were tested to observe the effect on MYXV gene expression and replication.

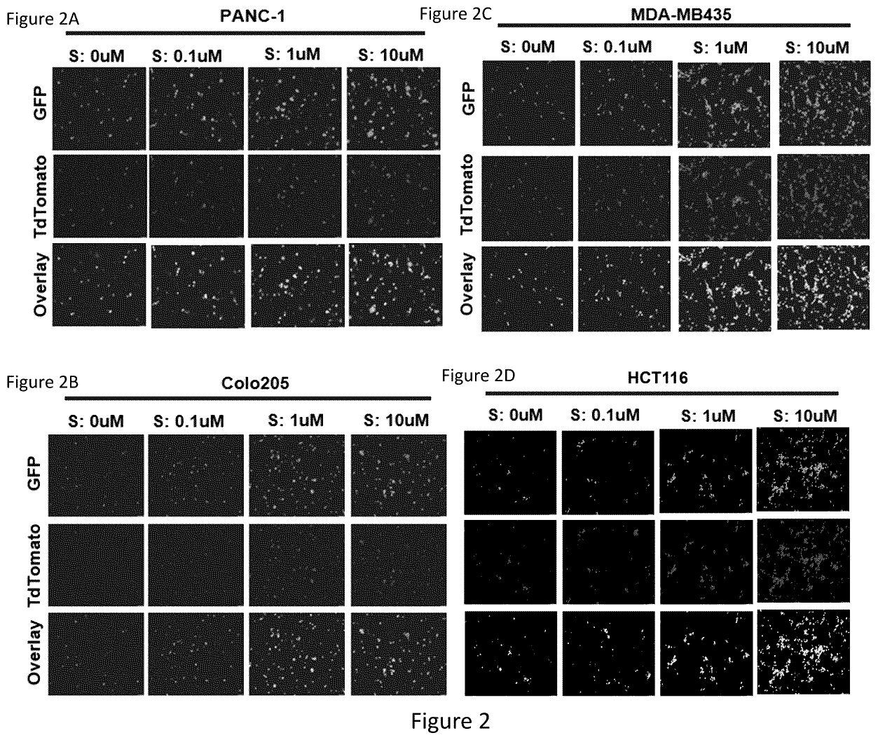

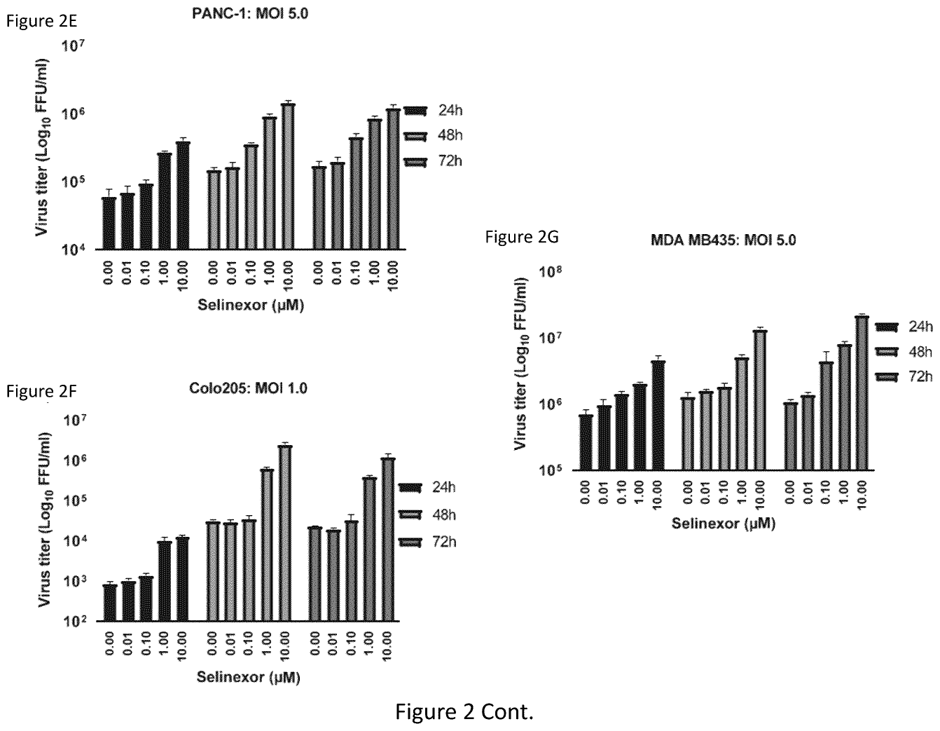

Human PANC-1 (FIG. 2A), Colo205 (FIG. 2B), MDA-MB435 (FIG. 2C), and HCT116 (FIG. 2D) cells were mock-treated or pre-treated with different concentrations of selinexor (0.1-10 μM) for one hour, and then infected with vMyx-GFP-TdTomato (wild type MYXV expressing GFP under poxvirus synthetic early/late promoter and TdTomato under poxvirus p11 late promoter) at a multiplicity of infection (MOI) of 5. The cells were observed under a fluorescence microscope at 48 hours post-infection to monitor the expression of GFP (expressed at early and late stages of the MYXV replication cycle) and TdTomato (expressed late in the MYXV replication cycle) for the progression of virus infection.

Enhanced GFP and TdTomato reporter protein expression was observed in the tested human cancer cell lines treated with Selinexor, indicating enhanced MYXV gene expression and replication. Treatment with Selinexor also allowed the formation of small foci in these restricted human cancer cells lines when infected at low MOI, however at a concentration of 10 μM or higher, selinexor alone caused enhanced cell death in all cancer cell lines tested.

Infected cells were collected at different time points to further assess virus formation and virus titration was performed using permissive rabbit RK13 cells. In PANC-1, Colo205, and MDA-MB435, a significant increase (10-100-fold) in virus production compared to infection with MYXV alone (FIG. 2E through FIG. 2G). These results demonstrate that selinexor enhances MYXV gene expression, replication, viral production where MYXV replication was restricted.

### VII. EXAMPLE 2: CRM1/XPO1 KNOCKDOWN ENHANCES MYXV REPLICATION IN RESTRICTED HUMAN CANCER CELLS AND REDUCES DHX9 GRANULES

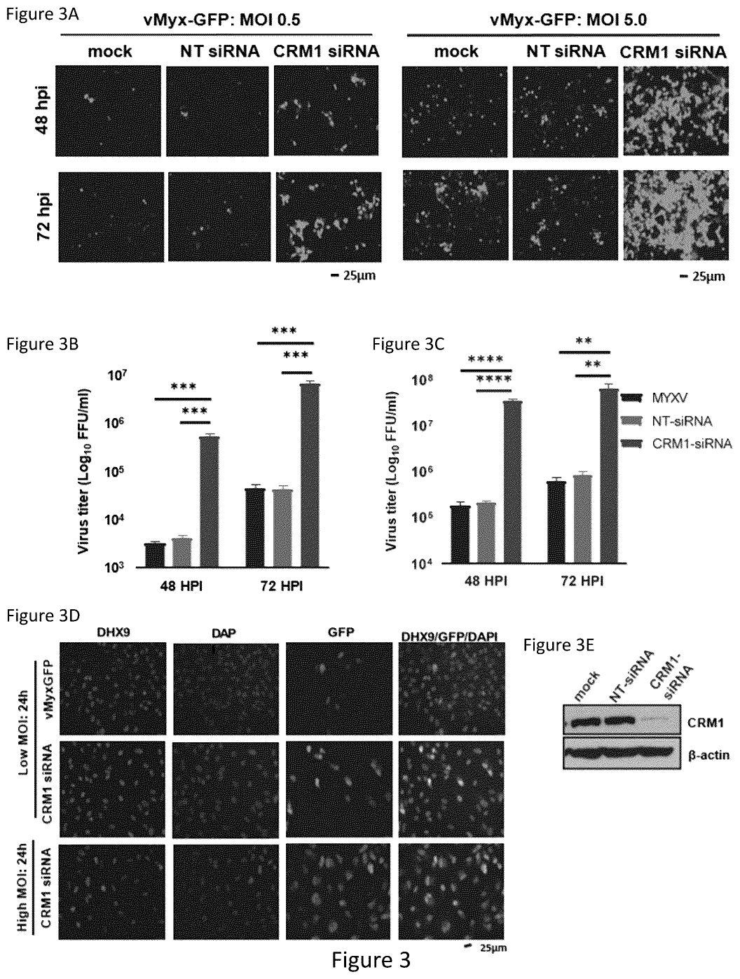

Since inhibition of the CRM1/XPO1 mediated nuclear export pathway via selinexor and other SINEs reduced the formation of DHX9-containing antiviral granules and subsequently enhanced MYXV replication, the observation was examined further by direct knockdown of CRM1 by siRNA. PANC-1 cells were transfected with CRM1 siRNA or control siRNA and infected with vMyx-GFP at an MOI of 0.5 or 5. CRM1 knockdown was confirmed by western blot analysis (FIG. 3E). After CRM1 knockdown, infection with an MOI of 0.5 resulted in the formation of distinct larger foci compared to cells infected with the virus alone or NT-siRNA (FIG. 3A, left top and bottom). Furthermore, infection with an MOI of 5 resulted in significantly higher GFP expression, as observed under the microscope (FIG. 3A, right top and bottom). To measure viral production, virus titrations were performed at 48 and 72 hours post-infection. Infection with an MOI of both 0.5 and 5 resulted in at least a 100-fold increase in virus titer (FIG. 3B and FIG. 3C).

It was then examined whether CRM1 knockdown reduced the formation of DHX9-containing antiviral granules in the cytoplasm after MYXV infection. Immunofluorescence microscopy demonstrated that, following CRM1 knockdown, DHX9 remained in the nucleus when infected at low or high MOI and formation of DHX9-containing granules was prevented (FIG. 3D). The retention of DHX9 in the nucleus also increased the number of GFP-positive infected cells, reflecting enhanced viral infection. These results confirm that the CRM1/XPO1 nuclear export pathway is mainly responsible for the transport of proteins that form antiviral granules and restrict MYXV replication in human cancer cells.

### VIII. EXAMPLE 3: SELINEXOR AND MYXV REDUCE CANCER CELL PROLIFERATION

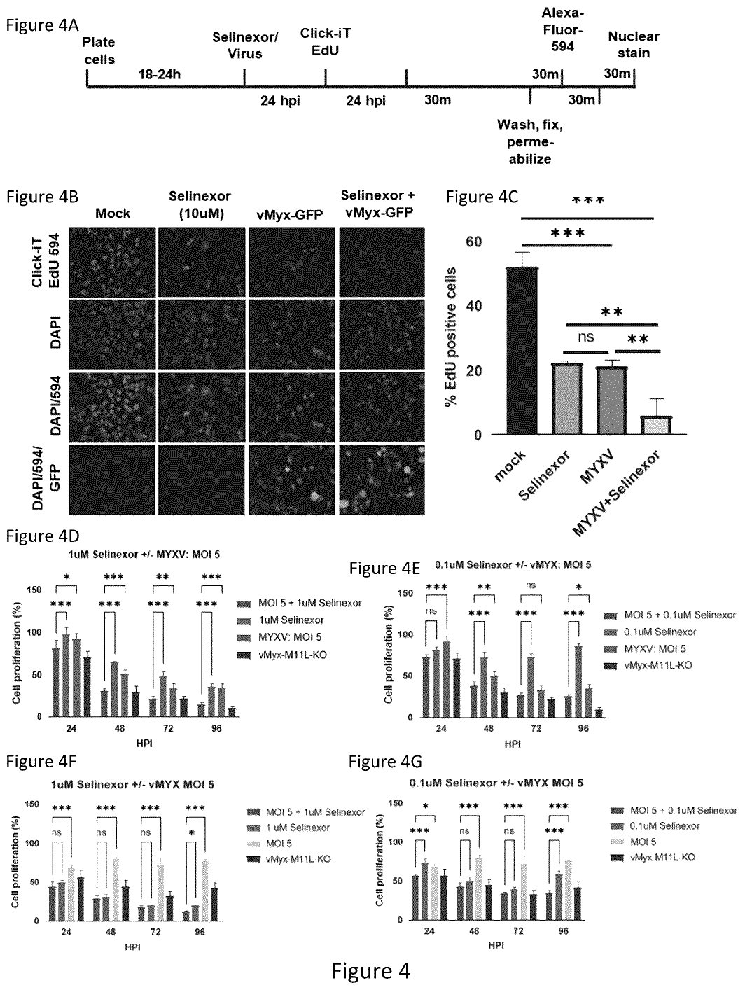

Selinexor was previously reported to reduce the proliferation of cancer cells, however, viral infection also stops cell proliferation (Zheng, Y., et al., 2014, Cancer Chemotherapy and Pharmacology, 74:487-495; Johnston, J. B., et al., 2005, Journal of Virology, 79:10750-10763). As such, the effect of selinexor and MYXV on human cancer cells was examined alone and in combination. To this end, two cell proliferation assays were performed. In the first methods, DNA synthesis was measured by the incorporation of EdU (5-ethynyl-2′-deoxyuridine), a nucleoside analog of thymidine, into DNA during active DNA synthesis (FIG. 4A) (Chehrehasa F., et al., 2009, Journal of Neuroscience Methods, 177:122-130). Using this method in uninfected PANC-1 cells, EdU incorporation was detected in more than 50% of dividing cells (FIG. 4B and FIG. 4C). When cells were treated with selinexor or MYXV for 24 hours, on the other hand, a significant reduction in proliferation was observed (20% EdU-positive cells). To observe the combination of selinexor and MYXV, cells were first treated with selinexor for one hour and then infected with MYXV for 24 hours in the presence of selinexor. This combination further significantly reduced cell proliferation compared to either single treatment and almost completely blocked EdU incorporation (FIG. 4B and FIG. 4C).

To further confirm these observations, a second cell proliferation assay, CyQUANT was performed, which measures DNA content in cells. Proliferation of PANC-1 (FIG. 4D and FIG. 4E) and Colo205 (FIG. 4F and FIG. 4G) cells was measured at different timepoints with different concentrations of selinexor and multiple MOI of MYXV. In this assay, selinexor and MYXV individually resulted in significant reductions in cell proliferation after 24 hours compared to mock treated cells, however, as previously observed, the combination reduced cell proliferation even further. These results demonstrate that the combination of selinexor and MYXV infection can virtually eliminate cancer cell proliferation.

### IX. EXAMPLE 4: SELINEXOR AND MYXV REDUCE CANCER CELL VIABILITY

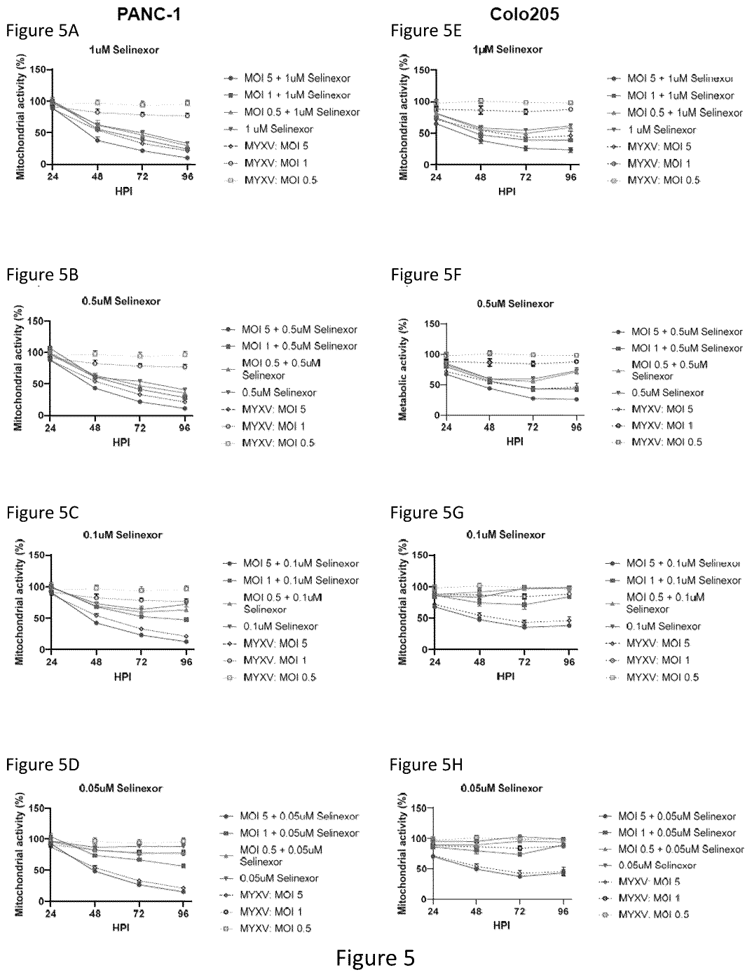

To further assess the inhibition of cell proliferation, and investigate if cell death is enhanced, cell viability assays were performed using an MTS assay to detect metabolic activity in active cells. For this assay, PANC-1 (FIG. 5A through FIG. 5D), Colo205 (FIG. 5E through FIG. 5H), HT29, and MDMB435 cells were treated with different concentrations of selinexor, infected with MYXB at different MOIs, or treated with a combination of selinexor and MYXB, and cell viability was measured at different time points. In all cells tested, when treated with 0.5 μM or 1 μM selinexor, cell viability reduced to 50% over time, however lower concentrations (0.1 μM or 0.05 μM), selinexor had no effect on cell viability. Similarly, MYXV infection at an MOI of 5 resulted in >50% reduced viability in all cell lines, whereas an MOI of 1 or 0.5 had no practical effect on cell viability in the non-permissive cancer cells.

As low MOIs of MYXV and low concentrations of selinexor demonstrated enhanced viral gene expression and replication (FIG. 1 and FIG. 2), combinations of selinexor at different concentrations and MYXV at different MOIs were examined for further reduction in cancer cell viability. Treatment of cells with different concentrations of selinexor with an MYXV MOI of 5 demonstrated significantly enhanced cell death compared to either treatment individually. In particular, cell viability was significantly reduced with 0.1 μM and 0.05 μM selinexor and MOI 5 infection while both selinexor treatments had almost no effect on their own. Similarly, infection with a low MOI of 1 or 0.5 and treatment with 1 M selinexor significantly increased cell death in all tested cell lines. These results suggest that a concentration of selinexor which normally has no effect on cell viability can enhance MYXV replication and reduce cancer cell viability by enhancing the oncolytic effect of MYXV.

### X. EXAMPLE 5: SELINEXOR ENHANCES MYXV REPLICATION IN 3D HUMAN CANCER CELL CULTURES

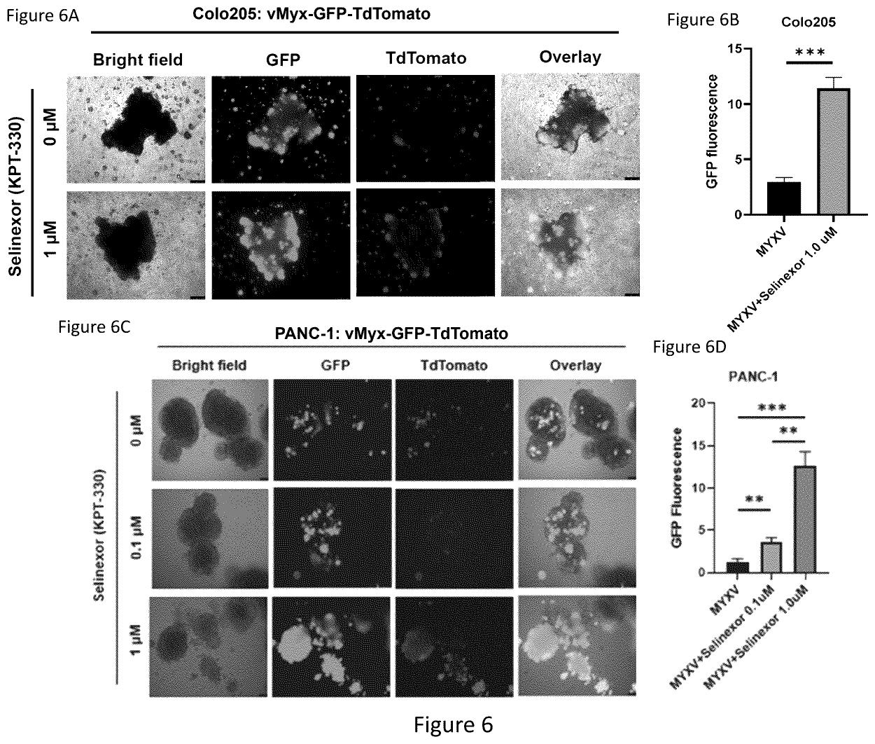

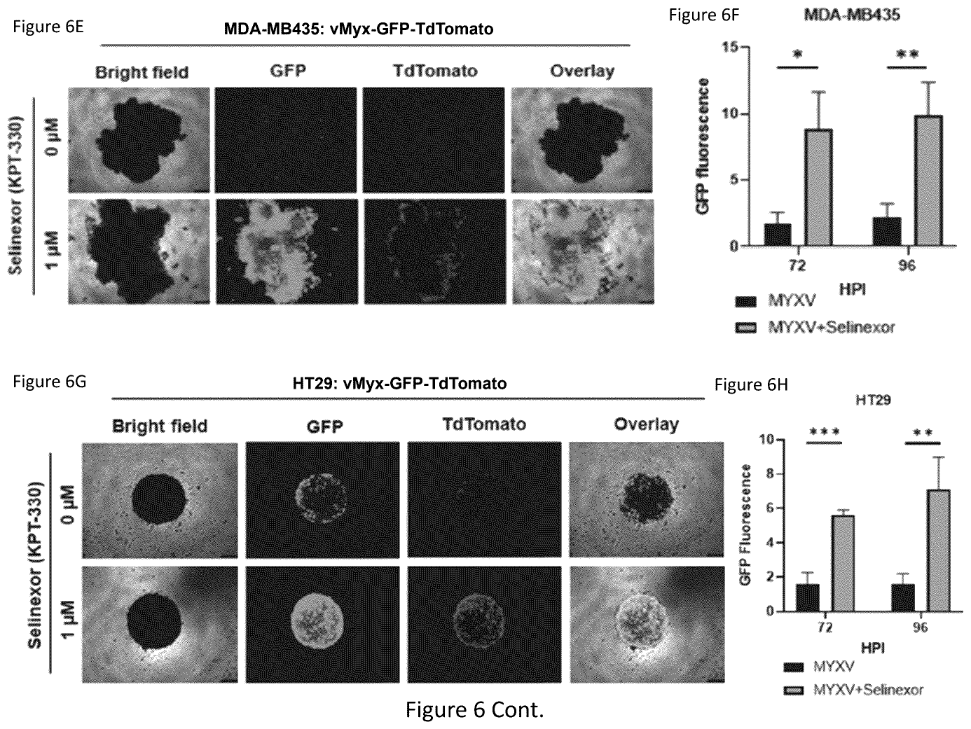

In vitro three-dimensional (3D) cell culture allows cells to contact each other and form a platform representing in vivo tumor masses (Lv, D., et al., 2017, Oncology Letters, 14:6999-7010). As an initial test whether selinexor can enhance MYXV replication in tumor masses, a 3D cell culture using type 1 collagen was established. The semi-permissive and non-permissive human cancer cells PANC-1 (FIG. 6A), MDA-MB435 (FIG. 6C), HT29 (FIG. 6E), and Colo205 (FIG. 6G) were used to form 3D spheroids in 96 well plates. After several days of incubation, the cells formed spheroids.

After reaching a desirable size, spheroids were mock-treated or treated with different concentrations of selinexor (e.g., 0.1 μM, 1 μM) for one hour then infected with vMyx-GFP-TdTomato (1×10⁷ FFU) in the presence of selinexor. Fluorescence microscopy demonstrated that GFP (early/late) and TdTomato (late) expression was enhanced in selinexor-treated spheroids in all tested cells lines (FIG. 6A, FIG. 6C, FIG. 6E, and FIG. 6G). Quantification of GFP fluorescence showed a significant increase in the level of GFP expression in selinexor-treated spheroids (FIG. 6B, FIG. 6D, FIG. 6F, and FIG. 6H). These results confirmed that selinexor enhances MYXV gene expression and replication in 3D spheroid cultures, prompting examination of in vivo models.

### XI. EXAMPLE 6: SELINEXOR ENHANCES MYXV REPLICATION IN XENOGRAFTED HUMAN TUMORS AND REDUCES TUMOR BURDEN

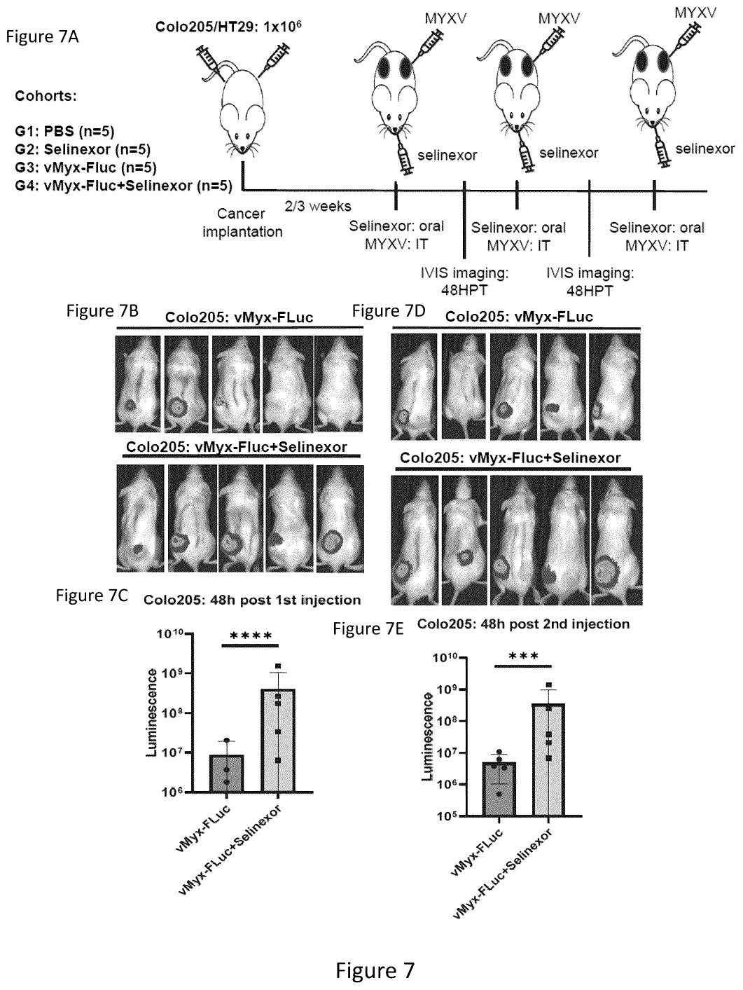

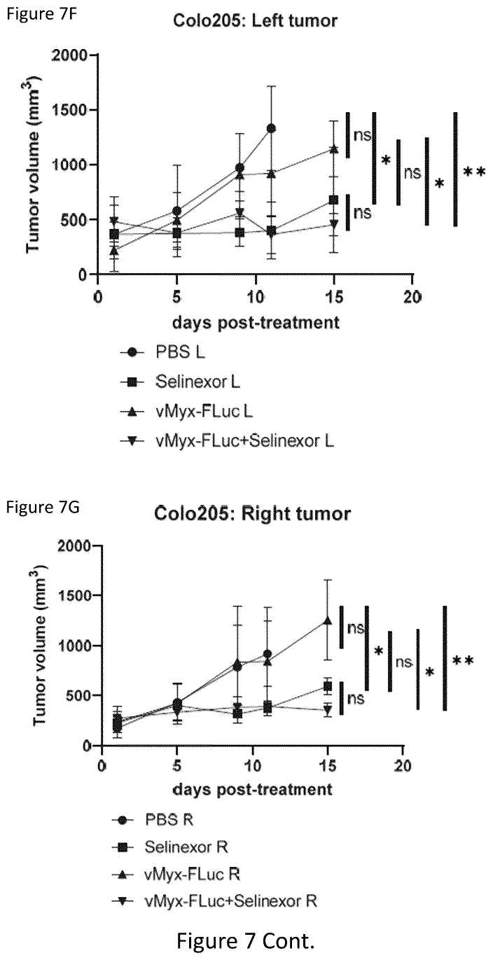

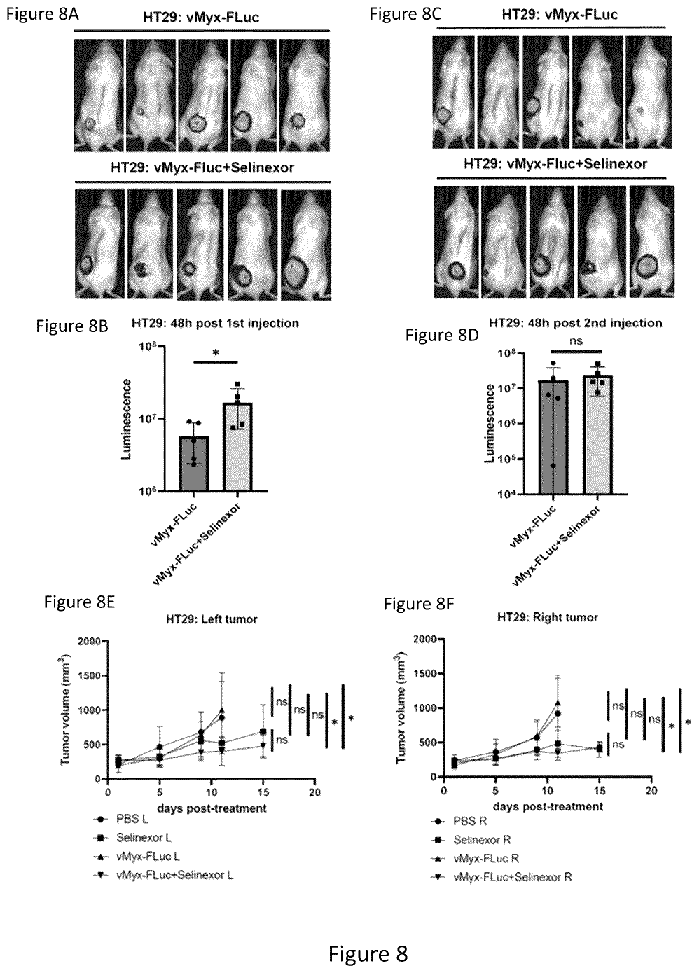

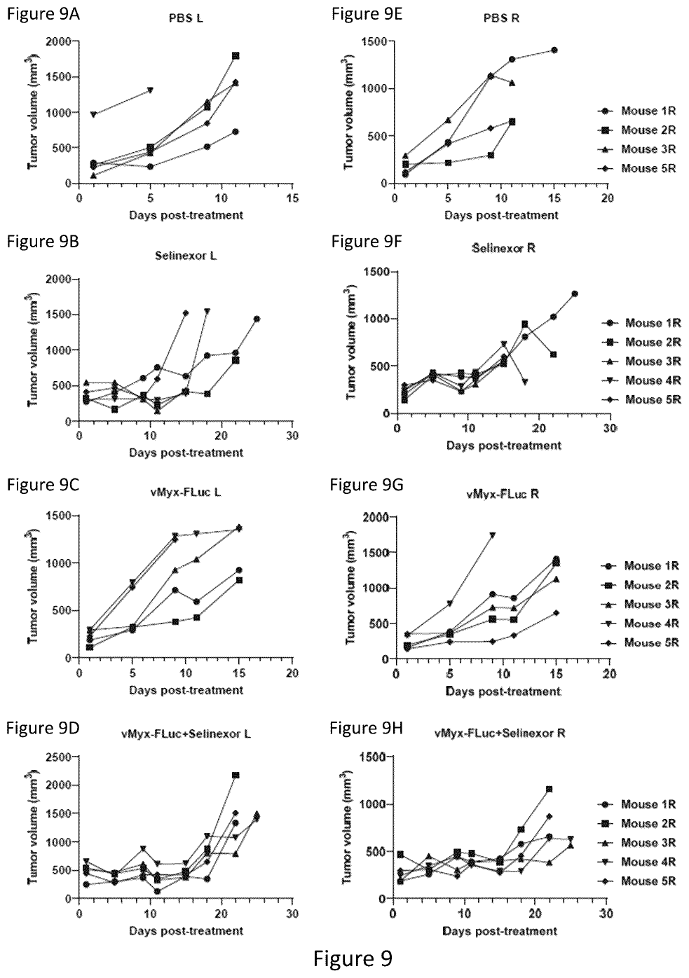

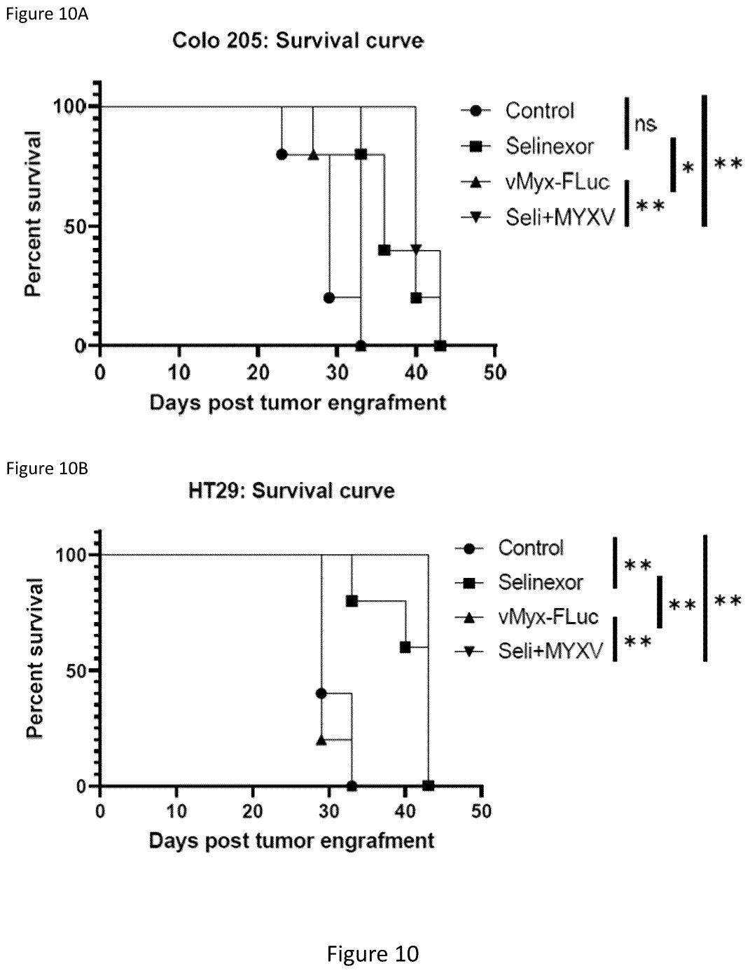

To test the ability of selinexor to enhance MYXV replication in vivo, a xenograft tumor model was established in immunodeficient NSG mice. Human Colo205 (FIG. 7) and HT29 (FIG. 8) cells were injected subcutaneously on both sides of the flank to generate tumors (FIG. 7A). After the tumors size reached between 100-200 mm³ (2-3 weeks), mice were separated into treatment groups to have approximately equal average tumor sizes. Animals (n=5) were treated with PBS (oral and IT right tumor), selinexor (oral, 15 mg/kg), vMyx-Fluc (wild-type MYXV expressing Firefly luciferase and TdTomato; IT left tumor, 1×10⁷ FFU), or selinexor (oral) and vMyx-Fluc (IT left tumor). After 48 hours post-treatment mice were imaged using an IVIS system for luciferase expression. Mice that received selinexor had significantly higher levels of luciferase than those that were injected with the virus alone (FIG. 7B, FIG. 7C, FIG. 8A, and FIG. 8B). Subsequently, mice received 2^{nd} and 3^{rd} doses of selinexor and a second IT injection of MYXV in the same tumor. IVIS imaging of the second treatment still showed a significantly enhanced level of luciferase signal (FIG. 7D, FIG. 7E, FIG. 8C, and FIG. 8D). Additionally, tumor burden was measured on both flanks during the course of treatment (FIG. 7F, FIG. 7G, FIG. 8E, and FIG. 8F). Treatment with selinexor alone, and a combination of selinexor+MYXV significantly reduced tumor burden compared to PBS or MYXV-only treatments on both flanks in Colo205 and HT29 xenograft models. While the size of selinexor+MYXV treated tumors was slightly smaller than selinexor-only tumors, it did not rise to the level of significance (FIG. 9). Following the survival of animals post-tumor engraftment in the different treatment groups, animals treated with selinexor+MYXV survived significantly longer in both tumor models relative to control and MYXV alone (FIG. 10A and FIG. 10B).

### XII. EXAMPLE 7: SELINEXOR WITH MYXV REDUCES TUMOR BURDEN AND EXTENDS SURVIVAL IN PANC-1 XENOGRAFT TUMORS

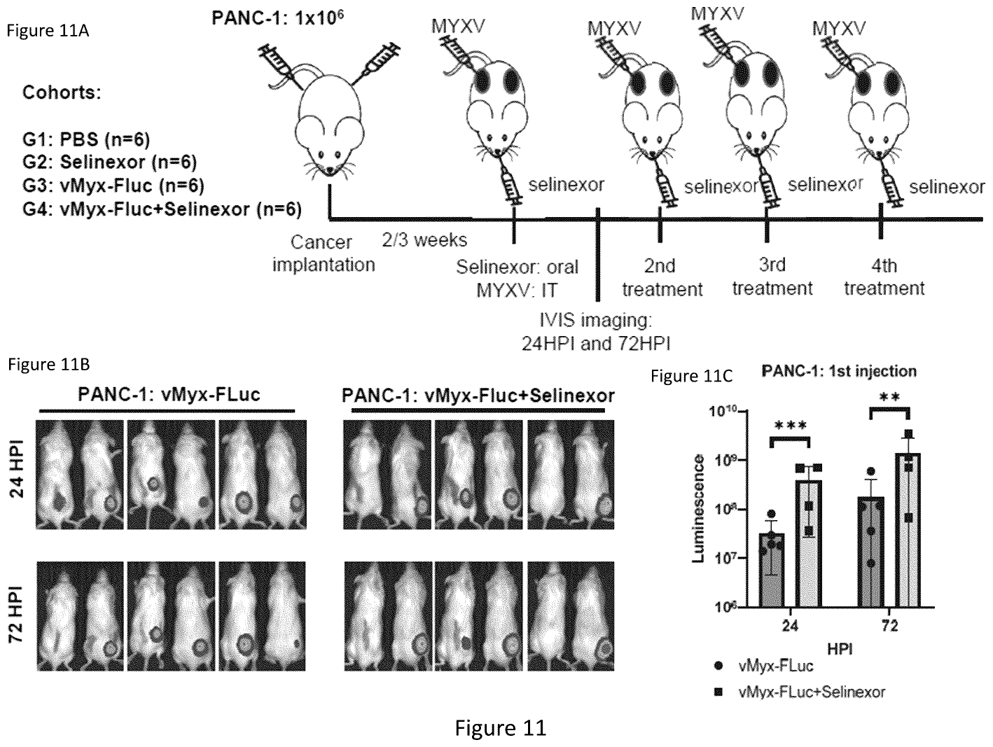

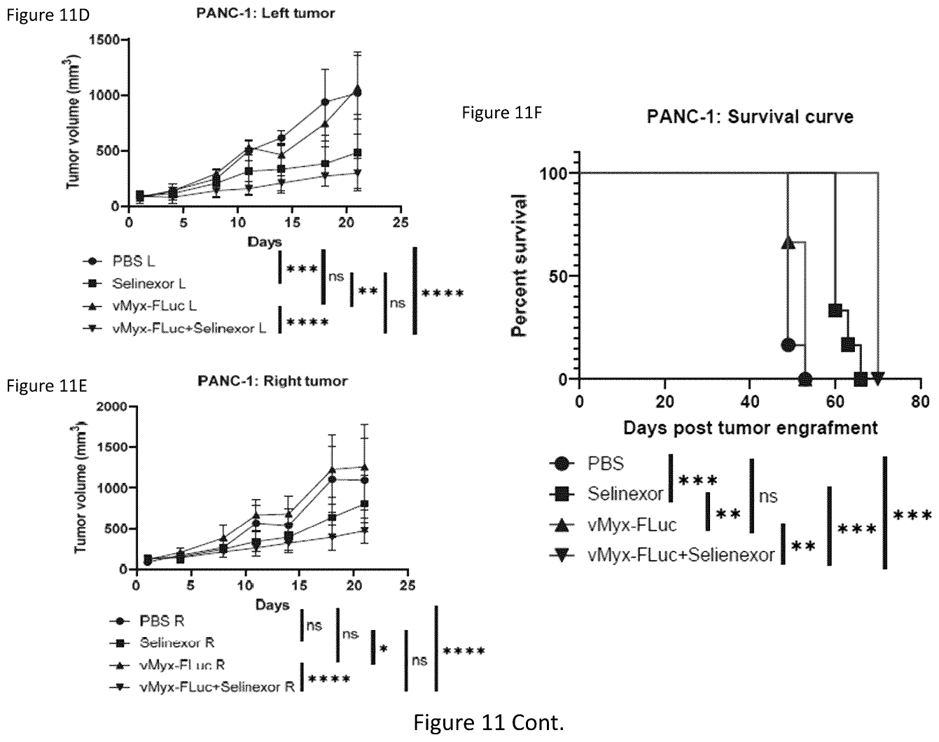

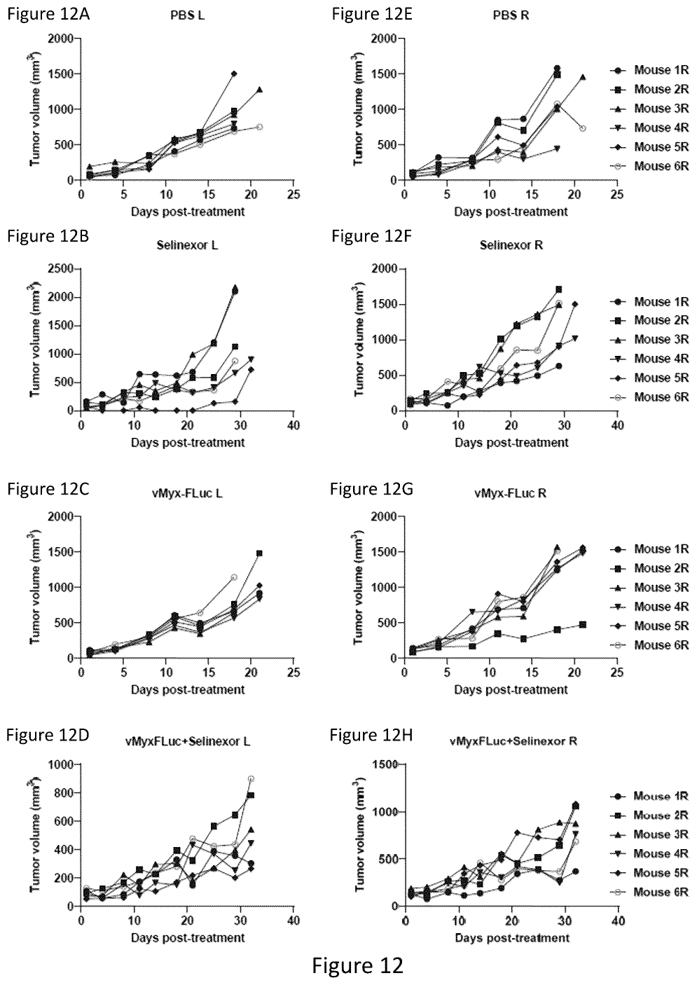

Based on the in vivo results showing selinexor enhances MYXV replication in the tumor bed and reduces the tumor burden in Colo205 and HT29 xenograft tumors, the treatment was examined in PANC-1 xenograft tumors. Human PANC-1 cells were injected subcutaneously on both sides of the flank to generate tumors (FIG. 11A). After the tumor size reached ˜50-100 mm³ (2-3 weeks), animals were randomly assigned to treatment groups to have approximately equal average tumor size. Animals (n=6) were then treated with PBS, selinexor, MYXV, or selinexor+MYXV. Animals received a total of four treatments within the first two weeks and tumor burden was measured 2-3 times every week. After the first treatment, mice were imaged using the IVIS system for luciferase expression at 24 and 72 hours post-treatment (FIG. 11B and FIG. 11C). Mice that received selinexor had significantly higher levels of luciferase signals than those injected with virus alone (FIG. 11C). After imaging and measuring luciferase signals, three additional treatments to test their therapeutic effect on tumor burden and survival were administered. A significant reduction in tumor burden was observed in the PANC-1 xenograft model after treatment with selinexor alone compared to PBS or MYXV alone (FIG. 11D and FIG. 11E). Treatment with selinexor+MYXV significantly reduced the tumor burden compared with PBS or MYXV alone. Although not significant, tumor burden was reduced more in mice treated with selinexor+MYXV over selinexor (FIG. 12). Survival of the animals was followed, and animals treated with selinexor or selinexor+MYXV survived significantly longer than animals treated with PBS or MYXV alone (FIG. 11F). Additionally, mice treated with selinexor+MYXV lived significantly longer than animals treated with selinexor alone.

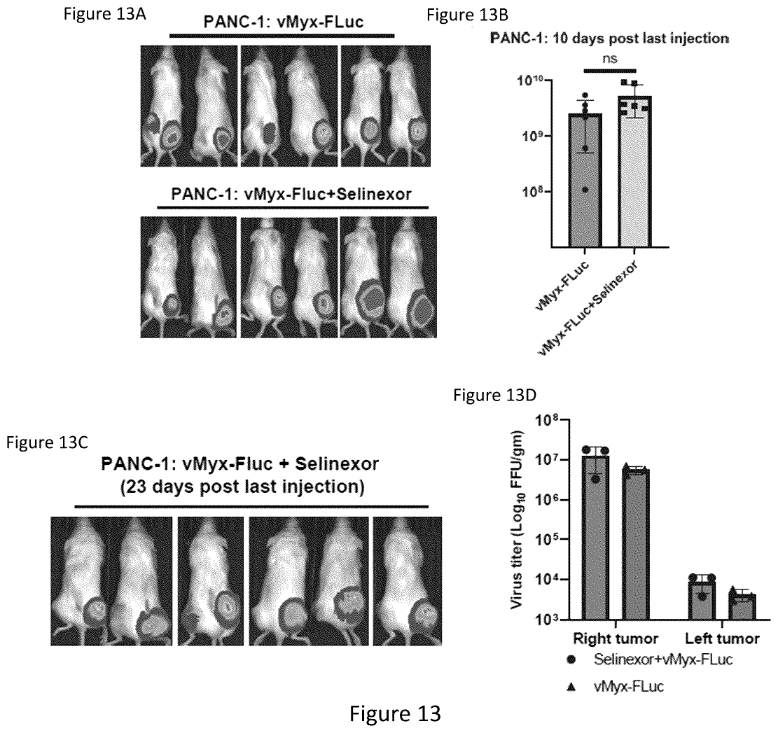

Luciferase signals in animals were also measured before the endpoint to confirm the presence of the virus in the tumor bed after the final (fourth) injection. Mice injected with MYXV alone (10 days after the last injection) showed high luciferase signals (FIG. 13A and FIG. 13B) and mice that received selinexor+MYXV also showed a higher, though not statistically significant, luciferase signals. Luciferase signal was measured again 23 days after the last viral injection, at which point high luciferase activity was still observed in each group (FIG. 13C). At the endpoint, tumors were collected and virus titration assays were performed (FIG. 13D). Interestingly, while luciferase signal was not detected in the un-injected tumor, low levels of the MYXV virus were detected in the un-injected tumor. In both tumors, although not significant, viral load was higher in selinexor+MYXV treated mice than mice treated with MYXV alone.

### XIII. EXAMPLE 8: SELINEXOR AND MYXV REDUCES TUMOR BURDEN IN AN IMMUNOCOMPETENT MOUSE MODEL OF CANCER

This example demonstrates superior efficacy of a combination of a nuclear export inhibitor with a myxoma virus expressing an immunomodulatory transgene, compared to either agent alone, in an immunocompetent animal cancer model.

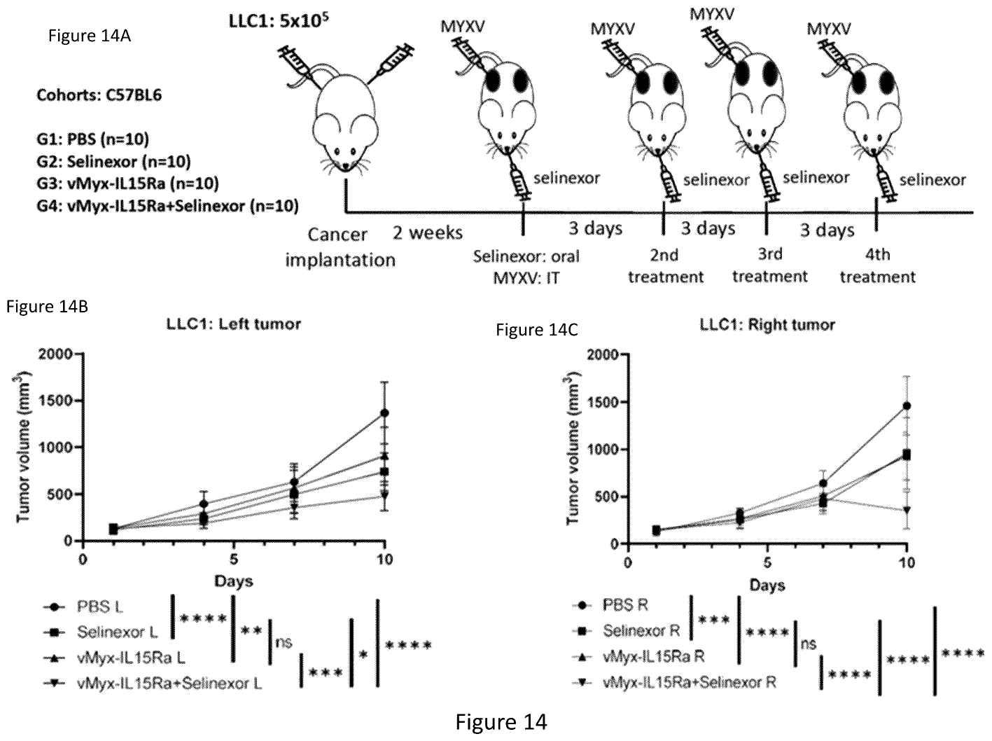

To verify that the combination of selinexor and MYXV is effective even in mice with a competent immune system, C57BL/6 mice were inoculated with mouse Lewis lung carcinoma (LLC1) cells (FIG. 14A). LLC1 cells were subcutaneously injected on both sides of the flank in C57BL/6 mice. Mice were separated into treatment groups and treated with PBS, selinexor, MYXV expressing IL15/IL15Ra (IT, 5×10⁷), or selinexor+MYXV expressing IL15/IL15Ra.

The tumor burden on each flank was measured twice every week. Selinexor or the MYXV alone significantly reduced tumor burden, however the combination therapy of selinexor+MYXV significantly reduced tumor burden to a greater degree than selinexor or MYXV treatment alone, on both left and right-side tumors (FIG. 14B and FIG. 14C). These results demonstrate that, in immunocompetent animals, inhibition of the nuclear export pathway enhances oncolytic MYXV therapeutic activity, and that oncolytic MYXV therapy enhances therapeutic activity of a nuclear export inhibitor.

### XIV. EXAMPLE 9: ALTERATION OF PROTEIN LEVELS

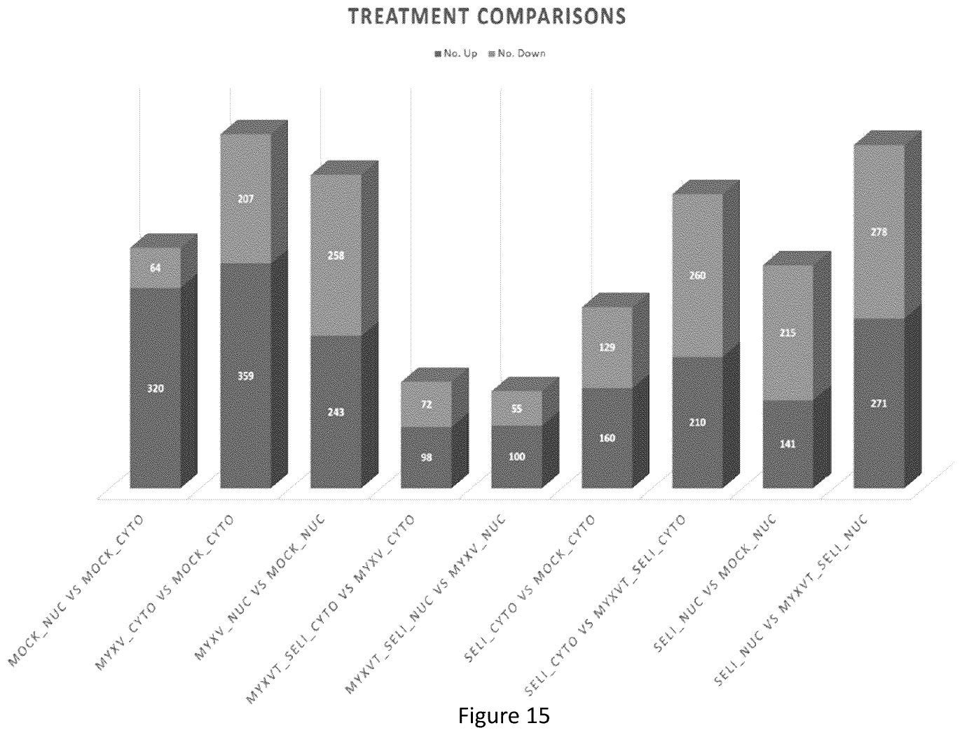

A global proteome analysis of the cytosol and nuclear compartment were performed to identify cellular and viral proteins that change with different treatment, and that may contribute to enhanced viral replication, reduced cell proliferation, and cell death. Colo205 cells were treated with PBS, selinexor, MYXV, or selinexor+MYXV and samples processed to prepare nuclear and cytosolic fractions. Approximately 5,000 cellular and viral proteins were identified by mass spectrometry, and the relative abundances in the nuclear and cytosolic fractions were calculated (FIG. 15 and Table 1 through Table 9). The most significant reduction in abundance of proteins was observed in the nuclear and cytosolic fractions of cells treated with selinexor+MYXV.

TABLE 1
Differential protein expression of control nucleus and control cytoplasm

| Accession | Gene Symbol | $\frac{Mock\_nuc}{Mock\_cyto}$ | P-value |
|---|---|---|---|
| Q96LT9 | RNPC3 | 100 | 2.77E−16 |
| P17010 | ZFX | 100 | 2.77E−16 |
| Q01826 | SATB1 | 100 | 2.77E−16 |
| Q86YC2 | PALB2 | 100 | 2.77E−16 |
| Q2KHR2 | RFX7 | 100 | 2.77E−16 |
| P54274 | TERF1 | 100 | 2.77E−16 |
| Q9C0A6 | SETD5 | 100 | 2.77E−16 |
| O95402 | MED26 | 100 | 2.77E−16 |
| Q9H4Z2 | ZNF335 | 100 | 2.77E−16 |
| Q15906 | VPS72 | 100 | 2.77E−16 |
| Q9GZN1 | ACTR6 | 100 | 2.77E−16 |
| Q9ULD4 | BRPF3 | 100 | 2.77E−16 |
| Q9C086 | INO80B | 100 | 2.77E−16 |
| Q9H2F5 | EPC1 | 100 | 2.77E−16 |
| P11055 | MYH3 | 100 | 2.77E−16 |
| O95625 | ZBTB11 | 100 | 2.77E−16 |
| Q6UB99 | ANKRD11 | 100 | 2.77E−16 |
| Q96BN2 | TADA1 | 100 | 2.77E−16 |
| Q9YJT4 | MA56 | 100 | 2.77E−16 |
| P38398 | BRCA1 | 100 | 2.77E−16 |
| P17676 | CEBPB | 100 | 2.77E−16 |
| P57071 | PRDM15 | 100 | 2.77E−16 |
| O00287 | RFXAP | 100 | 2.77E−16 |
| Q6ZUT6 | CCDC9B | 100 | 2.77E−16 |
| Q8TAK5 | GABPB2 | 100 | 2.77E−16 |
| O00716 | E2F3 | 100 | 2.77E−16 |
| Q9P2W1 | PSMC3IP | 100 | 2.77E−16 |
| Q969R5 | L3MBTL2 | 100 | 2.77E−16 |
| Q3T8J9 | GON4L | 100 | 2.77E−16 |
| B2CWP6 | m139R | 100 | 2.77E−16 |
| Q76L83 | ASXL2 | 100 | 2.77E−16 |
| P40424 | PBX1 | 100 | 2.77E−16 |
| Q9H582 | ZNF644 | 100 | 2.77E−16 |
| Q05BQ5 | MBTD1 | 100 | 2.77E−16 |
| Q5T6S3 | PHF19 | 100 | 2.77E−16 |
| O00470 | MEIS1 | 100 | 2.77E−16 |
| P35226 | BMI1 | 100 | 2.77E−16 |
| Q4VC05 | BCL7A | 100 | 2.77E−16 |
| Q5VYV7 | SLX4IP | 100 | 2.77E−16 |
| Q8N680 | ZBTB2 | 100 | 2.77E−16 |
| Q8WUB8 | PHF10 | 100 | 2.77E−16 |
| Q9P1U0 | POLR1H | 100 | 2.77E−16 |
| B2CWC6 | m020L | 100 | 2.77E−16 |
| O95696 | BRD1 | 100 | 2.77E−16 |
| P05204 | HMGN2 | 100 | 2.77E−16 |
| Q6ZTU2 | EP400P1 | 100 | 2.77E−16 |
| Q9NZI5 | GRHL1 | 100 | 2.77E−16 |
| Q8IV63 | VRK3 | 100 | 2.77E−16 |
| O60292 | SIPA1L3 | 100 | 2.77E−16 |
| Q9UQR0 | SCML2 | 100 | 2.77E−16 |
| O15265 | ATXN7 | 100 | 2.77E−16 |
| Q9BRL6 | SRSF8 | 100 | 2.77E−16 |
| Q9UL40 | ZNF346 | 100 | 2.77E−16 |
| Q86T24 | ZBTB33 | 100 | 2.77E−16 |
| O43513 | MED7 | 100 | 2.77E−16 |
| Q9HAJ7 | SAP30L | 100 | 2.77E−16 |
| Q14541 | HNF4G | 100 | 2.77E−16 |
| Q9Q8S2 | m020L | 100 | 2.77E−16 |
| Q9Y483 | MTF2 | 100 | 2.77E−16 |
| Q8N8D1 | PDCD7 | 100 | 2.77E−16 |
| Q96IZ7 | RSRC1 | 100 | 2.77E−16 |
| O14753 | OVOL1 | 100 | 2.77E−16 |
| Q15562 | TEAD2 | 100 | 2.77E−16 |
| Q9Y2X9 | ZNF281 | 100 | 2.77E−16 |
| Q8NG31 | KNL1 | 100 | 2.77E−16 |
| Q96MY1 | NOL4L | 100 | 2.77E−16 |
| O15417 | TNRC18 | 100 | 2.77E−16 |
| Q8IYH5 | ZZZ3 | 100 | 2.77E−16 |
| Q8NBZ0 | INO80E | 100 | 2.77E−16 |
| Q96L73 | NSD1 | 100 | 2.77E−16 |
| Q9NZI6 | TFCP2L1 | 100 | 2.77E−16 |
| Q15561 | TEAD4 | 100 | 2.77E−16 |
| Q14CW9 | ATXN7L3 | 100 | 2.77E−16 |
| Q9H254 | SPTBN4 | 100 | 2.77E−16 |
| Q12948 | FOXC1 | 100 | 2.77E−16 |
| P41212 | ETV6 | 100 | 2.77E−16 |
| Q5JSJ4 | INTS6L | 100 | 2.77E−16 |
| O43312 | MTSS1 | 100 | 2.77E−16 |
| Q01831 | XPC | 100 | 2.77E−16 |
| Q9P1Y6 | PHRF1 | 100 | 2.77E−16 |
| P17096 | HMGA1 | 100 | 2.77E−16 |
| Q15361 | TTF1 | 100 | 2.77E−16 |
| E2CZL6 | m127L | 100 | 2.77E−16 |
| Q8N488 | RYBP | 100 | 2.77E−16 |
| Q7Z5J4 | RAI1 | 100 | 2.77E−16 |
| P17275 | JUNB | 100 | 2.77E−16 |
| Q9Y261 | FOXA2 | 100 | 2.77E−16 |
| Q9HBM6 | TAF9B | 100 | 2.77E−16 |
| Q8NEZ4 | KMT2C | 100 | 2.77E−16 |
| Q9Q8M6 | m074R | 100 | 2.77E−16 |
| Q9Y3Y2 | CHTOP | 100 | 2.77E−16 |
| Q9UK61 | TASOR | 100 | 2.77E−16 |
| Q9ULV3 | CIZ1 | 100 | 2.77E−16 |
| Q9H6RO | DHX33 | 100 | 2.77E−16 |
| Q9NSB2 | KRT84 | 100 | 2.77E−16 |
| Q96HA1 | POM121 | 100 | 2.77E−16 |
| O15014 | ZNF609 | 100 | 2.77E−16 |
| Q9P2N6 | KANSL3 | 100 | 2.77E−16 |
| Q32P51 | HNRNPA1L2 | 100 | 2.77E−16 |
| PO2545 | LMNA | 100 | 2.77E−16 |
| P18846 | ATF1 | 100 | 2.77E−16 |
| Q9NV56 | MRGBP | 100 | 2.77E−16 |
| Q96E39 | RBMXL1 | 100 | 2.77E−16 |
| Q9P0M6 | MACROH2A2 | 100 | 2.77E−16 |
| O15523 | DDX3Y | 100 | 2.77E−16 |
| Q9UQ16 | DNM3 | 100 | 2.77E−16 |
| Q8N954 | GPATCH11 | 100 | 2.77E−16 |
| P17029 | ZKSCAN1 | 100 | 2.77E−16 |
| O75928 | PIAS2 | 100 | 2.77E−16 |
| Q96CB8 | INTS12 | 100 | 2.77E−16 |
| Q92738 | USP6NL | 100 | 2.77E−16 |
| Q8NCN4 | RNF169 | 100 | 2.77E−16 |
| Q9H6P5 | TASP1 | 100 | 2.77E−16 |
| Q96QT6 | PHF12 | 100 | 2.77E−16 |
| Q9H8G2 | CAAP1 | 100 | 2.77E−16 |
| E2CZJ6 | m107L | 100 | 2.77E−16 |
| B2CWL4 | | 100 | 2.77E−16 |
| Q8TDY2 | RB1CC1 | 100 | 2.77E−16 |
| Q53TQ3 | INO80D | 100 | 2.77E−16 |
| P15408 | FOSL2 | 100 | 2.77E−16 |
| Q9UER7 | DAXX | 100 | 2.77E−16 |
| Q15572 | TAF1C | 100 | 2.77E−16 |
| Q99594 | TEAD3 | 100 | 2.77E−16 |
| Q9Y388 | RBMX2 | 100 | 2.77E−16 |
| Q9H7N4 | SCAF1 | 100 | 2.77E−16 |
| Q8NDX5 | PHC3 | 100 | 2.77E−16 |
| Q9HBE1 | PATZ1 | 100 | 2.77E−16 |
| Q9BRX9 | WDR83 | 100 | 2.77E−16 |
| O14867 | BACH1 | 100 | 2.77E−16 |
| Q6PJE2 | POMZP3 | 100 | 2.77E−16 |
| O75529 | TAF5L | 100 | 2.77E−16 |
| Q9H160 | ING2 | 100 | 2.77E−16 |
| Q5VWQ0 | RSBN1 | 100 | 2.77E−16 |
| Q9ULM3 | YEATS2 | 100 | 2.77E−16 |
| Q9NS91 | RAD18 | 100 | 2.77E−16 |
| Q9BRG2 | SH2D3A | 100 | 2.77E−16 |
| Q9Q8K3 | m097R | 100 | 2.77E−16 |
| Q8IZL8 | PELP1 | 94.604 | 0.2109 |
| P18754 | RCC1 | 93.042 | 0.2912 |
| P16402 | H1-3 | 81.432 | 0.0126 |
| P16220 | CREB1 | 77.833 | 0.3227 |
| P43243 | MATR3 | 75.067 | 0.3845 |
| Q8NEY8 | PPHLN1 | 68.774 | 0.0536 |
| Q99986 | VRK1 | 68.347 | 0.3843 |
| Q8TAQ2 | SMARCC2 | 67.995 | 0.4312 |
| Q96JM3 | CHAMP1 | 65.773 | 0.5189 |
| Q92804 | TAF15 | 62.522 | 0.2663 |
| Q9H410 | RAD21L1 | 61.179 | 0.5658 |
| Q12996 | CSTF3 | 59.691 | 0.4366 |
| P35659 | DEK | 59.19 | 0.5023 |
| Q96N64 | PWWP2A | 57.662 | 0.0686 |
| P19793 | RXRA | 56.311 | 0.1095 |
| P20700 | LMNB1 | 54.997 | 0.5355 |
| Q13217 | DNAJC3 | 53.896 | 0.5471 |
| Q9Q8P8 | m048L | 52.672 | 0.3633 |
| P08621 | SNRNP70 | 51.233 | 0.5658 |
| P42166 | TMPO | 50.977 | 0.5658 |
| P51531 | SMARCA2 | 50.484 | 0.5658 |
| P23246 | SFPQ | 49.097 | 0.5819 |
| P22492 | H1-6 | 48.128 | 0.5902 |
| Q02539 | H1-1 | 48.128 | 0.5902 |
| Q9HCD5 | NCOA5 | 46.875 | 0.6442 |
| Q15424 | SAFB | 46.611 | 0.5935 |
| Q5SY16 | NOL9 | 46.476 | 0.5658 |
| P49756 | RBM25 | 46.16 | 0.6056 |
| Q96T37 | RBM15 | 46.151 | 0.6549 |
| Q9BV38 | WDR18 | 45.58 | 0.5658 |
| P14866 | HNRNPL | 44.495 | 0.6228 |
| Q8IX12 | CCAR1 | 44.231 | 0.6237 |
| P16401 | H1-5 | 44.221 | 0.6237 |
| Q12800 | TFCP2 | 42.607 | 0.6793 |
| P07305 | H1-0 | 42.592 | 0.4533 |
| O6O216 | RAD21 | 41.526 | 0.5716 |
| Q14980 | NUMA1 | 41.348 | 0.6442 |
| P51991 | HNRNPA3 | 40.906 | 0.6442 |
| Q9ULK4 | MED23 | 40.806 | 0.6228 |
| Q15233 | NONO | 40.57 | 0.6449 |
| Q92922 | SMARCC1 | 40.298 | 0.6044 |
| Q86YP4 | GATAD2A | 39.34 | 0.6310 |
| P35680 | HNF1B | 39.119 | 0.1437 |
| Q14839 | CHD4 | 38.988 | 0.6574 |
| P52756 | RBM5 | 38.574 | 0.2397 |
| Q9Y4W2 | LAS1L | 38.432 | 0.7061 |
| Q49A26 | GLYR1 | 37.898 | 0.7105 |
| Q03252 | LMNB2 | 37.448 | 0.5613 |
| P10412 | H1-4 | 37.242 | 0.6789 |
| Q13523 | PRPF4B | 37.083 | 0.6496 |
| Q7RTV0 | PHF5A | 37.041 | 0.5355 |
| Q96T88 | UHRF1 | 36.236 | 0.6310 |
| Q8WU68 | U2AF1L4 | 36.056 | 0.7101 |
| Q969G3 | SMARCE1 | 36.019 | 0.6325 |
| Q9NWB6 | ARGLU1 | 35.818 | 0.6993 |
| Q14004 | CDK13 | 35.586 | 0.2306 |
| Q9NXF1 | TEX10 | 35.557 | 0.6484 |
| Q96DI7 | SNRNP40 | 35.358 | 0.5526 |
| P55265 | ADAR | 35.326 | 0.6930 |
| P82979 | SARNP | 35.27 | 0.6930 |
| O60264 | SMARCA5 | 34.989 | 0.6949 |
| O14979 | HNRNPDL | 34.578 | 0.6549 |
| Q9BUQ8 | DDX23 | 34.217 | 0.7033 |
| Q9HOM0 | WWP1 | 34.155 | 0.5975 |
| Q8N684 | CPSF7 | 34.151 | 0.5658 |
| Q9Y614 | USP3 | 33.871 | 0.7009 |
| O43660 | PLRG1 | 33.854 | 0.6523 |
| O94906 | PRPF6 | 33.775 | 0.7061 |
| O00567 | NOP56 | 33.503 | 0.7061 |
| P07910 | HNRNPC | 33.466 | 0.7061 |
| Q14141 | SEPTIN6 | 33.373 | 0.6824 |
| Q96KR1 | ZFR | 33.328 | 0.5750 |
| Q9UKM9 | RALY | 33.294 | 0.5658 |
| P22626 | HNRNPA2B1 | 33.217 | 0.7094 |
| O75448 | MED24 | 33.056 | 0.7096 |
| O60812 | HNRNPCL1 | 32.972 | 0.6769 |
| B2RXH8 | HNRNPCL2 | 32.972 | 0.6769 |
| Q15554 | TERF2 | 32.969 | 0.6886 |
| Q85295 | M11L | 32.675 | 0.2306 |
| Q9Y2X3 | NOP58 | 32.377 | 0.7105 |
| Q86V15 | CASZ1 | 32.204 | 0.6228 |
| Q96ST3 | SIN3A | 31.932 | 0.5875 |
| Q92908 | GATA6 | 31.613 | 0.4664 |
| B7ZW38 | HNRNPCL3 | 31.44 | 0.6886 |
| Q15061 | WDR43 | 30.805 | 0.6264 |
| Q96MM3 | ZFP42 | 30.752 | 0.7105 |
| Q14149 | MORC3 | 30.667 | 0.5471 |
| Q15427 | SF3B4 | 30.628 | 0.6991 |
| Q9BY77 | POLDIP3 | 30.581 | 0.6200 |
| Q5VTL8 | PRPF38B | 30.468 | 0.6496 |
| Q9NRL2 | BAZ1A | 29.784 | 0.7094 |
| A6NHQ2 | FBLL1 | 29.765 | 0.7050 |
| Q96HW7 | INTS4 | 29.362 | 0.6895 |
| Q8NI51 | CTCFL | 29.322 | 0.6056 |
| Q15428 | SF3A2 | 29.257 | 0.6310 |
| Q99496 | RNF2 | 29.168 | 0.6886 |
| P25490 | YY1 | 29.1 | 0.7061 |
| O43290 | SART1 | 28.759 | 0.6791 |
| O60306 | AQR | 28.721 | 0.7102 |
| Q9H2P0 | ADNP | 28.364 | 0.6442 |
| Q8WXA9 | SREK1 | 28.324 | 0.7105 |
| Q8NI27 | THOC2 | 28.18 | 0.6442 |
| Q9H0E9 | BRD8 | 27.866 | 0.6264 |
| B2CWQ3 | m147R | 27.591 | 0.3074 |
| Q14684 | RRP1B | 27.58 | 0.6599 |
| P55347 | PKNOX1 | 26.706 | 0.0197 |
| O75362 | ZNF217 | 26.062 | 0.4533 |
| P48378 | RFX2 | 25.351 | 0.3969 |
| Q7Z7F0 | KHDC4 | 24.591 | 0.7105 |
| P11474 | ESRRA | 24.453 | 0.6825 |
| P14316 | IRF2 | 24.376 | 0.6373 |
| Q9UGU5 | HMGXB4 | 24.272 | 0.4952 |
| POC860 | MSL3P1 | 23.501 | 0.1091 |
| Q8N5Y2 | MSL3 | 23.501 | 0.3215 |
| Q5TAP6 | UTP14C | 23.235 | 0.4533 |
| Q9HCU9 | BRMS1 | 23.008 | 0.2566 |
| P10589 | NR2F1 | 22.848 | 0.3274 |
| P24468 | NR2F2 | 22.848 | 0.4264 |
| Q99549 | MPHOSPH8 | 22.725 | 0.5311 |
| P37231 | PPARG | 22.515 | 0.4255 |
| Q9NZC4 | EHF | 22.089 | 0.3155 |
| Q86UY6 | NAA40 | 21.58 | 0.3365 |
| O00566 | MPHOSPH10 | 21.389 | 0.6310 |
| O15550 | KDM6A | 21.13 | 0.3969 |
| Q96EK4 | THAP11 | 20.949 | 0.5069 |
| Q96ES7 | SGF29 | 20.494 | 0.4967 |
| P08651 | NFIC | 19.177 | 0.5562 |
| B7ZAP0 | RABGAP1L | 18.655 | 0.6200 |
| Q86TJ2 | TADA2B | 18.54 | 0.0535 |
| Q92793 | CREBBP | 18.323 | 0.5730 |
| P10242 | MYB | 18.201 | 0.6282 |
| P52945 | PDX1 | 17.224 | 0.6056 |
| Q9H967 | WDR76 | 17.097 | 0.6442 |
| Q9UIF9 | BAZ2A | 16.274 | 0.6449 |
| O14647 | CHD2 | 16.184 | 0.4312 |
| Q9NV31 | IMP3 | 15.898 | 0.4967 |
| Q9P2K3 | RCOR3 | 15.673 | 0.6769 |
| Q9NZI7 | UBP1 | 15.551 | 0.6449 |
| P24863 | CCNC | 15.53 | 0.5311 |
| P78549 | NTHL1 | 15.245 | 0.6767 |
| Q9BTA9 | WAC | 15.088 | 0.6314 |
| A6NKF1 | SAC3D1 | 15.069 | 0.1714 |
| Q9BYE7 | PCGF6 | 14.762 | 0.6886 |
| Q01664 | TFAP4 | 14.729 | 0.5355 |
| P57721 | PCBP3 | 14.626 | 0.7013 |
| Q9BQE9 | BCL7B | 14.617 | 0.6825 |
| Q99856 | ARID3A | 14.541 | 0.6793 |
| Q9H0H5 | RACGAP1 | 14.358 | 0.6373 |
| Q8IYL3 | C1orf174 | 14.265 | 0.6310 |
| B2CWQ7 | | 14.237 | 0.5730 |
| D2WN93 | m151R | 14.237 | 0.6442 |
| Q14938 | NFIX | 14.038 | 0.6793 |
| O15525 | MAFG | 13.747 | 0.5355 |
| Q92733 | PRCC | 13.51 | 0.6484 |
| O94762 | RECQL5 | 13.449 | 0.5819 |
| O43829 | ZBTB14 | 13.394 | 0.6314 |
| O43251 | RBFOX2 | 13.393 | 0.6056 |
| Q9Y657 | SPIN1 | 13.203 | 0.6895 |
| P17544 | ATF7 | 12.881 | 0.7105 |
| O95243 | MBD4 | 12.396 | 0.6886 |
| Q5C9Z4 | NOM1 | 12.341 | 0.7096 |
| Q15652 | JMJD1C | 12.334 | 0.5311 |
| Q15047 | SETDB1 | 12.057 | 0.2537 |
| Q9NVF7 | FBXO28 | 11.506 | 0.6442 |
| Q9Y4E5 | ZNF451 | 11.031 | 0.6789 |
| Q9BQ04 | RBM4B | 10.992 | 0.6668 |
| Q9BXK1 | KLF16 | 10.773 | 0.6930 |
| P46100 | | 9.585 | 0.7057 |
| Q9H201 | EPN3 | 9.523 | 0.4664 |
| P52739 | ZNF131 | 9.392 | 0.3427 |
| Q9UPQ0 | LIMCH1 | 9.169 | 0.3969 |
| Q96RR1 | TWNK | 8.183 | 0.4372 |
| Q8TD26 | CHD6 | 7.396 | 0.6051 |
| Q15723 | ELF2 | 7.023 | 0.5701 |
| Q6ZN18 | AEBP2 | 6.891 | 0.6310 |
| Q14188 | TFDP2 | 6.877 | 0.6687 |
| Q14147 | DHX34 | 6.837 | 0.6056 |
| Q9HBI1 | PARVB | 6.533 | 0.6939 |
| Q9NQB0 | TCF7L2 | 6.514 | 0.6574 |
| Q13889 | GTF2H3 | 6.492 | 0.5311 |
| Q96SB8 | SMC6 | 6.109 | 0.7009 |
| O95425 | SVIL | 6.106 | 0.6930 |
| P48594 | SERPINB4 | 5.849 | 0.7102 |
| P20930 | FLG | 5.814 | 0.6493 |
| Q9UBP9 | GULP1 | 5.759 | 0.7061 |
| P00519 | ABL1 | 5.703 | 0.6484 |
| Q7L9B9 | EEPD1 | 4.821 | 0.7061 |
| Q9GZY8 | MFF | 0.036 | 0.6056 |
| Q86TM6 | SYVN1 | 0.021 | 0.1441 |
| B2CWE0 | m033R | 0.01 | 2.77E−16 |
| P12259 | F5 | 0.01 | 2.77E−16 |
| P33908 | MAN1A1 | 0.01 | 2.77E−16 |
| B2CWG7 | m060R | 0.01 | 2.77E−16 |
| Q9Q8Q3 | m043L | 0.01 | 2.77E−16 |
| Q9UPQ3 | AGAP1 | 0.01 | 2.77E−16 |
| Q9Q8H9 | m122R | 0.01 | 2.77E−16 |
| Q9UHD2 | TBK1 | 0.01 | 2.77E−16 |
| D2WN26 | m090L | 0.01 | 2.77E−16 |
| Q05209 | PTPN12 | 0.01 | 2.77E−16 |
| Q8TE04 | PANK1 | 0.01 | 2.77E−16 |
| Q9H9C1 | VIPAS39 | 0.01 | 2.77E−16 |
| Q77P93 | m003.2L | 0.01 | 2.77E−16 |
| Q9Q8Q1 | | 0.01 | 2.77E−16 |
| P55211 | CASP9 | 0.01 | 2.77E−16 |
| O15327 | INPP4B | 0.01 | 2.77E−16 |
| Q8NI22 | MCFD2 | 0.01 | 2.77E−16 |
| Q96BW5 | PTER | 0.01 | 2.77E−16 |
| Q8N612 | FHIP1B | 0.01 | 2.77E−16 |
| O95989 | NUDT3 | 0.01 | 2.77E−16 |
| Q10472 | GALNT1 | 0.01 | 2.77E−16 |
| D2WN05 | m078R | 0.01 | 2.77E−16 |
| P17050 | NAGA | 0.01 | 2.77E−16 |
| P98172 | EFNB1 | 0.01 | 2.77E−16 |
| O60449 | LY75 | 0.01 | 2.77E−16 |
| P51151 | RAB9A | 0.01 | 2.77E−16 |
| A8K2U0 | A2ML1 | 0.01 | 2.77E−16 |
| Q6DKJ4 | NXN | 0.01 | 2.77E−16 |
| Q9Y6M5 | SLC30A1 | 0.01 | 2.77E−16 |
| P80188 | LCN2 | 0.01 | 2.77E−16 |
| Q96AG3 | SLC25A46 | 0.01 | 2.77E−16 |
| P40855 | PEX19 | 0.01 | 2.77E−16 |
| O95865 | DDAH2 | 0.01 | 2.77E−16 |
| O76015 | KRT38 | 0.01 | 2.77E−16 |
| O76013 | KRT36 | 0.01 | 2.77E−16 |
| Q9H008 | LHPP | 0.01 | 2.77E−16 |
| Q5TGY1 | TMCO4 | 0.01 | 2.77E−16 |
| P21980 | TGM2 | 0.01 | 2.77E−16 |
| P54098 | POLG | 0.01 | 2.77E−16 |
| Q96HD1 | CRELD1 | 0.01 | 2.77E−16 |
| P50583 | NUDT2 | 0.01 | 2.77E−16 |
| Q4KMQ1 | TPRN | 0.01 | 2.77E−16 |
| Q9BZ23 | PANK2 | 0.01 | 2.77E−16 |
| Q15643 | TRIP11 | 0.01 | 2.77E−16 |
| O43379 | WDR62 | 0.01 | 2.77E−16 |
| O95671 | ASMTL | 0.01 | 2.77E−16 |
| E2CZM0 | m131R | 0.01 | 2.77E−16 |
| Q9BVW5 | TIPIN | 0.01 | 2.77E−16 |
| Q8IY95 | TMEM192 | 0.01 | 2.77E−16 |
| Q5U3C3 | TMEM164 | 0.01 | 2.77E−16 |
| P10909 | CLU | 0.01 | 2.77E−16 |
| P23443 | RPS6KB1 | 0.01 | 2.77E−16 |
| P18859 | ATP5PF | 0.01 | 2.77E−16 |
| E2CZV1 | m056R | 0.01 | 2.77E−16 |
| Q9HBR0 | SLC38A10 | 0.01 | 2.77E−16 |
| P14209 | CD99 | 0.01 | 2.77E−16 |
| Q9UHX3 | ADGRE2 | 0.01 | 2.77E−16 |
| Q8TB72 | PUM2 | 0.01 | 2.77E−16 |
| P54753 | EPHB3 | 0.01 | 2.77E−16 |
| B2CWH3 | m066R | 0.01 | 2.77E−16 |
| P54762 | EPHB1 | 0.01 | 2.77E−16 |
| Q8TDQ7 | GNPDA2 | 0.01 | 2.77E−16 |
| P35504 | UGT1A5 | 0.01 | 2.77E−16 |
| P17066 | HSPA6 | 0.01 | 2.77E−16 |

TABLE 2
Differential protein expression of MYXV-treated nucleus and control nucleus

| Accession | Gene Symbol | $\frac{MYXV\_nuc}{Mock\_nuc}$ | P-value |
|---|---|---|---|
| B2CWB0 | | 100.00 | 4.87E−16 |
| B2CWK3 | | 100.00 | 4.87E−16 |
| Q9Q8Q1 | | 100.00 | 4.87E−16 |
| B2CWG1 | | 100.00 | 4.87E−16 |
| B2CWI0 | | 100.00 | 4.87E−16 |
| B2CWJ5 | | 100.00 | 4.87E−16 |
| A8K2U0 | A2ML1 | 100.00 | 4.87E−16 |
| Q9UHX3 | ADGRE2 | 100.00 | 4.87E−16 |
| Q9UPQ3 | AGAP1 | 100.00 | 4.87E−16 |
| P49759 | CLK1 | 100.00 | 4.87E−16 |
| O95865 | DDAH2 | 100.00 | 4.87E−16 |
| P12259 | F5 | 100.00 | 4.87E−16 |
| Q8N612 | FHIP1B | 100.00 | 4.87E−16 |
| Q10472 | GALNT1 | 100.00 | 4.87E−16 |
| Q8TDQ7 | GNPDA2 | 100.00 | 4.87E−16 |
| P80188 | LCN2 | 100.00 | 4.87E−16 |
| O60449 | LY75 | 100.00 | 4.87E−16 |
| Q77P93 | m003.2L | 100.00 | 4.87E−16 |
| Q83730 | m005R | 100.00 | 4.87E−16 |
| Q9Q8S8 | m013L | 100.00 | 4.87E−16 |
| Q9Q8R4 | m032R | 100.00 | 4.87E−16 |
| B2CWE0 | m033R | 100.00 | 4.87E−16 |
| Q9Q8Q3 | m043L | 100.00 | 4.87E−16 |
| K4J9W4 | m054R | 100.00 | 4.87E−16 |
| E2CZV1 | m056R | 100.00 | 4.87E−16 |
| B2CWG7 | m060R | 100.00 | 4.87E−16 |
| B2CWH3 | m066R | 100.00 | 4.87E−16 |
| Q9Q8N0 | m070R | 100.00 | 4.87E−16 |
| K4JKC2 | m073R | 100.00 | 4.87E−16 |
| D2WN05 | m078R | 100.00 | 4.87E−16 |
| E2CZH7 | m088L | 100.00 | 4.87E−16 |
| Q9Q8L1 | m089L | 100.00 | 4.87E−16 |
| D2WN26 | m090L | 100.00 | 4.87E−16 |
| Q9Q8J9 | m101L | 100.00 | 4.87E−16 |
| Q9Q8J5 | m105L | 100.00 | 4.87E−16 |
| Q9Q8J4 | m106L | 100.00 | 4.87E−16 |
| E2CZJ9 | m110L | 100.00 | 4.87E−16 |
| Q9Q817 | m113R | 100.00 | 4.87E−16 |
| Q9Q8H9 | m122R | 100.00 | 4.87E−16 |
| E2CZM0 | m131R | 100.00 | 4.87E−16 |
| Q9Q8G4 | m137R | 100.00 | 4.87E−16 |
| B2CWP9 | m142R | 100.00 | 4.87E−16 |
| B2CWQ5 | m149R | 100.00 | 4.87E−16 |
| B2CWR1 | m156R | 100.00 | 4.87E−16 |
| Q8NI22 | MCFD2 | 100.00 | 4.87E−16 |
| Q5VVJ2 | MYSM1 | 100.00 | 4.87E−16 |
| P17050 | NAGA | 100.00 | 4.87E−16 |
| P50583 | NUDT2 | 100.00 | 4.87E−16 |
| O95989 | NUDT3 | 100.00 | 4.87E−16 |
| Q6DKJ4 | NXN | 100.00 | 4.87E−16 |
| Q96BW5 | PTER | 100.00 | 4.87E−16 |
| Q05209 | PTPN12 | 100.00 | 4.87E−16 |
| Q8TB72 | PUM2 | 100.00 | 4.87E−16 |
| P51151 | RAB9A | 100.00 | 4.87E−16 |
| A1X283 | SH3PXD2B | 100.00 | 4.87E−16 |
| Q96AG3 | SLC25A46 | 100.00 | 4.87E−16 |
| Q9BVW5 | TIPIN | 100.00 | 4.87E−16 |
| Q5TGY1 | TMCO4 | 100.00 | 4.87E−16 |
| Q8IY95 | TMEM192 | 100.00 | 4.87E−16 |
| Q9Q8K4 | VETFL | 100.00 | 4.87E−16 |
| Q9H9C1 | VIPAS39 | 100.00 | 4.87E−16 |
| D3W0C4 | m130R | 99.39 | 4.87E−16 |
| Q9Q8Q8 | m038L | 98.49 | 4.87E−16 |
| B2CWG0 | m053R | 91.58 | 4.87E−16 |
| Q9Q8M5 | m075R | 90.47 | 4.87E−16 |
| Q9PX36 | m006L | 87.89 | 4.87E−16 |
| Q9YQ03 | mt6 | 87.89 | 4.87E−16 |
| P68544 | PAPS | 86.97 | 4.87E−16 |
| B2CWB5 | | 70.02 | 4.87E−16 |
| Q9Q8Q6 | m040L | 58.64 | 4.87E−16 |
| Q9Q8G6 | m135R | 46.53 | 4.87E−16 |
| B2CWM2 | m115L | 35.08 | 4.87E−16 |
| Q85297 | | 34.89 | 4.87E−16 |
| B2CWH6 | m069L | 34.89 | 4.87E−16 |
| Q9Q814 | m117L | 30.50 | 4.87E−16 |
| O10625 | M-T1 | 30.30 | 4.87E−16 |
| E2CZP2 | m001R | 30.30 | 4.87E−16 |
| B2CWD8 | | 29.61 | 4.87E−16 |
| Q9Q8R5 | m031R | 29.61 | 4.87E−16 |
| Q9Q8L8 | m082R | 28.00 | 4.87E−16 |
| B2CWI4 | m077L | 24.01 | 4.87E−16 |
| Q9Q8F0 | m154L | 23.77 | 4.87E−16 |
| B2CWL8 | m111R | 23.54 | 3.16E−14 |
| B2CWD4 | m027L | 23.50 | 7.32E−14 |
| B2CWA8 | | 23.17 | 4.87E−16 |
| O55698 | M-T4 | 23.17 | 4.87E−16 |
| Q9PXA3 | m004L | 23.17 | 4.87E−16 |
| B2CWF6 | | 23.10 | 4.87E−16 |
| Q9Q8P7 | m049R | 23.10 | 4.87E−16 |
| Q9Q8K3 | m097R | 22.92 | 4.87E−16 |
| D2WM37 | m012L | 22.81 | 4.87E−16 |
| Q9Q8J1 | m109L | 22.14 | 1.07E−14 |
| B2CWD6 | m029L | 18.79 | 4.87E−16 |
| B2CWB4 | m008.1L | 18.64 | 4.87E−16 |
| Q9Q8S0 | m026R | 15.31 | 1.06E−10 |
| D2WMA0 | m030L | 14.66 | 4.23E−11 |
| B2CWL4 | | 14.39 | 1.05E−12 |
| E2CZJ6 | m107L | 14.39 | 1.05E−12 |
| E2CZW3 | m068R | 13.26 | 2.13E−14 |
| Q9Q811 | m120L | 12.18 | 1.10E−08 |
| B2CWP6 | m139R | 11.93 | 2.21E−10 |
| E2CZR0 | m015L | 11.71 | 4.04E−13 |
| B2CWC1 | m015L | 11.71 | 4.04E−13 |
| B2CWE1 | | 11.36 | 1.79E−12 |
| Q9Q8R2 | m034L | 11.36 | 5.72E−13 |
| B2CWB2 | | 11.12 | 3.39E−13 |
| Q9PX24 | m007L | 11.12 | 3.39E−13 |
| B2CWI7 | m080R | 10.58 | 8.78E−12 |
| B2CWC8 | | 10.58 | 8.52E−08 |
| B2CWE3 | | 10.52 | 1.64E−09 |
| Q9Q8R0 | m036L | 10.52 | 3.35E−09 |
| B2CWI3 | m076R | 10.38 | 7.26E−13 |
| Q9Q8M6 | m074R | 10.37 | 7.63E−09 |
| B2CWG4 | m057L | 10.35 | 7.08E−09 |
| E2CZM2 | m133R | 10.03 | 1.45E−12 |
| Q77PB1 | m003.1L | 10.01 | 5.62E−08 |
| B2CWA6 | m003.1R | 10.01 | 5.62E−08 |
| B2CWK2 | m095L | 9.86 | 5.60E−12 |
| Q9Q8N5 | | 9.84 | 9.56E−09 |
| B2CWH0 | | 9.84 | 9.56E−09 |
| Q85296 | | 9.72 | 7.74E−12 |
| E2CZQ3 | m009L | 9.59 | 1.06E−08 |
| Q9Q8L9 | VETFS | 9.13 | 1.79E−07 |
| B2CWF4 | m047R | 9.04 | 2.14E−08 |
| Q9Q8K1 | m099L | 7.91 | 1.66E−09 |
| Q83604 | MF13 | 7.77 | 8.79E−06 |
| Q9Q8L3 | m087L | 7.65 | 4.22E−07 |
| Q96Q05 | TRAPPC9 | 6.97 | 1.34E−06 |
| Q969Q0 | RPL36AL | 6.69 | 2.00E−06 |
| B2CW16 | m079R | 6.52 | 5.70E−06 |
| B2CWG8 | m061R | 6.38 | 6.56E−06 |
| Q9GZY8 | MFF | 6.08 | 2.31E−05 |
| Q9Q8H2 | m129R | 5.96 | 4.88E−05 |
| E2CZR6 | m021L | 5.90 | 2.55E−05 |
| P01100 | FOS | 5.71 | 3.99E−05 |
| B2CWM1 | | 5.66 | 1.49E−05 |
| E2CZK3 | m114R | 5.66 | 1.49E−05 |
| Q9Q816 | m114R | 5.66 | 1.49E−05 |
| B2CWQ7 | | 5.60 | 7.69E−05 |
| Q9POM6 | MACROH2A2 | 5.22 | 8.78E−05 |
| Q9Q8P4 | m052L | 5.01 | 1.93E−04 |
| P05109 | S100A8 | 4.82 | 8.44E−05 |
| E2CZL6 | m127L | 4.81 | 1.09E−03 |
| B2CWJ3 | | 4.45 | 5.51E−04 |
| E2CZY1 | m086L | 4.45 | 5.51E−04 |
| B2CWC0 | | 4.09 | 2.06E−03 |
| E2CZQ9 | m014L | 4.09 | 2.06E−03 |
| O75367 | MACROH2A1 | 4.02 | 2.06E−03 |
| B2CWG5 | m058R | 3.95 | 5.73E−03 |
| Q9Q8N8 | m058R | 3.95 | 3.68E−03 |
| Q9Q8K7 | m093L | 3.93 | 2.42E−03 |
| P08729 | KRT7 | 3.78 | 6.47E−03 |
| P62805 | H4-16; H4C1; | 3.76 | 3.01E−04 |
| | H4C11; | | |
| | H4C12; | | |
| | H4C13; | | |
| | H4C14; | | |
| | H4C15; | | |
| | H4C2; H4C3; | | |
| | H4C4; H4C5; | | |
| | H4C6; H4C8; | | |
| | H4C9 | | |
| Q9Q8Q4 | m042L | 3.75 | 3.87E−03 |
| Q77PA1 | m138L | 3.71 | 1.09E−02 |
| B2CWH8 | | 3.59 | 5.60E−03 |
| Q9Q8M9 | m071L | 3.59 | 5.12E−03 |
| Q16204 | CCDC6 | 3.51 | 6.01E−03 |
| Q9Q8K6 | m094R | 3.47 | 8.01E−03 |
| Q6NXT2 | H3-5 | 3.45 | 2.27E−04 |
| Q5TEC6 | H3-7 | 3.45 | 2.27E−04 |
| P06899 | H2BC11 | 3.44 | 1.37E−02 |
| P23527 | H2BC17 | 3.44 | 1.37E−02 |
| Q6DRA6 | H2BC19P | 3.44 | 1.37E−02 |
| Q6DN03 | H2BC20P | 3.44 | 1.37E−02 |
| Q16778 | H2BC21 | 3.44 | 1.37E−02 |
| P33778 | H2BC3 | 3.44 | 1.37E−02 |
| Q8N257 | H2BU1 | 3.44 | 1.37E−02 |
| Q16695 | H3-4 | 3.43 | 1.04E−03 |
| Q9ULX9 | MAFF | 3.43 | 1.13E−02 |
| P68550 | | 3.42 | 1.00E−02 |
| P68431 | H3C1; | 3.39 | 1.19E−03 |
| | H3C10; | | |
| | H3C11; | | |
| | H3C12; | | |
| | H3C2; H3C3; | | |
| | H3C4; H3C6; | | |
| | H3C7; H3C8 | | |
| Q9Q8F9 | m144R | 3.37 | 2.71E−02 |
| D2WN93 | m151R | 3.34 | 1.51E−02 |
| P31151 | S100A7 | 3.31 | 2.66E−02 |
| B2CWQ3 | m147R | 3.29 | 2.49E−02 |
| E2CZW7 | m072L | 3.25 | 1.95E−02 |
| P35527 | KRT9 | 3.20 | 2.43E−03 |
| P62807 | H2BC10; | 3.18 | 2.50E−03 |
| | H2BC4; | | |
| | H2BC6; | | |
| | H2BC7; | | |
| | H2BC8 | | |
| O60814 | H2BC12 | 3.18 | 2.50E−03 |
| P57053 | H2BC12L | 3.18 | 2.50E−03 |
| Q99880 | H2BC13 | 3.18 | 2.50E−03 |
| Q99879 | H2BC14 | 3.18 | 2.50E−03 |
| Q99877 | H2BC15 | 3.18 | 2.50E−03 |
| Q5QNW6 | H2BC18 | 3.18 | 2.50E−03 |
| P58876 | H2BC5 | 3.18 | 2.50E−03 |
| Q93079 | H2BC9 | 3.18 | 2.50E−03 |
| Q96GA3 | LTV1 | 3.14 | 1.88E−02 |
| Q71DI3 | H3C13; | 3.08 | 3.80E−02 |
| | H3C14; | | |
| | H3C15 | | |
| P04908 | H2AC4; | 2.99 | 5.14E−03 |
| | H2AC8 | | |
| Q93077 | H2AC6 | 2.99 | 5.14E−03 |
| Q7L7L0 | H2AW | 2.99 | 5.14E−03 |
| Q96QV6 | H2AC1 | 2.95 | 3.57E−02 |
| Q8IUE6 | H2AC21 | 2.95 | 3.57E−02 |
| P16104 | H2AX | 2.95 | 3.57E−02 |
| Q9BPZ3 | PAIP2 | 2.95 | 3.86E−02 |
| B2CWC6 | m020L | 2.93 | 4.82E−02 |
| Q9Q8S2 | m020L | 2.93 | 4.82E−02 |
| Q04864 | REL | 2.92 | 4.04E−02 |
| Q8WZ42 | TTN | 2.92 | 3.57E−02 |
| Q9BW30 | TPPP3 | 2.89 | 9.02E−03 |
| Q96A08 | H2BC1 | 2.87 | 8.23E−03 |
| B2CWL5 | m108R | 2.82 | 9.58E−03 |
| Q99519 | NEU1 | 2.79 | 9.45E−02 |
| P62745 | RHOB | 2.76 | 1.72E−01 |
| Q5BJH7 | YIF1B | 2.72 | 1.55E−01 |
| Q96P63 | SERPINB12 | 2.72 | 1.47E−01 |
| O43312 | MTSS1 | 2.69 | 1.68E−01 |
| Q9Q8G2 | m140R | 2.64 | 8.33E−02 |
| Q9Y2S6 | TMA7 | 2.63 | 1.76E−01 |
| O43278 | SPINT1 | 2.58 | 8.02E−02 |
| A0A2R8Y619 | H2BK1 | 2.56 | 2.59E−02 |
| Q5VZP5 | STYXL2 | 2.56 | 1.15E−01 |
| Q7Z3Z0 | KRT25 | 2.56 | 2.17E−02 |
| Q7Z3Y8 | KRT27 | 2.56 | 2.17E−02 |
| B2CWH1 | m064R | 2.51 | 1.18E−01 |
| Q6ZUT6 | CCDC9B | 2.43 | 2.78E−01 |
| Q6FI13 | H2AC18; | 2.43 | 1.74E−01 |
| | H2AC19 | | |
| Q16777 | H2AC20 | 2.43 | 1.74E−01 |
| O95379 | TNFAIP8 | 2.42 | 3.38E−01 |
| Q7Z794 | KRT77 | 2.39 | 2.81E−01 |
| Q9BU89 | DOHH | 2.36 | 3.47E−01 |
| P24534 | EEF1B2 | 2.33 | 6.27E−02 |
| Q9BUH6 | PAXX | 2.33 | 1.79E−01 |
| Q53T59 | HS1BP3 | 2.31 | 4.07E−01 |
| P07942 | LAMB1 | 2.30 | 4.02E−01 |
| Q96ET8 | TVP23C | 2.29 | 2.27E−01 |
| P0C870 | JMJD7 | 2.28 | 3.44E−01 |
| Q01581 | HMGCS1 | 2.23 | 2.61E−01 |
| P04264 | KRT1 | 2.20 | 1.03E−01 |
| P26641 | EEF1G | 2.19 | 1.05E−01 |
| Q9NZJ0 | DTL | 2.15 | 2.81E−01 |
| P06703 | S100A6 | 2.11 | 1.45E−01 |
| B2CWF3 | m046L | 2.11 | 4.51E−01 |
| O75891 | ALDH1L1 | 2.10 | 4.94E−01 |
| Q9P246 | STIM2 | 2.09 | 3.15E−01 |
| Q15646 | OASL | 2.09 | 3.36E−01 |
| Q01518 | CAP1 | 2.09 | 1.53E−01 |
| Q7Z3Y7 | KRT28 | 2.08 | 1.45E−01 |
| P18827 | SDC1 | 2.06 | 3.15E−01 |
| Q504Q3 | PAN2 | 2.06 | 3.38E−01 |
| Q9BS40 | LXN | 2.03 | 3.80E−01 |
| Q96GZ6 | SLC41A3 | 2.01 | 3.72E−01 |
| P11177 | PDHB | 0.47 | 4.99E−01 |
| Q969Z0 | TBRG4 | 0.47 | 5.09E−01 |
| O00541 | PES1 | 0.47 | 4.79E−01 |
| P16401 | H1-5 | 0.47 | 4.82E−01 |
| P54886 | ALDH18A1 | 0.46 | 4.62E−01 |
| Q13263 | TRIM28 | 0.46 | 4.62E−01 |
| P10515 | DLAT | 0.45 | 4.05E−01 |
| Q9BZK7 | TBL1XR1 | 0.45 | 4.25E−01 |
| Q6UB99 | ANKRD11 | 0.45 | 5.14E−01 |
| Q01780 | EXOSC10 | 0.45 | 3.60E−01 |
| Q9Y618 | PXMP4 | 0.45 | 5.00E−01 |
| P51825 | AFF1 | 0.45 | 5.13E−01 |
| Q9BX16 | TBC1D10A | 0.45 | 4.99E−01 |
| Q9P2R6 | RERE | 0.44 | 5.16E−01 |
| O60907 | TBL1X | 0.44 | 5.19E−01 |
| Q70CQ2 | USP34 | 0.44 | 5.03E−01 |
| Q7L9B9 | EEPD1 | 0.44 | 5.17E−01 |
| Q12849 | GRSF1 | 0.44 | 4.96E−01 |
| Q13835 | PKP1 | 0.44 | 4.05E−01 |
| Q9Y2R9 | MRPS7 | 0.44 | 5.08E−01 |
| Q14444 | CAPRIN1 | 0.44 | 3.39E−01 |
| P55061 | TMBIM6 | 0.44 | 4.68E−01 |
| O00468 | AGRN | 0.43 | 4.19E−01 |
| P17544 | ATF7 | 0.43 | 5.03E−01 |
| Q85295 | M11L | 0.43 | 4.55E−01 |
| Q93074 | MED12 | 0.43 | 5.09E−01 |
| P52630 | STAT2 | 0.43 | 4.25E−01 |
| Q8N4C8 | MINK1 | 0.43 | 4.13E−01 |
| Q14686 | NCOA6 | 0.43 | 5.28E−01 |
| P11388 | TOP2A | 0.43 | 3.11E−01 |
| Q9NVW2 | RLIM | 0.43 | 4.84E−01 |
| Q9Y2X9 | ZNF281 | 0.43 | 3.94E−01 |
| P08559 | PDHA1 | 0.42 | 3.91E−01 |
| Q9Y6V7 | DDX49 | 0.42 | 4.61E−01 |
| P29317 | EPHA2 | 0.42 | 4.99E−01 |
| Q13049 | TRIM32 | 0.42 | 4.29E−01 |
| P18065 | IGFBP2 | 0.42 | 4.26E−01 |
| O15164 | TRIM24 | 0.42 | 5.09E−01 |
| O14578 | CIT | 0.42 | 3.70E−01 |
| P50747 | HLCS | 0.42 | 4.62E−01 |
| Q13227 | GPS2 | 0.42 | 4.96E−01 |
| O00391 | QSOX1 | 0.42 | 4.70E−01 |
| Q9NX57 | RAB20 | 0.42 | 4.45E−01 |
| P15529 | CD46 | 0.42 | 5.37E−01 |
| Q9NQW6 | ANLN | 0.42 | 5.03E−01 |
| P51398 | DAP3 | 0.42 | 4.30E−01 |
| Q9Y261 | FOXA2 | 0.42 | 4.96E−01 |
| Q7Z7H8 | MRPL10 | 0.42 | 4.65E−01 |
| P24863 | CCNC | 0.42 | 4.98E−01 |
| P62633 | CNBP | 0.42 | 1.79E−01 |
| Q9Y618 | NCOR2 | 0.42 | 4.96E−01 |
| P11166 | SLC2A1 | 0.42 | 3.33E−01 |
| P85037 | FOXK1 | 0.42 | 3.80E−01 |
| P03905 | MT-ND4 | 0.42 | 5.19E−01 |
| Q8NFJ5 | GPRC5A | 0.41 | 5.02E−01 |
| Q92759 | GTF2H4 | 0.41 | 3.59E−01 |
| Q15047 | SETDB1 | 0.41 | 3.58E−01 |
| P18074 | ERCC2 | 0.41 | 3.15E−01 |
| Q00341 | HDLBP | 0.41 | 2.35E−01 |
| Q8TD26 | CHD6 | 0.41 | 4.25E−01 |
| P37268 | FDFT1 | 0.41 | 4.10E−01 |
| Q9BXS6 | NUSAP1 | 0.41 | 4.98E−01 |
| O94955 | RHOBTB3 | 0.41 | 3.32E−01 |
| Q9Y4D1 | DAAM1 | 0.41 | 3.51E−01 |
| Q7Z5L9 | IRF2BP2 | 0.41 | 3.32E−01 |
| Q9Y2T7 | YBX2 | 0.41 | 4.20E−01 |
| P62273 | RPS29 | 0.41 | 3.47E−01 |
| P41229 | KDM5C | 0.40 | 3.83E−01 |
| Q9GZR7 | DDX24 | 0.40 | 3.14E−01 |
| Q96L73 | NSD1 | 0.40 | 3.40E−01 |
| Q9H300 | PARL | 0.40 | 3.53E−01 |
| Q9C0A6 | SETD5 | 0.40 | 3.44E−01 |
| Q8IZW8 | TNS4 | 0.40 | 3.37E−01 |
| Q96HP0 | DOCK6 | 0.40 | 4.65E−01 |
| Q9BZG8 | DPH1 | 0.40 | 3.68E−01 |
| Q7Z5J4 | RAI1 | 0.40 | 4.62E−01 |
| Q15750 | TAB1 | 0.40 | 2.98E−01 |
| Q9NRC8 | SIRT7 | 0.40 | 3.95E−01 |
| P28288 | ABCD3 | 0.40 | 3.33E−01 |
| O14647 | CHD2 | 0.40 | 3.58E−01 |
| Q9Y4F1 | FARP1 | 0.40 | 4.49E−01 |
| Q9HBU6 | ETNK1 | 0.39 | 3.04E−01 |
| Q9BY66 | KDM5D | 0.39 | 3.44E−01 |
| Q9UPN6 | SCAF8 | 0.39 | 2.78E−01 |
| Q9BTA9 | WAC | 0.39 | 2.36E−01 |
| Q92908 | GATA6 | 0.39 | 3.09E−01 |
| O43815 | STRN | 0.39 | 2.66E−01 |
| Q8IV63 | VRK3 | 0.39 | 4.20E−01 |
| P06493 | CDK1 | 0.39 | 1.76E−01 |
| Q13887 | KLF5 | 0.39 | 3.83E−01 |
| Q8IX01 | SUGP2 | 0.39 | 3.53E−01 |
| Q9H329 | EPB41L4B | 0.39 | 2.40E−01 |
| P0DPB5 | POLR1D | 0.39 | 3.83E−01 |
| Q6ZN18 | AEBP2 | 0.39 | 2.69E−01 |
| Q9H6E5 | TUT1 | 0.39 | 2.60E−01 |
| Q92995 | USP13 | 0.39 | 3.14E−01 |
| Q9GZT3 | SLIRP | 0.38 | 1.11E−01 |
| O95251 | KAT7 | 0.38 | 3.38E−01 |
| Q7L7X3 | TAOK1 | 0.38 | 4.19E−01 |
| P17676 | CEBPB | 0.38 | 3.58E−01 |
| Q9NRR4 | DROSHA | 0.38 | 2.19E−01 |
| Q9NYY8 | FASTKD2 | 0.38 | 3.14E−01 |
| P07478 | PRSS2 | 0.38 | 2.77E−01 |
| Q9H201 | EPN3 | 0.37 | 2.12E−01 |
| O75376 | NCOR1 | 0.37 | 2.52E−01 |
| P24468 | NR2F2 | 0.37 | 1.57E−01 |
| O00257 | CBX4 | 0.37 | 2.20E−01 |
| Q15398 | DLGAP5 | 0.37 | 2.07E−01 |
| Q9BZL1 | UBL5 | 0.37 | 3.97E−01 |
| Q9UIF9 | BAZ2A | 0.37 | 3.59E−01 |
| P42704 | LRPPRC | 0.37 | 1.12E−01 |
| Q8WWK9 | CKAP2 | 0.37 | 2.85E−01 |
| O43504 | LAMTOR5 | 0.37 | 2.44E−01 |
| P51114 | FXR1 | 0.37 | 2.40E−01 |
| Q13501 | SQSTM1 | 0.37 | 1.76E−01 |
| Q15572 | TAF1C | 0.37 | 2.10E−01 |
| Q03468 | ERCC6 | 0.37 | 2.40E−01 |
| Q96J94 | PIWIL1 | 0.36 | 2.11E−01 |
| Q9NP66 | HMG20A | 0.36 | 2.27E−01 |
| P19801 | AOC1 | 0.36 | 2.27E−01 |
| Q6P4A7 | SFXN4 | 0.36 | 2.27E−01 |
| Q14137 | BOP1 | 0.36 | 2.31E−01 |
| O14646 | CHD1 | 0.36 | 1.94E−01 |
| Q9NQC1 | JADE2 | 0.36 | 1.79E−01 |
| O95696 | BRD1 | 0.36 | 2.48E−01 |
| Q9ULD4 | BRPF3 | 0.36 | 1.84E−01 |
| O94887 | FARP2 | 0.36 | 2.64E−01 |
| Q7L2E3 | DHX30 | 0.36 | 2.64E−01 |
| O94880 | PHF14 | 0.36 | 1.81E−01 |
| Q14687 | GSE1 | 0.36 | 1.57E−01 |
| Q15345 | LRRC41 | 0.36 | 1.63E−01 |
| Q92817 | EVPL | 0.35 | 2.61E−01 |
| P20711 | DDC | 0.35 | 1.19E−01 |
| Q6PK04 | CCDC137 | 0.35 | 1.41E−01 |
| Q9H5V8 | CDCP1 | 0.35 | 1.53E−01 |
| Q15388 | TOMM20 | 0.35 | 1.68E−01 |
| O95297 | MPZL1 | 0.35 | 1.76E−01 |
| Q86WX3 | RPS19BP1 | 0.35 | 2.17E−01 |
| Q7L523 | RRAGA | 0.35 | 1.77E−01 |
| Q01664 | TFAP4 | 0.35 | 2.20E−01 |
| Q5T310 | GPATCH4 | 0.35 | 2.12E−01 |
| Q02556 | IRF8 | 0.35 | 1.81E−01 |
| P54274 | TERF1 | 0.35 | 1.05E−01 |
| O95071 | UBR5 | 0.34 | 1.15E−01 |
| Q9H8V3 | ECT2 | 0.34 | 2.10E−01 |
| Q9Y4E5 | ZNF451 | 0.34 | 1.76E−01 |
| Q99541 | PLIN2 | 0.34 | 1.87E−01 |
| P78545 | ELF3 | 0.34 | 1.72E−01 |
| Q8N2W9 | PIAS4 | 0.34 | 1.84E−01 |
| Q9NYT0 | PLEK2 | 0.34 | 1.68E−01 |
| Q02447 | SP3 | 0.34 | 1.69E−01 |
| Q9NZC4 | EHF | 0.33 | 1.64E−01 |
| Q6ZNB6 | NFXL1 | 0.33 | 8.39E−02 |
| P19634 | SLC9A1 | 0.33 | 1.41E−01 |
| Q2TB90 | HKDC1 | 0.33 | 3.83E−02 |
| Q9H582 | ZNF644 | 0.33 | 1.23E−01 |
| O43663 | PRC1 | 0.33 | 6.79E−02 |
| Q9NZI6 | TFCP2L1 | 0.33 | 1.15E−01 |
| O43823 | AKAP8 | 0.33 | 9.05E−02 |
| Q53RT3 | ASPRV1 | 0.33 | 9.29E−02 |
| Q06481 | APLP2 | 0.33 | 7.52E−02 |
| O15439 | ABCC4 | 0.32 | 9.45E−02 |
| P78325 | ADAM8 | 0.32 | 9.10E−02 |
| Q9Y287 | ITM2B | 0.32 | 1.15E−01 |
| P10242 | MYB | 0.32 | 1.10E−01 |
| Q99081 | TCF12 | 0.32 | 1.47E−01 |
| O15417 | TNRC18 | 0.32 | 1.15E−01 |
| Q5T6S3 | PHF19 | 0.32 | 6.38E−02 |
| Q9UPQ0 | LIMCH1 | 0.32 | 8.41E−02 |
| O00411 | POLRMT | 0.31 | 9.60E−02 |
| P24821 | TNC | 0.31 | 1.08E−01 |
| Q9ULW0 | TPX2 | 0.31 | 7.69E−02 |
| O95235 | KIF20A | 0.31 | 8.77E−02 |
| P36402 | TCF7 | 0.31 | 7.15E−02 |
| Q15562 | TEAD2 | 0.31 | 7.06E−02 |
| Q15561 | TEAD4 | 0.31 | 8.25E−02 |
| Q9H0H5 | RACGAP1 | 0.31 | 3.67E−02 |
| P04637 | TP53 | 0.31 | 5.33E−02 |
| POCG13 | CHTF8 | 0.30 | 9.05E−02 |
| Q8N531 | | 0.30 | 7.09E−02 |
| Q96QD8 | SLC38A2 | 0.30 | 4.92E−02 |
| O76080 | ZFAND5 | 0.30 | 4.81E−02 |
| P27930 | IL1R2 | 0.30 | 5.62E−02 |
| P43694 | GATA4 | 0.30 | 3.57E−02 |
| Q08211 | DHX9 | 0.30 | 1.36E−02 |
| Q9ULW3 | ABT1 | 0.29 | 6.74E−02 |
| P27105 | STOM | 0.29 | 3.62E−02 |
| Q9UER7 | DAXX | 0.29 | 5.54E−02 |
| Q9Y597 | KCTD3 | 0.29 | 6.33E−02 |
| Q9UPW6 | SATB2 | 0.29 | 5.95E−02 |
| P25774 | CTSS | 0.29 | 4.70E−02 |
| Q01826 | SATB1 | 0.29 | 4.82E−02 |
| Q13315 | ATM | 0.28 | 3.90E−02 |
| Q6DKI1 | RPL7L1 | 0.28 | 3.90E−02 |
| Q9Y4A0 | JRKL | 0.28 | 1.97E−02 |
| P41212 | ETV6 | 0.28 | 1.43E−02 |
| O00767 | SCD | 0.28 | 7.03E−03 |
| Q99856 | ARID3A | 0.28 | 7.35E−02 |
| Q9Y6H1 | CHCHD2 | 0.27 | 3.90E−02 |
| Q5T1J5 | CHCHD2P9 | 0.27 | 3.90E−02 |
| Q8WVX9 | FAR1 | 0.27 | 5.85E−02 |
| Q6ZN28 | MACC1 | 0.27 | 3.57E−02 |
| P61024 | CKS1B | 0.27 | 5.49E−02 |
| O14753 | OVOL1 | 0.27 | 1.51E−02 |
| Q14156 | EFR3A | 0.27 | 1.73E−02 |
| Q08945 | SSRP1 | 0.27 | 5.13E−03 |
| Q86V15 | CASZ1 | 0.27 | 2.24E−02 |
| Q9Y5B9 | SUPT16H | 0.27 | 2.06E−03 |
| P49454 | CENPF | 0.26 | 2.29E−02 |
| Q9UHL9 | GTF2IRD1 | 0.26 | 1.03E−02 |
| Q8NFA2 | NOXO1 | 0.25 | 1.15E−02 |
| P02795 | MT2A | 0.25 | 1.09E−02 |
| Q8WUJ3 | CEMIP | 0.24 | 1.43E−02 |
| Q7LFL8 | CXXC5 | 0.24 | 5.83E−03 |
| P37231 | PPARG | 0.23 | 1.16E−02 |
| Q14807 | KIF22 | 0.23 | 8.16E−03 |
| Q92786 | PROX1 | 0.23 | 6.61E−03 |
| Q9Y6G3 | MRPL42 | 0.22 | 6.96E−03 |
| Q8NI35 | PATJ | 0.22 | 2.30E−03 |
| Q9HOU9 | TSPYL1 | 0.22 | 1.59E−03 |
| Q92738 | USP6NL | 0.20 | 3.33E−03 |
| Q9P2B4 | CTTNBP2NL | 0.20 | 1.09E−03 |
| Q8N3R9 | PALS1 | 0.20 | 8.34E−04 |
| Q8WVM0 | TFB1M | 0.20 | 1.33E−03 |
| Q86WA8 | LONP2 | 0.19 | 1.06E−03 |
| Q9COK0 | BCL11B | 0.19 | 4.25E−04 |
| O14965 | AURKA | 0.18 | 1.12E−03 |
| Q96L93 | KIF16B | 0.18 | 1.05E−03 |
| Q9UNS1 | TIMELESS | 0.18 | 6.86E−04 |
| Q86T24 | ZBTB33 | 0.17 | 6.13E−04 |
| O95067 | CCNB2 | 0.17 | 7.39E−05 |
| Q460N5 | PARP14 | 0.17 | 5.93E−05 |
| Q8WV41 | SNX33 | 0.16 | 1.38E−04 |
| P06400 | RB1 | 0.16 | 2.17E−06 |
| P14635 | CCNB1 | 0.11 | 2.55E−06 |
| P20930 | FLG | 0.10 | 5.50E−07 |
| Q9NP58 | ABCB6 | 0.01 | 4.87E−16 |
| Q76L83 | ASXL2 | 0.01 | 4.87E−16 |
| O14493 | CLDN4 | 0.01 | 4.87E−16 |
| Q9UI42 | CPA4 | 0.01 | 4.87E−16 |
| Q96LJ7 | DHRS1 | 0.01 | 4.87E−16 |
| Q6IA86 | ELP2 | 0.01 | 4.87E−16 |
| 095714 | HERC2 | 0.01 | 4.87E−16 |
| Q32P51 | HNRNPA1L2 | 0.01 | 4.87E−16 |
| P98160 | HSPG2 | 0.01 | 4.87E−16 |
| Q9NSB2 | KRT84 | 0.01 | 4.87E−16 |
| Q86YC2 | PALB2 | 0.01 | 4.87E−16 |
| Q8IZ21 | PHACTR4 | 0.01 | 4.87E−16 |
| O43164 | PJA2 | 0.01 | 4.87E−16 |
| P53350 | PLK1 | 0.01 | 4.87E−16 |
| Q86VR2 | RETREG3 | 0.01 | 4.87E−16 |
| Q9UBS8 | RNF14 | 0.01 | 4.87E−16 |
| Q8ND24 | RNF214 | 0.01 | 4.87E−16 |
| Q5VWQ0 | RSBN1 | 0.01 | 4.87E−16 |
| Q5VWJ9 | SNX30 | 0.01 | 4.87E−16 |
| Q9H254 | SPTBN4 | 0.01 | 4.87E−16 |
| O75886 | STAM2 | 0.01 | 4.87E−16 |
| Q9BVT8 | TMUB1 | 0.01 | 4.87E−16 |
| A6NNY8 | USP27X | 0.01 | 4.87E−16 |

TABLE 3
Differential protein expression of MYXV-treated cytoplasm
and control cytoplasm

| Accession | Gene Symbol | $\frac{MYXV\_cyto}{Mock\_cyto}$ | P-value |
|---|---|---|---|
| Q5VWQ0 | RSBN1 | 100 | 2.81E−16 |
| Q9NSB2 | KRT84 | 100 | 2.81E−16 |
| D2WM37 | m012L | 100 | 2.81E−16 |
| E2CZV1 | m056R | 100 | 2.81E−16 |
| B2CWB2 | | 100 | 2.81E−16 |
| Q9PX24 | m007L | 100 | 2.81E−16 |
| B2CWK2 | m095L | 100 | 2.81E−16 |
| Q85296 | | 100 | 2.81E−16 |
| Q9Q8G6 | m135R | 100 | 2.81E−16 |
| Q9BRX9 | WDR83 | 100 | 2.81E−16 |
| P54274 | TERF1 | 100 | 2.81E−16 |
| Q9PM6 | MACROH2A2 | 100 | 2.81E−16 |
| Q9Q8K3 | m097R | 100 | 2.81E−16 |
| Q9GZN1 | ACTR6 | 100 | 2.81E−16 |
| Q9NV56 | MRGBP | 100 | 2.81E−16 |
| Q9NS91 | RAD18 | 100 | 2.81E−16 |
| Q9YJT4 | MA56 | 100 | 2.81E−16 |
| P38398 | BRCA1 | 100 | 2.81E−16 |
| B2CWP6 | m139R | 100 | 2.81E−16 |
| E2CZM0 | m131R | 100 | 2.81E−16 |
| Q9H6P5 | TASP1 | 100 | 2.81E−16 |
| Q8N954 | GPATCH11 | 100 | 2.81E−16 |
| Q6PJE2 | POMZP3 | 100 | 2.81E−16 |
| O00470 | MEIS1 | 100 | 2.81E−16 |
| Q9P1U0 | POLR1H | 100 | 2.81E−16 |
| B2CWC6 | m020L | 100 | 2.81E−16 |
| Q9H7N4 | SCAF1 | 100 | 2.81E−16 |
| O60292 | SIPA1L3 | 100 | 2.81E−16 |
| Q9UL40 | ZNF346 | 100 | 2.81E−16 |
| Q9HAJ7 | SAP30L | 100 | 2.81E−16 |
| Q9H8G2 | CAAP1 | 100 | 2.81E−16 |
| Q9Q8S2 | m020L | 100 | 2.81E−16 |
| Q9Y483 | MTF2 | 100 | 2.81E−16 |
| Q8NBZO | INO80E | 100 | 2.81E−16 |
| E2CZJ6 | m107L | 100 | 2.81E−16 |
| Q96LT9 | RNPC3 | 100 | 2.81E−16 |
| O43312 | MTSS1 | 100 | 2.81E−16 |
| B2CWL4 | | 100 | 2.81E−16 |
| Q9P1Y6 | PHRF1 | 100 | 2.81E−16 |
| Q9Y388 | RBMX2 | 100 | 2.81E−16 |
| Q8NCN4 | RNF169 | 100 | 2.81E−16 |
| Q9UER7 | DAXX | 100 | 2.81E−16 |
| E2CZL6 | m127L | 100 | 2.81E−16 |
| Q9Q8M6 | m074R | 100 | 2.81E−16 |
| Q9Y3Y2 | CHTOP | 100 | 2.81E−16 |
| Q8NDX5 | PHC3 | 100 | 2.81E−16 |
| Q9UK61 | TASOR | 100 | 2.81E−16 |
| O75529 | TAF5L | 100 | 2.81E−16 |
| Q9H6R0 | DHX33 | 100 | 2.81E−16 |
| Q96CB8 | INTS12 | 100 | 2.81E−16 |
| O15014 | ZNF609 | 100 | 2.81E−16 |
| Q9UQ16 | DNM3 | 100 | 2.81E−16 |
| Q96E39 | RBMXL1 | 100 | 2.81E−16 |
| O15523 | DDX3Y | 100 | 2.81E−16 |
| D3W0C4 | m130R | 100 | 2.81E−16 |
| O55698 | M-T4 | 100 | 2.81E−16 |
| B2CWA8 | | 100 | 2.81E−16 |
| Q9PXA3 | m004L | 100 | 2.81E−16 |
| B2CWD6 | m029L | 100 | 2.81E−16 |
| B2CWR1 | m156R | 100 | 2.81E−16 |
| Q5VVJ2 | MYSM1 | 100 | 2.81E−16 |
| Q9Q8G4 | m137R | 100 | 2.81E−16 |
| Q9Q8S8 | m013L | 100 | 2.81E−16 |
| Q83730 | m005R | 100 | 2.81E−16 |
| B2CWB0 | | 100 | 2.81E−16 |
| Q9Q8R4 | m032R | 100 | 2.81E−16 |
| Q9Q8K4 | VETFL | 100 | 2.81E−16 |
| B2CWK3 | | 100 | 2.81E−16 |
| B2CWQ5 | m149R | 100 | 2.81E−16 |
| Q9Q8J4 | m106L | 100 | 2.81E−16 |
| Q9Q8J5 | m105L | 100 | 2.81E−16 |
| Q9Q8N0 | m070R | 100 | 2.81E−16 |
| B2CWG1 | | 100 | 2.81E−16 |
| K4J9W4 | m054R | 100 | 2.81E−16 |
| B2CWM2 | m115L | 95.455 | 2.81E−16 |
| B2CWG8 | m061R | 94.847 | 2.81E−16 |
| Q9Q8K6 | m094R | 70.579 | 2.81E−16 |
| B2CWJ5 | | 57.491 | 2.81E−16 |
| E2CZH7 | m088L | 57.491 | 2.81E−16 |
| Q9YQ03 | mt6 | 55.666 | 2.81E−16 |
| B2CWG0 | m053R | 54.489 | 2.81E−16 |
| Q9Q8Q8 | m038L | 53.022 | 2.81E−16 |
| Q9Q8J9 | m101L | 40.89 | 2.81E−16 |
| E2CZP2 | m001R | 38.111 | 2.81E−16 |
| O10625 | M-T1 | 38.111 | 2.81E−16 |
| D2WMA0 | m030L | 37.506 | 2.81E−16 |
| Q9Q8P8 | m048L | 31.815 | 2.81E−16 |
| D2WN05 | m078R | 27.499 | 2.81E−16 |
| Q9PX36 | m006L | 27.02 | 2.81E−16 |
| E2CZR0 | m015L | 26.217 | 2.81E−16 |
| B2CWC1 | m015L | 26.217 | 2.81E−16 |
| B2CWI7 | m080R | 25.328 | 2.81E−16 |
| Q77P93 | m003.2L | 25.064 | 2.81E−16 |
| Q9Q8R2 | m034L | 24.114 | 2.81E−16 |
| B2CWE1 | | 24.114 | 2.81E−16 |
| B2CWB5 | | 23.767 | 2.81E−16 |
| E2CZQ3 | m009L | 23.767 | 2.81E−16 |
| Q85295 | M11L | 22.899 | 2.81E−16 |
| B2CWC8 | | 22.84 | 2.81E−16 |
| Q9Q8N5 | | 21.987 | 2.81E−16 |
| B2CWH0 | | 21.987 | 2.81E−16 |
| Q9Q8F0 | m154L | 19.646 | 2.81E−16 |
| Q83604 | MF13 | 19.529 | 2.81E−16 |
| Q9Q8L3 | m087L | 18.722 | 2.81E−16 |
| B2CWA6 | m003.1R | 17.331 | 2.81E−16 |
| Q77PB1 | m003.1L | 17.331 | 2.81E−16 |
| D2WN93 | m151R | 16.658 | 2.81E−16 |
| B2CWQ7 | | 16.658 | 2.81E−16 |
| Q9Q8H9 | m122R | 16.215 | 2.81E−16 |
| Q9Q8H2 | m129R | 15.466 | 2.81E−16 |
| P07942 | LAMB1 | 14.644 | 2.81E−16 |
| Q9Q8I4 | m117L | 14.634 | 2.81E−16 |
| B2CWH6 | m069L | 14.196 | 2.81E−16 |
| Q85297 | | 14.196 | 2.81E−16 |
| B2CWB4 | m008.1L | 13.719 | 2.81E−16 |
| Q9Q8K1 | m099L | 13.399 | 2.81E−16 |
| Q9Q8Q6 | m040L | 13.343 | 2.81E−16 |
| Q9Q8L8 | m082R | 13.227 | 2.81E−16 |
| P68544 | PAPS | 11.943 | 2.81E−16 |
| E2CZW3 | m068R | 11.311 | 2.81E−16 |
| Q92993 | KAT5 | 9.786 | 2.81E−16 |
| Q9Q8J1 | m109L | 9.336 | 2.81E−16 |
| B2CWI3 | m076R | 8.624 | 2.81E−16 |
| Q9Q8K7 | m093L | 8.506 | 2.81E−16 |
| E2CZW7 | m072L | 8.015 | 2.81E−16 |
| Q9Q8L9 | VETFS | 7.989 | 2.81E−16 |
| Q5VU97 | CACHD1 | 7.631 | 4.15E−08 |
| B2CWQ3 | m147R | 7.304 | 6.12E−15 |
| B2CWP1 | | 6.946 | 2.81E−16 |
| K4JGJ2 | m134R | 6.946 | 2.81E−16 |
| P43243 | MATR3 | 6.936 | 2.81E−16 |
| Q92804 | TAF15 | 6.931 | 2.81E−16 |
| B2CWD4 | m027L | 6.841 | 2.81E−16 |
| P38159 | RBMX | 6.621 | 2.81E−16 |
| Q9Y2K1 | ZBTB1 | 6.438 | 4.40E−13 |
| O75526 | RBMXL2 | 6.375 | 6.12E−15 |
| B2CWG7 | m060R | 6.24 | 7.13E−13 |
| O14979 | HNRNPDL | 6.226 | 2.81E−16 |
| B2CWJ3 | | 6.189 | 2.25E−12 |
| E2CZY1 | m086L | 6.189 | 2.25E−12 |
| Q71DI3 | H3C13; | 6.151 | 2.58E−09 |
| | H3C14; | | |
| | H3C15 | | |
| Q9Q8M5 | m075R | 5.955 | 2.81E−16 |
| P22626 | HNRNPA2B1 | 5.588 | 2.81E−16 |
| Q13595 | TRA2A | 5.471 | 5.24E−12 |
| Q9Q8P4 | m052L | 5.461 | 6.86E−13 |
| P51991 | HNRNPA3 | 5.387 | 2.81E−16 |
| P68550 | | 5.026 | 2.81E−16 |
| P31942 | HNRNPH3 | 4.983 | 2.81E−16 |
| Q15424 | SAFB | 4.907 | 4.69E−09 |
| O14493 | CLDN4 | 4.876 | 1.36E−08 |
| P08621 | SNRNP70 | 4.865 | 6.12E−15 |
| Q9Q8M9 | m071L | 4.784 | 6.12E−15 |
| B2CWH8 | | 4.598 | 6.09E−14 |
| D2WN26 | m090L | 4.598 | 2.06E−04 |
| P14866 | HNRNPL | 4.567 | 2.81E−16 |
| Q13242 | SRSF9 | 4.483 | 3.91E−10 |
| P07910 | HNRNPC | 4.478 | 2.81E−16 |
| O75494 | SRSF10 | 4.337 | 9.57E−10 |
| Q12996 | CSTF3 | 4.301 | 1.23E−09 |
| B7ZW38 | HNRNPCL3 | 4.203 | 5.82E−13 |
| O60812 | HNRNPCL1 | 4.203 | 1.01E−12 |
| B2RXH8 | HNRNPCL2 | 4.203 | 1.01E−12 |
| P62995 | TRA2B | 4.124 | 2.21E−12 |
| P16402 | H1-3 | 4.082 | 9.07E−04 |
| E2CZV0 | m055R | 4.078 | 2.46E−11 |
| Q9H0M0 | WWP1 | 4.058 | 2.37E−07 |
| Q12864 | CDH17 | 3.8 | 2.43E−09 |
| O43390 | HNRNPR | 3.727 | 2.79E−12 |
| P62316 | SNRPD2 | 3.714 | 1.89E−11 |
| B2CWI0 | | 3.623 | 6.85E−11 |
| K4JKC2 | m073R | 3.623 | 6.85E−11 |
| B2CWM1 | | 3.607 | 4.44E−10 |
| E2CZK3 | m114R | 3.607 | 4.44E−10 |
| Q9Q8I6 | m114R | 3.607 | 4.44E−10 |
| Q15428 | SF3A2 | 3.591 | 3.83E−10 |
| Q9Q8F9 | m144R | 3.514 | 5.82E−10 |
| P23246 | SFPQ | 3.454 | 7.40E−12 |
| Q8N684 | CPSF7 | 3.444 | 5.28E−08 |
| Q00526 | CDK3 | 3.412 | 2.18E−04 |
| Q8WU68 | U2AF1L4 | 3.374 | 1.45E−06 |
| Q8WXA9 | SREK1 | 3.305 | 1.12E−03 |
| O00566 | MPHOSPH10 | 3.277 | 5.38E−05 |
| P84077 | ARF1 | 3.272 | 3.04E−06 |
| Q15287 | RNPS1 | 3.247 | 4.50E−06 |
| Q9UKM9 | RALY | 3.213 | 5.94E−07 |
| Q9NWB6 | ARGLU1 | 3.18 | 7.49E−05 |
| P09651 | HNRNPA1 | 3.174 | 2.37E−10 |
| Q14004 | CDK13 | 3.156 | 5.25E−03 |
| Q9H307 | PNN | 3.14 | 1.32E−05 |
| P62314 | SNRPD1 | 3.128 | 2.86E−08 |
| Q9Q8I1 | m120L | 3.083 | 9.03E−06 |
| Q16629 | SRSF7 | 3.079 | 5.14E−08 |
| Q99729 | HNRNPAB | 3.068 | 1.03E−07 |
| Q9Y3B4 | SF3B6 | 3.066 | 3.91E−05 |
| B2CWI4 | m077L | 3.01 | 1.01E−05 |
| Q2TAY7 | SMU1 | 2.99 | 2.15E−07 |
| B2CWJ9 | m092L | 2.985 | 3.58E−08 |
| P62304 | SNRPE | 2.931 | 3.65E−06 |
| Q15233 | NONO | 2.914 | 6.03E−09 |
| Q9BQ04 | RBM4B | 2.911 | 3.88E−03 |
| Q99459 | CDC5L | 2.903 | 3.73E−07 |
| Q92841 | DDX17 | 2.884 | 8.80E−09 |
| P83876 | TXNL4A | 2.853 | 3.24E−04 |
| Q15717 | ELAVL1 | 2.828 | 6.14E−07 |
| Q9H4I0 | RAD21L1 | 2.821 | 1.90E−03 |
| P09012 | SNRPA | 2.796 | 1.44E−06 |
| P57721 | PCBP3 | 2.786 | 2.95E−02 |
| Q05519 | SRSF11 | 2.783 | 1.31E−06 |
| Q13151 | HNRNPA0 | 2.751 | 2.72E−06 |
| Q01081 | U2AF1 | 2.734 | 3.62E−06 |
| Q9Y3B2 | EXOSC1 | 2.732 | 5.90E−04 |
| P07305 | H1-0 | 2.726 | 2.10E−04 |
| Q8NEJ9 | NGDN | 2.723 | 2.01E−04 |
| O43818 | RRP9 | 2.72 | 1.93E−04 |
| O43660 | PLRG1 | 2.707 | 1.71E−05 |
| Q1KMD3 | HNRNPUL2 | 2.705 | 1.00E−06 |
| Q9UMS4 | PRPF19 | 2.703 | 3.79E−06 |
| Q9BW30 | TPPP3 | 2.694 | 4.84E−06 |
| A8MWD9 | SNRPGP15 | 2.658 | 2.56E−05 |
| P62308 | SNRPG | 2.658 | 2.84E−05 |
| P35580 | MYH10 | 2.654 | 1.63E−07 |
| P62306 | SNRPF | 2.647 | 2.69E−04 |
| Q9Y2P8 | RCL1 | 2.634 | 4.08E−04 |
| B2CWG5 | m058R | 2.631 | 3.18E−06 |
| Q9Q8N8 | m058R | 2.631 | 6.28E−06 |
| Q7RTV0 | PHF5A | 2.608 | 1.68E−04 |
| P22492 | H1-6 | 2.606 | 7.12E−06 |
| Q02539 | H1-1 | 2.606 | 7.12E−06 |
| Q9NYF8 | BCLAF1 | 2.601 | 1.26E−05 |
| Q9UQE7 | SMC3 | 2.588 | 9.63E−06 |
| P55795 | HNRNPH2 | 2.571 | 1.68E−05 |
| Q9Q8G2 | m140R | 2.569 | 7.74E−04 |
| B2CWG4 | m057L | 2.546 | 5.80E−02 |
| Q9NQT4 | EXOSC5 | 2.545 | 5.19E−04 |
| Q15397 | PUM3 | 2.542 | 3.86E−04 |
| Q5VTL8 | PRPF38B | 2.541 | 2.50E−02 |
| Q9Y2W1 | THRAP3 | 2.536 | 5.86E−06 |
| Q13523 | PRPF4B | 2.528 | 7.81E−04 |
| Q96DI7 | SNRNP40 | 2.525 | 6.20E−04 |
| P12830 | CDH1 | 2.518 | 3.49E−05 |
| Q12788 | TBL3 | 2.516 | 1.30E−03 |
| 060306 | AQR | 2.502 | 1.81E−05 |
| E2CZL2 | m123R | 2.49 | 1.58E−03 |
| P14678 | SNRPB | 2.477 | 4.21E−05 |
| P63162 | SNRPN | 2.477 | 4.21E−05 |
| Q8TB72 | PUM2 | 2.469 | 1.09E−02 |
| O75362 | ZNF217 | 2.454 | 6.07E−02 |
| Q96I25 | RBM17 | 2.435 | 2.13E−03 |
| P55769 | SNU13 | 2.425 | 2.06E−04 |
| Q5JTH9 | RRP12 | 2.424 | 2.84E−05 |
| P49756 | RBM25 | 2.421 | 4.29E−04 |
| Q5SXH7 | PLEKHS1 | 2.419 | 5.44E−03 |
| Q9NPD3 | EXOSC4 | 2.418 | 7.05E−04 |
| Q9BSC4 | NOL10 | 2.407 | 2.20E−02 |
| P35579 | MYH9 | 2.406 | 3.63E−06 |
| Q13601 | KRR1 | 2.404 | 1.27E−03 |
| Q8IX12 | CCAR1 | 2.402 | 5.67E−05 |
| 094762 | RECQL5 | 2.388 | 7.37E−02 |
| P16401 | H1-5 | 2.386 | 1.10E−04 |
| Q9HCD5 | NCOA5 | 2.383 | 6.03E−05 |
| Q15061 | WDR43 | 2.38 | 2.12E−03 |
| Q14151 | SAFB2 | 2.378 | 9.33E−04 |
| Q12874 | SF3A3 | 2.373 | 1.45E−04 |
| Q9BUQ8 | DDX23 | 2.368 | 6.53E−05 |
| O15226 | NKRF | 2.366 | 1.13E−03 |
| O14896 | IRF6 | 2.365 | 3.50E−03 |
| P10412 | H1-4 | 2.358 | 1.19E−04 |
| P84103 | SRSF3 | 2.357 | 1.20E−04 |
| Q96KR1 | ZFR | 2.349 | 1.88E−03 |
| O95453 | PARN | 2.341 | 4.64E−04 |
| Q9NWH9 | SLTM | 2.34 | 1.95E−03 |
| Q9NY12 | GAR1 | 2.337 | 7.42E−03 |
| O43809 | NUDT21 | 2.336 | 1.93E−04 |
| O75367 | MACROH2A1 | 2.311 | 5.96E−03 |
| Q6DN03 | H2BC20P | 2.298 | 2.13E−03 |
| Q6DRA6 | H2BC19P | 2.298 | 2.13E−03 |
| Q8N257 | H2BU1 | 2.298 | 2.13E−03 |
| Q16778 | H2BC21 | 2.298 | 2.13E−03 |
| P33778 | H2BC3 | 2.298 | 2.13E−03 |
| P06899 | H2BC11 | 2.298 | 2.13E−03 |
| P23527 | H2BC17 | 2.298 | 2.13E−03 |
| Q15050 | RRS1 | 2.257 | 7.80E−03 |
| Q15427 | SF3B4 | 2.255 | 4.00E−03 |
| Q9UDY4 | DNAJB4 | 2.255 | 1.07E−02 |
| Q9BTD8 | RBM42 | 2.242 | 2.83E−03 |
| Q13435 | SF3B2 | 2.238 | 1.59E−04 |
| Q9Q8Q1 | | 2.237 | 1.18E−01 |
| Q9NUK0 | MBNL3 | 2.232 | 3.41E−03 |
| Q9UPQ3 | AGAP1 | 2.229 | 1.37E−01 |
| O60341 | KDM1A | 2.227 | 1.21E−03 |
| P84243 | H3-3A; H3-3B | 2.226 | 4.16E−03 |
| Q9Y2X3 | NOP58 | 2.224 | 1.72E−04 |
| P19105 | MYL12A | 2.213 | 2.88E−04 |
| Q8TDN6 | BRIX1 | 2.213 | 1.15E−03 |
| Q06265 | EXOSC9 | 2.204 | 5.90E−03 |
| Q9NY61 | AATF | 2.196 | 4.26E−04 |
| Q9H7B2 | RPF2 | 2.196 | 3.36E−03 |
| O14950 | MYL12B | 2.192 | 3.56E−04 |
| P31943 | HNRNPH1 | 2.19 | 5.30E−05 |
| P98179 | RBM3 | 2.185 | 6.79E−04 |
| Q6P1J9 | CDC73 | 2.181 | 1.32E−03 |
| Q92979 | EMG1 | 2.175 | 8.76E−03 |
| Q96SI9 | STRBP | 2.174 | 6.74E−04 |
| P98175 | RBM10 | 2.174 | 5.11E−03 |
| Q5VT52 | RPRD2 | 2.174 | 1.22E−02 |
| Q9UPQ0 | LIMCH1 | 2.17 | 1.50E−01 |
| Q16630 | CPSF6 | 2.167 | 1.08E−03 |
| Q8N3U4 | STAG2 | 2.164 | 6.77E−03 |
| P26368 | U2AF2 | 2.159 | 6.55E−04 |
| O60216 | RAD21 | 2.158 | 1.00E−02 |
| Q9BYE7 | PCGF6 | 2.158 | 1.49E−01 |
| P62318 | SNRPD3 | 2.15 | 5.76E−04 |
| Q9UKV3 | ACIN1 | 2.149 | 2.56E−03 |
| Q7Z406 | MYH14 | 2.148 | 8.05E−04 |
| Q15459 | SF3A1 | 2.14 | 6.79E−04 |
| P61513 | RPL37A | 2.138 | 1.41E−03 |
| P07199 | CENPB | 2.137 | 1.76E−01 |
| Q9NVP1 | DDX18 | 2.124 | 1.02E−03 |
| P14649 | MYL6B | 2.123 | 5.65E−04 |
| Q8TED0 | UTP15 | 2.119 | 1.79E−01 |
| Q15024 | EXOSC7 | 2.109 | 7.59E−03 |
| Q8TF68 | ZNF384 | 2.107 | 2.09E−01 |
| P24844 | MYL9 | 2.092 | 1.32E−03 |
| Q13123 | IK | 2.089 | 1.19E−02 |
| Q9Q8Q3 | m043L | 2.085 | 2.86E−02 |
| Q00839 | HNRNPU | 2.083 | 1.94E−04 |
| O00567 | NOP56 | 2.083 | 1.32E−03 |
| O94906 | PRPF6 | 2.077 | 1.38E−03 |
| Q5RKV6 | EXOSC6 | 2.075 | 2.61E−03 |
| Q07955 | SRSF1 | 2.073 | 1.57E−03 |
| Q8WXF1 | PSPC1 | 2.064 | 1.27E−03 |
| A6NHQ2 | FBLL1 | 2.062 | 1.78E−02 |
| O43823 | AKAP8 | 2.062 | 6.36E−02 |
| P17844 | DDX5 | 2.06 | 2.53E−04 |
| Q5BKZ1 | ZNF326 | 2.06 | 1.94E−02 |
| Q6UN15 | FIP1L1 | 2.059 | 1.70E−02 |
| Q6P2Q9 | PRPF8 | 2.057 | 2.08E−03 |
| P22087 | FBL | 2.056 | 2.00E−03 |
| Q9NV31 | IMP3 | 2.053 | 2.22E−01 |
| Q9UKL0 | RCOR1 | 2.051 | 2.12E−02 |
| B2CWF3 | m046L | 2.048 | 1.50E−01 |
| P47813 | EIF1AX | 2.044 | 7.19E−02 |
| Q96EP5 | DAZAP1 | 2.042 | 2.02E−03 |
| B2CWP9 | m142R | 2.041 | 1.18E−02 |
| Q14683 | SMC1A | 2.037 | 1.59E−03 |
| Q13247 | SRSF6 | 2.034 | 2.32E−03 |
| Q8TAQ2 | SMARCC2 | 2.033 | 3.50E−04 |
| Q96T37 | RBM15 | 2.031 | 1.57E−02 |
| O43172 | PRPF4 | 2.025 | 1.78E−03 |
| O94776 | MTA2 | 2.022 | 1.74E−03 |
| Q15059 | BRD3 | 2.021 | 4.64E−03 |
| Q8IY37 | DHX37 | 2.018 | 1.87E−02 |
| Q8NI36 | WDR36 | 2.012 | 4.03E−02 |
| Q6PD62 | CTR9 | 2.007 | 4.41E−03 |
| Q9BWJ5 | SF3B5 | 2.006 | 1.46E−02 |
| O43395 | PRPF3 | 2.002 | 4.57E−03 |
| P09234 | SNRPC | 2.002 | 6.77E−03 |
| Q8N6T7 | SIRT6 | 2.002 | 2.63E−01 |
| Q9UH17 | APOBEC3B | 2.001 | 2.42E−01 |
| Q9NPF2 | CHST11 | 0.498 | 8.77E−02 |
| Q9H9F9 | ACTR5 | 0.496 | 1.17E−01 |
| P13674 | P4HA1 | 0.495 | 6.74E−02 |
| Q9BV57 | ADI1 | 0.494 | 2.88E−03 |
| P61769 | B2M | 0.494 | 5.31E−03 |
| Q9BYM8 | RBCK1 | 0.494 | 1.05E−01 |
| P17066 | HSPA6 | 0.494 | 2.23E−01 |
| P54760 | EPHB4 | 0.493 | 2.14E−01 |
| Q8WVX9 | FAR1 | 0.486 | 3.02E−02 |
| P11388 | TOP2A | 0.485 | 3.03E−03 |
| 015391 | YY2 | 0.484 | 1.25E−01 |
| Q7Z7K6 | CENPV | 0.484 | 2.93E−01 |
| P16989 | YBX3 | 0.483 | 4.32E−03 |
| P98082 | DAB2 | 0.483 | 7.32E−02 |
| P54278 | PMS2 | 0.483 | 2.70E−01 |
| Q9C0C9 | UBE20 | 0.482 | 3.27E−03 |
| Q96BJ8 | ELMO3 | 0.482 | 4.55E−02 |
| P01034 | CST3 | 0.481 | 2.36E−03 |
| Q8IWV8 | UBR2 | 0.481 | 6.81E−02 |
| Q9UHD9 | UBQLN2 | 0.48 | 1.84E−02 |
| P04181 | OAT | 0.479 | 2.01E−03 |
| Q96L93 | KIF16B | 0.478 | 2.84E−01 |
| P30825 | SLC7A1 | 0.476 | 2.87E−03 |
| Q8WTV0 | SCARB1 | 0.476 | 2.97E−02 |
| Q8N5J2 | MINDY1 | 0.476 | 6.90E−02 |
| P52732 | KIF11 | 0.473 | 1.55E−01 |
| Q9BRK5 | SDF4 | 0.472 | 9.79E−03 |
| P25490 | YY1 | 0.472 | 7.37E−02 |
| P20823 | HNF1A | 0.471 | 1.37E−01 |
| Q16656 | NRF1 | 0.468 | 1.38E−01 |
| P26358 | DNMT1 | 0.466 | 8.95E−04 |
| Q9NV92 | NDFIP2 | 0.466 | 7.26E−02 |
| Q9NX18 | SDHAF2 | 0.465 | 3.57E−02 |
| Q9BZG8 | DPH1 | 0.465 | 2.54E−01 |
| P07602 | PSAP | 0.464 | 1.85E−03 |
| Q9NUW8 | TDP1 | 0.463 | 2.52E−01 |
| Q99808 | SLC29A1 | 0.462 | 9.89E−03 |
| Q15582 | TGFBI | 0.462 | 4.68E−02 |
| Q9UJC3 | HOOK1 | 0.461 | 1.02E−02 |
| Q96MM3 | ZFP42 | 0.46 | 1.51E−01 |
| Q01650 | SLC7A5 | 0.458 | 1.85E−03 |
| P13498 | CYBA | 0.458 | 1.84E−02 |
| Q9HOU9 | TSPYL1 | 0.456 | 2.24E−01 |
| Q8IZW8 | TNS4 | 0.455 | 8.74E−04 |
| P08195 | SLC3A2 | 0.454 | 2.04E−04 |
| Q13029 | PRDM2 | 0.454 | 1.56E−01 |
| Q99595 | TIMM17A | 0.453 | 1.78E−02 |
| Q06546 | GABPA | 0.453 | 2.55E−02 |
| Q9H0X4 | FAM234A | 0.451 | 7.04E−03 |
| Q9Y287 | ITM2B | 0.451 | 4.36E−02 |
| P78325 | ADAM8 | 0.449 | 2.11E−01 |
| P11802 | CDK4 | 0.448 | 1.07E−01 |
| O94916 | NFAT5 | 0.448 | 1.90E−01 |
| Q9BZD4 | NUF2 | 0.447 | 9.40E−02 |
| Q8IXJ6 | SIRT2 | 0.445 | 1.03E−01 |
| Q9Y508 | RNF114 | 0.443 | 9.79E−03 |
| Q15800 | MSMO1 | 0.441 | 2.73E−02 |
| Q9Y653 | ADGRG1 | 0.441 | 2.78E−02 |
| P51956 | NEK3 | 0.441 | 9.67E−02 |
| Q9Y5X3 | SNX5 | 0.44 | 4.80E−04 |
| Q8NFA2 | NOXO1 | 0.44 | 2.01E−01 |
| Q96PZ2 | FAM111A | 0.434 | 1.74E−01 |
| Q9UK76 | JPT1 | 0.433 | 4.31E−04 |
| Q9Y2A9 | B3GNT3 | 0.433 | 1.78E−02 |
| Q92817 | EVPL | 0.431 | 6.75E−02 |
| Q8TDB6 | DTX3L | 0.426 | 2.97E−02 |
| Q9H999 | PANK3 | 0.425 | 2.19E−02 |
| Q9H0H5 | RACGAP1 | 0.424 | 1.40E−01 |
| Q13049 | TRIM32 | 0.423 | 1.44E−01 |
| P32519 | ELF1 | 0.422 | 1.14E−02 |
| Q86UW9 | DTX2 | 0.42 | 1.76E−02 |
| Q9P2B4 | CTTNBP2NL | 0.419 | 1.37E−01 |
| P25311 | AZGP1 | 0.418 | 1.20E−04 |
| P56524 | HDAC4 | 0.418 | 2.98E−03 |
| Q13952 | NFYC | 0.417 | 5.33E−02 |
| Q9H2K8 | TAOK3 | 0.412 | 1.59E−03 |
| P00533 | EGFR | 0.412 | 6.01E−03 |
| Q9Y240 | CLEC11A | 0.41 | 1.21E−04 |
| P05204 | HMGN2 | 0.41 | 9.35E−02 |
| O14777 | NDC80 | 0.409 | 1.04E−01 |
| Q92876 | KLK6 | 0.407 | 4.07E−05 |
| Q7Z4H7 | HAUS6 | 0.404 | 1.17E−01 |
| Q8IXQ6 | PARP9 | 0.4 | 6.67E−02 |
| Q6DKI1 | RPL7L1 | 0.399 | 2.32E−04 |
| Q8N6R0 | METTL13 | 0.399 | 1.01E−02 |
| Q96J94 | PIWIL1 | 0.397 | 3.74E−03 |
| P08243 | ASNS | 0.395 | 4.28E−06 |
| P04626 | ERBB2 | 0.395 | 7.35E−04 |
| Q8NBJ4 | GOLM1 | 0.394 | 1.58E−04 |
| Q8TD30 | GPT2 | 0.394 | 7.15E−04 |
| P02795 | MT2A | 0.393 | 4.86E−03 |
| P18065 | IGFBP2 | 0.393 | 6.32E−03 |
| O76080 | ZFAND5 | 0.393 | 4.09E−02 |
| Q63HN8 | RNF213 | 0.392 | 5.82E−03 |
| Q9BU68 | PRR15L | 0.39 | 3.41E−02 |
| O43286 | B4GALT5 | 0.389 | 3.38E−02 |
| P26006 | ITGA3 | 0.383 | 5.49E−03 |
| Q14258 | TRIM25 | 0.382 | 1.56E−06 |
| Q15517 | CDSN | 0.379 | 7.37E−02 |
| Q9BZL1 | UBL5 | 0.373 | 3.25E−04 |
| Q16539 | MAPK14 | 0.37 | 3.63E−04 |
| Q15375 | EPHA7 | 0.369 | 3.57E−04 |
| O00468 | AGRN | 0.369 | 1.35E−03 |
| P10909 | CLU | 0.368 | 6.12E−02 |
| Q96Q05 | TRAPPC9 | 0.367 | 6.11E−02 |
| Q8N3R9 | PALS1 | 0.362 | 5.52E−02 |
| Q15853 | USF2 | 0.362 | 5.80E−02 |
| P48380 | RFX3 | 0.361 | 2.40E−02 |
| O14965 | AURKA | 0.36 | 1.32E−02 |
| P36402 | TCF7 | 0.359 | 1.50E−03 |
| P31350 | RRM2 | 0.356 | 2.75E−06 |
| Q5T1J5 | CHCHD2P9 | 0.352 | 1.15E−03 |
| Q9Y6H1 | CHCHD2 | 0.352 | 1.15E−03 |
| P37268 | FDFT1 | 0.351 | 7.04E−07 |
| P48436 | SOX9 | 0.346 | 1.22E−03 |
| Q8IVH2 | FOXP4 | 0.343 | 1.02E−04 |
| O75976 | CPD | 0.341 | 2.93E−07 |
| Q9Y4A0 | JRKL | 0.337 | 3.28E−02 |
| Q96QD8 | SLC38A2 | 0.336 | 3.87E−04 |
| Q8WUJ3 | CEMIP | 0.335 | 6.68E−04 |
| Q9UPU5 | USP24 | 0.329 | 2.16E−05 |
| Q6PJ69 | TRIM65 | 0.327 | 8.07E−04 |
| P33908 | MAN1A1 | 0.326 | 1.28E−03 |
| P98160 | HSPG2 | 0.324 | 2.49E−02 |
| P54753 | EPHB3 | 0.318 | 2.16E−02 |
| P54098 | POLG | 0.317 | 2.00E−02 |
| O43570 | CA12 | 0.304 | 2.47E−04 |
| P05067 | APP | 0.302 | 3.15E−07 |
| P55061 | TMBIM6 | 0.299 | 8.36E−05 |
| P08047 | SP1 | 0.283 | 1.00E−03 |
| O00767 | SCD | 0.279 | 2.86E−09 |
| Q9UH92 | MLX | 0.278 | 2.45E−03 |
| P14635 | CCNB1 | 0.274 | 5.67E−05 |
| Q9NPA3 | MID1IP1 | 0.273 | 3.67E−05 |
| P06400 | RB1 | 0.271 | 2.20E−09 |
| E2CZL5 | m126R | 0.263 | 1.27E−06 |
| Q06481 | APLP2 | 0.26 | 1.82E−06 |
| P07478 | PRSS2 | 0.252 | 1.82E−11 |
| Q460N5 | PARP14 | 0.247 | 6.54E−07 |
| Q6SPF0 | SAMD1 | 0.244 | 8.74E−04 |
| Q13033 | STRN3 | 0.24 | 6.41E−06 |
| Q13950 | RUNX2 | 0.239 | 9.57E−04 |
| Q99541 | PLIN2 | 0.235 | 5.42E−10 |
| O00762 | UBE2C | 0.234 | 1.68E−06 |
| P17516 | AKR1C4 | 0.231 | 1.95E−04 |
| Q8WV41 | SNX33 | 0.215 | 4.25E−04 |
| P78545 | ELF3 | 0.214 | 1.91E−05 |
| P25774 | CTSS | 0.195 | 5.84E−11 |
| P08910 | ABHD2 | 0.172 | 2.57E−09 |
| Q15011 | HERPUD1 | 0.157 | 4.26E−07 |
| Q9BZC7 | ABCA2 | 0.108 | 3.12E−08 |
| O00628 | PEX7 | 0.09 | 1.03E−13 |
| Q9P2R6 | RERE | 0.01 | 2.81E−16 |
| P17535 | JUND | 0.01 | 2.81E−16 |
| P05090 | APOD | 0.01 | 2.81E−16 |
| Q9ULH7 | MRTFB | 0.01 | 2.81E−16 |
| Q15398 | DLGAP5 | 0.01 | 2.81E−16 |
| P53567 | CEBPG | 0.01 | 2.81E−16 |
| Q14686 | NCOA6 | 0.01 | 2.81E−16 |
| O75052 | NOS1AP | 0.01 | 2.81E−16 |
| Q5VZL5 | ZMYM4 | 0.01 | 2.81E−16 |
| Q9NQV6 | PRDM10 | 0.01 | 2.81E−16 |
| Q9NVW2 | RLIM | 0.01 | 2.81E−16 |
| Q96K58 | ZNF668 | 0.01 | 2.81E−16 |
| Q8NB78 | KDM1B | 0.01 | 2.81E−16 |
| Q9H3F6 | KCTD10 | 0.01 | 2.81E−16 |
| A8MW92 | PHF20L1 | 0.01 | 2.81E−16 |
| Q08999 | RBL2 | 0.01 | 2.81E−16 |
| Q99081 | TCF12 | 0.01 | 2.81E−16 |
| P40426 | PBX3 | 0.01 | 2.81E−16 |
| Q9H334 | FOXP1 | 0.01 | 2.81E−16 |
| Q6NZ67 | MZT2B | 0.01 | 2.81E−16 |
| Q6P582 | MZT2A | 0.01 | 2.81E−16 |
| Q6E0U4 | DMKN | 0.01 | 2.81E−16 |
| Q9NQC1 | JADE2 | 0.01 | 2.81E−16 |
| A8K2U0 | A2ML1 | 0.01 | 2.81E−16 |
| P22415 | USF1 | 0.01 | 2.81E−16 |
| Q8TD26 | CHD6 | 0.01 | 2.81E−16 |
| Q9HB58 | SP110 | 0.01 | 2.81E−16 |
| Q6MZP7 | LIN54 | 0.01 | 2.81E−16 |
| Q14207 | NPAT | 0.01 | 2.81E−16 |
| P40425 | PBX2 | 0.01 | 2.81E−16 |
| P56270 | MAZ | 0.01 | 2.81E−16 |
| Q15723 | ELF2 | 0.01 | 2.81E−16 |
| Q02086 | SP2 | 0.01 | 2.81E−16 |
| P08670 | VIM | 0.01 | 2.81E−16 |
| Q9NQB0 | TCF7L2 | 0.01 | 2.81E−16 |
| Q9NZC4 | EHF | 0.01 | 2.81E−16 |
| Q2KHR3 | QSER1 | 0.01 | 2.81E−16 |
| Q96PU4 | UHRF2 | 0.01 | 2.81E−16 |
| Q9UNY4 | TTF2 | 0.01 | 2.81E−16 |
| P61244 | MAX | 0.01 | 2.81E−16 |
| Q92793 | CREBBP | 0.01 | 2.81E−16 |
| P10242 | MYB | 0.01 | 2.81E−16 |
| P52945 | PDX1 | 0.01 | 2.81E−16 |
| Q8N5Y2 | MSL3 | 0.01 | 2.81E−16 |
| P35680 | HNF1B | 0.01 | 2.81E−16 |
| P08651 | NFIC | 0.01 | 2.81E−16 |
| P55347 | PKNOX1 | 0.01 | 2.81E−16 |
| Q14141 | SEPTIN6 | 0.01 | 2.81E−16 |
| POC860 | MSL3P1 | 0.01 | 2.81E−16 |
| P19793 | RXRA | 0.01 | 2.81E−16 |
| O15525 | MAFG | 0.01 | 2.81E−16 |
| O95671 | ASMTL | 0.01 | 2.81E−16 |
| Q9UHD2 | TBK1 | 0.01 | 2.81E−16 |
| P53350 | PLK1 | 0.01 | 2.81E−16 |
| Q8ND24 | RNF214 | 0.01 | 2.81E−16 |

TABLE 4
Differential protein expression of MYXV + selinexor-treated
cytoplasm and MYXV-treated cytoplasm

| Accession | Gene Symbol | $\frac{MYXV + seli\_cyto}{MYXV\_cyto}$ | P-value |
|---|---|---|---|
| P17010 | ZFX | 100 | 4.42E−16 |
| E2CZJ9 | m110L | 100 | 4.42E−16 |
| O75052 | NOS1AP | 100 | 4.42E−16 |
| P55347 | PKNOX1 | 100 | 4.42E−16 |
| O14867 | BACH1 | 100 | 4.42E−16 |
| Q9H3F6 | KCTD10 | 100 | 4.42E−16 |
| Q6NZ67 | MZT2B | 100 | 4.42E−16 |
| Q96K58 | ZNF668 | 100 | 4.42E−16 |
| O00716 | E2F3 | 100 | 4.42E−16 |
| Q3T8J9 | GON4L | 100 | 4.42E−16 |
| A8MW92 | PHF20L1 | 100 | 4.42E−16 |
| Q9H582 | ZNF644 | 100 | 4.42E−16 |
| Q6P582 | MZT2A | 100 | 4.42E−16 |
| Q9H160 | ING2 | 100 | 4.42E−16 |
| Q9NVW2 | RLIM | 100 | 4.42E−16 |
| P0C860 | MSL3P1 | 100 | 4.42E−16 |
| Q9NQV6 | PRDM10 | 100 | 4.42E−16 |
| P08651 | NFIC | 100 | 4.42E−16 |
| P05090 | APOD | 100 | 4.42E−16 |
| Q9UQR0 | SCML2 | 100 | 4.42E−16 |
| Q6EOU4 | DMKN | 100 | 4.42E−16 |
| P56270 | MAZ | 100 | 4.42E−16 |
| Q9NQC1 | JADE2 | 100 | 4.42E−16 |
| O15525 | MAFG | 100 | 4.42E−16 |
| Q8IYH5 | ZZZ3 | 100 | 4.42E−16 |
| Q8N5Y2 | MSL3 | 100 | 4.42E−16 |
| P40426 | PBX3 | 100 | 4.42E−16 |
| P52945 | PDX1 | 100 | 4.42E−16 |
| Q8TDY2 | RB1CC1 | 100 | 4.42E−16 |
| Q14686 | NCOA6 | 100 | 4.42E−16 |
| Q9HB58 | SP110 | 100 | 4.42E−16 |
| P41212 | ETV6 | 100 | 4.42E−16 |
| Q5JSJ4 | INTS6L | 100 | 4.42E−16 |
| Q01831 | XPC | 100 | 4.42E−16 |
| P22415 | USF1 | 100 | 4.42E−16 |
| Q8N488 | RYBP | 100 | 4.42E−16 |
| P61244 | MAX | 100 | 4.42E−16 |
| Q9Y261 | FOXA2 | 100 | 4.42E−16 |
| Q9H334 | FOXP1 | 100 | 4.42E−16 |
| P40425 | PBX2 | 100 | 4.42E−16 |
| Q2KHR3 | QSER1 | 100 | 4.42E−16 |
| P17029 | ZKSCAN1 | 100 | 4.42E−16 |
| Q6MZP7 | LIN54 | 100 | 4.42E−16 |
| Q9ULM3 | YEATS2 | 100 | 4.42E−16 |
| P19793 | RXRA | 100 | 4.42E−16 |
| Q15398 | DLGAP5 | 100 | 4.42E−16 |
| E2CZV0 | m055R | 7.365 | 4.42E−16 |
| Q9BZC7 | ABCA2 | 6.159 | 4.42E−16 |
| O76015 | KRT38 | 5.752 | 4.52E−13 |
| O76013 | KRT36 | 5.752 | 4.52E−13 |
| Q9Q8S3 | m019L | 5.245 | 4.42E−16 |
| B2CWK7 | m100R | 5.018 | 4.42E−16 |
| Q9Q8J4 | m106L | 4.783 | 4.42E−16 |
| B2CWH6 | m069L | 4.333 | 4.42E−16 |
| Q85297 | | 4.333 | 4.42E−16 |
| B2CWF3 | m046L | 4.221 | 4.42E−16 |
| E2CZJ6 | m107L | 4.116 | 4.42E−16 |
| B2CWL4 | | 4.116 | 4.42E−16 |
| Q9Q8S0 | m026R | 4.067 | 4.42E−16 |
| Q9UL40 | ZNF346 | 4.038 | 5.12E−08 |
| Q96RK0 | CIC | 4.038 | 1.22E−06 |
| B2CWG1 | | 3.844 | 6.65E−14 |
| K4J9W4 | m054R | 3.844 | 6.65E−14 |
| Q9Q8N0 | m070R | 3.729 | 4.42E−16 |
| B2CWI4 | m077L | 3.515 | 4.42E−16 |
| Q9Q8K1 | m099L | 3.493 | 4.42E−16 |
| Q9Q8P8 | m048L | 3.457 | 4.42E−16 |
| B2CWC6 | m020L | 3.362 | 3.00E−13 |
| Q9Q8S2 | m020L | 3.362 | 3.00E−13 |
| B2CWG7 | m060R | 3.324 | 4.42E−16 |
| D3W0C4 | m130R | 3.257 | 4.42E−16 |
| Q9Q8J5 | m105L | 3.192 | 695E−13 |
| Q9Q8M9 | m071L | 3.114 | 4.42E−16 |
| Q96Q05 | TRAPPC9 | 3.105 | 1.01E−05 |
| B2CWH8 | | 3.05 | 4.42E−16 |
| B2CWE0 | m033R | 3.026 | 1.52E−10 |
| Q9NS91 | RAD18 | 2.77 | 3.00E−04 |
| P17066 | HSPA6 | 2.766 | 3.17E−06 |
| E2CZL6 | m127L | 2.752 | 1.97E−03 |
| Q9NSB2 | KRT84 | 2.731 | 1.98E−07 |
| Q9Q8P4 | m052L | 2.73 | 4.42E−16 |
| Q77P93 | m003.2L | 2.699 | 4.42E−16 |
| Q9Q8H9 | m122R | 2.643 | 4.42E−16 |
| Q9YJT4 | MA56 | 2.578 | 4.42E−16 |
| Q9Q8M6 | m074R | 2.527 | 2.18E−10 |
| Q9Q8K7 | m093L | 2.41 | 4.42E−16 |
| B2CWJ9 | m092L | 2.406 | 4.42E−16 |
| B2CWG4 | m057L | 2.361 | 1.89E−06 |
| B2CWM2 | m115L | 2.305 | 4.42E−16 |
| Q14919 | DRAP1 | 2.287 | 1.44E−04 |
| O95817 | BAG3 | 2.258 | 1.53E−05 |
| O95425 | SVIL | 2.253 | 0.0710 |
| Q14188 | TFDP2 | 2.23 | 0.0708 |
| Q9Q8Q3 | m043L | 2.154 | 1.12E−04 |
| O94992 | HEXIM1 | 2.117 | 4.43E−07 |
| Q14574 | DSC3 | 2.041 | 0.0966 |
| B2CWB4 | m008.1L | 2.033 | 1.20E−12 |
| B2CWG5 | m058R | 2.026 | 4.83E−14 |
| Q86WA8 | LONP2 | 0.499 | 0.0245 |
| Q5C9Z4 | NOM1 | 0.498 | 0.0107 |
| Q9POM6 | MACROH2A2 | 0.492 | 0.0199 |
| P78536 | ADAM17 | 0.491 | 0.0229 |
| P01034 | CST3 | 0.488 | 5.12E−11 |
| Q9UL18 | AGO1 | 0.486 | 0.0581 |
| A6NF01 | POM121B | 0.471 | 0.0228 |
| Q9BYX7 | POTEKP | 0.47 | 2.94E−09 |
| P0CG38 | POTEI | 0.47 | 2.94E−09 |
| Q9BQ13 | KCTD14 | 0.46 | 0.0199 |
| Q5T1J5 | CHCHD2P9 | 0.437 | 6.85E−06 |
| Q9Y6H1 | CHCHD2 | 0.437 | 6.85E−06 |
| Q9BZL1 | UBL5 | 0.401 | 1.87E−08 |
| P18065 | IGFBP2 | 0.398 | 2.73E−05 |
| O14770 | MEIS2 | 0.393 | 0.0177 |
| Q9NZJ0 | DTL | 0.39 | 8.18E−06 |
| P31350 | RRM2 | 0.369 | 4.42E−16 |
| Q9BV68 | RNF126 | 0.362 | 6.20E−06 |
| P14555 | PLA2G2A | 0.36 | 4.42E−16 |
| P43694 | GATA4 | 0.359 | 3.89E−14 |
| Q92786 | PROX1 | 0.349 | 1.19E−06 |
| Q9NZI7 | UBP1 | 0.346 | 3.13E−04 |
| O14980 | XPO1 | 0.309 | 4.42E−16 |
| Q68DK2 | ZFYVE26 | 0.288 | 4.42E−16 |
| Q8IZW8 | TNS4 | 0.285 | 4.42E−16 |
| Q9NSV4 | DIAPH3 | 0.266 | 4.42E−16 |
| O14493 | CLDN4 | 0.255 | 4.42E−16 |
| Q69Y17 | NAIF1 | 0.099 | 4.42E−16 |
| Q15047 | SETDB1 | 0.01 | 4.42E−16 |
| Q9NPA3 | MID1IP1 | 0.01 | 4.42E−16 |
| Q5VU97 | CACHD1 | 0.01 | 4.42E−16 |
| P54274 | TERF1 | 0.01 | 4.42E−16 |
| Q9GZN1 | ACTR6 | 0.01 | 4.42E−16 |
| P78325 | ADAM8 | 0.01 | 4.42E−16 |
| O14607 | UTY | 0.01 | 4.42E−16 |
| P54098 | POLG | 0.01 | 4.42E−16 |
| Q5VWQ0 | RSBN1 | 0.01 | 4.42E−16 |
| A1L390 | PLEKHG3 | 0.01 | 4.42E−16 |
| Q13950 | RUNX2 | 0.01 | 4.42E−16 |
| P38398 | BRCA1 | 0.01 | 4.42E−16 |
| O95067 | CCNB2 | 0.01 | 4.42E−16 |
| Q5VWJ9 | SNX30 | 0.01 | 4.42E−16 |
| O43379 | WDR62 | 0.01 | 4.42E−16 |
| Q8WV41 | SNX33 | 0.01 | 4.42E−16 |
| P15336 | ATF2 | 0.01 | 4.42E−16 |
| 000470 | MEIS1 | 0.01 | 4.42E−16 |
| Q92993 | KAT5 | 0.01 | 4.42E−16 |
| Q9P1U0 | POLR1H | 0.01 | 4.42E−16 |
| Q9BRR8 | GPATCH1 | 0.01 | 4.42E−16 |
| O60292 | SIPA1L3 | 0.01 | 4.42E−16 |
| P10589 | NR2F1 | 0.01 | 4.42E−16 |
| Q9ULX9 | MAFF | 0.01 | 4.42E−16 |
| Q9UPW0 | FOXJ3 | 0.01 | 4.42E−16 |
| Q8NBZ0 | INO80E | 0.01 | 4.42E−16 |
| P29375 | KDM5A | 0.01 | 4.42E−16 |
| Q8TB72 | PUM2 | 0.01 | 4.42E−16 |
| P14316 | IRF2 | 0.01 | 4.42E−16 |
| A0A1B0GTU1 | ZC3H11B | 0.01 | 4.42E−16 |
| O43312 | MTSS1 | 0.01 | 4.42E−16 |
| Q9H967 | WDR76 | 0.01 | 4.42E−16 |
| Q13887 | KLF5 | 0.01 | 4.42E−16 |
| P54762 | EPHB1 | 0.01 | 4.42E−16 |
| Q9H0H5 | RACGAP1 | 0.01 | 4.42E−16 |
| P49116 | NR2C2 | 0.01 | 4.42E−16 |
| P24468 | NR2F2 | 0.01 | 4.42E−16 |
| Q8NDX5 | PHC3 | 0.01 | 4.42E−16 |
| Q99856 | ARID3A | 0.01 | 4.42E−16 |
| Q96EZ8 | MCRS1 | 0.01 | 4.42E−16 |
| Q9H6R0 | DHX33 | 0.01 | 4.42E−16 |
| Q9H8M2 | BRD9 | 0.01 | 4.42E−16 |
| O15014 | ZNF609 | 0.01 | 4.42E−16 |
| Q9UIF9 | BAZ2A | 0.01 | 4.42E−16 |

TABLE 5
Differential protein expression of MYXV + selinexor-treated
nucleus and MYXV-treated nucleus

| Accession | Gene Symbol | $\frac{MYXV + seli\_nuc}{MYXV\_nuc}$ | P-Value |
|---|---|---|---|
| O76015 | KRT38 | 100 | 8.68E−16 |
| O76013 | KRT36 | 100 | 8.68E−16 |
| P55211 | CASP9 | 100 | 8.68E−16 |
| O95714 | HERC2 | 100 | 8.68E−16 |
| P98160 | HSPG2 | 100 | 8.68E−16 |
| Q6IA86 | ELP2 | 100 | 8.68E−16 |
| A6NNY8 | USP27X | 100 | 8.68E−16 |
| Q96HD1 | CRELD1 | 100 | 8.68E−16 |
| P33908 | MAN1A1 | 100 | 8.68E−16 |
| O43164 | PJA2 | 100 | 8.68E−16 |
| P10909 | CLU | 100 | 8.68E−16 |
| Q9BVT8 | TMUB1 | 100 | 8.68E−16 |
| P14209 | CD99 | 100 | 8.68E−16 |
| Q96LJ7 | DHRS1 | 100 | 8.68E−16 |
| Q9H254 | SPTBN4 | 100 | 8.68E−16 |
| Q9NSB2 | KRT84 | 100 | 8.68E−16 |
| P17066 | HSPA6 | 100 | 8.68E−16 |
| Q13501 | SQSTM1 | 9.458 | 8.68E−16 |
| E2CZJ9 | m110L | 9.215 | 8.68E−16 |
| B2CWF3 | m046L | 8.485 | 8.68E−16 |
| Q9Q8S3 | m019L | 7.966 | 8.68E−16 |
| Q9Q8N0 | m070R | 7.614 | 8.68E−16 |
| E2CZJ6 | m107L | 7.428 | 8.68E−16 |
| B2CWL4 | | 7.428 | 8.68E−16 |
| B2CWG1 | | 7.195 | 8.68E−16 |
| K4J9W4 | m054R | 7.195 | 8.68E−16 |
| Q9Q8J4 | m106L | 6.538 | 8.68E−16 |
| P05090 | APOD | 6.35 | 8.68E−16 |
| B2CWH6 | m069L | 6.21 | 8.68E−16 |
| Q85297 | | 6.21 | 8.68E−16 |
| Q9Q8P4 | m052L | 4.487 | 8.68E−16 |
| Q9Q8S0 | m026R | 4.42 | 8.68E−16 |
| B2CWM2 | m115L | 4.253 | 8.68E−16 |
| B2CWK7 | m100R | 4.167 | 6.77E−12 |
| Q9Q8Q3 | m043L | 3.897 | 8.53E−13 |
| Q9Q8P8 | m048L | 3.832 | 3.87E−13 |
| Q9Q8K1 | m099L | 3.756 | 8.68E−16 |
| B2CWI4 | m077L | 3.708 | 2.25E−11 |
| Q9Q8J5 | m105L | 3.676 | 9.49E−12 |
| Q9UMX5 | NENF | 3.668 | 1.29E−05 |
| P29803 | PDHA2 | 3.604 | 1.46E−07 |
| Q9Q8M9 | m071L | 3.568 | 8.68E−16 |
| B2CWE0 | m033R | 3.522 | 7.57E−07 |
| B2CWG7 | m060R | 3.467 | 8.06E−10 |
| B2CWH8 | | 3.429 | 1.9E−14 |
| O95989 | NUDT3 | 3.318 | 3E−06 |
| Q9Q8H9 | m122R | 3.152 | 1.58E−09 |
| Q9Q8M6 | m074R | 3.143 | 2.63E−08 |
| P68133 | ACTA1 | 3.121 | 0.00096 |
| E2CZL6 | m127L | 3.053 | 8.15E−06 |
| Q9Y4B4 | RAD54L2 | 3.027 | 2.69E−06 |
| Q8WVM0 | TFB1M | 3.004 | 0.00053 |
| B2CWG4 | m057L | 2.812 | 4.75E−07 |
| Q96GA3 | LTV1 | 2.748 | 2.16E−06 |
| Q9UNS1 | TIMELESS | 2.669 | 0.00548 |
| Q77P93 | m003.2L | 2.662 | 1.16E−06 |
| Q9Q8K7 | m093L | 2.639 | 4.95E−07 |
| B2CWC6 | m020L | 2.633 | 0.00017 |
| Q9Q8S2 | m020L | 2.633 | 0.00017 |
| P02795 | MT2A | 2.619 | 0.00119 |
| P55347 | PKNOX1 | 2.601 | 0.00086 |
| Q5BJH7 | YIF1B | 2.6 | 0.00067 |
| P52732 | KIF11 | 2.595 | 0.00245 |
| P78325 | ADAM8 | 2.572 | 0.00443 |
| Q2NL82 | TSR1 | 2.527 | 1.41E−06 |
| P02792 | FTL | 2.454 | 0.00985 |
| Q8TE73 | DNAH5 | 2.441 | 0.00226 |
| B2CWJ9 | m092L | 2.414 | 8.82E−07 |
| Q13895 | BYSL | 2.39 | 3.81E−07 |
| Q9NRX1 | PNO1 | 2.386 | 4.74E−06 |
| B2CWG5 | m058R | 2.364 | 6.91E−07 |
| P49759 | CLK1 | 2.344 | 0.00319 |
| Q9Q8N8 | m058R | 2.293 | 1.43E−06 |
| D2WN05 | m078R | 2.28 | 3.92E−05 |
| Q9Q8J9 | m101L | 2.273 | 2.26E−06 |
| Q9BXY0 | MAK16 | 2.225 | 0.00113 |
| Q96CX6 | LRRC58 | 2.213 | 0.06704 |
| Q9H2K0 | MTIF3 | 2.198 | 0.00058 |
| Q5VVJ2 | MYSM1 | 2.174 | 0.01301 |
| O94921 | CDK14 | 2.165 | 0.09672 |
| Q00536 | CDK16 | 2.165 | 0.09672 |
| Q92574 | TSC1 | 2.162 | 0.12296 |
| E2CZH7 | m088L | 2.157 | 1.36E−05 |
| B2CWJ5 | | 2.155 | 1.4E−05 |
| O95817 | BAG3 | 2.148 | 0.01274 |
| B2CWC8 | | 2.142 | 4.93E−05 |
| P50747 | HLCS | 2.126 | 0.13787 |
| Q9Q8R4 | m032R | 2.122 | 0.07844 |
| Q83604 | MF13 | 2.122 | 5.78E−05 |
| P07942 | LAMB1 | 2.11 | 0.02077 |
| B2CWQ5 | m149R | 2.107 | 0.02107 |
| Q13823 | GNL2 | 2.075 | 0.00090 |
| B2CWC0 | | 2.064 | 0.00937 |
| E2CZQ9 | m014L | 2.064 | 0.00193 |
| Q6ZSZ5 | ARHGEF18 | 2.059 | 0.09521 |
| Q9Y618 | PXMP4 | 2.023 | 0.07673 |
| Q9H7N4 | SCAF1 | 2.021 | 0.03400 |
| Q16777 | H2AC20 | 2.008 | 0.00337 |
| Q6FI13 | H2AC18; | 2.008 | 0.00337 |
| | H2AC19 | | |
| Q9H9G7 | AGO3 | 2.002 | 0.17075 |
| P14555 | PLA2G2A | 0.498 | 0.00445 |
| Q9Q8S8 | m013L | 0.495 | 0.05418 |
| P37231 | PPARG | 0.484 | 0.09636 |
| P10586 | PTPRF | 0.477 | 0.03968 |
| O00193 | SMAP | 0.471 | 0.00445 |
| P31151 | S100A7 | 0.467 | 0.04816 |
| Q8WUP2 | FBLIM1 | 0.464 | 0.02310 |
| Q15154 | PCM1 | 0.464 | 0.02658 |
| Q86YZ3 | HRNR | 0.462 | 0.0088 |
| Q9BXS6 | NUSAP1 | 0.454 | 0.02471 |
| Q96J88 | EPSTI1 | 0.452 | 0.05985 |
| P36402 | TCF7 | 0.452 | 0.00511 |
| Q9NZJ0 | DTL | 0.451 | 0.00809 |
| P01034 | CST3 | 0.45 | 0.00418 |
| Q92786 | PROX1 | 0.449 | 0.00113 |
| Q9BZL1 | UBL5 | 0.448 | 0.00257 |
| Q13835 | PKP1 | 0.441 | 0.01320 |
| P78536 | ADAM17 | 0.415 | 0.03336 |
| P15104 | GLUL | 0.412 | 0.03968 |
| P06400 | RB1 | 0.363 | 2.23E−08 |
| P31350 | RRM2 | 0.357 | 2.42E−06 |
| P80188 | LCN2 | 0.348 | 0.00163 |
| P78545 | ELF3 | 0.342 | 4.93E−05 |
| Q9NSV4 | DIAPH3 | 0.328 | 5.78E−05 |
| P14635 | CCNB1 | 0.303 | 4.53E−05 |
| Q8IZW8 | TNS4 | 0.251 | 1.34E−12 |
| P10242 | MYB | 0.129 | 7.48E−14 |
| Q96QD8 | SLC38A2 | 0.01 | 8.68E−16 |
| Q5T1J5 | CHCHD2P9 | 0.01 | 8.68E−16 |
| O43570 | CA12 | 0.01 | 8.68E−16 |
| Q9P2R6 | RERE | 0.01 | 8.68E−16 |
| Q96Q05 | TRAPPC9 | 0.01 | 8.68E−16 |
| Q5TGY1 | TMCO4 | 0.01 | 8.68E−16 |
| P11802 | CDK4 | 0.01 | 8.68E−16 |
| Q15011 | HERPUD1 | 0.01 | 8.68E−16 |
| P11055 | MYH3 | 0.01 | 8.68E−16 |
| Q6P1R4 | DUS1L | 0.01 | 8.68E−16 |
| Q6IA69 | NADSYN1 | 0.01 | 8.68E−16 |
| Q8NI22 | MCFD2 | 0.01 | 8.68E−16 |
| Q9Y6H1 | CHCHD2 | 0.01 | 8.68E−16 |
| Q9BVW5 | TIPIN | 0.01 | 8.68E−16 |
| Q15517 | CDSN | 0.01 | 8.68E−16 |
| Q9H9C1 | VIPAS39 | 0.01 | 8.68E−16 |
| Q6V1X1 | DPP8 | 0.01 | 8.68E−16 |
| Q8NCN5 | PDPR | 0.01 | 8.68E−16 |
| Q13227 | GPS2 | 0.01 | 8.68E−16 |
| Q9NRC8 | SIRT7 | 0.01 | 8.68E−16 |
| O00461 | GOLIM4 | 0.01 | 8.68E−16 |
| Q86TM6 | SYVN1 | 0.01 | 8.68E−16 |
| P18065 | IGFBP2 | 0.01 | 8.68E−16 |
| Q15723 | ELF2 | 0.01 | 8.68E−16 |
| Q05209 | PTPN12 | 0.01 | 8.68E−16 |
| P01111 | NRAS | 0.01 | 8.68E−16 |
| Q969Q0 | RPL36AL | 0.01 | 8.68E−16 |
| Q9Y3L5 | RAP2C | 0.01 | 8.68E−16 |
| P01112 | HRAS | 0.01 | 8.68E−16 |

TABLE 6
Differential protein expression of selinexor-
treated cytoplasm and control cytoplasm

| Accession | Gene Symbol | $\frac{Seli\_cyto}{Mock\_cyto}$ | P-value |
|---|---|---|---|
| Q9BRX9 | WDR83 | 100 | 4.88E−16 |
| Q9NRL3 | STRN4 | 100 | 4.88E−16 |
| Q15572 | TAF1C | 100 | 4.88E−16 |
| Q9P0M6 | MACROH2A2 | 100 | 4.88E−16 |
| Q9Q8K3 | m097R | 100 | 4.88E−16 |
| Q92738 | USP6NL | 100 | 4.88E−16 |
| Q9NV56 | MRGBP | 100 | 4.88E−16 |
| Q9NS91 | RAD18 | 100 | 4.88E−16 |
| Q5VWQ0 | RSBN1 | 100 | 4.88E−16 |
| O14867 | BACH1 | 100 | 4.88E−16 |
| Q9Q8R4 | m032R | 100 | 4.88E−16 |
| Q53TQ3 | INO80D | 100 | 4.88E−16 |
| Q9H6P5 | TASP1 | 100 | 4.88E−16 |
| Q8N954 | GPATCH11 | 100 | 4.88E−16 |
| Q9BRG2 | SH2D3A | 100 | 4.88E−16 |
| Q6PJE2 | POMZP3 | 100 | 4.88E−16 |
| Q9H160 | ING2 | 100 | 4.88E−16 |
| Q9H7N4 | SCAF1 | 100 | 4.88E−16 |
| O75928 | PIAS2 | 100 | 4.88E−16 |
| B2CWQ5 | m149R | 100 | 4.88E−16 |
| Q9H8G2 | CAAP1 | 100 | 4.88E−16 |
| Q96QT6 | PHF12 | 100 | 4.88E−16 |
| E2CZJ6 | m107L | 100 | 4.88E−16 |
| Q96LT9 | RNPC3 | 100 | 4.88E−16 |
| Q8TDY2 | RB1CC1 | 100 | 4.88E−16 |
| Q9HBE1 | PATZ1 | 100 | 4.88E−16 |
| B2CWL4 | | 100 | 4.88E−16 |
| Q9Y388 | RBMX2 | 100 | 4.88E−16 |
| P15408 | FOSL2 | 100 | 4.88E−16 |
| Q8NCN4 | RNF169 | 100 | 4.88E−16 |
| Q9UER7 | DAXX | 100 | 4.88E−16 |
| P18846 | ATF1 | 100 | 4.88E−16 |
| P17029 | ZKSCAN1 | 100 | 4.88E−16 |
| Q99594 | TEAD3 | 100 | 4.88E−16 |
| Q8NDX5 | PHC3 | 100 | 4.88E−16 |
| O75529 | TAF5L | 100 | 4.88E−16 |
| Q9ULM3 | YEATS2 | 100 | 4.88E−16 |
| Q96CB8 | INTS12 | 100 | 4.88E−16 |
| Q9UQ16 | DNM3 | 100 | 4.88E−16 |
| Q96E39 | RBMXL1 | 100 | 4.88E−16 |
| O15523 | DDX3Y | 100 | 4.88E−16 |
| P29508 | SERPINB3 | 34.048 | 4.88E−16 |
| P16402 | H1-3 | 14.354 | 4.88E−16 |
| O14493 | CLDN4 | 10.645 | 4.88E−16 |
| Q92993 | KAT5 | 7.08 | 1.07E−14 |
| P05090 | APOD | 7.079 | 4.88E−16 |
| P48594 | SERPINB4 | 5.894 | 3.75E−07 |
| Q12864 | CDH17 | 5.81 | 4.88E−16 |
| P43243 | MATR3 | 5.731 | 4.88E−16 |
| Q9UJU2 | LEF1 | 5.627 | 3.56E−11 |
| Q9HCS4 | TCF7L1 | 5.627 | 3.56E−11 |
| Q9UM11 | FZR1 | 4.964 | 1.31E−10 |
| Q3SY84 | KRT71 | 4.935 | 2.67E−09 |
| Q7RTS7 | KRT74 | 4.935 | 2.67E−09 |
| Q86Y46 | KRT73 | 4.935 | 2.67E−09 |
| O43278 | SPINT1 | 4.255 | 1.6E−12 |
| P08779 | KRT16 | 4.248 | 4.88E−16 |
| Q85295 | M11L | 4.074 | 7.48E−06 |
| P12830 | CDH1 | 4.064 | 4.17E−12 |
| Q15517 | CDSN | 3.988 | 3.25E−07 |
| P01040 | CSTA | 3.956 | 2.53E−07 |
| E2CZP2 | m001R | 3.839 | 0.00046 |
| O10625 | M-T1 | 3.839 | 0.00046 |
| Q96CX6 | LRRC58 | 3.753 | 1.87E−06 |
| Q08174 | PCDH1 | 3.649 | 1.62E−07 |
| P05109 | S100A8 | 3.524 | 3.74E−06 |
| A8K2U0 | A2ML1 | 3.444 | 0.00164 |
| Q9UK58 | CCNL1 | 3.386 | 0.00135 |
| Q9UDY4 | DNAJB4 | 3.313 | 1.35E−05 |
| P84243 | H3-3A; H3-3B | 3.295 | 3.85E−06 |
| O94992 | HEXIM1 | 3.29 | 1.49E−07 |
| P02533 | KRT14 | 3.261 | 2.97E−11 |
| Q71DI3 | H3C13; H3C14; H3C15 | 3.19 | 0.00155 |
| Q96P63 | SERPINB12 | 3.145 | 3.34E−05 |
| O95989 | NUDT3 | 3.124 | 3.87E−05 |
| E2CZW7 | m072L | 3.124 | 0.00519 |
| P02538 | KRT6A | 3.082 | 1.78E−12 |
| B7ZW38 | HNRNPCL3 | 2.963 | 1.46E−07 |
| P07910 | HNRNPC | 2.948 | 3.48E−13 |
| Q9Y5Y6 | ST14 | 2.934 | 8.32E−07 |
| O60812 | HNRNPCL1 | 2.92 | 2.76E−07 |
| B2RXH8 | HNRNPCL2 | 2.92 | 2.76E−07 |
| Q9H190 | SDCBP2 | 2.885 | 2.33E−06 |
| Q5JTJ3 | COA6 | 2.872 | 7.98E−05 |
| Q15388 | TOMM20 | 2.862 | 0.00077 |
| Q8N1N4 | KRT78 | 2.84 | 8.17E−05 |
| O00566 | MPHOSPH10 | 2.761 | 0.00321 |
| O00560 | SDCBP | 2.743 | 1.12E−05 |
| Q8WU68 | U2AF1L4 | 2.738 | 6.4E−06 |
| Q2M2I5 | KRT24 | 2.716 | 3.69E−07 |
| Q92804 | TAF15 | 2.706 | 2.58E−05 |
| Q13601 | KRR1 | 2.698 | 0.00036 |
| Q9Q8J9 | m101L | 2.679 | 0.02055 |
| P18827 | SDC1 | 2.678 | 4.09E−06 |
| P31944 | CASP14 | 2.655 | 0.00049 |
| Q7Z3Z0 | KRT25 | 2.607 | 1.72E−10 |
| Q7Z3Y8 | KRT27 | 2.607 | 1.72E−10 |
| Q9H7B2 | RPF2 | 2.588 | 4.15E−05 |
| P32320 | CDA | 2.571 | 6.88E−05 |
| Q8TB36 | GDAP1 | 2.559 | 0.03989 |
| Q9H0M0 | WWP1 | 2.549 | 0.00075 |
| P31151 | S100A7 | 2.547 | 0.01597 |
| Q9NUK0 | MBNL3 | 2.509 | 0.00016 |
| P28799 | GRN | 2.493 | 0.00014 |
| P15104 | GLUL | 2.48 | 0.04139 |
| O14896 | IRF6 | 2.466 | 0.00104 |
| Q7Z3Y7 | KRT28 | 2.439 | 3.64E−09 |
| O75531 | BANF1 | 2.438 | 0.02959 |
| Q9BW30 | TPPP3 | 2.408 | 5.09E−05 |
| P62316 | SNRPD2 | 2.406 | 5.19E−05 |
| P14649 | MYL6B | 2.392 | 3.06E−07 |
| O15551 | CLDN3 | 2.357 | 0.00034 |
| Q6ZVX7 | NCCRP1 | 2.342 | 0.09813 |
| E2CZV0 | m055R | 2.342 | 0.06419 |
| P57721 | PCBP3 | 2.306 | 0.10326 |
| Q9Q8P8 | m048L | 2.287 | 0.03988 |
| Q9UPQ3 | AGAP1 | 2.271 | 0.12289 |
| P13987 | CD59 | 2.269 | 0.00113 |
| P19105 | MYL12A | 2.269 | 2.25E−07 |
| P24844 | MYL9 | 2.253 | 1.02E−06 |
| O14950 | MYL12B | 2.249 | 2.49E−07 |
| P12036 | NEFH | 2.248 | 0.00498 |
| Q01546 | KRT76 | 2.236 | 1.17E−06 |
| P35580 | MYH10 | 2.213 | 2.3E−07 |
| Q2TAY7 | SMU1 | 2.21 | 0.00088 |
| Q01081 | U2AF1 | 2.205 | 0.00029 |
| Q7L5N7 | LPCAT2 | 2.194 | 0.02714 |
| Q14004 | CDK13 | 2.192 | 0.12104 |
| P62304 | SNRPE | 2.173 | 0.00257 |
| P62306 | SNRPF | 2.172 | 0.00288 |
| P00973 | OAS1 | 2.17 | 0.11268 |
| Q96AQ6 | PBXIP1 | 2.168 | 0.01351 |
| Q9HCK5 | AGO4 | 2.165 | 0.02488 |
| Q15050 | RRS1 | 2.155 | 0.04893 |
| P22735 | TGM1 | 2.137 | 0.13261 |
| P61218 | POLR2F | 2.135 | 0.09695 |
| P84077 | ARF1 | 2.133 | 0.02900 |
| P14555 | PLA2G2A | 2.128 | 0.00046 |
| P13645 | KRT10 | 2.123 | 1.1E−06 |
| Q14574 | DSC3 | 2.069 | 0.20083 |
| P51991 | HNRNPA3 | 2.069 | 0.00097 |
| P62314 | SNRPD1 | 2.056 | 0.00184 |
| Q9H329 | EPB41L4B | 2.055 | 0.04234 |
| P35579 | MYH9 | 2.051 | 3.98E−06 |
| P27930 | IL1R2 | 2.05 | 0.02210 |
| P37059 | HSD17B2 | 2.045 | 0.00711 |
| Q08554 | DSC1 | 2.044 | 0.01002 |
| P14866 | HNRNPL | 2.044 | 3.04E−05 |
| O75362 | ZNF217 | 2.042 | 0.20042 |
| P13647 | KRT5 | 2.042 | 7.62E−06 |
| Q5SXH7 | PLEKHS1 | 2.039 | 0.10983 |
| Q9UKM9 | RALY | 2.03 | 0.00730 |
| Q92614 | MYO18A | 2.026 | 0.00461 |
| P01033 | TIMP1 | 2.021 | 0.00826 |
| Q9NVH0 | EXD2 | 2.016 | 0.10576 |
| Q969Q4 | ARL11 | 2.014 | 0.19298 |
| Q7Z406 | MYH14 | 2.01 | 2.23E−05 |
| P53778 | MAPK12 | 2.006 | 0.05210 |
| O15264 | MAPK13 | 2.006 | 0.06265 |
| Q96FX8 | PERP | 2.002 | 0.00928 |
| Q13029 | PRDM2 | 0.499 | 0.1908 |
| Q9BPW8 | NIPSNAP1 | 0.499 | 0.00696 |
| Q8WW01 | TSEN15 | 0.497 | 0.06905 |
| Q9Y666 | SLC12A7 | 0.497 | 0.01002 |
| P54753 | EPHB3 | 0.496 | 0.22585 |
| O00468 | AGRN | 0.494 | 0.03604 |
| Q9Y5J1 | UTP18 | 0.493 | 0.01672 |
| Q9UHX3 | ADGRE2 | 0.491 | 0.04024 |
| P35527 | KRT9 | 0.486 | 0.00017 |
| P20839 | IMPDH1 | 0.485 | 0.00849 |
| Q9Y4B6 | DCAF1 | 0.482 | 0.05905 |
| O76080 | ZFAND5 | 0.48 | 0.07924 |
| Q14188 | TFDP2 | 0.48 | 0.21240 |
| P07478 | PRSS2 | 0.479 | 0.00169 |
| Q9HBM1 | SPC25 | 0.479 | 0.06296 |
| Q9Y4A0 | JRKL | 0.474 | 0.21136 |
| Q9C0H2 | TTYH3 | 0.47 | 0.08907 |
| Q9Y617 | PSAT1 | 0.469 | 5.59E−05 |
| Q6DKI1 | RPL7L1 | 0.463 | 0.00193 |
| O00762 | UBE2C | 0.459 | 0.03106 |
| Q9NVP2 | ASF1B | 0.458 | 0.00434 |
| P56524 | HDAC4 | 0.458 | 0.00506 |
| P11388 | TOP2A | 0.458 | 0.00068 |
| O15525 | MAFG | 0.453 | 0.19152 |
| Q9UH92 | MLX | 0.453 | 0.06713 |
| P37268 | FDFT1 | 0.453 | 0.00059 |
| P11802 | CDK4 | 0.445 | 0.03995 |
| Q9UJY4 | GGA2 | 0.442 | 0.00046 |
| P98160 | HSPG2 | 0.441 | 0.13590 |
| P43007 | SLC1A4 | 0.439 | 0.00033 |
| Q9BX40 | LSM14B | 0.437 | 0.00075 |
| Q9UMY1 | NOL7 | 0.431 | 0.01705 |
| Q86WX3 | RPS19BP1 | 0.426 | 0.09879 |
| Q6PK04 | CCDC137 | 0.426 | 0.00439 |
| Q6MZP7 | LIN54 | 0.426 | 0.12289 |
| P26358 | DNMT1 | 0.424 | 0.00024 |
| P61024 | CKS1B | 0.422 | 0.00245 |
| P00374 | DHFR | 0.419 | 0.00023 |
| Q8TD30 | GPT2 | 0.411 | 0.00063 |
| Q9ULX3 | NOB1 | 0.407 | 0.0001 |
| Q99808 | SLC29A1 | 0.406 | 0.00071 |
| Q15545 | TAF7 | 0.402 | 0.06039 |
| P28749 | RBL1 | 0.395 | 0.04702 |
| Q13685 | AAMP | 0.385 | 6.65E−05 |
| O43731 | KDELR3 | 0.379 | 0.04291 |
| Q9NP71 | MLXIPL | 0.379 | 0.00826 |
| P14635 | CCNB1 | 0.377 | 0.00090 |
| Q96GM5 | SMARCD1 | 0.376 | 0.01351 |
| Q69Y17 | NAIF1 | 0.374 | 0.00031 |
| P08243 | ASNS | 0.353 | 8.47E−10 |
| Q9NX18 | SDHAF2 | 0.346 | 0.00024 |
| Q9UJX6 | ANAPC2 | 0.336 | 4.9E−05 |
| O14980 | XPO1 | 0.335 | 6.98E−11 |
| Q53GS7 | GLE1 | 0.329 | 0.01048 |
| O00767 | SCD | 0.297 | 1.24E−09 |
| Q96GA3 | LTV1 | 0.288 | 8.2E−06 |
| P31350 | RRM2 | 0.284 | 3.4E−09 |
| P30825 | SLC7A1 | 0.272 | 2.25E−10 |
| P54098 | POLG | 0.27 | 0.00038 |
| Q96QD8 | SLC38A2 | 0.262 | 3.45E−08 |
| Q86TJ2 | TADA2B | 0.246 | 0.00046 |
| Q8TCG1 | CIP2A | 0.239 | 4.28E−06 |
| Q9NSV4 | DIAPH3 | 0.233 | 5.16E−08 |
| E2CZL5 | m126R | 0.193 | 5.32E−14 |
| Q13950 | RUNX2 | 0.192 | 2.05E−06 |
| P48380 | RFX3 | 0.152 | 7.4E−10 |
| A9QM74 | KPNA7 | 0.149 | 4.88E−16 |
| Q6E0U4 | DMKN | 0.02 | 4.88E−16 |
| Q15047 | SETDB1 | 0.01 | 4.88E−16 |
| P52739 | ZNF131 | 0.01 | 4.88E−16 |
| Q8NB78 | KDM1B | 0.01 | 4.88E−16 |
| O75052 | NOS1AP | 0.01 | 4.88E−16 |
| Q9UPQ0 | LIMCH1 | 0.01 | 4.88E−16 |
| O95425 | SVIL | 0.01 | 4.88E−16 |
| B2CWE0 | m033R | 0.01 | 4.88E−16 |
| O14607 | UTY | 0.01 | 4.88E−16 |
| P55347 | PKNOX1 | 0.01 | 4.88E−16 |
| Q8NEY8 | PPHLN1 | 0.01 | 4.88E−16 |
| Q96K58 | ZNF668 | 0.01 | 4.88E−16 |
| Q7LFL8 | CXXC5 | 0.01 | 4.88E−16 |
| A8MW92 | PHF20L1 | 0.01 | 4.88E−16 |
| P15336 | ATF2 | 0.01 | 4.88E−16 |
| O43829 | ZBTB14 | 0.01 | 4.88E−16 |
| Q9NZC4 | EHF | 0.01 | 4.88E−16 |
| O75182 | SIN3B | 0.01 | 4.88E−16 |
| O94864 | SUPT7L | 0.01 | 4.88E−16 |
| P08651 | NFIC | 0.01 | 4.88E−16 |
| Q96SB8 | SMC6 | 0.01 | 4.88E−16 |
| Q00526 | CDK3 | 0.01 | 4.88E−16 |
| O14770 | MEIS2 | 0.01 | 4.88E−16 |
| Q14207 | NPAT | 0.01 | 4.88E−16 |
| P56270 | MAZ | 0.01 | 4.88E−16 |
| P53567 | CEBPG | 0.01 | 4.88E−16 |
| P53350 | PLK1 | 0.01 | 4.88E−16 |
| Q9NQB0 | TCF7L2 | 0.01 | 4.88E−16 |
| Q02086 | SP2 | 0.01 | 4.88E−16 |
| P62745 | RHOB | 0.01 | 4.88E−16 |
| P32455 | GBP1 | 0.01 | 4.88E−16 |
| Q9Q8K6 | m094R | 0.01 | 4.88E−16 |
| P29375 | KDM5A | 0.01 | 4.88E−16 |
| P40426 | PBX3 | 0.01 | 4.88E−16 |
| Q14686 | NCOA6 | 0.01 | 4.88E−16 |
| Q9HB58 | SP110 | 0.01 | 4.88E−16 |
| Q9UJF2 | RASAL2 | 0.01 | 4.88E−16 |
| Q9Q8Q4 | m042L | 0.01 | 4.88E−16 |
| Q9POU3 | SENP1 | 0.01 | 4.88E−16 |
| Q8TD26 | CHD6 | 0.01 | 4.88E−16 |
| Q15723 | ELF2 | 0.01 | 4.88E−16 |
| P52756 | RBM5 | 0.01 | 4.88E−16 |
| Q8N2W9 | PIAS4 | 0.01 | 4.88E−16 |
| P01100 | FOS | 0.01 | 4.88E−16 |
| Q9Q8Q3 | m043L | 0.01 | 4.88E−16 |
| B2CWJ3 | | 0.01 | 4.88E−16 |
| Q9H334 | FOXP1 | 0.01 | 4.88E−16 |
| E2CZY1 | m086L | 0.01 | 4.88E−16 |
| B2CWF4 | m047R | 0.01 | 4.88E−16 |
| Q6WCQ1 | MPRIP | 0.01 | 4.88E−16 |
| B2CWG7 | m060R | 0.01 | 4.88E−16 |
| Q96EZ8 | MCRS1 | 0.01 | 4.88E−16 |
| Q9H8M2 | BRD9 | 0.01 | 4.88E−16 |
| Q8TF68 | ZNF384 | 0.01 | 4.88E−16 |
| Q5VZL5 | ZMYM4 | 0.01 | 4.88E−16 |
| Q6IBW4 | NCAPH2 | 0.01 | 4.88E−16 |
| B2CWB5 | | 0.01 | 4.88E−16 |
| Q96N64 | PWWP2A | 0.01 | 4.88E−16 |
| Q9H0E9 | BRD8 | 0.01 | 4.88E−16 |
| P07199 | CENPB | 0.01 | 4.88E−16 |
| E2CZQ3 | m009L | 0.01 | 4.88E−16 |
| Q9Q8L1 | m089L | 0.01 | 4.88E−16 |
| Q9YQ03 | mt6 | 0.01 | 4.88E−16 |

TABLE 7
Differential protein expression of selinexor-treated
nucleus and control nucleus

| Accession | Gene Symbol | $\frac{seli\_nuc}{Mock\_nuc}$ | P-value |
|---|---|---|---|
| P80188 | LCN2 | 100 | 7.71E−16 |
| O15327 | INPP4B | 100 | 7.71E−16 |
| Q96BW5 | PTER | 100 | 7.71E−16 |
| B2CWR1 | m156R | 100 | 7.71E−16 |
| Q8N612 | FHIP1B | 100 | 7.71E−16 |
| Q6DKJ4 | NXN | 100 | 7.71E−16 |
| P98172 | EFNB1 | 100 | 7.71E−16 |
| P55211 | CASP9 | 100 | 7.71E−16 |
| D2WN26 | m090L | 100 | 7.71E−16 |
| O95989 | NUDT3 | 100 | 7.71E−16 |
| Q8TE04 | PANK1 | 100 | 7.71E−16 |
| P12259 | F5 | 100 | 7.71E−16 |
| P33908 | MAN1A1 | 100 | 7.71E−16 |
| Q9Q8S8 | m013L | 100 | 7.71E−16 |
| Q8NI22 | MCFD2 | 100 | 7.71E−16 |
| Q9UPQ3 | AGAP1 | 100 | 7.71E−16 |
| A1X283 | SH3PXD2B | 100 | 7.71E−16 |
| Q9UHD2 | TBK1 | 100 | 7.71E−16 |
| Q9H9C1 | CIPAS39 | 100 | 7.71E−16 |
| P49759 | CLK1 | 100 | 7.71E−16 |
| Q9Q8K4 | VETFL | 100 | 7.71E−16 |
| Q96AG3 | SLC25A46 | 100 | 7.71E−16 |
| B2CWK3 | m096L | 100 | 7.71E−16 |
| P40855 | PEX19 | 100 | 7.71E−16 |
| O60449 | LY75 | 100 | 7.71E−16 |
| Q10472 | GALNT1 | 100 | 7.71E−16 |
| P51151 | RAB9A | 100 | 7.71E−16 |
| P17050 | NAGA | 100 | 7.71E−16 |
| Q9Q8Q1 | m045L | 100 | 7.71E−16 |
| O95865 | DDAH2 | 100 | 7.71E−16 |
| Q05209 | PTPN12 | 100 | 7.71E−16 |
| Q77P93 | m003.2L | 100 | 7.71E−16 |
| B2CWG1 | m054R | 100 | 7.71E−16 |
| K4J9W4 | m054R | 100 | 7.71E−16 |
| Q9Q8Q3 | m043L | 100 | 7.71E−16 |
| A8K2U0 | A2ML1 | 100 | 7.71E−16 |
| Q9Y6M5 | SLC30A1 | 100 | 7.71E−16 |
| Q9Q8H9 | m122R | 100 | 7.71E−16 |
| D2WN05 | m078R | 100 | 7.71E−16 |
| Q13501 | SQSTM1 | 14.425 | 7.71E−16 |
| Q969Q0 | RPL36AL | 13.627 | 7.71E−16 |
| Q96GA3 | LTV1 | 8.511 | 7.71E−16 |
| Q6EOU4 | DMKN | 7.406 | 7.71E−16 |
| Q92997 | DVL3 | 7.167 | 7.71E−16 |
| P18827 | SDC1 | 6.105 | 7.71E−16 |
| Q9P0M6 | MACROH2A2 | 5.955 | 7.71E−16 |
| Q14596 | NBR1 | 4.965 | 1.69E−11 |
| O75367 | MACROH2A1 | 4.8 | 3.38E−14 |
| Q2NL82 | TSR1 | 4.385 | 9.24E−11 |
| P0C870 | JMJD7 | 4.28 | 2.15E−07 |
| Q3SY84 | KRT71 | 4.215 | 3.11E−07 |
| Q7RTS7 | KRT74 | 4.215 | 3.11E−07 |
| Q86Y46 | KRT73 | 4.215 | 3.11E−07 |
| Q71DI3 | H3C15 | 4.057 | 7.77E−11 |
| O43278 | SPINT1 | 3.797 | 1.76E−09 |
| P41743 | PRKCI | 3.735 | 2.16E−09 |
| Q9BVS4 | RIOK2 | 3.696 | 2.1E−09 |
| Q9Q8S0 | m026R | 3.612 | 5.39E−06 |
| Q5TEC6 | H3-7 | 3.559 | 1.92E−09 |
| Q6NXT2 | H3-5 | 3.559 | 1.92E−09 |
| O75323 | NIPSNAP2 | 3.521 | 1.8E−09 |
| Q7Z794 | KRT77 | 3.514 | 3.46E−09 |
| P68431 | H3C1 | 3.511 | 2.95E−09 |
| Q6DN03 | H2BC20P | 3.428 | 3.88E−09 |
| Q6DRA6 | H2BC19P | 3.428 | 3.88E−09 |
| Q8N257 | H2BC26 | 3.428 | 3.88E−09 |
| Q16778 | H2B21 | 3.428 | 3.88E−09 |
| P33778 | H2BC3 | 3.428 | 3.88E−09 |
| P06899 | H2BC11 | 3.428 | 3.88E−09 |
| P23527 | H2BV17 | 3.428 | 3.88E−09 |
| Q16695 | H3-4 | 3.385 | 8.99E−09 |
| P35527 | KRT9 | 3.328 | 1.55E−08 |
| P08779 | KRT16 | 3.311 | 1.22E−07 |
| P62805 | H4C1 | 3.248 | 3.3E−08 |
| Q13895 | BYSL | 3.24 | 3.54E−08 |
| Q9ULX3 | NOB1 | 3.222 | 3.57E−08 |
| E9PRG8 | C11orf98 | 3.204 | 6.77E−08 |
| Q96P63 | SERPINB12 | 3.163 | 5.49E−05 |
| Q9Y5Y6 | ST14 | 3.067 | 1.33E−06 |
| Q9NVN8 | CNL3L | 3.05 | 2.68E−06 |
| Q9BPW8 | NIPSNAP1 | 2.979 | 4.46E−07 |
| P57053 | H2BC12L | 2.944 | 5.86E−07 |
| P31151 | S100A7 | 2.93 | 0.00025 |
| Q5QNW6 | H2BC18 | 2.923 | 6.71E−07 |
| Q99879 | H2BC14 | 2.923 | 6.71E−07 |
| P58876 | H2BC5 | 2.923 | 6.71E−07 |
| Q93079 | H2BV9 | 2.923 | 6.71E−07 |
| O60814 | H2BC12 | 2.923 | 6.71E−07 |
| Q99880 | H2BC13 | 2.923 | 6.71E−07 |
| P62807 | H2BC4 | 2.923 | 6.71E−07 |
| Q99877 | H2BC15 | 2.923 | 6.71E−07 |
| P12036 | NEFH | 2.836 | 2.99E−06 |
| P60520 | GABARAPL2 | 2.828 | 9.51E−07 |
| Q96A08 | H2BC1 | 2.811 | 1.95E−06 |
| Q96CX6 | LRRC58 | 2.747 | 0.000616 |
| Q9H190 | SDCBP2 | 2.735 | 1.52E−05 |
| Q9NTX5 | ECHDC1 | 2.716 | 0.001474 |
| A0A2R8Y619 | H2BK1 | 2.69 | 6.37E−06 |
| Q9NRX1 | PNO1 | 2.639 | 4.68E−05 |
| Q32P51 | HNRNPA1L2 | 2.623 | 0.001362 |
| O94916 | NFAT5 | 2.586 | 0.001312 |
| P84243 | H3-3A | 2.584 | 6.28E−05 |
| P08729 | KRT7 | 2.576 | 2.86E−05 |
| Q9BW30 | TPPP3 | 2.549 | 0.00011 |
| Q93077 | H2AC6 | 2.544 | 2.66E−05 |
| Q7L7L0 | H2AC25 | 2.544 | 2.66E−05 |
| P04908 | H2AC4 | 2.544 | 2.66E−05 |
| Q13162 | PRDX4 | 2.477 | 0.00029 |
| P00973 | OAS1 | 2.45 | 0.005053 |
| P05109 | S100A8 | 2.396 | 0.004552 |
| Q13823 | GNL2 | 2.38 | 0.000672 |
| Q9NZT1 | CALML5 | 2.328 | 0.007475 |
| Q16777 | H2AC20 | 2.322 | 0.000198 |
| Q6FI13 | H2AC18 | 2.322 | 0.000198 |
| Q9BUH6 | PAXX | 2.321 | 0.001258 |
| P04264 | KRT1 | 2.317 | 0.000246 |
| Q9NUJ1 | ABHD10 | 2.289 | 0.001313 |
| Q9UNF1 | MAGED2 | 2.271 | 0.001313 |
| Q9Y2S6 | TMA7 | 2.251 | 0.000356 |
| Q6JQN1 | ACAD10 | 2.239 | 0.011934 |
| Q96QV6 | H2AC1 | 2.215 | 0.002278 |
| Q8IUE6 | H2AC21 | 2.215 | 0.002278 |
| P16104 | H2AX | 2.215 | 0.002278 |
| Q13464 | ROCK1 | 2.201 | 0.002711 |
| P01040 | CSTA | 2.196 | 0.012269 |
| Q2M2I5 | KRT24 | 2.19 | 0.001289 |
| Q9UM11 | FXR1 | 2.17 | 0.02243 |
| Q7Z3Z0 | KRT25 | 2.158 | 0.001132 |
| Q7Z3Y8 | KRT27 | 2.158 | 0.001132 |
| P02533 | KRT14 | 2.157 | 0.004006 |
| Q9ULX9 | MAFF | 2.133 | 0.02243 |
| Q504Q3 | PAN2 | 2.108 | 0.026767 |
| Q9UQN3 | CHMP2B | 2.092 | 0.007463 |
| Q58A45 | PAN3 | 2.073 | 0.005144 |
| Q08999 | RBL2 | 2.063 | 0.03036 |
| Q7Z3Y7 | KRT28 | 2.058 | 0.002711 |
| Q96JH7 | VCPIP1 | 2.057 | 0.029202 |
| P01100 | FOS | 2.044 | 0.026766 |
| Q9NVU7 | SDAD1 | 2.021 | 0.011071 |
| P47929 | LGALS7 | 2.013 | 0.041023 |
| Q9H479 | FN3K | 2.007 | 0.005075 |
| Q56VL3 | OCIAD2 | 0.5 | 0.035337 |
| Q13505 | MTX1 | 0.499 | 0.043161 |
| O14578 | CIT | 0.498 | 0.047938 |
| Q9Q8K7 | m093L | 0.498 | 0.320197 |
| O94905 | ERLIN2 | 0.498 | 0.089781 |
| Q15388 | TOMM20 | 0.497 | 0.327948 |
| Q9Y666 | SLC12A7 | 0.496 | 0.236956 |
| Q02978 | SLC25A11 | 0.496 | 0.041606 |
| Q8TEQ6 | GEMIN5 | 0.496 | 0.040006 |
| P19367 | HK1 | 0.496 | 0.048436 |
| Q15561 | TEAD4 | 0.494 | 0.309008 |
| Q9Y2W6 | TDRKH | 0.492 | 0.282139 |
| Q86Y39 | NDUFA11 | 0.492 | 0.047938 |
| Q02241 | KIF23 | 0.492 | 0.052692 |
| Q9NQC3 | RTN4 | 0.491 | 0.029309 |
| Q16718 | NDUFA5 | 0.488 | 0.084828 |
| P23921 | RRM1 | 0.488 | 0.036288 |
| Q15562 | TEAD2 | 0.487 | 0.264475 |
| O00217 | NDUFS8 | 0.487 | 0.046951 |
| Q86TB9 | PATL1 | 0.487 | 0.07392 |
| P30876 | POLR2B | 0.486 | 0.03784 |
| O43808 | SLC25A17 | 0.484 | 0.288382 |
| Q9NXR1 | NDE1 | 0.484 | 0.274298 |
| Q5XKP0 | MICOS13 | 0.483 | 0.032467 |
| P27635 | RPL10 | 0.483 | 0.0141 |
| O75746 | SLC25A12 | 0.483 | 0.048796 |
| Q9GZY4 | COA1 | 0.481 | 0.246329 |
| Q9UQ90 | SPG7 | 0.481 | 0.274348 |
| Q9GZM8 | NDEL1 | 0.481 | 0.246329 |
| Q8WUP2 | FBLIM1 | 0.481 | 0.246329 |
| Q8TD22 | SFXN5 | 0.48 | 0.264746 |
| Q96NB2 | SFXN2 | 0.478 | 0.227391 |
| 075489 | NDUFS3 | 0.478 | 0.016567 |
| Q9UDW1 | UQCR10 | 0.477 | 0.076201 |
| Q5T6S3 | PHF19 | 0.477 | 0.259256 |
| P67809 | YBX1 | 0.477 | 0.015235 |
| Q9NRG9 | AAAS | 0.477 | 0.033125 |
| Q8TDN6 | BRIX1 | 0.475 | 0.018215 |
| Q99828 | CIB1 | 0.474 | 0.247058 |
| Q3KQZ1 | SLC25A35 | 0.474 | 0.251356 |
| Q8WXD5 | GEMIN6 | 0.473 | 0.200258 |
| Q96L21 | RPL10L | 0.473 | 0.02256 |
| P05386 | RPLP1 | 0.472 | 0.024879 |
| Q00526 | CDK3 | 0.472 | 0.184473 |
| Q659C4 | LARP1B | 0.472 | 0.235171 |
| P06493 | CDK1 | 0.47 | 0.010364 |
| Q9BTX1 | NDC1 | 0.469 | 0.017445 |
| P62273 | RPS29 | 0.468 | 0.232769 |
| Q96JP2 | MYO15B | 0.468 | 0.016247 |
| Q9Y3D7 | PAM16 | 0.467 | 0.233233 |
| P52732 | KIF11 | 0.467 | 0.215668 |
| Q8TEQ8 | PIGO | 0.467 | 0.221259 |
| P16989 | YBX3 | 0.467 | 0.013826 |
| Q8NC56 | LEMD2 | 0.466 | 0.172826 |
| Q8NG31 | KNL1 | 0.466 | 0.222654 |
| Q9C0H2 | TTYH3 | 0.465 | 0.224291 |
| Q8NE86 | MCU | 0.465 | 0.014268 |
| Q9Y617 | PSAT1 | 0.464 | 0.01911 |
| O95139 | NDUFB6 | 0.46 | 0.050259 |
| P33897 | ABCD1 | 0.46 | 0.205627 |
| Q6IAA8 | LAMTOR1 | 0.46 | 0.036323 |
| Q86YN1 | DOLPP1 | 0.459 | 0.176077 |
| P55011 | SLC12A2 | 0.459 | 0.009342 |
| Q9P0J0 | NDUFA13 | 0.458 | 0.017286 |
| P10242 | MYB | 0.457 | 0.081705 |
| Q8NBM4 | UBAC2 | 0.457 | 0.023505 |
| Q9BQ95 | ECSIT | 0.457 | 0.015076 |
| Q15758 | SLC1A5 | 0.457 | 0.014368 |
| Q08380 | LGALS3BP | 0.457 | 0.012791 |
| O96000 | NDUFB10 | 0.455 | 0.016296 |
| Q9Y6G1 | TMEM14A | 0.454 | 0.164944 |
| Q99595 | TIMM17A | 0.454 | 0.132165 |
| P98160 | HSPG2 | 0.453 | 0.188444 |
| Q8WVX9 | FAR1 | 0.45 | 0.016247 |
| O95049 | TJP3 | 0.45 | 0.023013 |
| O94886 | TMEM63A | 0.449 | 0.158587 |
| P03905 | MT-ND4 | 0.448 | 0.043141 |
| Q8WVJ2 | NUDCD2 | 0.448 | 0.180554 |
| Q8N0U8 | VKORC1L1 | 0.446 | 0.176077 |
| Q96FF7 | MISP3 | 0.445 | 0.011038 |
| Q5T1M5 | FKBP15 | 0.445 | 0.17928 |
| P53007 | SLC25A1 | 0.445 | 0.01141 |
| Q96FF9 | CDC5 | 0.444 | 0.049996 |
| Q9ULH7 | MRTFB | 0.443 | 0.155385 |
| Q9Y672 | ALG6 | 0.442 | 0.14583 |
| Q9BSK2 | SLC25A33 | 0.442 | 0.151313 |
| Q15154 | PCM1 | 0.441 | 0.066637 |
| Q8NFJ5 | GPRC5A | 0.441 | 0.004338 |
| P24821 | TNC | 0.44 | 0.10127 |
| P07478 | PRSS2 | 0.437 | 0.006645 |
| Q5TGZ0 | MICOS10 | 0.437 | 0.006208 |
| O14548 | COX7A2L | 0.434 | 0.025589 |
| Q13257 | MAD2L1 | 0.433 | 0.008293 |
| O75477 | ERLIN1 | 0.433 | 0.137096 |
| P37231 | PPARG | 0.432 | 0.013826 |
| Q6ZNB6 | NFXL1 | 0.432 | 0.139636 |
| Q9BRV3 | SLC50A1 | 0.431 | 0.124781 |
| Q53FV1 | ORMDL2 | 0.428 | 0.129254 |
| Q9P032 | NDUFAF4 | 0.428 | 0.12546 |
| Q9H0H5 | RACGAP1 | 0.428 | 0.00531 |
| E2CZL5 | m126R | 0.428 | 0.005956 |
| O75381 | PEX14 | 0.428 | 0.017542 |
| Q9BUR5 | APOO | 0.427 | 0.005514 |
| Q9NVI7 | ATAD3A | 0.426 | 0.001555 |
| P60604 | UBE2G2 | 0.425 | 0.076201 |
| Q9Q8G2 | m140R | 0.425 | 0.103688 |
| Q9NVI1 | FANCI | 0.422 | 0.001386 |
| Q9Y2Q5 | LAMTOR2 | 0.417 | 0.102884 |
| Q5SNT2 | TMEM201 | 0.416 | 0.101923 |
| Q9Y5A9 | YTHDF2 | 0.415 | 0.013826 |
| Q8WUY1 | THEM6 | 0.415 | 0.002028 |
| Q96F86 | EDC3 | 0.414 | 0.001945 |
| P49454 | CENPF | 0.413 | 0.003539 |
| Q9NPF2 | CHST11 | 0.412 | 0.007657 |
| P56282 | POLE2 | 0.412 | 0.018701 |
| P36402 | TCF7 | 0.411 | 0.002542 |
| Q9UKR5 | ERG28 | 0.409 | 0.080974 |
| O00411 | POLRMST | 0.409 | 0.006146 |
| P04183 | TK1 | 0.407 | 0.005724 |
| Q16740 | CLPP | 0.407 | 0.002532 |
| Q99643 | SDHC | 0.406 | 0.076243 |
| Q53RT3 | ASPRV1 | 0.405 | 0.06519 |
| P03891 | MT-ND2 | 0.403 | 0.05161 |
| B2CWF4 | m047R | 0.403 | 0.075074 |
| Q07864 | POLE | 0.403 | 0.001289 |
| O95235 | KIF20A | 0.4 | 0.004794 |
| Q86VU5 | COMTD1 | 0.397 | 0.00358 |
| Q9ULW0 | TPX2 | 0.397 | 0.002028 |
| P38398 | BRCA1 | 0.396 | 0.057586 |
| Q6ZRQ5 | MMS22L | 0.395 | 0.057061 |
| P28288 | ABCD3 | 0.392 | 0.001817 |
| P08243 | ASNS | 0.391 | 0.000252 |
| Q9H300 | PARL | 0.39 | 0.058051 |
| P37268 | FDFT1 | 0.39 | 0.000969 |
| Q99623 | PHB2 | 0.39 | 0.000246 |
| P0CG13 | CHTF8 | 0.389 | 0.052351 |
| Q6SJ93 | FAM111B | 0.388 | 0.041422 |
| Q9NP79 | VTA1 | 0.387 | 0.00358 |
| P03886 | MT-ND1 | 0.385 | 0.04999 |
| Q5T9A4 | ATAD3B | 0.385 | 0.000287 |
| O00330 | PDHX | 0.384 | 0.000887 |
| Q6UXV4 | APOOL | 0.382 | 0.00187 |
| P52790 | HK3 | 0.38 | 0.004107 |
| P29803 | PDHA2 | 0.376 | 0.001721 |
| Q9H3S7 | PTPN23 | 0.376 | 0.011049 |
| Q9Y487 | ATP6V0A2 | 0.375 | 0.038051 |
| O14965 | AURKA | 0.374 | 0.001617 |
| P61024 | CKS1B | 0.373 | 0.000741 |
| Q9NR77 | PXMP2 | 0.372 | 0.036257 |
| P35232 | PHB1 | 0.37 | 6.87E−05 |
| Q92609 | TBC1D5 | 0.366 | 0.030617 |
| P08559 | PDHA1 | 0.363 | 0.000234 |
| Q96J94 | PIWIL1 | 0.362 | 0.028689 |
| O15439 | ABCC4 | 0.359 | 0.024527 |
| Q9BXW9 | FANCD2 | 0.354 | 4.83E−05 |
| Q9NVP2 | ASF1B | 0.353 | 0.000616 |
| Q08378 | GOLGA3 | 0.341 | 0.00982 |
| Q9BX40 | LSM14B | 0.341 | 4.43E−05 |
| P31350 | RRM2 | 0.34 | 5.03E−05 |
| P11177 | PDHB | 0.34 | 2.27E−05 |
| Q9BYM8 | RBCK1 | 0.334 | 0.007669 |
| Q6ZN28 | MACC1 | 0.333 | 0.000188 |
| O95067 | CCNB2 | 0.324 | 0.008731 |
| P52789 | HK2 | 0.323 | 9.88E−06 |
| Q6P4A7 | SFXN4 | 0.32 | 0.006658 |
| P78325 | ADAM8 | 0.319 | 0.005592 |
| Q99808 | SLC29A1 | 0.316 | 0.002056 |
| P30825 | SLC7A1 | 0.314 | 1.54E−05 |
| Q9UHL9 | GTF2IRD1 | 0.312 | 2.47E−05 |
| P10515 | DLAT | 0.306 | 5.43E−07 |
| O43663 | PRC1 | 0.299 | 5.35E−06 |
| P43007 | SLC1A4 | 0.289 | 3.52E−07 |
| Q86WA8 | LONP2 | 0.28 | 9.55E−07 |
| Q01664 | TFAP4 | 0.279 | 3.63E−06 |
| P53350 | PLK1 | 0.275 | 0.00075 |
| O00767 | SCD | 0.275 | 2.15E−08 |
| Q9NSV4 | DIAPH3 | 0.27 | 1.5E−06 |
| Q14807 | KIF22 | 0.269 | 8.87E−08 |
| Q86VI3 | IQGAP3 | 0.268 | 0.00065 |
| O60830 | TIMM17B | 0.263 | 0.000513 |
| Q6P2E9 | EDC4 | 0.263 | 2.06E−08 |
| Q9NPI6 | DCP1A | 0.259 | 7.93E−08 |
| P15104 | GLUL | 0.258 | 0.00031 |
| P14635 | CCNB1 | 0.251 | 3.59E−09 |
| Q9Y2T7 | YBX2 | 0.224 | 5.57E−10 |
| Q9UNS1 | TIMELESS | 0.2 | 1.43E−06 |
| Q6ZN18 | AEBP2 | 0.193 | 1.03E−06 |
| Q8TCG1 | CIP2A | 0.192 | 1.46E−06 |
| P20930 | FLG | 0.183 | 3.11E−07 |
| O43570 | CA12 | 0.172 | 1.12E−07 |
| Q68DK2 | ZFYVE26 | 0.083 | 7.71E−16 |
| Q96QD8 | SLC38A2 | 0.01 | 7.71E−16 |
| Q9Q8K3 | m097R | 0.01 | 7.71E−16 |
| Q15011 | HERPUD1 | 0.01 | 7.71E−16 |
| O00584 | RNASET2 | 0.01 | 7.71E−16 |
| Q58719 | SFT2D3 | 0.01 | 7.71E−16 |
| E2CZV4 | m059R | 0.01 | 7.71E−16 |
| Q8IZ21 | PHACTR4 | 0.01 | 7.71E−16 |
| Q9UBS8 | RNF14 | 0.01 | 7.71E−16 |
| Q86WV6 | STING1 | 0.01 | 7.71E−16 |
| Q6IA69 | NADSYN1 | 0.01 | 7.71E−16 |
| O14493 | CLDN4 | 0.01 | 7.71E−16 |
| Q6V1X1 | DPP8 | 0.01 | 7.71E−16 |
| Q86VR2 | RETREG3 | 0.01 | 7.71E−16 |
| P05090 | APOD | 0.01 | 7.71E−16 |
| Q86TM6 | SYVN1 | 0.01 | 7.71E−16 |
| O75886 | STAM2 | 0.01 | 7.71E−16 |
| Q96LJ7 | DHRS1 | 0.01 | 7.71E−16 |
| Q9Q8K6 | m094R | 0.01 | 7.71E−16 |
| B2CWI4 | m077L | 0.01 | 7.71E−16 |
| B2CWG8 | m061R | 0.01 | 7.71E−16 |
| B2CWJ3 | m086L | 0.01 | 7.71E−16 |
| B2CWG4 | m057L | 0.01 | 7.71E−16 |
| E2CZY1 | m086L | 0.01 | 7.71E−16 |
| Q12913 | PTPRJ | 0.01 | 7.71E−16 |
| P17516 | AKR1C4 | 0.01 | 7.71E−16 |

TABLE 8
Differential protein expression of selinexor-treated cytoplasm
and MYXV + selinexor-treated cytoplasm

| Accession | Gene Symbol | $\frac{seli\_cyto}{MYXV + seli\_cyto}$ | P-value |
|---|---|---|---|
| Q9NPA3 | MID1IP1 | 100 | 2.31E−16 |
| Q9NRL3 | STRN4 | 100 | 2.31E−16 |
| P10242 | MYB | 100 | 2.31E−16 |
| Q9P2R6 | RERE | 100 | 2.31E−16 |
| Q5VU97 | CACHD1 | 100 | 2.31E−16 |
| Q15572 | TAF1C | 100 | 2.31E−16 |
| P78325 | ADAM8 | 100 | 2.31E−16 |
| Q92738 | USP6NL | 100 | 2.31E−16 |
| P54098 | POLG | 100 | 2.31E−16 |
| Q5VWQ0 | RSBN1 | 100 | 2.31E−16 |
| A1L390 | PLEKHG3 | 100 | 2.31E−16 |
| Q13950 | RUNX2 | 100 | 2.31E−16 |
| O95067 | CCNB2 | 100 | 2.31E−16 |
| Q5VWJ9 | SNX30 | 100 | 2.31E−16 |
| O43379 | WDR62 | 100 | 2.31E−16 |
| Q53TQ3 | INO80D | 100 | 2.31E−16 |
| O95671 | ASMTL | 100 | 2.31E−16 |
| Q8WV41 | SNX33 | 100 | 2.31E−16 |
| Q9BRG2 | SH2D3A | 100 | 2.31E−16 |
| Q9UHD2 | TBK1 | 100 | 2.31E−16 |
| Q08999 | RBL2 | 100 | 2.31E−16 |
| Q8ND24 | RNF214 | 100 | 2.31E−16 |
| Q92993 | KAT5 | 100 | 2.31E−16 |
| P08670 | VIM | 100 | 2.31E−16 |
| Q9BRR8 | GPATCH1 | 100 | 2.31E−16 |
| O75928 | PIAS2 | 100 | 2.31E−16 |
| P10589 | NR2F1 | 100 | 2.31E−16 |
| Q9ULX9 | MAFF | 100 | 2.31E−16 |
| Q9ULH7 | MRTFB | 100 | 2.31E−16 |
| Q99081 | TCF12 | 100 | 2.31E−16 |
| Q9UPW0 | FOXJ3 | 100 | 2.31E−16 |
| P35680 | HNF1B | 100 | 2.31E−16 |
| Q96QT6 | PHF12 | 100 | 2.31E−16 |
| Q8TB72 | PUM2 | 100 | 2.31E−16 |
| P14316 | IRF2 | 100 | 2.31E−16 |
| A0A1B0GTU1 | ZC3H11B | 100 | 2.31E−16 |
| Q9HBE1 | PATZ1 | 100 | 2.31E−16 |
| Q9H967 | WDR76 | 100 | 2.31E−16 |
| P15408 | FOSL2 | 100 | 2.31E−16 |
| Q13887 | KLF5 | 100 | 2.31E−16 |
| P54762 | EPHB1 | 100 | 2.31E−16 |
| Q9H0H5 | RACGAP1 | 100 | 2.31E−16 |
| P49116 | NR2C2 | 100 | 2.31E−16 |
| P24468 | NR2F2 | 100 | 2.31E−16 |
| P18846 | ATF1 | 100 | 2.31E−16 |
| A8K2U0 | A2ML1 | 100 | 2.31E−16 |
| Q96PU4 | UHRF2 | 100 | 2.31E−16 |
| P17535 | JUND | 100 | 2.31E−16 |
| Q99594 | TEAD3 | 100 | 2.31E−16 |
| Q8NDX5 | PHC3 | 100 | 2.31E−16 |
| Q99856 | ARID3A | 100 | 2.31E−16 |
| Q9UNY4 | TTF2 | 100 | 2.31E−16 |
| Q9UIF9 | BAZ2A | 100 | 2.31E−16 |
| Q14141 | SEPTIN6 | 100 | 2.31E−16 |
| Q92793 | CREBBP | 100 | 2.31E−16 |
| Q6EOU4 | DMKN | 37.932 | 2.31E−16 |
| P25774 | CTSS | 12.174 | 2.31E−16 |
| Q9NQC1 | JADE2 | 7.731 | 1.52E−14 |
| Q460N5 | PARP14 | 7.543 | 2.31E−16 |
| P08910 | ABHD2 | 7.527 | 2.31E−16 |
| O14493 | CLDN4 | 6.633 | 5.08E−15 |
| Q69Y17 | NAIF1 | 6.505 | 4.24E−12 |
| Q8IZW8 | TNS4 | 6.33 | 2.31E−16 |
| Q14258 | TRIM25 | 6.287 | 2.31E−16 |
| P06400 | RB1 | 6.184 | 2.31E−16 |
| Q5T1J5 | CHCHD2P9 | 5.859 | 5.95E−12 |
| Q9Y6H1 | CHCHD2 | 5.859 | 5.95E−12 |
| Q9UI42 | CPA4 | 5.774 | 4.78E−07 |
| Q15517 | CDSN | 5.745 | 4.3E−12 |
| P29508 | SERPINB3 | 5.695 | 1.41E−07 |
| Q9UK58 | CCNL1 | 5.598 | 9.91E−08 |
| P05090 | APOD | 5.423 | 6.13E−12 |
| Q06481 | APLP2 | 5.16 | 2.31E−16 |
| Q8WUJ3 | CEMIP | 4.975 | 5.44E−10 |
| P18065 | IGFBP2 | 4.967 | 6.53E−10 |
| Q08174 | PCDH1 | 4.895 | 4.5E−10 |
| P07602 | PSAP | 4.865 | 2.31E−16 |
| P01033 | TIMP1 | 4.709 | 3.86E−13 |
| Q9Y287 | ITM2B | 4.576 | 2.05E−08 |
| Q9H160 | ING2 | 4.383 | 0.000342 |
| Q9BZL1 | UBL5 | 4.213 | 1.86E−11 |
| Q15011 | HERPUD1 | 4.173 | 3.08E−05 |
| P48436 | SOX9 | 4.125 | 6.93E−10 |
| Q3SY84 | KRT71 | 4.123 | 1.1E−07 |
| Q7RTS7 | KRT74 | 4.123 | 1.1E−07 |
| Q86Y46 | KRT73 | 4.123 | 1.1E−07 |
| P14555 | PLA2G2A | 4.04 | 2.31E−16 |
| P28799 | GRN | 3.937 | 7.05E−14 |
| Q8TDB6 | DTX3L | 3.905 | 2.31E−07 |
| Q8NI35 | PATJ | 3.857 | 1.92E−07 |
| P18827 | SDC1 | 3.823 | 2.31E−16 |
| P78545 | ELF3 | 3.731 | 9.89E−05 |
| P55061 | TMBIM6 | 3.725 | 2.55E−09 |
| P27930 | IL1R2 | 3.715 | 6.96E−07 |
| Q8IXQ6 | PARP9 | 3.648 | 3.56E−05 |
| Q9UM11 | FZR1 | 3.606 | 2.31E−06 |
| O43291 | SPINT2 | 3.595 | 2.31E−16 |
| P16402 | H1-3 | 3.582 | 8.74E−05 |
| Q9NV92 | NDFIP2 | 3.54 | 2.49E−06 |
| Q9BU68 | PRR15L | 3.533 | 4.59E−05 |
| P07225 | PROS1 | 3.532 | 1.49E−06 |
| Q9Y653 | ADGRG1 | 3.53 | 1.17E−08 |
| Q9BV68 | RNF126 | 3.519 | 1.78E−05 |
| Q7L7X3 | TAOK1 | 3.501 | 0.000901 |
| P01040 | CSTA | 3.413 | 3.53E−06 |
| Q8N3R9 | PALS1 | 3.389 | 4.63E−05 |
| P01034 | CST3 | 3.387 | 5.08E−15 |
| Q13033 | STRN3 | 3.37 | 7.4E−06 |
| P05067 | APP | 3.312 | 5.85E−10 |
| Q8NFA2 | NOXO1 | 3.281 | 0.001302 |
| P25311 | AZGP1 | 3.261 | 8.02E−14 |
| Q08554 | DSC1 | 3.227 | 1.09E−06 |
| Q96P63 | SERPINB12 | 3.209 | 1.11E−05 |
| O43286 | B4GALT5 | 3.202 | 8.83E−05 |
| Q96L93 | KIF16B | 3.159 | 0.001012 |
| P04626 | ERBB2 | 3.073 | 9.95E−10 |
| P61769 | B2M | 3.006 | 1.74E−12 |
| P17096 | HMGA1 | 2.986 | 6.53E−05 |
| Q92817 | EVPL | 2.975 | 6.18E−05 |
| O00391 | QSOX1 | 2.971 | 2.54E−08 |
| P48594 | SERPINB4 | 2.922 | 0.00676 |
| Q8IXJ6 | SIRT2 | 2.892 | 0.000227 |
| Q8WV24 | PHLDA1 | 2.841 | 0.000212 |
| P19801 | AOC1 | 2.802 | 0.003146 |
| Q9UDY4 | DNAJB4 | 2.8 | 0.000254 |
| P10909 | CLU | 2.797 | 0.006894 |
| P15104 | GLUL | 2.765 | 0.021723 |
| P31944 | CASP14 | 2.762 | 0.000396 |
| Q14289 | PTK2B | 2.729 | 0.011976 |
| P15529 | CD46 | 2.706 | 9.85E−08 |
| Q15375 | EPHA7 | 2.7 | 2.81E−06 |
| A0JLT2 | MED19 | 2.7 | 0.041956 |
| O95989 | NUDT3 | 2.688 | 0.000523 |
| Q9UPU5 | USP24 | 2.688 | 2.75E−06 |
| O94955 | RHOBTB3 | 2.675 | 0.00102 |
| Q9BYX7 | POTEKP | 2.667 | 8.91E−07 |
| POCG38 | POTEI | 2.667 | 8.91E−07 |
| Q99595 | TIMM17A | 2.645 | 1.56E−05 |
| E2CZL5 | m126R | 2.63 | 0.000342 |
| Q86WA8 | LONP2 | 2.621 | 0.006905 |
| P54764 | EPHA4 | 2.617 | 0.033287 |
| Q8N1N4 | KRT78 | 2.596 | 0.000813 |
| Q6ZVX7 | NCCRP1 | 2.586 | 0.020452 |
| P08047 | SP1 | 2.577 | 0.003653 |
| Q8N6R0 | METTL13 | 2.569 | 0.0009 |
| Q96CX6 | LRRC58 | 2.564 | 0.000913 |
| O14965 | AURKA | 2.531 | 0.02953 |
| P31151 | S100A7 | 2.525 | 0.006789 |
| Q5JTJ3 | COA6 | 2.52 | 0.001055 |
| P10321 | HLA-C | 2.516 | 2.53E−05 |
| Q96HP0 | DOCK6 | 2.508 | 6.46E−08 |
| P38571 | LIPA | 2.5 | 4.26E−05 |
| Q63HN8 | RNF213 | 2.499 | 0.000116 |
| Q13049 | TRIM32 | 2.47 | 0.028785 |
| Q6PJ69 | TRIM65 | 2.468 | 0.009733 |
| Q7Z3Y7 | KRT28 | 2.466 | 9.03E−13 |
| Q7Z3Z0 | KRT25 | 2.459 | 1.07E−12 |
| Q7Z3Y8 | KRT27 | 2.459 | 1.07E−12 |
| P61626 | LYZ | 2.457 | 8.37E−10 |
| Q2M215 | KRT24 | 2.415 | 7.18E−08 |
| P00533 | EGFR | 2.38 | 0.000306 |
| O43815 | STRN | 2.373 | 0.000119 |
| Q13835 | PKP1 | 2.373 | 0.004464 |
| Q92876 | KLK6 | 2.358 | 1.1E−05 |
| O00468 | AGRN | 2.334 | 0.005651 |
| Q5D862 | FLG2 | 2.328 | 0.000332 |
| O75976 | CPD | 2.317 | 0.000134 |
| P04439 | HLA-A | 2.288 | 5.59E−07 |
| Q7Z417 | LIMS2 | 2.282 | 0.004061 |
| Q15582 | TGFBI | 2.25 | 0.009005 |
| Q9NS91 | RAD18 | 2.218 | 0.008748 |
| Q9HBU6 | ETNK1 | 2.216 | 0.010407 |
| P02533 | KRT14 | 2.208 | 3.63E−07 |
| Q9H3M7 | TXNIP | 2.207 | 0.049346 |
| Q53RY4 | KRTCAP3 | 2.195 | 0.010892 |
| P20823 | HNF1A | 2.193 | 0.023272 |
| P36402 | TCF7 | 2.183 | 0.010892 |
| P29323 | EPHB2 | 2.177 | 2.13E−06 |
| P15924 | DSP | 2.173 | 1.47E−09 |
| Q9Y6M5 | SLC30A1 | 2.167 | 0.001287 |
| P98172 | EFNB1 | 2.162 | 0.101421 |
| P54753 | EPHB3 | 2.147 | 0.111289 |
| P29317 | EPHA2 | 2.144 | 0.000885 |
| Q9H5V8 | CDCP1 | 2.142 | 0.076449 |
| Q01546 | KRT76 | 2.139 | 9.17E−09 |
| Q8N5J2 | MINDY1 | 2.131 | 0.018502 |
| Q9UIW2 | PLXNA1 | 2.125 | 0.112856 |
| Q99538 | LGMN | 2.12 | 0.000298 |
| P84243 | H3-3A; H3-3B | 2.116 | 0.02019 |
| P52747 | ZNF143 | 2.114 | 0.203034 |
| P07478 | PRSS2 | 2.112 | 4.89E−05 |
| Q9BQ13 | KCTD14 | 2.111 | 0.115682 |
| Q96J94 | PIWIL1 | 2.111 | 0.02061 |
| Q96CB8 | INTS12 | 2.108 | 0.082677 |
| P31350 | RRM2 | 2.101 | 0.00043 |
| Q9BX16 | TBC1D10A | 2.099 | 0.081 |
| P80188 | LCN2 | 2.091 | 0.021794 |
| Q5C9Z4 | NOM1 | 2.078 | 0.099977 |
| Q04912 | MST1R | 2.076 | 0.024501 |
| P09601 | HMOX1 | 2.072 | 0.019666 |
| Q6KB66 | KRT80 | 2.072 | 0.000273 |
| O43278 | SPINT1 | 2.061 | 0.000236 |
| O43570 | CA12 | 2.052 | 0.04074 |
| Q96MM3 | ZFP42 | 2.05 | 0.131488 |
| Q86UW9 | DTX2 | 2.046 | 0.027908 |
| Q16539 | MAPK14 | 2.045 | 0.000862 |
| P00973 | OAS1 | 2.031 | 0.126133 |
| Q9H329 | EPB41L4B | 2.017 | 0.033287 |
| Q8WWK9 | CKAP2 | 2.005 | 0.03633 |
| O15391 | YY2 | 2 | 0.106481 |
| Q13435 | SF3B2 | 0.5 | 8.35E−06 |
| Q9NP71 | MLXIPL | 0.495 | 0.098145 |
| Q9UKD2 | MRTO4 | 0.494 | 6.75E−05 |
| Q9BVI4 | NOC4L | 0.494 | 0.000777 |
| Q86VM9 | ZC3H18 | 0.493 | 0.009238 |
| Q9NRX1 | PNO1 | 0.492 | 0.001163 |
| Q15459 | SF3A1 | 0.492 | 3.41E−06 |
| Q8TAD8 | SNIP1 | 0.49 | 0.157953 |
| Q14690 | PDCD11 | 0.489 | 0.003422 |
| P62314 | SNRPD1 | 0.487 | 1.1E−05 |
| Q9NVF7 | FBXO28 | 0.486 | 0.137962 |
| Q13330 | MTA1 | 0.486 | 0.133705 |
| Q9BWJ5 | SF3B5 | 0.485 | 0.025581 |
| Q96GA3 | LTV1 | 0.484 | 0.018502 |
| Q96ST3 | SIN3A | 0.48 | 0.00072 |
| Q9Q8Q2 | NPH2 | 0.478 | 0.003805 |
| B2CWF1 | | 0.478 | 0.003805 |
| Q13151 | HNRNPA0 | 0.477 | 7.48E−06 |
| P62995 | TRA2B | 0.474 | 4.91E−05 |
| Q9UKM9 | RALY | 0.472 | 0.000128 |
| Q96T37 | RBM15 | 0.472 | 0.017447 |
| Q15233 | NONO | 0.472 | 1.54E−08 |
| P48380 | RFX3 | 0.47 | 0.076077 |
| Q9Y4B6 | DCAF1 | 0.47 | 0.012338 |
| Q9HCD5 | NCOA5 | 0.469 | 0.000713 |
| Q15717 | ELAVL1 | 0.469 | 9.04E−07 |
| Q9NWH9 | SLTM | 0.468 | 0.000856 |
| O60306 | AQR | 0.468 | 8.83E−08 |
| O95983 | MBD3 | 0.466 | 0.027654 |
| Q15287 | RNPS1 | 0.465 | 0.001145 |
| Q15428 | SF3A2 | 0.462 | 1.56E−05 |
| O60341 | KDM1A | 0.462 | 0.00023 |
| Q86U42 | PABPN1 | 0.46 | 0.000891 |
| Q99459 | CDC5L | 0.452 | 4.77E−06 |
| P35527 | KRT9 | 0.451 | 1.29E−09 |
| B2CWP9 | m142R | 0.45 | 0.000429 |
| Q9P2K3 | RCOR3 | 0.449 | 0.079486 |
| O43390 | HNRNPR | 0.449 | 1.07E−09 |
| Q9H307 | PNN | 0.447 | 9.08E−05 |
| P08621 | SNRNP70 | 0.444 | 1.34E−06 |
| Q9Y580 | RBM7 | 0.442 | 5.04E−05 |
| Q9BRT6 | LLPH | 0.435 | 0.000743 |
| Q14188 | TFDP2 | 0.431 | 0.066268 |
| Q9H7N4 | SCAF1 | 0.431 | 0.037559 |
| Q9Y2W1 | THRAP3 | 0.43 | 5.97E−06 |
| Q6KC79 | NIPBL | 0.428 | 0.007089 |
| Q9UKV3 | ACIN1 | 0.426 | 3.56E−05 |
| Q9Q8I7 | m113R | 0.422 | 0.002199 |
| 075494 | SRSF10 | 0.421 | 0.000234 |
| Q01581 | HMGCS1 | 0.42 | 1.96E−10 |
| P12270 | TPR | 0.412 | 6.17E−08 |
| P07910 | HNRNPC | 0.41 | 5.68E−12 |
| O94880 | PHF14 | 0.409 | 0.035878 |
| Q12996 | CSTF3 | 0.409 | 6.75E−05 |
| P23246 | SFPQ | 0.409 | 4.69E−12 |
| Q9Q8G2 | m140R | 0.403 | 0.001268 |
| Q9Q8S0 | m026R | 0.402 | 2.47E−06 |
| Q92804 | TAF15 | 0.4 | 3.53E−05 |
| Q99729 | HNRNPAB | 0.399 | 7.24E−12 |
| P09651 | HNRNPA1 | 0.393 | 3.78E−13 |
| P49756 | RBM25 | 0.392 | 6.1E−06 |
| P28749 | RBL1 | 0.387 | 0.042626 |
| Q9UPP1 | PHF8 | 0.376 | 0.005017 |
| Q9Q8J1 | m109L | 0.372 | 2.02E−06 |
| Q13595 | TRA2A | 0.37 | 3.96E−05 |
| O60812 | HNRNPCL1 | 0.363 | 1.58E−10 |
| B2RXH8 | HNRNPCL2 | 0.363 | 1.58E−10 |
| B7ZW38 | HNRNPCL3 | 0.361 | 1.4E−10 |
| B2CWL5 | m108R | 0.36 | 6.33E−11 |
| Q14151 | SAFB2 | 0.351 | 7.18E−08 |
| Q13242 | SRSF9 | 0.345 | 7.83E−07 |
| Q8IUH3 | RBM45 | 0.34 | 0.000373 |
| O15226 | NKRF | 0.337 | 7.85E−08 |
| E2CZV4 | m059R | 0.329 | 1.26E−06 |
| E2CZM2 | m133R | 0.321 | 1.45E−08 |
| P22626 | HNRNPA2B1 | 0.307 | 2.31E−16 |
| Q15397 | PUM3 | 0.306 | 2.58E−09 |
| B2CWE3 | | 0.302 | 7.78E−07 |
| Q9Q8R0 | m036L | 0.302 | 9.09E−08 |
| Q5BKZ1 | ZNF326 | 0.299 | 1.57E−07 |
| P51991 | HNRNPA3 | 0.295 | 2.31E−16 |
| O14979 | HNRNPDL | 0.293 | 2.18E−12 |
| P31942 | HNRNPH3 | 0.289 | 2.31E−16 |
| Q15424 | SAFB | 0.288 | 2.75E−08 |
| Q9UHL9 | GTF2IRD1 | 0.282 | 0.000793 |
| B2CWP1 | | 0.269 | 2.31E−16 |
| K4JGJ2 | m134R | 0.269 | 2.31E−16 |
| B2CWQ3 | m147R | 0.268 | 1.01E−07 |
| Q9Y2K1 | ZBTB1 | 0.229 | 2.01E−12 |
| Q96E39 | RBMXL1 | 0.222 | 2.24E−05 |
| 075526 | RBMXL2 | 0.221 | 1.35E−13 |
| P38159 | RBMX | 0.22 | 2.31E−16 |
| Q15853 | USF2 | 0.208 | 8.81E−06 |
| Q9Q8Q1 | | 0.158 | 2.04E−09 |
| B2CWD4 | m027L | 0.158 | 2.31E−16 |
| B2CWG5 | m058R | 0.149 | 2.31E−16 |
| Q9Q8N8 | m058R | 0.149 | 2.31E−16 |
| E2CZW7 | m072L | 0.131 | 2.31E−16 |
| E2CZP2 | m001R | 0.129 | 2.31E−16 |
| O10625 | M-T1 | 0.129 | 2.31E−16 |
| P68550 | | 0.129 | 2.31E−16 |
| P68544 | PAPS | 0.127 | 2.31E−16 |
| Q9Q8S3 | m019L | 0.126 | 2.31E−16 |
| B2CWF3 | m046L | 0.124 | 2.31E−16 |
| O76015 | KRT38 | 0.123 | 3.32E−12 |
| O76013 | KRT36 | 0.123 | 3.32E−12 |
| Q85295 | M11L | 0.123 | 2.31E−16 |
| B2CWQ5 | m149R | 0.12 | 2.31E−16 |
| A9QM74 | KPNA7 | 0.114 | 2.31E−16 |
| B2CWI7 | m080R | 0.114 | 2.31E−16 |
| Q9Q8L8 | m082R | 0.105 | 2.31E−16 |
| Q9Q8P4 | m052L | 0.103 | 2.31E−16 |
| B2CWB4 | m008.1L | 0.102 | 2.31E−16 |
| Q9Q8I1 | m120L | 0.095 | 2.31E−16 |
| E2CZW3 | m068R | 0.088 | 2.31E−16 |
| Q9Q8F9 | m144R | 0.087 | 2.31E−16 |
| B2CWA6 | m003.1R | 0.085 | 2.31E−16 |
| Q77PB1 | m003.1L | 0.085 | 2.31E−16 |
| B2CWH8 | | 0.082 | 2.31E−16 |
| Q9Q8M9 | m071L | 0.082 | 2.31E−16 |
| Q9Q8L9 | VETFS | 0.081 | 2.31E−16 |
| B2CWI4 | m077L | 0.078 | 2.31E−16 |
| B2CWJ9 | m092L | 0.076 | 2.31E−16 |
| B2CWI3 | m076R | 0.062 | 2.31E−16 |
| B2CWM1 | | 0.06 | 2.31E−16 |
| E2CZK3 | m114R | 0.06 | 2.31E−16 |
| Q9Q8I6 | m114R | 0.06 | 2.31E−16 |
| Q9Q8Q6 | m040L | 0.057 | 2.31E−16 |
| E2CZV0 | m055R | 0.047 | 2.31E−16 |
| Q9PX36 | m006L | 0.047 | 2.31E−16 |
| Q9Q8H2 | m129R | 0.046 | 2.31E−16 |
| Q9Q8K7 | m093L | 0.045 | 2.31E−16 |
| E2CZRO | m015L | 0.041 | 2.31E−16 |
| B2CWC1 | m015L | 0.041 | 2.31E−16 |
| P07942 | LAMB1 | 0.039 | 2.31E−16 |
| D2WN05 | m078R | 0.038 | 2.31E−16 |
| Q9Q814 | m117L | 0.038 | 2.31E−16 |
| D2WMA0 | m030L | 0.037 | 2.31E−16 |
| Q9Q8N5 | | 0.034 | 2.31E−16 |
| B2CWH0 | | 0.034 | 2.31E−16 |
| B2CWQ7 | | 0.033 | 2.31E−16 |
| D2WN93 | m151R | 0.033 | 2.31E−16 |
| Q9Q8F0 | m154L | 0.032 | 2.31E−16 |
| Q9Q8M5 | m075R | 0.031 | 2.31E−16 |
| Q83604 | MF13 | 0.029 | 2.31E−16 |
| B2CWM2 | m115L | 0.028 | 2.31E−16 |
| B2CWG0 | m053R | 0.028 | 2.31E−16 |
| Q9Q8H9 | m122R | 0.026 | 2.31E−16 |
| B2CWE1 | | 0.026 | 2.31E−16 |
| Q9Q8R2 | m034L | 0.026 | 2.31E−16 |
| Q77P93 | m003.2L | 0.025 | 2.31E−16 |
| B2CWC8 | | 0.021 | 2.31E−16 |
| Q9Q8K1 | m099L | 0.02 | 2.31E−16 |
| Q9Q8P8 | m048L | 0.019 | 2.31E−16 |
| Q9Q8L3 | m087L | 0.019 | 2.31E−16 |
| B2CWD6 | m029L | 0.018 | 2.31E−16 |
| B2CWJ5 | | 0.017 | 2.31E−16 |
| E2CZH7 | m088L | 0.017 | 2.31E−16 |
| Q9Q8Q8 | m038L | 0.016 | 2.31E−16 |
| B2CWH6 | m069L | 0.015 | 2.31E−16 |
| Q85297 | | 0.015 | 2.31E−16 |
| O55698 | M-T4 | 0.014 | 2.31E−16 |
| B2CWA8 | | 0.014 | 2.31E−16 |
| Q9PXA3 | m004L | 0.014 | 2.31E−16 |
| E2CZJ6 | m107L | 0.012 | 2.31E−16 |
| B2CWL4 | | 0.012 | 2.31E−16 |
| P52739 | ZNF131 | 0.01 | 2.31E−16 |
| P17010 | ZFX | 0.01 | 2.31E−16 |
| B2CWR1 | m156R | 0.01 | 2.31E−16 |
| E2CZJ9 | m110L | 0.01 | 2.31E−16 |
| O75052 | NOS1AP | 0.01 | 2.31E−16 |
| Q5VVJ2 | MYSM1 | 0.01 | 2.31E−16 |
| Q9UPQ0 | LIMCH1 | 0.01 | 2.31E−16 |
| O95425 | SVIL | 0.01 | 2.31E−16 |
| B2CWE0 | m033R | 0.01 | 2.31E−16 |
| P55347 | PKNOX1 | 0.01 | 2.31E−16 |
| Q8NEY8 | PPHLN1 | 0.01 | 2.31E−16 |
| Q9Q8G4 | m137R | 0.01 | 2.31E−16 |
| Q9YJT4 | MA56 | 0.01 | 2.31E−16 |
| Q96K58 | ZNF668 | 0.01 | 2.31E−16 |
| Q9Q8S8 | m013L | 0.01 | 2.31E−16 |
| O00716 | E2F3 | 0.01 | 2.31E−16 |
| Q83730 | m005R | 0.01 | 2.31E−16 |
| B2CWB0 | | 0.01 | 2.31E−16 |
| Q3T8J9 | GON4L | 0.01 | 2.31E−16 |
| B2CWP6 | m139R | 0.01 | 2.31E−16 |
| Q7LFL8 | CXXC5 | 0.01 | 2.31E−16 |
| A8MW92 | PHF20L1 | 0.01 | 2.31E−16 |
| E2CZM0 | m131R | 0.01 | 2.31E−16 |
| Q9H582 | ZNF644 | 0.01 | 2.31E−16 |
| O43829 | ZBTB14 | 0.01 | 2.31E−16 |
| O75182 | SIN3B | 0.01 | 2.31E−16 |
| B2CWC6 | m020L | 0.01 | 2.31E−16 |
| O94864 | SUPT7L | 0.01 | 2.31E−16 |
| Q9Q8K4 | VETFL | 0.01 | 2.31E−16 |
| B2CWK3 | | 0.01 | 2.31E−16 |
| E2CZV1 | m056R | 0.01 | 2.31E−16 |
| P08651 | NFIC | 0.01 | 2.31E−16 |
| Q9UQR0 | SCML2 | 0.01 | 2.31E−16 |
| Q96SB8 | SMC6 | 0.01 | 2.31E−16 |
| Q00526 | CDK3 | 0.01 | 2.31E−16 |
| O14770 | MEIS2 | 0.01 | 2.31E−16 |
| P56270 | MAZ | 0.01 | 2.31E−16 |
| Q9UL40 | ZNF346 | 0.01 | 2.31E−16 |
| Q9HAJ7 | SAP30L | 0.01 | 2.31E−16 |
| Q9Q8J4 | m106L | 0.01 | 2.31E−16 |
| P62745 | RHOB | 0.01 | 2.31E−16 |
| Q9Q8S2 | m020L | 0.01 | 2.31E−16 |
| Q9Y483 | MTF2 | 0.01 | 2.31E−16 |
| P32455 | GBP1 | 0.01 | 2.31E−16 |
| Q8IYH5 | ZZZ3 | 0.01 | 2.31E−16 |
| Q9Q8K6 | m094R | 0.01 | 2.31E−16 |
| P40426 | PBX3 | 0.01 | 2.31E−16 |
| Q9Q8J5 | m105L | 0.01 | 2.31E−16 |
| Q14686 | NCOA6 | 0.01 | 2.31E−16 |
| Q9HB58 | SP110 | 0.01 | 2.31E−16 |
| Q9Q8N0 | m070R | 0.01 | 2.31E−16 |
| Q9UJF2 | RASAL2 | 0.01 | 2.31E−16 |
| P41212 | ETV6 | 0.01 | 2.31E−16 |
| Q9Q8Q4 | m042L | 0.01 | 2.31E−16 |
| Q5JSJ4 | INTS6L | 0.01 | 2.31E−16 |
| Q9P0U3 | SENP1 | 0.01 | 2.31E−16 |
| Q01831 | XPC | 0.01 | 2.31E−16 |
| Q9P1Y6 | PHRF1 | 0.01 | 2.31E−16 |
| P52756 | RBM5 | 0.01 | 2.31E−16 |
| B2CWG8 | m061R | 0.01 | 2.31E−16 |
| B2CWG1 | | 0.01 | 2.31E−16 |
| Q8N2W9 | PIAS4 | 0.01 | 2.31E−16 |
| P01100 | FOS | 0.01 | 2.31E−16 |
| K4J9W4 | m054R | 0.01 | 2.31E−16 |
| E2CZL6 | m127L | 0.01 | 2.31E−16 |
| Q9Q8Q3 | m043L | 0.01 | 2.31E−16 |
| Q8N488 | RYBP | 0.01 | 2.31E−16 |
| B2CWJ3 | | 0.01 | 2.31E−16 |
| Q9Y261 | FOXA2 | 0.01 | 2.31E−16 |
| Q9H334 | FOXP1 | 0.01 | 2.31E−16 |
| E2CZY1 | m086L | 0.01 | 2.31E−16 |
| Q9Q8M6 | m074R | 0.01 | 2.31E−16 |
| Q9Y3Y2 | CHTOP | 0.01 | 2.31E−16 |
| B2CWF4 | m047R | 0.01 | 2.31E−16 |
| Q9UK61 | TASOR | 0.01 | 2.31E−16 |
| Q6WCQ1 | MPRIP | 0.01 | 2.31E−16 |
| B2CWG7 | m060R | 0.01 | 2.31E−16 |
| Q9Q8G6 | m135R | 0.01 | 2.31E−16 |
| Q8TF68 | ZNF384 | 0.01 | 2.31E−16 |
| Q9NSB2 | KRT84 | 0.01 | 2.31E−16 |
| Q6IBW4 | NCAPH2 | 0.01 | 2.31E−16 |
| B2CWB5 | | 0.01 | 2.31E−16 |
| Q96N64 | PWWP2A | 0.01 | 2.31E−16 |
| Q9H0E9 | BRD8 | 0.01 | 2.31E−16 |
| D2WM37 | m012L | 0.01 | 2.31E−16 |
| P07199 | CENPB | 0.01 | 2.31E−16 |
| D3W0C4 | m130R | 0.01 | 2.31E−16 |
| E2CZQ3 | m009L | 0.01 | 2.31E−16 |
| Q9Q8L1 | m089L | 0.01 | 2.31E−16 |
| Q9Q8J9 | m101L | 0.01 | 2.31E−16 |
| Q9YQ03 | mt6 | 0.01 | 2.31E−16 |
| Q85296 | | 0.01 | 2.31E−16 |
| B2CWB2 | | 0.01 | 2.31E−16 |
| Q9PX24 | m007L | 0.01 | 2.31E−16 |
| B2CWK2 | m095L | 0.01 | 2.31E−16 |

TABLE 9
Differential protein expression of selinexor-treated nucleus and
MYXV + selinexor-treated nucleus

| Accession | Gene Symbol | $\frac{seli\_nuc}{MYXV + seli\_nuc}$ | P-value |
|---|---|---|---|
| O15327 | INPP4B | 100 | 0.992536 |
| Q5T1J5 | CHCHD2P9 | 100 | 0.966048 |
| O43570 | CA12 | 100 | 0.999872 |
| Q9P2R6 | RERE | 100 | 2.56E−16 |
| Q86YC2 | PALB2 | 100 | 2.56E−16 |
| Q96Q05 | TRAPPC9 | 100 | 0.999575 |
| P98172 | EFNB1 | 100 | 0.988059 |
| P11802 | CDK4 | 100 | 0.999872 |
| P11055 | MYH3 | 100 | 2.56E−16 |
| Q8TE04 | PANK1 | 100 | 0.968062 |
| Q5VWQ0 | RSBN1 | 100 | 2.56E−16 |
| Q6P1R4 | DUS1L | 100 | 0.966048 |
| Q8NI22 | MCFD2 | 100 | 0.966048 |
| Q5VWJ9 | SNX30 | 100 | 2.56E−16 |
| Q76L83 | ASXL2 | 100 | 2.56E−16 |
| Q9Y6H1 | CHCHD2 | 100 | 0.966048 |
| Q9UHD2 | TBK1 | 100 | 2.56E−16 |
| Q15517 | CDSN | 100 | 0.984925 |
| Q9H9C1 | VIPAS39 | 100 | 0.999872 |
| Q8ND24 | RNF214 | 100 | 2.56E−16 |
| Q9UI42 | CPA4 | 100 | 0.968062 |
| Q8NCN5 | PDPR | 100 | 0.984925 |
| P40855 | PEX19 | 100 | 0.966048 |
| Q9NP58 | ABCB6 | 100 | 0.968062 |
| Q13227 | GPS2 | 100 | 0.966048 |
| Q9NRC8 | SIRT7 | 100 | 0.966048 |
| P53350 | PLK1 | 100 | 2.56E−16 |
| O00461 | GOLIM4 | 100 | 0.98118 |
| P18065 | IGFBP2 | 100 | 0.966048 |
| Q15723 | ELF2 | 100 | 2.56E−16 |
| Q05209 | PTPN12 | 100 | 0.999872 |
| P01111 | NRAS | 100 | |
| Q969Q0 | RPL36AL | 100 | 0.984133 |
| Q9Y3L5 | RAP2C | 100 | 0.999872 |
| Q9Y6M5 | SLC30A1 | 100 | 0.988059 |
| P01112 | HRAS | 100 | |
| Q32P51 | HNRNPA1L2 | 100 | 2.56E−16 |
| P06400 | RB1 | 15.663 | 0.124803 |
| Q460N5 | PARP14 | 14.323 | 0.489999 |
| P10242 | MYB | 11.112 | 2.56E−16 |
| Q8IZW8 | TNS4 | 7.853 | 0.966048 |
| Q8WV41 | SNX33 | 7.383 | 2.56E−16 |
| Q13835 | PKP1 | 6.269 | 0.966048 |
| P14635 | CCNB1 | 6.147 | 0.966048 |
| Q92786 | PROX1 | 6.039 | 0.966048 |
| Q86V15 | CASZ1 | 6.002 | 0.966048 |
| P08910 | ABHD2 | 5.702 | 0.966048 |
| Q13315 | ATM | 5.61 | 0.966048 |
| Q92997 | DVL3 | 5.571 | 0.966048 |
| Q9C0K0 | BCL11B | 5.327 | 0.966048 |
| Q9Y5B9 | SUPT16H | 4.665 | 0.966048 |
| Q7LFL8 | CXXC5 | 4.661 | 0.943058 |
| Q13501 | SQSTM1 | 4.615 | 0.650292 |
| Q08945 | SSRP1 | 4.573 | 0.966048 |
| P78545 | ELF3 | 4.522 | 0.578015 |
| Q9H0U9 | TSPYL1 | 4.447 | 0.928093 |
| Q8NI35 | PATJ | 4.359 | 0.966048 |
| P04637 | TP53 | 4.328 | 0.966048 |
| P01033 | TIMP1 | 4.323 | 0.966048 |
| Q86T24 | ZBTB33 | 4.315 | 2.56E−16 |
| Q96J88 | EPSTI1 | 4.258 | 0.966048 |
| P37231 | PPARG | 4.239 | 0.966048 |
| O43815 | STRN | 4.23 | 0.984925 |
| Q15345 | LRRC41 | 4.129 | 0.736231 |
| Q14687 | GSE1 | 3.937 | 0.965708 |
| Q92817 | EVPL | 3.903 | 0.966048 |
| Q02556 | IRF8 | 3.83 | 0.966048 |
| P18827 | SDC1 | 3.781 | 0.999254 |
| Q06481 | APLP2 | 3.75 | 0.988059 |
| O94955 | RHOBTB3 | 3.747 | 0.966048 |
| Q7L7X3 | TAOK1 | 3.739 | 0.378937 |
| P41212 | ETV6 | 3.645 | 0.928053 |
| Q9BX16 | TBC1D10A | 3.644 | 0.966048 |
| Q92738 | USP6NL | 3.62 | 2.56E−16 |
| P61626 | LYZ | 3.586 | 0.966048 |
| Q9H3M7 | TXNIP | 3.551 | 0.966048 |
| Q9NQC1 | JADE2 | 3.533 | 0.966048 |
| Q9BZL1 | UBL5 | 3.528 | 0.966048 |
| P54274 | TERF1 | 3.48 | 2.56E−16 |
| O15417 | TNRC18 | 3.452 | 2.56E−16 |
| Q08999 | RBL2 | 3.448 | 2.56E−16 |
| O95071 | UBR5 | 3.446 | 0.966048 |
| P36402 | TCF7 | 3.438 | 0.966048 |
| Q9BU68 | PRR15L | 3.417 | 0.966048 |
| Q8WUJ3 | CEMIP | 3.405 | 0.966048 |
| Q99856 | ARID3A | 3.384 | 2.56E−16 |
| Q9H582 | ZNF644 | 3.378 | 0.850436 |
| P14555 | PLA2G2A | 3.36 | 0.966048 |
| P27930 | IL1R2 | 3.355 | 0.971263 |
| Q96L93 | KIF16B | 3.343 | 0.966048 |
| Q9H5V8 | CDCP1 | 3.339 | 0.966048 |
| Q02447 | SP3 | 3.331 | 0.965708 |
| P01034 | CST3 | 3.323 | 0.98279 |
| Q9Y597 | KCTD3 | 3.301 | 0.966048 |
| P13987 | CD59 | 3.238 | 0.966048 |
| Q9H3F6 | KCTD10 | 3.228 | 0.920814 |
| Q8WV24 | PHLDA1 | 3.167 | 0.966048 |
| O75376 | NCOR1 | 3.167 | 0.966048 |
| Q9NZI6 | TFCP2L1 | 3.108 | 2.56E−16 |
| 043291 | SPINT2 | 3.068 | 0.999575 |
| Q9P2B4 | CTTNBP2NL | 3.067 | 0.966048 |
| P48436 | SOX9 | 3.033 | 0.966048 |
| O76080 | ZFAND5 | 3.01 | 0.966048 |
| Q3SY84 | KRT71 | 2.966 | 0.966048 |
| Q7RTS7 | KRT74 | 2.966 | 0.966048 |
| Q86Y46 | KRT73 | 2.966 | 0.966048 |
| Q9GZR7 | DDX24 | 2.958 | 0.966048 |
| Q08174 | PCDH1 | 2.952 | 0.966048 |
| Q9BXS6 | NUSAP1 | 2.949 | 0.966048 |
| P46100 | | 2.903 | 0.966048 |
| P20930 | FLG | 2.903 | 0.999575 |
| Q5K651 | SAMD9 | 2.888 | 0.988059 |
| Q92908 | GATA6 | 2.882 | 0.966048 |
| 000391 | QSOX1 | 2.874 | 0.966048 |
| P85037 | FOXK1 | 2.871 | 0.966048 |
| Q08211 | DHX9 | 2.87 | 0.964321 |
| Q86WB0 | ZC3HC1 | 2.857 | 0.966048 |
| P12036 | NEFH | 2.819 | 0.966048 |
| Q7Z5L9 | IRF2BP2 | 2.78 | 0.966048 |
| Q8N3R9 | PALS1 | 2.764 | 0.966048 |
| O14753 | OVOL1 | 2.731 | 2.56E−16 |
| Q9Y287 | ITM2B | 2.73 | 0.984925 |
| Q9Y4E5 | ZNF451 | 2.725 | 0.924564 |
| P41743 | PRKCI | 2.72 | 0.966048 |
| O94887 | FARP2 | 2.719 | 0.966048 |
| P00973 | OAS1 | 2.711 | 0.966048 |
| Q9UKJ3 | GPATCH8 | 2.704 | 0.966048 |
| P61769 | B2M | 2.695 | 0.991282 |
| P24468 | NR2F2 | 2.677 | 2.56E−16 |
| O14965 | AURKA | 2.671 | 0.966048 |
| Q8N163 | CCAR2 | 2.644 | 0.966048 |
| Q8IY67 | RAVER1 | 2.631 | 0.968062 |
| Q99081 | TCF12 | 2.615 | 2.56E−16 |
| O15379 | HDAC3 | 2.615 | 0.966048 |
| Q04726 | TLE3 | 2.601 | 0.988059 |
| P42704 | LRPPRC | 2.601 | 0.997712 |
| P09758 | TACSTD2 | 2.596 | 0.979557 |
| Q8TDY2 | RB1CC1 | 2.581 | 0.966048 |
| Q5VZF2 | MBNL2 | 2.57 | 0.966048 |
| Q96JH7 | VCPIP1 | 2.568 | 0.999254 |
| Q9ULW3 | ABT1 | 2.563 | 0.966048 |
| Q9NVW2 | RLIM | 2.558 | 0.966048 |
| Q9BZK7 | TBL1XR1 | 2.556 | 0.966048 |
| Q9UPQ0 | LIMCH1 | 2.54 | 0.874158 |
| Q93074 | MED12 | 2.54 | 0.966048 |
| Q8N3E9 | PLCD3 | 2.531 | 0.966048 |
| Q03468 | ERCC6 | 2.53 | 0.984133 |
| Q96PZ2 | FAM111A | 2.529 | 0.966048 |
| Q15572 | TAF1C | 2.527 | 2.56E−16 |
| P10589 | NR2F1 | 2.525 | 2.56E−16 |
| Q14596 | NBR1 | 2.525 | 0.966048 |
| O43286 | B4GALT5 | 2.511 | 0.968062 |
| O43688 | PLPP2 | 2.495 | 0.966048 |
| Q9UPW6 | SATB2 | 2.492 | 0.984925 |
| Q8IUH3 | RBM45 | 2.485 | 0.966048 |
| Q9BTA9 | WAC | 2.48 | 0.966048 |
| Q9H254 | SPTBN4 | 2.478 | 2.56E−16 |
| Q9UPN6 | SCAF8 | 2.478 | 0.966048 |
| P15336 | ATF2 | 2.477 | 2.56E−16 |
| P15529 | CD46 | 2.476 | 0.984925 |
| P08779 | KRT16 | 2.475 | 0.966048 |
| Q71F56 | MED13L | 2.468 | 0.966048 |
| Q13887 | KLF5 | 2.456 | 2.56E−16 |
| P19801 | AOC1 | 2.452 | 0.966048 |
| E9PRG8 | C11orf98 | 2.451 | 0.968062 |
| Q86VM9 | ZC3H18 | 2.442 | 0.966048 |
| Q9UNN8 | PROCR | 2.439 | 0.988059 |
| P82673 | MRPS35 | 2.434 | 0.999872 |
| Q96L73 | NSD1 | 2.423 | 2.56E−16 |
| P29317 | EPHA2 | 2.421 | 0.966048 |
| O75323 | NIPSNAP2 | 2.405 | 0.966048 |
| Q9BQ87 | TBL1Y | 2.403 | 0.966048 |
| Q96EL2 | MRPS24 | 2.378 | 0.999872 |
| O75925 | PIAS1 | 2.376 | 0.920814 |
| Q14258 | TRIM25 | 2.362 | 0.966048 |
| O60907 | TBL1X | 2.361 | 0.966048 |
| Q9BPW8 | NIPSNAP1 | 2.345 | 0.979194 |
| Q96KR1 | ZFR | 2.316 | 0.966048 |
| Q8WXD5 | GEMIN6 | 2.314 | 0.999872 |
| Q9HCM4 | EPB41L5 | 2.312 | 0.966048 |
| Q9H6W3 | RIOX1 | 2.311 | 0.966048 |
| Q86YW9 | MED12L | 2.311 | 0.966048 |
| Q9UBP9 | GULP1 | 2.308 | 0.966048 |
| P61764 | STXBP1 | 2.304 | 0.975186 |
| P55061 | TMBIM6 | 2.301 | 0.999872 |
| Q7Z2T5 | TRMT1L | 2.284 | 0.966048 |
| Q15020 | SART3 | 2.283 | 0.966048 |
| P07602 | PSAP | 2.282 | 0.971263 |
| Q9Y618 | NCOR2 | 2.278 | 0.966048 |
| Q9Y4H2 | IRS2 | 2.267 | 0.966048 |
| Q15582 | TGFBI | 2.258 | 0.966048 |
| Q8N4C8 | MINK1 | 2.251 | 0.966048 |
| Q16540 | MRPL23 | 2.248 | 0.988059 |
| O15164 | TRIM24 | 2.246 | 0.966048 |
| Q9Y6G3 | MRPL42 | 2.241 | 0.996552 |
| Q08554 | DSC1 | 2.239 | 0.968062 |
| Q92876 | KLK6 | 2.233 | 0.996552 |
| P49336 | CDK8 | 2.224 | 0.966048 |
| P52630 | STAT2 | 2.213 | 0.988059 |
| Q96JP5 | ZFP91 | 2.205 | 0.966048 |
| Q15773 | MLF2 | 2.201 | 0.986056 |
| Q6ZVX7 | NCCRP1 | 2.199 | 0.966048 |
| Q6YHK3 | CD109 | 2.193 | 0.988059 |
| Q8IV08 | PLD3 | 2.191 | 0.966048 |
| Q12830 | BPTF | 2.189 | 0.966048 |
| P09001 | MRPL3 | 2.188 | 0.988059 |
| Q9NZC4 | EHF | 2.183 | 2.56E−16 |
| Q9Y639 | NPTN | 2.181 | 0.999872 |
| P17676 | CEBPB | 2.18 | 2.56E−16 |
| P41229 | KDM5C | 2.173 | 0.966048 |
| P55899 | FCGRT | 2.166 | 0.999872 |
| P25774 | CTSS | 2.161 | 0.966048 |
| Q9H329 | EPB41L4B | 2.153 | 0.966048 |
| Q14197 | MRPL58 | 2.153 | 0.979557 |
| Q8NCR9 | CLRN3 | 2.15 | 0.966048 |
| O95067 | CCNB2 | 2.146 | 2.56E−16 |
| Q9BY66 | KDM5D | 2.145 | 0.966048 |
| P62633 | CNBP | 2.134 | 0.968062 |
| Q01780 | EXOSC10 | 2.134 | 0.966048 |
| Q8NFA2 | NOXO1 | 2.132 | 0.966048 |
| P51825 | AFF1 | 2.132 | 0.966048 |
| Q6PCB5 | RSBN1L | 2.131 | 0.966048 |
| Q9NQ50 | MRPL40 | 2.13 | 0.988059 |
| Q9GZT3 | SLIRP | 2.129 | 0.998036 |
| Q9HCY8 | S100A14 | 2.128 | 0.966048 |
| P56524 | HDAC4 | 2.128 | 0.999872 |
| Q8N2W9 | PIAS4 | 2.127 | 0.966048 |
| P15408 | FOSL2 | 2.123 | 2.56E−16 |
| Q9NVS2 | MRPS18A | 2.123 | 0.991493 |
| P25311 | AZGP1 | 2.122 | 0.996552 |
| Q9UKV3 | ACIN1 | 2.122 | 0.965708 |
| Q9Y261 | FOXA2 | 2.119 | 0.622097 |
| Q9Y676 | MRPS18B | 2.119 | 0.999575 |
| O95235 | KIF20A | 2.109 | 0.966048 |
| Q9BYC8 | MRPL32 | 2.107 | 0.988059 |
| Q15287 | RNPS1 | 2.103 | 0.966048 |
| P41235 | HNF4A | 2.103 | 0.966048 |
| Q9BZE1 | MRPL37 | 2.102 | 0.988059 |
| Q9BWU1 | CDK19 | 2.101 | 0.934365 |
| Q9NP92 | MRPS30 | 2.101 | 0.988059 |
| P38571 | LIPA | 2.095 | 0.999872 |
| O95251 | KAT7 | 2.091 | 0.966048 |
| Q7Z7F0 | KHDC4 | 2.088 | 0.667684 |
| A4D1E9 | GTPBP10 | 2.088 | 0.98118 |
| Q9BYD2 | MRPL9 | 2.082 | 0.988059 |
| Q9BYD1 | MRPL13 | 2.076 | 0.988059 |
| P80188 | LCN2 | 2.075 | 0.976566 |
| P17275 | JUNB | 2.073 | 2.56E−16 |
| Q9H8V3 | ECT2 | 2.072 | 0.966048 |
| O00468 | AGRN | 2.072 | 0.968062 |
| Q96DV4 | MRPL38 | 2.068 | 0.988059 |
| P56199 | ITGA1 | 2.066 | 0.966048 |
| Q6P1K8 | GTF2H2C; | 2.055 | 0.966048 |
| | GTF2H2C_2 | | |
| Q9Y3A3 | MOB4 | 2.049 | 0.999872 |
| Q7Z7M9 | GALNT5 | 2.049 | 0.988059 |
| Q15375 | EPHA7 | 2.048 | 0.988059 |
| Q96EY7 | PTCD3 | 2.047 | 0.999872 |
| Q96HP0 | DOCK6 | 2.045 | 0.966048 |
| O43823 | AKAP8 | 2.044 | 0.966048 |
| Q96S59 | RANBP9 | 2.04 | 0.966048 |
| Q9H0H5 | RACGAP1 | 2.039 | 2.56E−16 |
| P05067 | APP | 2.039 | 0.999872 |
| P82675 | MRPS5 | 2.039 | 0.999872 |
| P62873 | GNB1 | 2.028 | 0.979557 |
| Q13888 | GTF2H2 | 2.027 | 0.966048 |
| Q9UHL9 | GTF2IRD1 | 2.015 | 0.966048 |
| Q9NYT0 | PLEK2 | 2.011 | 0.966048 |
| Q9H307 | PNN | 2.011 | 0.966048 |
| Q13616 | CUL1 | 2.008 | 0.966048 |
| Q96A35 | MRPL24 | 2.004 | 0.988059 |
| Q14807 | KIF22 | 2.003 | 0.76765 |
| Q93045 | STMN2 | 0.498 | 0.934365 |
| Q92621 | NUP205 | 0.498 | 0.966048 |
| P49770 | EIF2B2 | 0.497 | 0.968062 |
| Q96SK2 | TMEM209 | 0.495 | 0.966048 |
| O15084 | ANKRD28 | 0.494 | 0.972344 |
| Q13144 | EIF2B5 | 0.493 | 0.987346 |
| Q9BU61 | NDUFAF3 | 0.492 | 0.966048 |
| B2CWD2 | | 0.492 | 0.999872 |
| Q9UKM9 | RALY | 0.492 | 0.966048 |
| P20700 | LMNB1 | 0.492 | 0.451362 |
| Q9P1F3 | ABRACL | 0.49 | 0.966048 |
| Q9NX62 | BPNT2 | 0.49 | 0.966048 |
| Q27J81 | INF2 | 0.489 | 0.966048 |
| Q9P012 | EMC3 | 0.487 | 0.966048 |
| Q9BRX5 | GINS3 | 0.485 | 0.966048 |
| O00401 | WASL | 0.484 | 0.999575 |
| P42025 | ACTR1B | 0.48 | 0.966048 |
| O00303 | EIF3F | 0.478 | 0.966048 |
| P49589 | CARS1 | 0.476 | 0.988059 |
| P0CG08 | GPR89B | 0.473 | 0.966048 |
| Q8ND56 | LSM14A | 0.47 | 0.999872 |
| Q9UI10 | EIF2B4 | 0.469 | 0.98118 |
| Q6P3R8 | NEK5 | 0.468 | 0.966048 |
| Q96AT9 | RPE | 0.467 | 0.966048 |
| Q13155 | AIMP2 | 0.466 | 0.966048 |
| Q9HD67 | MYO10 | 0.465 | 0.999872 |
| Q99519 | NEU1 | 0.465 | 0.999254 |
| P34059 | GALNS | 0.464 | 0.966048 |
| Q96HV5 | TMEM41A | 0.462 | 0.966048 |
| Q9NW68 | BSDC1 | 0.462 | 0.966048 |
| Q5SNT2 | TMEM201 | 0.458 | 0.966048 |
| Q03519 | TAP2 | 0.458 | 0.966048 |
| Q8WY22 | BRI3BP | 0.458 | 0.999872 |
| Q83607 | MF16 | 0.457 | 0.966048 |
| Q9ULX9 | MAFF | 0.456 | 2.56E−16 |
| Q9BXY0 | MAK16 | 0.455 | 0.966048 |
| Q659C4 | LARP1B | 0.455 | 0.988059 |
| O43324 | EEF1E1 | 0.455 | 0.966048 |
| Q96GQ5 | RUSF1 | 0.454 | 0.966048 |
| P35232 | PHB1 | 0.452 | 0.966048 |
| Q9UKZ1 | CNOT11 | 0.451 | 0.988059 |
| Q9Y2T3 | GDA | 0.45 | 0.966048 |
| P30153 | PPP2R1A | 0.449 | 0.999872 |
| Q8NE86 | MCU | 0.448 | 0.966048 |
| Q9NXE4 | SMPD4 | 0.447 | 0.966048 |
| Q9P0U3 | SENP1 | 0.445 | 0.966048 |
| Q9GZY8 | MFF | 0.443 | 0.966048 |
| P22694 | PRKACB | 0.441 | 0.966048 |
| O94901 | | 0.441 | 0.968062 |
| Q6PJ69 | TRIM65 | 0.439 | 0.999872 |
| Q7Z7H5 | TMED4 | 0.439 | 0.966048 |
| O43808 | SLC25A17 | 0.438 | 0.971611 |
| O75477 | ERLIN1 | 0.437 | 0.966048 |
| Q9UNS1 | TIMELESS | 0.436 | 0.966048 |
| Q9UJ72 | ANXA10 | 0.436 | 0.966048 |
| Q99623 | PHB2 | 0.432 | 0.966048 |
| Q9BTX1 | NDC1 | 0.431 | 0.966048 |
| Q7RTS9 | DYM | 0.43 | 0.966048 |
| B7ZAP0 | RABGAP1L | 0.43 | 0.548146 |
| Q96R06 | SPAG5 | 0.429 | 0.988059 |
| Q9NVI1 | FANCI | 0.429 | 0.988059 |
| Q5T1M5 | FKBP15 | 0.424 | 0.999575 |
| Q01518 | CAP1 | 0.423 | 0.966048 |
| Q9UM00 | TMCO1 | 0.421 | 0.966048 |
| Q15050 | RRS1 | 0.419 | 0.971263 |
| Q8N0U8 | VKORC1L1 | 0.416 | 0.966048 |
| P28749 | RBL1 | 0.415 | 0.966048 |
| Q9NRG9 | AAAS | 0.414 | 0.984133 |
| Q9BXW9 | FANCD2 | 0.413 | 0.966048 |
| Q6P2E9 | EDC4 | 0.409 | 0.994092 |
| Q08378 | GOLGA3 | 0.408 | 0.966048 |
| P24390 | KDELR1 | 0.407 | 0.966048 |
| Q68CZ6 | HAUS3 | 0.406 | 0.966048 |
| Q8NC56 | LEMD2 | 0.404 | 0.966048 |
| O43312 | MTSS1 | 0.404 | 2.56E−16 |
| Q86UP2 | KTN1 | 0.4 | 0.966048 |
| Q8TE73 | DNAH5 | 0.397 | 0.966048 |
| P07910 | HNRNPC | 0.396 | 0.966048 |
| P26641 | EEF1G | 0.396 | 0.984925 |
| Q9NPI6 | DCP1A | 0.395 | 0.988059 |
| Q12981 | BNIP1 | 0.392 | 0.934365 |
| O60830 | TIMM17B | 0.392 | 0.966048 |
| P78325 | ADAM8 | 0.384 | 2.56E−16 |
| Q9H115 | NAPB | 0.382 | 0.966048 |
| P30154 | PPP2R1B | 0.382 | 0.993144 |
| Q9HCE1 | MOV10 | 0.38 | 0.988059 |
| Q96DR4 | STARD4 | 0.378 | 0.966048 |
| P52732 | KIF11 | 0.378 | 0.979557 |
| B2CWA5 | | 0.375 | 0.966048 |
| P29825 | | 0.375 | 0.966048 |
| Q92600 | CNOT9 | 0.374 | 0.988059 |
| Q14739 | LBR | 0.374 | 0.966048 |
| Q9ULE6 | PALD1 | 0.373 | 0.966048 |
| Q99871 | | 0.372 | 0.966048 |
| Q9Y3Y2 | CHTOP | 0.372 | 0.934365 |
| B7ZW38 | HNRNPCL3 | 0.371 | 0.966048 |
| Q92609 | TBC1D5 | 0.368 | 0.966048 |
| O60812 | HNRNPCL1 | 0.366 | 0.966048 |
| B2RXH8 | HNRNPCL2 | 0.366 | 0.966048 |
| P54577 | YARS1 | 0.365 | 0.968062 |
| O95989 | NUDT3 | 0.362 | 0.996683 |
| A8MYJ7 | TTC34 | 0.358 | 0.966048 |
| Q9UMX5 | NENF | 0.348 | 0.966048 |
| B2CWQ3 | m147R | 0.344 | 0.966048 |
| O94923 | GLCE | 0.339 | 0.987198 |
| P49759 | CLK1 | 0.338 | 2.56E−16 |
| Q9UKR5 | ERG28 | 0.338 | 0.966048 |
| O95379 | TNFAIP8 | 0.334 | 0.966048 |
| Q01581 | HMGCS1 | 0.332 | 0.966048 |
| P29692 | EEF1D | 0.331 | 0.999872 |
| P04626 | ERBB2 | 0.33 | 0.999872 |
| Q9Q8Q4 | m042L | 0.328 | 0.968062 |
| Q77PA1 | m138L | 0.324 | 0.966048 |
| Q9Q8K4 | VETFL | 0.322 | 0.999575 |
| B2CWK3 | | 0.322 | 0.999575 |
| E2CZL5 | m126R | 0.322 | 0.966048 |
| P24534 | EEF1B2 | 0.32 | 0.988059 |
| Q8WZ42 | TTN | 0.304 | 0.988059 |
| Q9Q8Q2 | NPH2 | 0.292 | 0.984925 |
| B2CWF1 | | 0.292 | 0.984925 |
| Q04864 | REL | 0.29 | 0.966048 |
| P26640 | VARS1 | 0.282 | 0.968062 |
| E2CZL2 | m123R | 0.277 | 0.984925 |
| D2WN93 | m151R | 0.255 | 0.966048 |
| Q6SJ93 | FAM111B | 0.253 | 0.968062 |
| Q16204 | CCDC6 | 0.249 | 0.988059 |
| B2CWH8 | | 0.232 | 0.966048 |
| Q9Q8M9 | m071L | 0.232 | 0.966048 |
| B2CWQ7 | | 0.229 | 0.966048 |
| Q9P000 | COMMD9 | 0.213 | 0.999872 |
| P01100 | FOS | 0.211 | 0.966048 |
| B2CWH1 | m064R | 0.211 | 0.966048 |
| E2CZW7 | m072L | 0.211 | 0.966048 |
| Q9UJ68 | MSRA | 0.202 | 0.966048 |
| B2CWC6 | m020L | 0.191 | 0.979557 |
| Q9Q8S2 | m020L | 0.191 | 0.979557 |
| Q9Q8F9 | m144R | 0.179 | 0.966048 |
| B2CWG5 | m058R | 0.176 | 0.966048 |
| Q9Q8N8 | m058R | 0.176 | 0.966048 |
| Q9Q8S8 | m013L | 0.172 | 0.999254 |
| P68550 | | 0.168 | 0.966048 |
| Q9Q8G2 | m140R | 0.165 | 0.968062 |
| Q5BJH7 | YIF1B | 0.161 | 0.984925 |
| B2CWJ9 | m092L | 0.16 | 0.966048 |
| B2CWA6 | m003.1R | 0.145 | 0.966048 |
| Q77PB1 | m003.1L | 0.145 | 0.966048 |
| E2CZR6 | m021L | 0.133 | 0.966048 |
| E2CZQ3 | m009L | 0.132 | 0.999254 |
| B2CWI6 | m079R | 0.131 | 0.999872 |
| P07942 | LAMB1 | 0.128 | 0.780158 |
| Q9Q8F0 | m154L | 0.125 | 0.966048 |
| Q85296 | | 0.124 | 0.924564 |
| O55698 | M-T4 | 0.122 | 0.966048 |
| B2CWA8 | | 0.122 | 0.966048 |
| Q9PXA3 | m004L | 0.122 | 0.966048 |
| Q9Q8H2 | m129R | 0.119 | 0.956924 |
| B2CWF3 | m046L | 0.111 | 0.966048 |
| B2CWL8 | m111R | 0.101 | |
| Q9Q8R0 | m036L | 0.099 | 0.966048 |
| D2WN26 | m090L | 0.097 | 0.984925 |
| B2CWI3 | m076R | 0.096 | 0.966048 |
| Q68DK2 | ZFYVE26 | 0.092 | 0.920814 |
| B2CWC0 | | 0.092 | 0.966048 |
| E2CZQ9 | m014L | 0.092 | 0.966048 |
| B2CWL5 | m108R | 0.088 | 0.968062 |
| Q9Q8L9 | VETFS | 0.087 | 0.966048 |
| Q9PX24 | m007L | 0.087 | 0.853218 |
| B2CWM1 | | 0.083 | 0.966048 |
| E2CZK3 | m114R | 0.083 | 0.966048 |
| Q9Q8I6 | m114R | 0.083 | 0.966048 |
| B2CWM2 | m115L | 0.077 | 0.966048 |
| Q9Q8N5 | | 0.069 | 0.966048 |
| B2CWH0 | | 0.069 | 0.966048 |
| Q9Q8L3 | m087L | 0.069 | 0.966048 |
| Q9Q8Q3 | m043L | 0.067 | 0.966048 |
| B2CWE3 | | 0.064 | 0.966048 |
| Q9Q8Q1 | | 0.059 | 0.966048 |
| D2WM37 | m012L | 0.058 | 2.56E−16 |
| B2CWC8 | | 0.058 | 0.966048 |
| Q83604 | MF13 | 0.058 | 0.966048 |
| D2WMA0 | m030L | 0.057 | 0.966048 |
| B2CWB2 | | 0.055 | 0.281853 |
| E2CZV0 | m055R | 0.052 | 0.966048 |
| E2CZW3 | m068R | 0.052 | 0.966048 |
| B2CWF6 | | 0.051 | 0.966048 |
| Q9Q8P7 | m049R | 0.051 | 0.966048 |
| Q77P93 | m003.2L | 0.049 | 0.619096 |
| E2CZM2 | m133R | 0.048 | 0.966048 |
| Q9Q8M6 | m074R | 0.047 | 0.966048 |
| B2CWE1 | | 0.047 | 0.966048 |
| Q9Q8R2 | m034L | 0.047 | 0.966048 |
| B2CWD4 | m027L | 0.045 | 0.966048 |
| B2CWB4 | m008.1L | 0.045 | 0.965708 |
| P68544 | PAPS | 0.044 | 0.966048 |
| B2CWP6 | m139R | 0.04 | 0.966048 |
| E2CZR0 | m015L | 0.04 | 2.56E−16 |
| B2CWC1 | m015L | 0.04 | 2.56E−16 |
| Q9Q8S0 | m026R | 0.039 | 0.975186 |
| B2CWF4 | m047R | 0.039 | 0.966048 |
| Q9Q8K7 | m093L | 0.039 | 2.56E−16 |
| Q9Q817 | m113R | 0.037 | 0.966048 |
| Q9Q8L8 | m082R | 0.037 | 0.756075 |
| E2CZP2 | m001R | 0.037 | 0.966048 |
| O10625 | M-T1 | 0.037 | 0.966048 |
| B2CWB5 | | 0.036 | 0.364764 |
| B2CWI7 | m080R | 0.036 | 0.789543 |
| Q9PX36 | m006L | 0.035 | 0.966048 |
| Q9Q8J1 | m109L | 0.032 | 0.966048 |
| Q9YQ03 | mt6 | 0.031 | 0.966048 |
| Q9Q8K1 | m099L | 0.03 | 0.782236 |
| Q9Q8P4 | m052L | 0.029 | 0.966048 |
| B2CWK2 | m095L | 0.029 | 2.56E−16 |
| Q9Q8I4 | m117L | 0.027 | 0.934365 |
| E2CZL6 | m127L | 0.025 | 0.966048 |
| Q9Q8I1 | m120L | 0.024 | 0.966048 |
| B2CWD6 | m029L | 0.023 | 0.966048 |
| Q9Q8Q8 | m038L | 0.023 | 0.393578 |
| B2CWR1 | m156R | 0.022 | 0.803501 |
| Q9Q8H9 | m122R | 0.022 | 0.920814 |
| Q9Q8G6 | m135R | 0.021 | 0.619142 |
| D2WN05 | m078R | 0.021 | 0.482739 |
| B2CWI0 | | 0.021 | 0.999872 |
| K4JKC2 | m073R | 0.021 | 0.999872 |
| Q9Q8Q6 | m040L | 0.011 | 0.449876 |
| B2CWD8 | | 0.011 | 0.966048 |
| Q9Q8R5 | m031R | 0.011 | 0.966048 |
| O76015 | KRT38 | 0.01 | 2.56E−16 |
| O76013 | KRT36 | 0.01 | 2.56E−16 |
| E2CZJ9 | m110L | 0.01 | 2.56E−16 |
| Q5VVJ2 | MYSM1 | 0.01 | 2.56E−16 |
| Q9Q8K3 | m097R | 0.01 | 2.56E−16 |
| B2CWE0 | m033R | 0.01 | 2.56E−16 |
| O00584 | RNASET2 | 0.01 | 2.56E−16 |
| Q58719 | SFT2D3 | 0.01 | 2.56E−16 |
| Q9Q8G4 | m137R | 0.01 | 2.56E−16 |
| Q96HD1 | CRELD1 | 0.01 | 2.56E−16 |
| E2CZV4 | m059R | 0.01 | 2.56E−16 |
| P50583 | NUDT2 | 0.01 | 2.56E−16 |
| Q86WV6 | STING1 | 0.01 | 2.56E−16 |
| Q83730 | m005R | 0.01 | 2.56E−16 |
| B2CWB0 | | 0.01 | 2.56E−16 |
| Q9Q8R4 | m032R | 0.01 | 2.56E−16 |
| E2CZM0 | m131R | 0.01 | 2.56E−16 |
| Q8IY95 | TMEM192 | 0.01 | 2.56E−16 |
| P10909 | CLU | 0.01 | 2.56E−16 |
| E2CZV1 | m056R | 0.01 | 2.56E−16 |
| P05090 | APOD | 0.01 | 2.56E−16 |
| B2CWQ5 | m149R | 0.01 | 2.56E−16 |
| Q9Q8J4 | m106L | 0.01 | 2.56E−16 |
| P14209 | CD99 | 0.01 | 2.56E−16 |
| Q96LJ7 | DHRS1 | 0.01 | 2.56E−16 |
| Q9UHX3 | ADGRE2 | 0.01 | 2.56E−16 |
| E2CZJ6 | m107L | 0.01 | 0.680302 |
| Q9Q8K6 | m094R | 0.01 | 2.56E−16 |
| Q9Q8J5 | m105L | 0.01 | 2.56E−16 |
| Q8TB72 | PUM2 | 0.01 | |
| Q9Q8N0 | m070R | 0.01 | 2.56E−16 |
| B2CWI4 | m077L | 0.01 | 2.56E−16 |
| B2CWL4 | | 0.01 | 0.680302 |
| B2CWG8 | m061R | 0.01 | 2.56E−16 |
| B2CWH3 | m066R | 0.01 | 2.56E−16 |
| B2CWJ3 | | 0.01 | 2.56E−16 |
| B2CWG4 | m057L | 0.01 | 2.56E−16 |
| E2CZY1 | m086L | 0.01 | 2.56E−16 |
| Q12913 | PTPRJ | 0.01 | 2.56E−16 |
| B2CWG7 | m060R | 0.01 | 2.56E−16 |
| Q9Q8M5 | m075R | 0.01 | 0.768355 |
| P17516 | AKR1C4 | 0.01 | 2.56E−16 |
| Q8TDQ7 | GNPDA2 | 0.01 | 2.56E−16 |
| B2CWH6 | m069L | 0.01 | 0.901903 |
| Q85297 | | 0.01 | 0.901903 |
| D3W0C4 | m130R | 0.01 | 2.56E−16 |
| Q9Q8L1 | m089L | 0.01 | 2.56E−16 |
| P17066 | HSPA6 | 0.01 | 2.56E−16 |
| Q9Q8J9 | m101L | 0.01 | 2.56E−16 |
| B2CWG0 | m053R | 0.01 | 0.653738 |
| B2CWP9 | m142R | 0.01 | 0.966048 |
| B2CWJ5 | | 0.01 | 2.56E−16 |
| E2CZH7 | m088L | 0.01 | 2.56E−16 |

### XV. EXAMPLE 10: COMBINATION OF SELINEXOR AND MYXV FOR CANCER THERAPY

Cancer is the second leading cause of death, and the global number of cancer-related deaths is increasing. Therefore, novel treatment strategies are needed to improve therapeutic outcomes. Among the many new cancer treatment approaches, OVs have shown tremendous potential in preclinical animal models and clinical trials, allowing the approval of only a few OVs for patients (Rahman, M. M., et al., 2021, Cancers, 13(21):5452). However, there are still limitations to OVs that need to be addressed to obtain more widespread enhanced therapeutic benefits from this treatment approach. One such area of potential development is the understanding of how OVs and cancer cells interact. This is mainly because of the heterogeneity and complexity of the cancer cells in the tumor bed, which can alter the ability of OVs to replicate in cancer cells. Here, it is shown for the first time that targeting the nuclear export pathway can enhance the replication of the oncolytic MYXV in normally restricted human cancer cells (defined as either semi-permissive or non-permissive), thereby enhancing its oncolytic ability in preclinical animal models. Like other poxviruses, the oncolytic MYXV can promiscuously bind, enter, and initiate infection of most cancer cell types from different tissues and species. However, successful productive replication that leads to progeny virus production and eventual killing of cancer cells largely depends on the viral manipulation of multiple intracellular signaling pathways (Rahman, M. M., et al., 2020, Journal of Clinical Medicine, 9(1):171; Rahman, M. M., et al., 2020, Vaccines, 8(2):244; Advances in Virus Research, 71:135-171). Every cancer cell has a unique spectrum of deficiencies in their cellular innate defense pathways that normally attempt to restrict viral infections; therefore, human cancer cells belong to three general classes with respect to susceptibility to infection and killing by MYXV: fully permissive (i.e., produce viral progeny at levels comparable to rabbit cells), semi-permissive (i.e., produce at least an order of magnitude reduced levels of viral progeny), and non-permissive (little or no viral progeny). This work referred to MYXV tropism in human cancer cells in the latter two categories.

Among the known cellular factors in cancer cells, several members of the DEAD-box RNA helicases regulate MYXV replication levels in human cancer cells (Rahman, M. M., et al., 2017, Scientific Reports, 7:15710). These RNA helicases either inhibit MYXV replication (i.e., antiviral) or are required for optimal virus replication (i.e., proviral). It has previously been reported that DHX9/RNA helicase A (RHA) forms unique antiviral granules in the cytoplasm, which inhibit MYXV replication in human cancer cells (Rahman, M. M., et al., 2021, Journal of Virology, 95:e0015121). DHX9 antiviral granules in the cytoplasm function by reducing viral late protein synthesis and progeny virus formation. DHX9 knockdown in restricted human cancer cells significantly enhanced MYXV gene expression, progeny virus production, cell-to-cell spread, and foci formation. Apart from MYXV, DHX9 is known to also have either proviral or antiviral roles against diverse RNA and DNA viruses (Ullah, R., et al., 2022, Virus Research, 309:198658; Guo, F., et al., 2021, Virus Research, 291:198206). However, the diverse functions of DHX9 depend on the cell type and localization of the protein in the infected cells.

Similar to many other nuclear RNA helicases, DHX9 shuttles between the nuclear and cytosolic compartments to perform cellular functions (Tang, H., et al., 1999, Molecular and Cellular Biology, 19:3540-3550; Fujita, H., et al., 2005, International Journal of Molecular Medicine, 15:555-560). For example, DHX9 is imported via the classical importin-alpha/beta-dependent pathway (Aranti, S., et al., 2006, Biochemical and Biophysical Research Communications, 340:125-133). However, during RNA virus replication, DHX9 is also detected in the cytoplasm of the infected cells (Jefferson, M., et al., 2014, Journal of Virology, 88:10340-10353; Liu, L., et al., 2016, Journal of Virology, 90:5384-5398). Based on the observation that DHX9 shuttles between the nuclear and cytosolic compartments, nuclear export inhibitors that target XPO1/exportin1/CRM1 were tested for their ability to block the nuclear export of proteins in MYXV-infected human cancer cells. Surprisingly, unlike RNA viruses, blocking the nuclear export pathway using the XPO1 inhibitor leptomycin B (LMB) in human cancer cells significantly increased MYXV replication, similar to what was observed with the knockdown of DHX9.

Additionally, LMB treatment significantly reduced the formation of DHX9 antiviral granules in the cytoplasm of the MYXV-infected cells. To further confirm this enhanced virus replication XPO1/CRM1 specific, the expression of CRM1 was knocked down using siRNA. Similar to LMB treatment, CRM1 knockdown significantly enhanced MYXV replication in normally restrictive human cancer cells and reduced the formation of DHX9 antiviral granules in the cytoplasm. These results suggest that the cellular restriction proteins exported using CRM1 have inhibitory effects on cytoplasmic replication of MYXV. This is the first report that blocking the CRM1-mediated nuclear export pathway can enhance the replication of any virus and is opposite to what has been reported for many RNA viruses, such as HIV-1, influenza, respiratory syncytial virus (RSV), dengue virus, rabies virus, and human cytomegalovirus (HCMV), all of which depend on the CRM1 nuclear export pathway for replication (Mathew, C., et al., 2017, Frontiers in Microbiology, 8:1171).

Since LMB is relatively toxic to mammalian cells and unsuitable for in vivo studies in preclinical animal models, multiple synthetic derivatives were developed and tested as potential anticancer drugs with minimal toxicity (Mutka, S. C., et al., 2009, Cancer Research, 69:510-517; Newlands, E. S., et al., 1996, British Journal of Cancer, 74:648-649). One such LMB derivative, selinexor (KPT330), has been approved by the FDA and is suitable for in vivo studies (Richard, S., et al., 2020, Future Oncology, 16:1331-1350). Similar to LMB, selinexor also significantly enhanced MYXV replication in all human cancer cell lines tested, where replication of MYXV is normally restricted. In addition to enhancing virus production, the combination of Selinexor with MYXV significantly reduced cell proliferation and enhanced cancer cell death. More importantly, these results showed that selinexor, which has minimal toxicity to cells, can dramatically increase viral replication and cytotoxicity against cancer cells. Thus, these results demonstrate for the first time that selinexor enhances the oncolytic activity of MYXV. Next, it was tested whether selinexor could enhance MYXV infection and replication in 3D organoid-like cultures of human cancer cells, where virus replication is restricted to the outer shell of cell spheroids. A three-dimensional (3D) culture method was developed with multiple MYXV-restricted human cancer cell lines. When treated with selinexor and infected with MYXV, a significant increase in viral early and late gene expression was observed compared to MYXV infection alone, and greater penetration into the spheroid interior. These positive results from the 3D organoid-like culture motivated testing selinexor and MYXV in vivo using animal models.

In 2019, the FDA approved selinexor for hematological malignancies, such as multiple myeloma and lymphoma (Richard, S., et al., 2020, Future Oncology, 16:1331-1350). However, selinexor has also shown promising results against solid tumors in preclinical animal models and clinical trials (Ho, J., et al., 2022, Therapeutic Advances in Medical Oncology, 14:17588359221087555; Landes, J. R., et al., 2022, Journal of Cancer Research and Clinical Oncology, s00432-022-04247-z; Thirasastr, P., et al., 2022, Therapeutic Advances in Medical Oncology, 14:17588359221081073). Selinexor is delivered orally; thus, it has the potential to be combined with OV delivered either intratumorally or systemically. To test whether selinexor enhances MYXV replication and oncolytic activity in vivo, a xenograft model was established using human cancer cells subcutaneously implanted in NSG mice. The in vivo studies with three different MYXV-restricted human cancer cell lines, Colo205, HT29, and PANC-1, clearly demonstrated that selinexor significantly enhanced the replication of MYXV, as observed by measuring virus-derived luciferase signals in situ. To assess the therapeutic effect of MYXV, Selinexor, or Selinexor+MYXV, the virus was injected intratumorally into one of the two flanked tumors and selinexor systemically delivered by oral gavage multiple times. Tumor burden was measured during treatment; however, since there were tumors on both sides of the flank, mice were sacrificed when either one of the tumors reached the endpoint criteria. Selinexor alone significantly reduced the tumor burden bilaterally in all the tested xenograft models compared to the PBS control or MYXV-only treatment. More importantly, treatment with selinexor+MYXV further reduced the tumor burden in both the virus-injected and non-injected tumors compared to treatment with selinexor alone. From these studies in NSG mice, which are defective for any virus-induced acquired immunity against tumors, it was surprising that tumors that were not intratumorally injected with MYXV also showed a greater reduction in tumor burden than those treated with selinexor alone. To test whether MYXV was present in the un-injected tumors, the tumors were collected from PANC-1 xenograft mice, and virus titration showed the presence of MYXV in the un-injected tumor, but only at a very low level. Currently, it is difficult to conclude whether the presence of migrated MYXV, innate immune cells, or a combination of both contributes to this apparent abscopal tumor reduction. Another key finding was that in NSG mice, persistence of the virus was observed in the injected tumor bed for a relatively prolonged time due to the absence of an active antiviral immune system. This also contributed to the overall reduction in tumor burden, which was reflected in the PANC-1 xenograft model when the endpoint survival study was performed, where selinexor+MYXV treatment significantly enhanced the overall survival of the animals. Overall, enhanced therapeutic effects were observed in mice treated with selinexor+MYXV compared to treatment with selinexor or MYXV alone.

Finally, proteomic analyses of the human colorectal cancer cell line Colo205 was performed after treatment with selinexor, MYXV, and a combination of selinexor and MYXV to determine the global expression level changes in the cellular and viral proteins in the nuclear and cytosolic compartments. Comparing the different treatments and the relative abundance of proteins in the two cellular compartments, both cellular and viral proteins that were upregulated or downregulated by different treatments were identified.

The methods employed are described herein.

Cells: The rabbit cell line RK13 (ATCC #CCL-37), non-human primate Vero cells (ATCC #CCL-81), human cell lines A549 (ATCC #CCL-185), PANC-1 (ATCC #RCL-1469), and MDA-MB435 (ATCC #HTB-129) were cultured in Dulbecco's minimum essential medium (DMEM) supplemented with 10% fetal bovine serum, 2 mM glutamine, and 100 μg penicillin-streptomycin. Human colorectal cancer cell lines HT29 (ATCC #HTB-38) and Colo205 (ATCC #CCL-222) were cultured in McCoy's 5 medium and RPMI1640 media respectively, supplemented with 10% fetal bovine serum, 2 mM glutamine, and 100 μg penicillin-streptomycin. All cultures were maintained at 37° C. in a 5% humidified 5% incubator. Cells were regularly checked for Mycoplasma contamination using a universal Mycoplasma detection kit (ATCC 30-1012K).

Reagents and Antibodies: Rabbit polyclonal antibodies against DHX9 and CRM1, and mouse monoclonal antibodies against β-actin were purchased from Thermo Fisher Scientific. HRP-conjugated goat anti-rabbit and anti-mouse IgG antibodies were purchased from Jackson Immuno Research Laboratories. All secondary antibodies conjugated to Alexa Fluor 488, 594, 568, and 647 were purchased from Thermo Fisher Scientific. Selinexor (KPT330) was purchased from Apex Bio. Leptomycin A, Leptomycin B, Ratijadone A, and Anguinomycin A were purchased from Santa Cruz Biotechnology.

Viruses and Viral Replication: Wild-type myxoma virus constructs vMyx-GFP (WT-MYXV that express GFP under a poxvirus synthetic early/late promoter (sE/L), vMyx-GFP-TdTomato (WT-MYXV that express GFP under a poxvirus sE/L promoter and TdTomato under poxvirus p11 late promoter), vMyx-FLuc (WT-MYXV that express firefly luciferase under a poxvirus sE/L promoter and TdTomato under poxvirus p11 late promoter), and vMyx-M11L-KO (WT-MYXV lacking the M11L gene) were used (Rahman, M. M., et al., 2021, Journal of Virology, 95:e0015121; Pisklakova, A., et al., 2016, Neruo-Oncology, 18(8):1088-1098). All myxoma viruses were grown in Vero cells. Virus stocks were prepared using sucrose gradient purification (Smallwood, S. E., et al., 2010, Current Protocols in Microbiology, Chapter 14, Unit 14A.1).

Viral titers in different human cancer cell lines were determined using a viral replication assay. Cells were seeded in 24-well plates (2×10⁵ cells/well). The next day, the cells were treated with different concentrations of leptomycin B (LMB) or selinexor diluted in DMEM for 1 hour. MYXV was added to the cells and incubated for one hour at 37° C. After 1 hour, the unbound viruses were washed away using DMEM, and DMEM with LMB or selinexor was added to the cells. Cells were harvested in DMEM without LMB or selinexor at the indicated time points. After harvesting the cells, they were stored at −80° C. until processing. Samples were subjected to three freeze/thaw cycles and one-minute sonification to lyse cells and release the viral particles. Afterwards, different dilutions were prepared in DMEM and plated on rabbit RK13 and foci were counted after 48 hours using a fluorescent microscope. All assays and dilutions were performed in triplicate.

Spheroid Generation and Virus Infection: Different cancer cell lines were grown and maintained as previously described and used for spheroid generation within 2-5 passages. 96-well plates were prepared with rat tail collagen I to form the surface for spheroid culture. On the day of cell seeding for spheroid generation, the cells were dissociated with TrypLE, the TrypLE neutralized with fresh complete media, the cells spun down, and resuspended in fresh complete media. After making a single cell suspension, cells were counted using a Countess II automated cell counter and 1000 cells in 100 μL were plated on the surface of the collagen matrix. The cells were observed daily for spheroid formation. After 5-7 days, when spheroids reached the desired size, they were treated with selinexor and infected with vMyx-GFP-TdTomato.

Immunofluorescence: Cells (5×10⁵-1×10⁶/dish) were seeded onto glass bottom 35 mm petri dishes overnight. Depending on the experiment, the next day, cells were transfected with siRNA for 48 hours or treated with a nuclear export inhibitor, MYXV, or a combination of both. At different time points after treatment, the cells were washed with PBS three times, fixed with 2% paraformaldehyde in PBS for 12 minutes at room temperature, washed with PBS three times, and permeabilized in 0.1% Triton™ X-100 in PBS for 90 seconds at room temperature. Fixed cells were washed with PBS three times and then blocked with 3% BSA in PBS for 30 minutes at 37° C., incubated with primary antibody (1:300 dilution) for 30 minutes at 37° C., washed with PBS six times, and incubated with secondary antibodies conjugated to different Alexa Fluor. After washing again with PBS six times, samples were mounted on glass slides with Vecta Shield (Vectorlabs) containing DAPI (4′,6′-diamidino-2-phenylindole) to stain DNA in the nuclei and viral production. Images were captured using a fluorescence microscope (Leica).

siRNA Transfection: ON-TARGETplus™ SMART pool siRNAs for CRM1/XPO1 and a non-targeting control (NT siRNA) were purchased from Dharmacon (Horizon Discovery). 24-well plates were seeded with 40-50% confluence, left overnight for adherence, and transfected with siRNAs (50 nM) using Lipofectamine™ RNAiMAX (Invitrogen) transfection reagent. After 48 hours of transfections, the cells were infected with different MOI of vMYX-GFP for one hour, washed to remove the unbound virus, and incubated with complete media. At the indicated time points, cells were either observed under a fluorescence microscope to monitor and record the expression of fluorescent proteins or harvested and processed for titration of progeny virions.

Click-iT EdU Cell Proliferation Assay: To visualize and measure cell proliferation, a Click-iT™ EdU cell proliferation assay (Thermo Fisher) was used according to the manufacturer's instructions. Briefly, cells (5×10⁵/dish) were seeded on glass-bottom dishes and allowed to adhere by incubation overnight at 37° C. The next day the cells were treated with selinexor, MYXV, or a combination of both for 24 hours. Subsequently, EdU reagent (10 μM) was added and the cells were incubated for another 24 hours. To visualize EdU incorporation in dividing cells, the cells were fixed with 3.7% formaldehyde in PBS and permeabilized with 0.5% Triton™ X-100 in PBS. Cells were then incubated with the Click-iT™ EdU reaction cocktail with Alexa Fluor-594 for 30 minutes at room temperature and protected from light. The cells were washed with PBS and stained with Nuclear Mask Blue for nuclear staining. Fluorescence images were obtained using a fluorescence microscope and fluorescence signals were analyzed using ImageJ software.

Cell Proliferation Assay: To measure cancer cell proliferation based on the amount of cellular DNA, the CyQuant™ NF Cell Proliferation Assay Kit (Invitrogen) was used according to the manufacturer's instructions. Briefly, PANC-1, HT29, MDA-MB435, and Colo205 cells were seeded in a 96-well plate (1×10⁴ cells/well) and left to attach to the wells overnight. The next day, the medium was removed and replaced with 50 μL medium containing different concentrations of selinexor (0-1 μM). After an hour of incubation with selinexor, the virus was added to different MOIs (0.5-5), bringing the end volume of every well up to 100 μL. A 1× dye binding solution was prepared by adding 9 μL of the CyQuant™ NF Dye reagent in 4.5 mL Hank's Balanced Salt Solution (HBSS) buffer (Invitrogen). After 24, 48, 72, and 96 hours of incubation, the medium was removed from the cells and 50 μL 1× dye solution was added to all wells. The microplate was covered to protect it from light and was incubated for 30-60 minutes in a 5% CO₂ incubator at 37° C. Subsequently, cell proliferation was quantified by measuring fluorescence with an excitation of 485 nM and emission of 530 nM in a VarioSkan Lux Microplate reader (Thermo Fisher). All experiments were performed in quadruple and normalized to mock-treated cells.

Cell Viability Assay: To assess the viability of different human cancer cells after selinexor treatment or MYXV infection, 10,000 cells were seeded into each well of a 96-well plate. The next day, cells were treated with different concentrations of selinexor, infected with different MOIs of MYXV, or treated with different concentrations of selinexor for 1 hour followed by infection with different MOIs of MYXV. A minimum of four wells were used for each treatment condition, and untreated cells (mock) served as controls. Cell viability was assessed at 24, 48, 72, and 96 hours using an MTS assay.

Animal Studies: Male and female NSG mice were purchased from Jackson laboratory at 6-8 weeks of age. Animals were housed under sterile conditions. The animals were acclimatized for at least seven days before tumor implantation or any experimental procedures. Cells (1×10⁶/mouse in 100 μL PBS) were subcutaneously injected into the flanks of NSG mice. When the average tumor volumes reached 50-200 mm³, the mice were randomized into different treatment groups of five or six animals such that each treatment group had approximately the same average tumor volume. Tumor volume was measured two or three times per week as follows: volume=(length×width²)/2. When the tumor volume reached 1.5-2 cm³ animals were euthanized and tumors were collected for histology or processed for virus titration. To detect MYXV replication in the tumor bed luciferin was injected via IP delivery and bioluminescence images taken (Xenogen IVIS 2000).

Nucleus-Cytoplasm Fractionation and Proteomics: Colo205 cells were collected 48 hours after treatment with selinexor, MYXV infection, of selinexor+MYXV, and nuclear and cytosolic fractions were prepared using NE-PER™ nuclear and cytoplasmic extraction reagents (Thermo Scientific). The purity of the fractions was confirmed by Western blot analysis of tubulin (cytoplasmic) and histone H3 (nuclear). These fractions were used for LC-MS analysis at the Biosciences Mass Spectrometry Core Facility at Arizona State University. For LC-MS/MS, solubilized proteins were quantified (Thermo Fisher EZQ Protein Quantitation Kit or Pierce BCA). Proteins were reduced with 50 mM dithiothreitol (Sigma-Aldrich) at 95° C. for 10 minutes and alkylated for 30 minutes with 30 nM iodoacetamide (Pierce). Proteins were digested using 2.0 μg of MS-grade porcine trypsin (Pierce) and peptides were recovered using S-trap Micro Columns (ProtiFi) per manufacturer directions. Recovered peptides were dried via speed vac and resuspended in 30 μL of 0.1% formic acid.

LC-MS and LC-MS/MS analysis: All data-dependent mass spectra were collected in positive mode using an orbitrap Fusion Lumos mass spectrometer (Thermo Scientific) coupled with an UltiMate 3000 UHPLC (Thermo Scientific). One μL of the peptide was fractionated using an Easy-Spray LC column (500 mm×75 μm ID, PepMap C18, 2 μm particles, 100 Å pore size, Thermo Scientific) with an upstream 300 μm×5 mm trap column. Electrospray potential was set to 1.6 kV and the ion transfer tube temperature was 300° C. The mass spectra were collected using the “Universal” method optimized for peptide analysis provided by Thermo Scientific. Full MS scans (375-1500 m/z range) were acquired in profile mode with the following settings: Orbitrap resolution: 120,000 (at 200 m/z); cycle time: 3 seconds; mass range: “Normal”; RF Lens: 30%, AGC: “Standard”, Maximum Ion Accumulation: “Auto”; Monoisotopic Peak Determination (MIPS): “Peptide”; Included Charge States: 2-7; Dynamic Exclusion: 60 seconds; Mass Tolerance: 10 ppm; Minimum Intensity Threshold: 5.0×10³; MS/MS Acquisition Mode: Centroid; Quadrupole Isolation Window: 1.6 m/z; Collision Induced Fragmentation (CID) Energy: 35%; Activation Time: 10 seconds. Spectra were acquired over a 240 minute gradient with a flow rate of 0.250 μL/min as follows: 2% B for 3 minutes after injection, increased linearly to 15% B at 75 minutes, increased linearly to 30% B at 180 minutes, increased linearly to 35% B at 220 minutes, increased linearly to 80% B at 230 min, and decreased linearly to 5% at 240 minutes.

Label-Free Quantification (LFQ): Raw spectra were loaded into Proteome Discover 2.4 (Thermo Scientific) and protein abundances were determined using the UniProt Homo sapiens database (Hsap UP000005640.fasta). Protein abundances were determined using raw files and were searched using the following parameters: Digest Enzyme: Trypsin; Maximum Missed Cleavage Sites: 3; Minimum/Maximum Peptide Length: 6/144; Precursor Ion (MS1) Mass Tolerance: 20 ppm; Fragment Mass Tolerance: 0.5 Da; Minimum Peptides Identified: 1; Fixed Modification: Carbamidomethyl (C); Dynamic Modifications: acetyl, Met-loss at N-terminus, and oxidation of Met. A concatenated target/decoy strategy and a false-discovery rate (FDR) set to 1.0% were calculated using Percolator. Accurate mass and retention time of detected ions (features), determined using the Minora Feature Detector algorithm, were used to determine the area-under-the-curve (AUC) of the selected ion chromatograms of aligned features across all runs, after which the relative abundances were calculated. Differential abundances between treatments were determined using protein abundance ratio t-tests (background based) as implemented in Proteome Discoverer 2.4.

Statistical Analysis: Statistical analyses were performed using GraphPad Prism software. Values are represented as mean±SD for at least two independent experiments. A paired two-tailed Student's t-test was used to determine the significance between two groups. Kaplan-Meier analysis of mouse survival was performed using GraphPad Prism software, and the log-rank (Mantel-Cox) test was performed to compare survival curves and perform statistical analyses.

### EMBODIMENTS

The invention includes at least the following numbered embodiments:

1. A method of treating cancer in a subject in need thereof, the method comprising administering to the subject an effective amount of a myxoma virus (MYXV) and an effective amount of a nuclear export inhibitor, wherein the nuclear export inhibitor is administered orally.

2. A method of treating cancer in a subject in need thereof, the method comprising administering to the subject an effective amount of a myxoma virus (MYXV) and an effective amount of a nuclear export inhibitor, wherein the MYXV is genetically modified to express a heterologous transgene.

3. The method of embodiment 1 or embodiment 2, wherein the nuclear export inhibitor is a selective inhibitor of nuclear export (SINE).

4. The method of any one of embodiments 1-3, wherein the nuclear export inhibitor binds to and/or inhibits exportin 1 (XPO1/CRM1).

5. The method of any one of embodiments 1-4, wherein the nuclear export inhibitor binds to and/or inhibits a factor that binds to a nuclear export signal.

6. The method of any one of embodiments 1-5, wherein the nuclear export inhibitor binds to and/or inhibits a factor that binds to RAN, RAN-GTP, and/or RAN-GDP.

7. The method of any one of embodiments 1-6, wherein the nuclear export inhibitor binds to and/or inhibits a factor that docks to the nuclear pore complex.

8. The method of any one of embodiments 1-7, wherein the nuclear export inhibitor binds to and/or inhibits a factor that mediates leucine-rich nuclear export signal (NES)-dependent protein transport.

9. The method of any one of embodiments 1-8, wherein the nuclear export inhibitor is selinexor.

10. The method of embodiments 1, wherein the nuclear export inhibitor is Leptomycin A, Leptomycin B, Ratjadone A, Ratjadone B, Ratjadone C, Ratjadone D, Anguinomycin A, Goniothalamin, piperlongumine, plumbagin, curcumin, valtrate, acetoxychavicol acetate, prenylcoumarin osthol, KOS 2464, PKF050-638, or CBS9106.

11. The method of any one of embodiments 1-10, wherein the nuclear export inhibitor is not rapamycin or a structural analog thereof.

12. A method of treating cancer in a subject in need thereof, the method comprising administering to the subject an effective amount of a myxoma virus (MYXV) and an effective amount of a nuclear export inhibitor, wherein the nuclear export inhibitor is selinexor and is administered at a dose per kilogram of subject body weight of between about 0.001 mg/kg and about 1000 mg/kg.

13. The method of any one of embodiments 1-12, wherein the nuclear export inhibitor is administered in a tablet or a capsule.

14. The method of any one of embodiments 1-13, wherein the nuclear export inhibitor is administered at a dose per kilogram of subject body weight of between about 0.01 mg/kg and about 100 mg/kg.

15. The method of any one of embodiments 1-14, wherein at least two doses of the nuclear export inhibitor are administered.

16. The method of any one of embodiments 1-15, wherein the MYXV is administered locally.

17. The method of any one of embodiments 1-15, wherein the MYXV is administered systemically.

18. The method of any one of embodiments 1-17, wherein the MYXV is administered via injection or infusion.

19. The method of any one of embodiments 1-18, wherein the MYXV is administered intravenously.

20. The method of any one of embodiments 1-18, wherein the MYXV is administered intratumorally.

21. The method of any one of embodiments 1-20, wherein the MYXV is administered at a dose of from about 1×10³ focus-forming units (FFU) to about 1×10¹⁴ FFU.

22. The method of any one of embodiments 1-21, wherein at least two doses of the MYXV are administered.

23. The method of any one of embodiments 1-22, wherein the MYXV and the nuclear export inhibitor are administered simultaneously.

24. The method of any one of embodiments 1-22, wherein the MYXV and the nuclear export inhibitor are administered sequentially.

25. The method of embodiment 24, wherein the MYXV is administered before the nuclear export inhibitor.

26. The method of embodiment 24, wherein the nuclear export inhibitor is administered before the MYXV.

27. The method of any one of embodiments 1-26, wherein the method increases replication of the MYXV in cancer cells of the subject by at least 10%.

28. The method of any one of embodiments 1-27, wherein the method is effective to reduce average cancer load by at least 10% relative to an otherwise comparable treatment regimen that lacks the nuclear export inhibitor as determined by a cohort study.

29. The method of any one of embodiments 1-27, wherein the method is effective to reduce average cancer load by at least 10% relative to an otherwise comparable treatment regimen that lacks the MYXV as determined by a cohort study.

30. The method of embodiment 28 or embodiment 29, wherein the cancer load comprises a tumor volume.

31. The method of embodiment 28 or embodiment 29, wherein the cancer load comprises concentration of circulating hematological cancer cells.

32. The method of any one of embodiments 1-27, wherein the method is effective to prolong average survival by at least 5% relative to an otherwise comparable treatment regimen that lacks the nuclear export inhibitor as determined by a cohort study.

33. The method of any one of embodiments 1-27, wherein the method is effective to prolong average survival by at least 5% relative to an otherwise comparable treatment regimen that lacks the MYXV as determined by a cohort study.

34. The method of any one of embodiments 1-16 and 18-33, wherein upon local administration of the MYXV, the MYXV reduces cancer growth at a site distal from the site of administration at least 10% more than in a corresponding method that lacks the nuclear export inhibitor as determined by a cohort study.

35. The method of any one of embodiments 1-16 and 18-34, wherein upon local administration of the MYXV, the MYXV reduces incidence of metastasis at a site distal from the site of administration at least 10% more than in a corresponding method that lacks the nuclear export inhibitor as determined by a cohort study.

36. The method of any one of embodiments 1 and 3-35, wherein the MYXV is genetically modified.

37. The method of any one of embodiments 1 and 3-35, wherein the MYXV is genetically modified to express a heterologous transgene.

38. The method of embodiments 37, wherein the heterologous transgene encodes a cytokine or a functional fragment thereof.

39. The method of embodiment 37 or embodiment 38, wherein the heterologous transgene encodes an interleukin or a functional fragment thereof.

40. The method of any one of embodiments 37-39, wherein the heterologous transgene encodes a cell matrix protein or a functional fragment thereof.

41. The method of any one of embodiments 37-40, wherein the heterologous transgene encodes an antibody or a functional fragment thereof.

42. The method of any one of embodiments 37-41, wherein the heterologous transgene encodes an anti-PD-L1 antibody, decorin, IL-12, LIGHT, p14 FAST, TNF-α, a functional fragment thereof, or a combination thereof.

43. The method of any one of embodiments 37-42, wherein the heterologous transgene encodes a checkpoint inhibitor or a functional fragment thereof.

44. The method of any one of embodiments 37-43, wherein the heterologous transgene encodes a multi-specific immune cell engager.

45. The method of any one of embodiments 37-44, wherein the heterologous transgene encodes a bispecific killer cell engager (BiKE) or a bispecific T cell engager (BiTE).

46. The method of any one of embodiments 1-45, wherein the cancer is a solid tumor.

47. The method of any one of embodiments 1-45, wherein the cancer is a hematological tumor.

48. The method of any one of embodiments 1-46, wherein the cancer is a sarcoma or a carcinoma.

49. The method of any one of embodiments 1-46, wherein the cancer is a fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, or retinoblastoma.

50. The method of any one of embodiments 1-46, wherein the cancer is colorectal adenocarcinoma, pancreatic cancer, or melanoma.

51. The method of any one of embodiments 1-50, wherein the subject is immunocompetent.

52. The method of any one of embodiments 1-50, wherein the subject is immunocompromised or immunodeficient.

53. The method of any one of embodiments 1-52, wherein the subject is a mammal.

54. The method of any one of embodiments 1-53, wherein the subject is a human.

55. The method of any one of embodiments 1-54, further comprising adsorbing the MYXV to a leukocyte ex vivo and administering the leukocyte to the subject.

56. A therapeutic regimen comprising administering a myxoma virus (MYXV) and a nuclear export inhibitor to a subject with cancer, wherein the therapeutic regimen is effective to reduce average cancer load by at least 5% relative to an otherwise comparable treatment regimen that lacks the nuclear export inhibitor as determined by a cohort study.

57. A therapeutic regimen comprising administering a myxoma virus (MYXV) and a nuclear export inhibitor to a subject with cancer, wherein the therapeutic regimen is effective to reduce average cancer load by at least 5% relative to an otherwise comparable treatment regimen that lacks the MYXV as determined by a cohort study.

58. A therapeutic regimen comprising administering a myxoma virus (MYXV) and a nuclear export inhibitor to a subject with cancer, wherein the therapeutic regimen is effective to prolong average survival by at least 5% relative to an otherwise comparable treatment regimen that lacks the nuclear export inhibitor as determined by a cohort study.

59. A therapeutic regimen comprising administering a myxoma virus (MYXV) and a nuclear export inhibitor to a subject with cancer, wherein the therapeutic regimen is effective to prolong average survival by at least 5% relative to an otherwise comparable treatment regimen that lacks the MYXV as determined by a cohort study.

60. The therapeutic regimen of any one of embodiments 56-59, wherein the nuclear export inhibitor is administered orally.

61. The therapeutic regimen of any one of embodiments 56-60, wherein the nuclear export inhibitor is administered at a dose of between about 0.01 mg/kg and about 100 mg/kg.

62. The therapeutic regimen of any one of embodiments 56-61, wherein the MYXV is administered locally.

63. The therapeutic regimen of any one of embodiments 56-61, wherein the MYXV is administered systemically.

64. The therapeutic regimen of any one of embodiments 56-63, wherein the MYXV is administered intravenously.

65. The therapeutic regimen of any one of embodiments 56-63, wherein the MYXV is administered intratumorally.

66. The therapeutic regimen of any one of embodiments 56-65, wherein the MYXV is administered at a dose of from about 1×10\hat{ }3 focus-forming units (FFU) to about 1×10\hat{ }14 FFU.

67. The therapeutic regimen of any one of embodiments 56-66, wherein the MYXV and the nuclear export inhibitor are administered simultaneously.

68. The therapeutic regimen of any one of embodiments 56-66, wherein the MYXV and the nuclear export inhibitor are administered sequentially.

69. The therapeutic regimen of any one of embodiments 56-68, wherein the cancer load comprises a tumor volume.

70. The therapeutic regimen of any one of embodiments 56-69, wherein the cancer load comprises a concentration of circulating hematological cancer cells.

71. The therapeutic regimen of any one of embodiments 56-70, wherein the therapeutic regimen is effective to reduce the average cancer load by at least 20% relative to the otherwise comparable treatment regimen.

72. The therapeutic regimen of any one of embodiments 56-71, wherein the therapeutic regimen is effective to prolong average survival by at least 20% relative to the otherwise comparable treatment regimen.

73. The therapeutic regimen of any one of embodiments 56-62 and 64-72, wherein the MYXV is administered locally and therapeutic regimen reduces cancer growth at a site distal from the site of administration at least 10% more than in a corresponding treatment regimen that lacks the nuclear export inhibitor as determined by a cohort study.

74. The therapeutic regimen of any one of embodiments 56-73, wherein the therapeutic regimen reduces incidence of metastasis at least 10% more than in a corresponding treatment regimen that lacks the nuclear export inhibitor as determined by a cohort study.

75. The therapeutic regimen of any one of embodiments 56-74, wherein the nuclear export inhibitor is a selective inhibitor of nuclear export (SINE).

76. The therapeutic regimen of any one of embodiments 56-75, wherein the nuclear export inhibitor binds to and/or inhibits exportin 1 (XPO1/CRM1).

77. The therapeutic regimen of any one of embodiments 56-76, wherein the nuclear export inhibitor binds to and/or inhibits a factor that binds to a nuclear export signal.

78. The therapeutic regimen of any one of embodiments 56-77, wherein the nuclear export inhibitor binds to and/or inhibits a factor that binds to RAN, RAN-GTP, and/or RAN-GDP.

79. The therapeutic regimen of any one of embodiments 56-78, wherein the nuclear export inhibitor binds to and/or inhibits a factor that docks to the nuclear pore complex.

80. The therapeutic regimen of any one of embodiments 56-79, wherein the nuclear export inhibitor binds to and/or inhibits a factor that mediates leucine-rich nuclear export signal (NES)-dependent protein transport.

81. The therapeutic regimen of any one of embodiments 56-80, wherein the nuclear export inhibitor is selinexor.

82. The therapeutic regimen of any one of embodiments 56-74, wherein the nuclear export inhibitor is Leptomycin A, Leptomycin B, Ratjadone A, Ratjadone B, Ratjadone C, Ratjadone D, Anguinomycin A, Goniothalamin, piperlongumine, plumbagin, curcumin, valtrate, acetoxychavicol acetate, prenylcoumarin osthol, KOS 2464, PKF050-638, or CBS9106.

83. The therapeutic regimen of any one of embodiments 56-82, wherein the nuclear export inhibitor is not rapamycin or a structural analog thereof.

84. The therapeutic regimen of any one of embodiments 56-83, wherein the MYXV is genetically modified.

85. The therapeutic regimen of any one of embodiments 56-84, wherein the MYXV is genetically modified to express a heterologous transgene.

86. The therapeutic regimen of any one of embodiments 56-85, wherein the cancer is a solid tumor.

87. The therapeutic regimen of any one of embodiments 56-85, wherein the cancer is a hematological tumor.

88. The therapeutic regimen of any one of embodiments 56-85, wherein the cancer is a sarcoma or a carcinoma.

89. The therapeutic regimen of any one of embodiments 56-85, wherein the cancer is a fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, or retinoblastoma.

90. The therapeutic regimen of any one of embodiments 56-85, wherein the cancer is colorectal adenocarcinoma, pancreatic cancer, or melanoma.

91. The therapeutic regimen of any one of embodiments 56-90, wherein the subject is immunocompetent.

92. The therapeutic regimen of any one of embodiments 56-90, wherein the subject is immunocompromised or immunodeficient.

93. The therapeutic regimen of any one of embodiments 56-92, wherein the subject is a mammal.

94. The therapeutic regimen of any one of embodiments 56-93, wherein the subject is a human.

95. The therapeutic regimen of any one of embodiments 56-94, wherein the therapeutic regimen further comprises adsorbing the MYXV to a leukocyte ex vivo and administering the leukocyte to the subject.

1. A method of treating cancer in a subject in need thereof, the method comprising administering to the subject an effective amount of a myxoma virus (MYXV) and an effective amount of a nuclear export inhibitor, wherein the nuclear export inhibitor is administered orally and/or the MYXV is genetically modified to express a heterologous transgene.

2. The method of claim 1, wherein the nuclear export inhibitor:
a) is a selective inhibitor of nuclear export (SINE);
b) binds to and/or inhibits exportin 1 (XPO1/CRM1);
c) binds to and/or inhibits a factor that binds to a nuclear export signal (NES);
d) binds to and/or inhibits a factor that binds to RAN, RAN-GTP, and/or RAN-GDP;
e) binds to and/or inhibits a factor that docks to the nuclear pore complex;
f) binds to and/or inhibits a factor that mediates leucine-rich NES-dependent protein transport;
g) is not rapamycin or a structural analog thereof; or
h) a combination thereof.

3. (canceled)

4. The method of claim 1, wherein the nuclear export inhibitor is one or more selected from the group consisting of selinexor, leptomycin A, leptomycin B, ratjadone A, ratjadone B, ratjadone C, ratjadone D, anguinomycin A, goniothalamin, piperlongumine, plumbagin, curcumin, valtrate, acetoxychavicol acetate, prenylcoumarin osthol, KOS 2464, PKF050-638, and CBS9106.

5. The method of claim 4, wherein the nuclear export inhibitor is selinexor and is administered at a dose per kilogram of subject body weight of between about 0.001 mg/kg and about 1000 mg/kg.

6. The method of claim 5, wherein the selinexor is administered a) in a tablet or a capsule, b) in at least two doses, or c) a combination thereof.

7. (canceled)

8. The method of claim 1, wherein the MYXV is administered locally, systemically, intratumorally, intravenously, via injection, or via infusion.

9. The method of claim 5, wherein the MYXV is administered a) at a dose of from about 1×10³ focus-forming units (FFU) to about 1×10¹⁴ FFU, b) in at least two doses, or c) a combination thereof.

10. (canceled)

11. The method of claim 5, wherein the MYXV and the selinexor are administered simultaneously or sequentially.

12. The method of claim 5, wherein the method a) increases replication of the MYXV in cancer cells of the subject by at least 10%, b) is effective to reduce average cancer load by at least 5%, c) prolongs average survival by at least 5% relative to an otherwise comparable treatment regimen that lacks either the MYXV or the nuclear export inhibitor as determined by a cohort study, d) reduces cancer growth at a site distal from the site of administration at least 10% more than in a corresponding method that lacks either the MYXV or the nuclear export inhibitor as determined by a cohort study, or e) a combination thereof.

13. (canceled)

14. The method of claim 12, wherein the cancer load comprises a tumor volume or circulating hematological cancer cells.

15. (canceled)

16. The method of claim 5, wherein the heterologous transgene encodes a cytokine, interleukin, cell matrix protein, antibody, a checkpoint inhibitor, a multi-specific immune cell engager, or a functional fragment thereof.

17. The method of claim 16, wherein the heterologous transgene encodes an anti-PD-L1 antibody, decorin, IL-12, LIGHT, p14 FAST, TNF-α, a functional fragment thereof, or a combination thereof and/or wherein the multi-specific immune cell engager is a bispecific killer cell engager (BiKE) or a bispecific T cell engager (BiTE).

18. (canceled)

19. The method of claim 5, wherein the cancer is selected from the group consisting of a solid tumor, hematological tumor, sarcoma, carcinoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, retinoblastoma, colorectal adenocarcinoma, pancreatic cancer, and melanoma.

20. The method of claim 5, wherein the subject is immunocompetent, immunocompromised, immunodeficient, a mammal, a human, or a combination thereof.

21-22. (canceled)

23. The method of claim 5, further comprising adsorbing the MYXV to a leukocyte ex vivo and administering the leukocyte to the subject.

24. A therapeutic regimen comprising administering a myxoma virus (MYXV) and a nuclear export inhibitor to a subject with cancer, wherein the therapeutic regimen is effective to reduce average cancer load by at least 5% and/or prolong average survival by at least 5% relative to an otherwise comparable treatment regimen that lacks either the MYXV or the nuclear export inhibitor as determined by a cohort study.

25-30. (canceled)

31. The therapeutic regimen of claim 24, wherein the nuclear export inhibitor is selinexor and is administered at a dose per kilogram of subject body weight of between about 0.001 mg/kg and about 1000 mg/kg.

32-36. (canceled)

37. The therapeutic regimen of claim 31, wherein the therapeutic regimen is effective to reduce the average cancer load by at least 20% and/or prolong average survival by at least 20% relative to the otherwise comparable treatment regimen.

38. The therapeutic regimen of claim 31, wherein the MYXV is administered locally and the therapeutic regimen reduces incidence of metastasis at least 10% more than in a corresponding treatment regimen that lacks the selinexor as determined by a cohort study and/or reduces cancer growth at a site distal from the site of administration at least 10% more than in a corresponding treatment regimen that lacks the selinexor as determined by a cohort study.

39-43. (canceled)
